(19) United States
(12) Patent Application Publication
(10) Pub. No.: US 2004/0210289 A1
(43) Pub. Date: Oct. 21, 2004

(54) Novel nanomagnetic particles

(76) Inventors: Xingwu Wang, Wellsville, NY (US); Howard J. Greenwald, Rochester, NY (US)

Correspondence Address: HOWARD J. GREENWALD P.C., 349 W. COMMERCIAL STREET SUITE 2490, EAST ROCHESTER, NY, 14445-2408, US

(21) Appl. No.: 10/808,618

(22) Filed: Mar. 24, 2004

Related U.S. Application Data
(63) Continuation-in-part of application No. 10/366,082, filed on Feb. 13, 2003.
Application No. 10/366,082 is a continuation-in-part of application No. 10/324,773, filed on Dec. 18, 2002.
(63) Continuation-in-part of application No. 10/090,553, filed on Mar. 4, 2002.
(63) Continuation-in-part of application No. 10/229,183, filed on Aug. 26, 2002.
(63) Continuation-in-part of application No. 10/242,969, filed on Sep. 13, 2002.
(63) Continuation-in-part of application No. 10/260,247, filed on Sep. 30, 2002, now Pat. No. 6,673,999.
(63) Continuation-in-part of application No. 10/273,738, filed on Oct. 18, 2002.
(63) Continuation-in-part of application No. 10/303,264, filed on Nov. 25, 2002, now Pat. No. 6,713,671.
(63) Continuation-in-part of application No. 10/313,847, filed on Dec. 7, 2002.
(63) Continuation-in-part of application No. 10/303,264, filed on Nov. 25, 2002, now Pat. No. 6,713,671.

(51) Int. Cl. A61N 1/05

(52) U.S. Cl. 607/116

(57) ABSTRACT

A composition containing nanomagnetic particles. The, nanomagnetic particles have an average particle size of less than about 100 nanometers, a saturation magnetization of from about 2 to about 2,000 electromagnetic units per cubic centimeter, a phase transition temperature of from about 40 to about 200 degrees Celsius, and a squareness of from about 0.05 to about 1.0; the average coherence length between adjacent nanomagnetic particles is less than about 100 nanometers; and the nanomagnetic particles are at least triatomic.

### CROSS-REFERENCE TO RELATED PATENT APPLICATION

This patent application is a continuation-in-part of applicants' copending patent application U.S. Ser. No. 10/366,082, filed on Feb. 13, 2003, which in turn was a continuation-in-part of applicants' copending patent application Ser. No. 10/324,773, filed on Dec. 18, 2002. The entire disclosure of each of these United States patent applications is hereby incorporated by reference into this specification.

This patent application is also a continuation-in-part of applicants' copending patent applications U.S. Ser. No. 10/090,553, filed on Mar. 4, 2002, U.S. Ser. No. 10/229,183, filed on Aug. 26, 2002, U.S. Ser. No. 10/242,969, filed on Sep. 13, 2002, U.S. Ser. No. 10/260,247, filed on Sep. 30, 2002, U.S. Ser. No. 10/273,738, filed on Oct. 18, 2002, U.S. Ser. No. 10/303,264, filed on Nov. 25, 2002, and U.S. Ser. No. 10/313,847, filed on Dec. 7, 2002. The entire disclosure of each of these United States patent applications is hereby incorporated by reference to this specification.

This patent application is also a continuation-in-part of applicants' copending patent application U.S. Ser. No. 10/303,264, filed on Nov. 25, 2002, now U.S. Pat. No. 6,713,671.

### FIELD OF THE INVENTION

A collection of nanomagentic particles with an average particle size of less than about 100 naometers. The average coheence length between adjacent nanomagnetic particles is less than about 100 nanometers. The nanomagnetic particles have a saturation magentization of from about 2 to about 2000 electromagnetic units per cubic centimeter, and a phase transition temperature of from about 40 to about 200 degrees Celsius.

### BACKGROUND OF THE INVENTION

Applicants' U.S. Pat. No. 6,502,972 describes and claims a magnetically shielded conductor assembly comprised of a first conductor disposed within an insulating matrix, and a layer comprised of nanomagnetic material disposed around said first conductor, provided that such nanomagnetic material is not contiguous with said first conductor. In this assembly, the first conductor has a resistivity at 20 degrees Centigrade of from about 1 to about 100 micro ohm-centimeters, the insulating matrix is comprised of nano-sized particles wherein at least about 90 weight percent of said particles have a maximum dimension of from about 10 to about 100 nanometers, the insulating matrix has a resistivity of from about 1,000,000,000 to about 10,000,000,000,000 ohm-centimeter, the nanomagnetic material has an average particle size of less than about 100 nanometers, the layer of nanomagnetic material has a saturation magnetization of from about 200 to about 26,000 Gauss and a thickness of less than about 2 microns, and the magnetically shielded conductor assembly is flexible, having a bend radius of less than 2 centimeters. The entire disclosure of this United States patent is hereby incorporated by reference into this specification.

The nanomagnetic film disclosed in U.S. Pat. No. 6,506,972 may be used to shield medical devices from external electromagnetic fields; and, when so used, it provides a certain degree of shielding. The medical devices so shielded may be coated with one or more drug formulations.

It is an object of this invention to provide an improved nanomagnetic particle that may be used to coating a medical device.

### SUMMARY OF THE INVENTION

In accordance with this invention, there is provided a collection of nanomagentic particles with an average particle size of less than about 100 naometers, wherein the average coheence length between adjacent nanomagnetic particles is less than about 100 nanometers, wherein the nanomagnetic particles have a saturation magentization of from about 2 to about 2000 electromagnetic units per cubic centimeter, and wherein the nanomagnetic particles have a phase transition temperature of from about 40 to about 200 degrees Celsius.

### BRIEF DESCRIPTION OF THE DRAWINGS

The present invention will be more fully understood by reference to the following detailed thereof, when read in conjunction with the attached drawings, wherein like reference numerals refer to like elements, and wherein:

FIG. 1 is a schematic illustration of one preferred embodiment of the process of the invention;

FIG. 1A is a schematic illustration of a process in which nanomagnetic particles are collected upon a cooled collector;

FIG. 2 is a schematic illustration of another preferred embodiment of the process of the invention;

FIG. 3 is a phase diagram of a preferred nanomagnetic material;

FIG. 3A is a schematic illustration of the nanomagnetic material of FIG. 3 disposed within a cell and being heated up to its phase transition temperature;

FIG. 3B is a schematic illustration of what occurs when the nanomagnetic material of FIG. 3 is heated beyond its phase transition temperature;

FIG. 3C is a graph illustrating how the nanomagnetic material of FIGS. 3A and 3B acts like a magnetic switch;

FIG. 4 is a schematic of the spacing between components of the nanomagnetic material of FIG. 3;

FIG. 4A is a schematic of the spacing between adjacent particles of nanomagnetic material;

FIG. 5 is a schematic representation of a magnetic shield;

FIG. 6A through 6E are schematics of several preferred magnetically shielded assemblies;

FIG. 7 is a schematic of a circuit for cooling a substrate that is subjected to electromagnetic radiation;

FIG. 8 is a schematic illustration of one preferred assembly for shielding cardiac tissue from the adverse effects of electromagnetic radiation;

FIG. 9 is a flow diagram of a preferred process for shielding biological tissue from electromagnetic radiation;

FIG. 10 is a schematic diagram illustrating a preferred sputtering process for making one magnetically shielded assembly of the invention;

FIGS. 11 and 11A are partial schematic views of a stent coated with a film made by the process of the invention;

FIG. 12 is a schematic view of the stent of FIG. 11 illustrating how it responds to the electromagnetic radiation present in a magnetic resonance imaging (MRI) field;

FIGS. 13, 14, and 15 are graphs illustrating how the stent of FIG. 13, the coating of the stent of FIG. 13, and the coated stent of FIG. 13 react to the electromagnetic radiation present in an MRI field in terms their magnetizations, their reactances, and their image clarities;

FIG. 16 is a schematic illustration of a cylindrical coated substrate;

FIGS. 17A, 17B, and 17C are schematic views of a coated catheter assembly;

FIGS. 18A, 18B, 18C, 18D, 18E, 18F, and 18G are schematic views of a coated catheter assembly comprised of multiple concentric elements;

FIGS. 19A, 19B, and 19C are schematic views of a coated guide wire assembly;

FIGS. 20A and 20B are schematic views of a coated medical stent assembly;

FIG. 21 is a schematic view of a coated biopsy probe assembly;

FIGS. 22A and 22B are schematic views of a coated flexible tube endoscope tube assembly;

FIG. 23A is a schematic view of a sheath assembly;

FIG. 23B is a schematic illustration of a process for making the sheath assembly of FIG. 23A;

FIG. 24 is a phase diagram illustrating certain preferred compositions of the invention;

FIG. 25 is a schematic view of a coated substrate comprised of nanoelectrical particles;

FIG. 26 is a schematic view of a sensor assembly;

FIGS. 27A and 27B are illustrations of a sputtering process for making doped aluminum nitride

FIG. 28 is a schematic representation of a film orientation <002> of aluminum nitride;

FIG. 29 is a schematic illustration of a preferred sputtering process;

FIGS. 30 and 31 are schematic illustrations of an aluminum nitride construct;

FIGS. 32A and 32B are sectional and top views, respectively, of a coated substrate assembly whose coating has a morphological density of at least about 98 percent;

FIGS. 33A, 33B, and 33C illustrate the MRI images obtained with several of the coated constructs of this invention;

FIG. 34A illustrates a coated substrate comprised of a hydrophobic coating;

FIG. 34B illustrates a coated substrate comprised of a hydrophilic coating; and

FIG. 35 is a schematic illustration of a coating bonded to a substrate through an interfacial layer disposed between the coating and the substrate.

FIG. 36 is a sectional schematic view of a coated substrate and, binded thereoto, a layer of nano-sized particles;

FIG. 36A is a partial schematic view of a coating comprised of an indentation within which is disposed a recogniton molecule;

FIG. 36B is a schematic of an electromagnetic coil set aligned to an axis that creates a magnetic standing wave;

FIG. 36C is a three-dimensional schematic illustrating the results of using three sets of magnetic coils arranged orthogonally;

FIG. 37 is a schematic illustration of a process for preparing a coating with morphological indentations;

FIG. 38 is a schematic illustration of a drug molecule disposed inside an indentation of a coating;

FIG. 39 is a schematic of a process for administering paclitaxel to a patient;

FIG. 40 is a schematic of a preferred binding process of the invention;

FIG. 41 is a partial schematic of a binding process;

FIG. 42 is a graph of a typical response of a magnetic drug particle to an applied magnetic field;

FIGS. 43A and 43B illustrate the effect of applied fields upon a nanomagnetic coating and magnetic drug particles;

FIG. 44 is a graph of a preferred nanomagnetic material and its response to an applied electromagnetic field, in which the applied field is applied against the magnetic moment of the nanomagnetic material;

FIG. 45 is a schematic illustrating the forces acting upon magnetic drug particles as it approaches nanomagnetic material;

FIG. 46 is a schematic illustrating the forces acting upon magnetic drug particles after they have migrated into a layer of polymeric material and an external magnetic field is applied; and

FIG. 47 is a schematic illustrating the forces acting upon the magnetic drug particles after they have migrated into a layer of polymeric material and no external magnetic field is applied.

### DESCRIPTION OF THE PREFERRED EMBODIMENTS

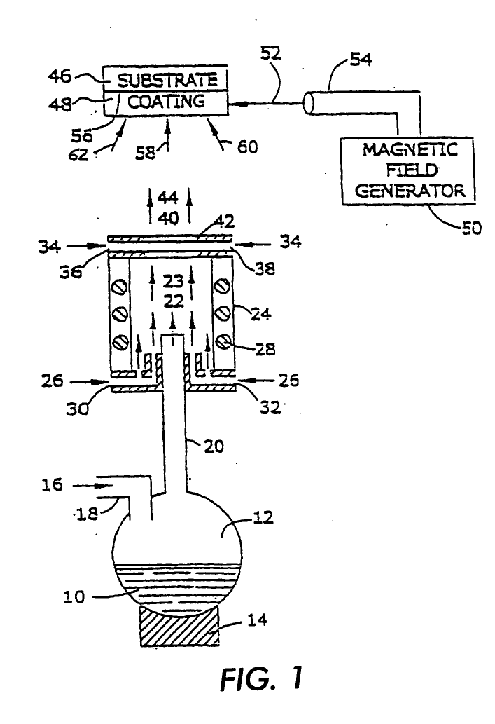

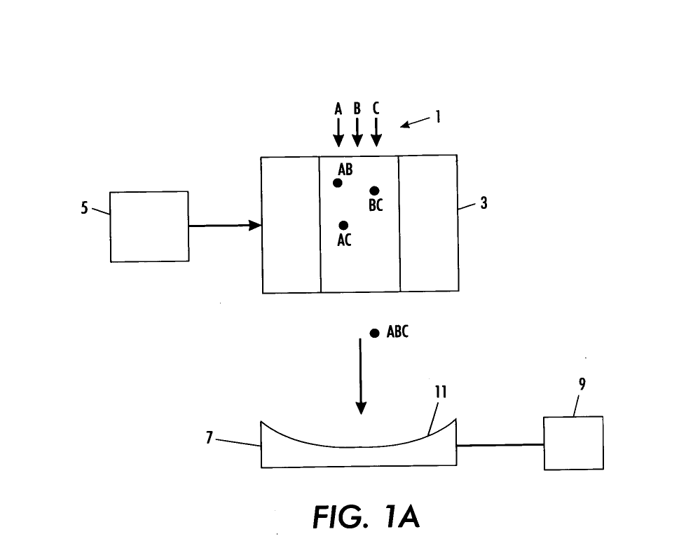

FIG. 1 is a schematic illustration of one process of the invention that may be used to make nanomagnetic material. This FIG. 1 is similar in many respects to the FIG. 1 of U.S. Pat. No. 5,213,851, the entire disclosure of which is hereby incorporated by reference into this specification.

Referring to FIG. 1, and in the preferred embodiment depicted therein, it is preferred that the reagents charged into misting chamber 12 will be sufficient to form a nano-sized ferrite in the process. The term ferrite, as used in this specification, refers to a material that exhibits ferromagnetism. Ferromagnetism is a property, exhibited by certain metals, alloys, and compounds of the transition (iron group) rare earth and actinide elements, in which the internal magnetic moments spontaneously organize in a common direction; ferromagnetism gives rise to a permeability considerably greater than that of vacuum and to magnetic hysteresis. See, e.g, page 706 of Sybil B. Parker's “McGraw-Hill Dictionary of Scientific and Technical Terms,” Fourth Edition (McGraw-Hill Book Company, New York, N.Y., 1989).

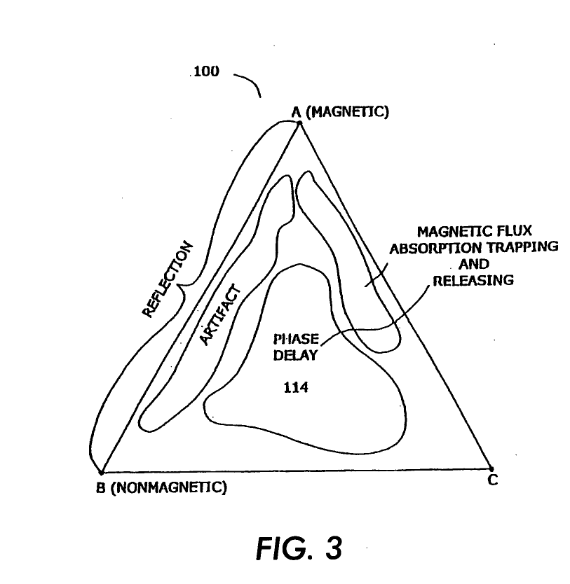

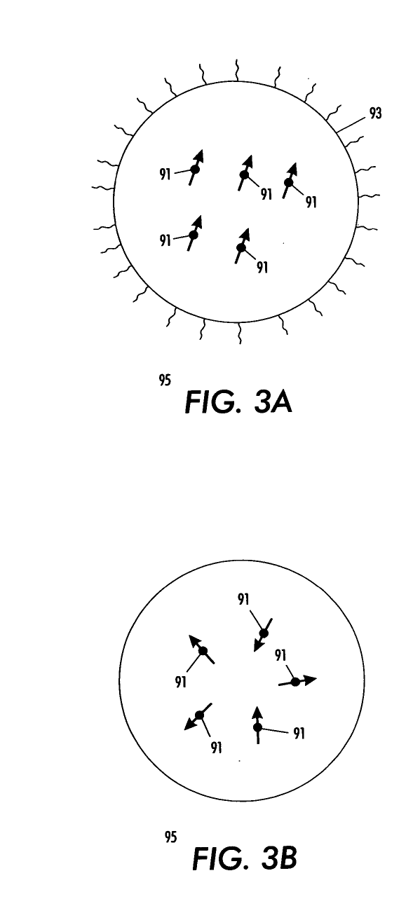

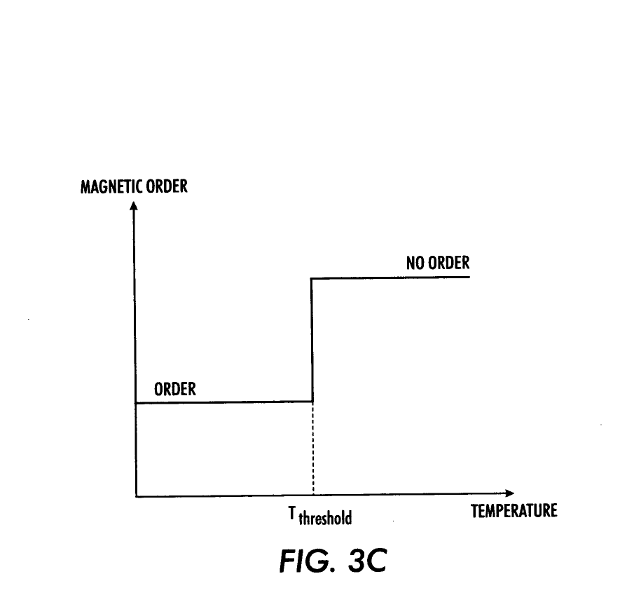

As will be apparent to those skilled in the art, in addition to making nano-sized ferrites by the process depicted in FIG. 1, one may also make other nano-sized materials such as, e.g., nano-sized nitrides and/or nano-sized oxides containing moieties A, B, and C (see FIG. 3 et seq. and its accompanying discussion). For the sake of simplicity of description, and with regard to FIG. 1, a discussion will be had regarding the preparation of ferrites, it being understood that, e.g., other materials may also be made by such process.

Referring again to FIG. 1, and to the production of ferrites by such process, in one embodiment, the ferromagnetic material contains Fe₂O₃. See, for example, U.S. Pat. No. 3,576,672 of Harris et al., the entire disclosure of which is hereby incorporated by reference into this specification. As will be apparent, the corresponding nitrides also may be made.

In one embodiment, the ferromagnetic material contains garnet. Pure iron garnet has the formula M₃Fe₅O₁₂; see, e.g., pages 65-256 of Wilhelm H. Von Aulock's “Handbook of Microwave Ferrite Materials” (Academic Press, New York, 1965). Garnet ferrites are also described, e.g., in U.S. Pat. No. 4,721,547, the disclosure of which is hereby incorporated by reference into this specification. As will be apparent, the corresponding nitrides also may be made.

In another embodiment, the ferromagnetic material contains a spinel ferrite. Spinel ferrites usually have the formula MFe₂O₄, wherein M is a divalent metal ion and Fe is a trivalent iron ion. M is typically selected from the group consisting of nickel, zinc, magnesium, manganese, and like. These spinel ferrites are well known and are described, for example, in U.S. Pat. Nos. 5,001,014, 5,000,909, 4,966,625, 4,960,582, 4,957,812, 4,880,599, 4,862,117, 4,855,205, 4,680,130, 4,490,268, 3,822,210, 3,635,898, 3,542,685, 3,421,933, and the like. The disclosure of each of these patents is hereby incorporated by reference into this specification. Reference may also be had to pages 269-406 of the Von Aulock book for a discussion of spinel ferrites. As will be apparent, the corresponding nitrides also may be made.

In yet another embodiment, the ferromagnetic material contains a lithium ferrite. Lithium ferrites are often described by the formula (Li_{0.5} Fe_{0.5})2+(Fe₂)3+O₄. Some illustrative lithium ferrites are described on pages 407-434 of the aforementioned Von Aulock book and in U.S. Pat. Nos. 4,277,356, 4,238,342, 4,177,438, 4,155,963, 4,093,781, 4,067,922, 3,998,757, 3,767,581, 3,640,867, and the like. The disclosure of each of these patents is hereby incorporated by reference into this specification. As will be apparent, the corresponding nitrides also may be made.

In yet another embodiment, the ferromagnetic material contains a hexagonal ferrite. These ferrites are well known and are disclosed on pages 451-518 of the Von Aulock book and also in U.S. Pat. Nos. 4,816,292, 4,189,521, 5,061,586, 5,055,322, 5,051,201, 5,047,290, 5,036,629, 5,034,243, 5,032,931, and the like. The disclosure of each of these patents is hereby incorporated by reference into this specification. As will be apparent, the corresponding nitrides also may be made.

In yet another embodiment, the ferromagnetic material contains one or more of the moieties A, B, and C disclosed in the phase diagram of FIG. 3 and discussed elsewhere in this specification.

Referring again to FIG. 1, and in the preferred embodiment depicted therein, it will be appreciated that the solution 10 will preferably comprise reagents necessary to form the required magnetic material. For example, in one embodiment, in order to form the spinel nickel ferrite of the formula NiFe₂O₄, the solution should contain nickel and iron, which may be present in the form of nickel nitrate and iron nitrate. By way of further example, one may use nickel chloride and iron chloride to form the same spinel. By way of further example, one may use nickel sulfate and iron sulfate.

It will be apparent to skilled chemists that many other combinations of reagents, both stoichiometric and nonstoichiometric, may be used in applicants' process to make many different magnetic materials.

In one preferred embodiment, the solution 10 contains the reagent needed to produce a desired ferrite in stoichiometric ratio. Thus, to make the NiFe₂O₄ ferrite in this embodiment, one mole of nickel nitrate may be charged with every two moles of iron nitrate.

In one embodiment, the starting materials are powders with purities exceeding 99 percent.

In one embodiment, compounds of iron and the other desired ions are present in the solution in the stoichiometric ratio.

In one preferred embodiment, ions of nickel, zinc, and iron are present in a stoichiometric ratio of 0.5/0.5/2.0, respectively. In another preferred embodiment, ions of lithium and iron are present in the ratio of 0.5/2.5. In yet another preferred embodiment, ions of magnesium and iron are present in the ratio of 1.0/2.0. In another embodiment, ions of manganese and iron are present in the ratio 1.0/2.0. In yet another embodiment, ions of yttrium and iron are present in the ratio of 3.0/5.0. In yet another embodiment, ions of lanthanum, yttrium, and iron are present in the ratio of 0.5/2.5/5.0. In yet another embodiment, ions of neodymium, yttrium, gadolinium, and iron are present in the ratio of 1.0/1.07/0.93/5.0, or 1.0/1.1/0.9/5.0, or 1/1.12/0.88/5.0. In yet another embodiment, ions of samarium and iron are present in the ratio of 3.0/5.0. In yet another embodiment, ions of neodymium, samarium, and iron are present in the ratio of 0.1/2.9/5.0, or 0.25/2.75/5.0, or 0.375/2.625/5.0. In yet another embodiment, ions of neodymium, erbium, and iron are present in the ratio of 1.5/1.5/5.0. In yet another embodiment, samarium, yttrium, and iron ions are present in the ratio of 0.51/2.49/5.0, or 0.84/2.16/5.0, or 1.5/1.5/5.0. In yet another embodiment, ions of yttrium, gadolinium, and iron are present in the ratio of 2.25/0.75/5.0, or 1.5/1.5/5.0, or 0.75/2.25/5.0. In yet another embodiment, ions of terbium, yttrium, and iron are present in the ratio of 0.8/2.2/5.0, or 1.0/2.0/5.0. In yet another embodiment, ions of dysprosium, aluminum, and iron are present in the ratio of 3/x/5−x, when x is from 0 to 1.0. In yet another embodiment, ions of dysprosium, gallium, and iron are also present in the ratio of 3/x/5−x. In yet another embodiment, ions of dysprosium, chromium, and iron are also present in the ratio of 3/x/5−x.

The ions present in the solution, in one embodiment, may be holmium, yttrium, and iron, present in the ratio of z/3−z/5.0, where z is from about 0 to 1.5.

The ions present in the solution may be erbium, gadolinium, and iron in the ratio of 1.5/1.5/5.0. The ions may be erbium, yttrium, and iron in the ratio of 1.5/1.5/1.5, or 0.5/2.5/5.0.

The ions present in the solution may be thulium, yttrium, and iron, in the ratio of 0.06/2.94/5.0.

The ions present in the solution may be ytterbium, yttrium, and iron, in the ratio of 0.06/2.94/5.0.

The ions present in the solution may be lutetium, yttrium, and iron in the ratio of y/3−y/5.0, wherein y is from 0 to 3.0.

The ions present in the solution may be iron, which can be used to form Fe₆O₈ (two formula units of Fe₃O₄). The ions present may be barium and iron in the ratio of 1.0/6.0, or 2.0/8.0. The ions present may be strontium and iron, in the ratio of 1.0/12.0. The ions present may be strontium, chromium, and iron in the ratio of 1.0/1.0/10.0, or 1.0/6.0/6.0. The ions present may be suitable for producing a ferrite of the formula (Meₓ)₃+Ba₁₋ₓFe₁₂O₁₉, wherein Me is a rare earth selected from the group consisting of lanthanum, promethium, neodymium, samarium, europium, and mixtures thereof.

The ions present in the solution may contain barium, either lanthanum or promethium, iron, and cobalt in the ratio of 1−a/a/12−a/a, wherein a is from 0.0 to 0.8.

The ions present in the solution may contain barium, cobalt, titanium, and iron in the ratio of 1.0/b/b/12−2b, wherein b is from 0.0 to 1.6.

The ions present in the solution may contain barium, nickel or cobalt or zinc, titanium, and iron in the ratio of 1.0/c/c/12−2c, wherein c is from 0.0 to 1.5.

The ions present in the solution may contain barium, iron, iridium, and zinc in the ratio of 1.0/12−2d/d/d, wherein d is from 0.0 to 0.6.

The ions present in the solution may contain barium, nickel, gallium, and iron in the ratio of 1.0/2.0/7.0/9.0, or 1.0/2.0/5.0/11.0. Alternatively, the ions may contain barium, zinc, gallium or aluminum, and iron in the ratio of 1.0/2.0/3.0/13.0.

Each of these ferrites is well known to those in the ferrite art and is described, e.g., in the aforementioned Von Aulock book.

The ions described above are preferably available in solution 10 in water-soluble form, such as, e.g., in the form of water-soluble salts. Thus, e.g., one may use the nitrates or the chlorides or the sulfates or the phosphates of the cations. Other anions which form soluble salts with the cation(s) also may be used.

Alternatively, one may use salts soluble in solvents other than water. Some of these other solvents which may be used to prepare the material include nitric acid, hydrochloric acid, phosphoric acid, sulfuric acid, and the like. As is well known to those skilled in the art, many other suitable solvents may be used; see, e.g., J. A. Riddick et al., “Organic Solvents, Techniques of Chemistry,” Volume II, 3rd edition (Wiley-Interscience, New York, N.Y., 1970).

In one preferred embodiment, where a solvent other than water is used, each of the cations is present in the form of one or more of its oxides. For example, one may dissolve iron oxide in nitric acid, thereby forming a nitrate. For example, one may dissolve zinc oxide in sulfuric acid, thereby forming a sulfate. One may dissolve nickel oxide in hydrochloric acid, thereby forming a chloride. Other means of providing the desired cation(s) will be readily apparent to those skilled in the art.

In general, as long as the desired cation(s) are present in the solution, it is not significant how the solution was prepared.

In general, one may use commercially available reagent grade materials. Thus, by way of illustration and not limitation, one may use the following reagents available in the 1988-1989 Aldrich catalog (Aldrich Chemical Company, Inc., Milwaukee, Wis.): barium chloride, catalog number 31,866-3; barium nitrate, catalog number 32,806-5; barium sulfate, catalog number 20,276-2; strontium chloride hexhydrate, catalog number 20,466-3; strontium nitrate, catalog number 20,449-8; yttrium chloride, catalog number 29,826-3; yttrium nitrate tetrahydrate, catalog number 21,723-9; yttrium sulfate octahydrate, catalog number 20,493-5. This list is merely illustrative, and other compounds that can be used will be readily apparent to those skilled in the art. Thus, any of the desired reagents also may be obtained from the 1989-1990 AESAR catalog (Johnson Matthey/AESAR Group, Seabrook, N.H.), the 1990/1991 Alfa catalog (Johnson Matthey/Alfa Products, Ward Hill, Ma.), the Fisher 88 catalog (Fisher Scientific, Pittsburgh, Pa.), and the like.

As long as the metals present in the desired ferrite material are present in solution 10 in the desired stoichiometry, it does not matter whether they are present in the form of a salt, an oxide, or in another form. In one embodiment, however, it is preferred to have the solution contain either the salts of such metals, or their oxides.

The solution 10 of the compounds of such metals preferably will be at a concentration of from about 0.01 to about 1,000 grams of said reagent compounds per liter of the resultant solution. As used in this specification, the term liter refers to 1,000 cubic centimeters.

In one embodiment, it is preferred that solution 10 have a concentration of from about 1 to about 300 grams per liter and, preferably, from about 25 to about 170 grams per liter. It is even more preferred that the concentration of said solution 10 be from about 100 to about 160 grams per liter. In an even more preferred embodiment, the concentration of said solution 10 is from about 140 to about 160 grams per liter.

In one preferred embodiment, aqueous solutions of nickel nitrate, and iron nitrate with purities of at least 99.9 percent are mixed in the molar ratio of 1:2 and then dissolved in distilled water to form a solution with a concentration of 150 grams per liter.

In one preferred embodiment, aqueous solutions of nickel nitrate, zinc nitrate, and iron nitrate with purities of at least 99.9 percent are mixed in the molar ratio of 0.5:0.5:2 and then dissolved in distilled water to form a solution with a concentration of 150 grams per liter.

In one preferred embodiment, aqueous solutions of zinc nitrate, and iron nitrate with purities of at least 99.9 percent are mixed in the molar ratio of 1:2 and then dissolved in distilled water to form a solution with a concentration of 150 grams per liter.

In one preferred embodiment, aqueous solutions of nickel chloride, and iron chloride with purities of at least 99.9 percent are mixed in the molar ratio of 1:2 and then dissolved in distilled water to form a solution with a concentration of 150 grams per liter.

In one preferred embodiment, aqueous solutions of nickel chloride, zinc chloride, and iron chloride with purities of at least 99.9 percent are mixed in the molar ratio of 0.5:0.5:2 and then dissolved in distilled water to form a solution with a concentration of 150 grams per liter.

In one preferred embodiment, aqueous solutions of zinc chloride, and iron chloride with purities of at least 99.9 percent are mixed in the molar ratio of 1:2 and then dissolved in distilled water to form a solution with a concentration of 150 grams per liter.

In one embodiment, mixtures of chlorides and nitrides may be used. Thus, for example, in one preferred embodiment, the solution is comprised of both iron chloride and nickel nitrate in the molar ratio of 2.0/1.0.

Referring again to FIG. 1, and to the preferred embodiment depicted therein, the solution 10 in misting chamber 12 is preferably caused to form into an aerosol, such as a mist.

The term aerosol, as used in this specification, refers to a suspension of ultramicroscopic solid or liquid particles in air or gas, such as smoke, fog, or mist. See, e.g., page 15 of “A dictionary of mining, mineral, and related terms,” edited by Paul W. Thrush (U.S. Department of the Interior, Bureau of Mines, 1968), the disclosure of which is hereby incorporated by reference into this specification.

As used in this specification, the term mist refers to gas-suspended liquid particles which have diameters less than 10 microns.

The aerosol/mist consisting of gas-suspended liquid particles with diameters less than 10 microns may be produced from solution 10 by any conventional means that causes sufficient mechanical disturbance of said solution. Thus, one may use mechanical vibration. In one preferred embodiment, ultrasonic means are used to mist solution 10. As is known to those skilled in the art, by varying the means used to cause such mechanical disturbance, one can also vary the size of the mist particles produced.

As is known to those skilled in the art, ultrasonic sound waves (those having frequencies above 20,000 hertz) may be used to mechanically disturb solutions and cause them to mist. Thus, by way of illustration, one may use the ultrasonic nebulizer sold by the DeVilbiss Health Care, Inc. of Somerset, Pa.; see, e.g., the “Instruction Manual” for the “Ultra-Neb 99 Ultrasonic Nebulizer, publication A-850-C (published by DeVilbiss, Somerset, Pa., 1989).

In the embodiment shown in FIG. 1, the oscillators of ultrasonic nebulizer 14 are shown contacting an exterior surface of misting chamber 12. In this embodiment, the ultrasonic waves produced by the oscillators are transmitted via the walls of the misting chamber 12 and effect the misting of solution 10.

In another embodiment, not shown, the oscillators of ultrasonic nebulizer 14 are in direct contact with solution 10.

In one embodiment, it is preferred that the ultrasonic power used with such machine is in excess of one watt and, more preferably, in excess of 10 watts. In one embodiment, the power used with such machine exceeds about 50 watts.

During the time solution 10 is being caused to mist, it is preferably contacted with carrier gas to apply pressure to the solution and mist. It is preferred that a sufficient amount of carrier gas be introduced into the system at a sufficiently high flow rate so that pressure on the system is in excess of atmospheric pressure. Thus, for example, in one embodiment wherein chamber 12 has a volume of about 200 cubic centimeters, the flow rate of the carrier gas was from about 100 to about 150 milliliters per minute.

In one embodiment, the carrier gas 16 is introduced via feeding line 18 at a rate sufficient to cause solution 10 to mist at a rate of from about 0.5 to about 20 milliliters per minute. In one embodiment, the misting rate of solution 10 is from about 1.0 to about 3.0 milliliters per minute.

Substantially any gas that facilitates the formation of plasma may be used as carrier gas 16. Thus, by way of illustration, one may use oxygen, air, argon, nitrogen, and the like. It is preferred that the carrier gas used be a compressed gas under a pressure in excess 760 millimeters of mercury. In this embodiment, the use of the compressed gas facilitates the movement of the mist from the misting chamber 12 to the plasma region 22.

The misting container 12 may be any reaction chamber conventionally used by those skilled in the art and preferably is constructed out of such acid-resistant materials such as glass, plastic, and the like.

The mist from misting chamber 12 is fed via misting outlet line 20 into the plasma region 22 of plasma reactor 24. In plasma reactor 24, the mist is mixed with plasma generated by plasma gas 26 and subjected to radio frequency radiation provided by a radio-frequency coil 28.

The plasma reactor 24 provides energy to form plasma and to cause the plasma to react with the mist. Any of the plasmas reactors well known to those skilled in the art may be used as plasma reactor 24. Some of these plasma reactors are described in J. Mort et al.'s “Plasma Deposited Thin Films” (CRC Press Inc., Boca Raton, Fla., 1986); in “Methods of Experimental Physics,” Volume 9—Parts A and B, Plasma Physics (Academic Press, New York, 1970/1971); and in N. H. Burlingame's “Glow Discharge Nitriding of Oxides,” Ph.D. thesis (Alfred University, Alfred, N.Y., 1985), available from University Microfilm International, Ann Arbor, Mich.

In one preferred embodiment, the plasma reactor 24 is a “model 56 torch” available from the TAFA Inc. of Concord, N.H. It is preferably operated at a frequency of about 4 megahertz and an input power of 30 kilowatts.

Referring again to FIG. 1, and to the preferred embodiment depicted therein, it will be seen that into feeding lines 30 and 32 is fed plasma gas 26. As is known to those skilled in the art, a plasma can be produced by passing gas into a plasma reactor. A discussion of the formation of plasma is contained in B. Chapman's “Glow Discharge Processes” (John Wiley & Sons, New York, 1980)

In one preferred embodiment, the plasma gas used is a mixture of argon and oxygen. In another embodiment, the plasma gas is a mixture of nitrogen and oxygen. In yet another embodiment, the plasma gas is pure argon or pure nitrogen.

When the plasma gas is pure argon or pure nitrogen, it is preferred to introduce into the plasma reactor at a flow rate of from about 5 to about 30 liters per minute.

When a mixture of oxygen and either argon or nitrogen is used, the concentration of oxygen in the mixture preferably is from about 1 to about 40 volume percent and, more preferably, from about 15 to about 25 volume percent. When such a mixture is used, the flow rates of each gas in the mixture should be adjusted to obtain the desired gas concentrations. Thus, by way of illustration, in one embodiment that uses a mixture of argon and oxygen, the argon flow rate is 15 liters per minute, and the oxygen flow rate is 40 liters per minute.

In one embodiment, auxiliary oxygen 34 is fed into the top of reactor 24, between the plasma region 22 and the flame region 40, via lines 36 and 38. In this embodiment, the auxiliary oxygen is not involved in the formation of plasma but is involved in the enhancement of the oxidation of the ferrite material.

Radio frequency energy is applied to the reagents in the plasma reactor 24, and it causes vaporization of the mist.

In general, the energy is applied at a frequency of from about 100 to about 30,000 kilohertz. In one embodiment, the radio frequency used is from about 1 to 20 megahertz. In another embodiment, the radio frequency used is from about 3 to about 5 megahertz.

As is known to those skilled in the art, such radio frequency alternating currents may be produced by conventional radio frequency generators. Thus, by way of illustration, said TAPA Inc. “model 56 torch” may be attached to a radio frequency generator rated for operation at 35 kilowatts which manufactured by Lepel Company (a division of TAFA Inc.) and which generates an alternating current with a frequency of 4 megaherz at a power input of 30 kilowatts. Thus, e.g., one may use an induction coil driven at 2.5-5.0 megahertz that is sold as the “PLASMOC 2” by ENI Power Systems, Inc. of Rochester, N.Y.

The use of these type of radio-frequency generators is described in the Ph.D. theses entitled (1) “Heat Transfer Mechanisms in High-Temperature Plasma Processing of Glasses,” Donald M. McPherson (Alfred University, Alfred, N.Y., January, 1988) and (2) the aforementioned Nicholas H. Burlingame's “Glow Discharge Nitriding of Oxides.”

The plasma vapor 23 formed in plasma reactor 24 is allowed to exit via the aperture 42 and can be visualized in the flame region 40. In this region, the plasma contacts air that is at a lower temperature than the plasma region 22, and a flame is visible. A theoretical model of the plasma/flame is presented on pages 88 et seq. of said McPherson thesis.

The vapor 44 present in flame region 40 is propelled upward towards substrate 46. Any material onto which vapor 44 will condense may be used as a substrate. Thus, by way of illustration, one may use nonmagnetic materials such alumina, glass, gold-plated ceramic materials, and the like. In one embodiment, substrate 46 consists essentially of a magnesium oxide material such as single crystal magnesium oxide, polycrystalline magnesium oxide, and the like.

In another embodiment, the substrate 46 consists essentially of zirconia such as, e.g., yttrium stabilized cubic zirconia.

In another embodiment, the substrate 46 consists essentially of a material selected from the group consisting of strontium titanate, stainless steel, alumina, sapphire, and the like.

The aforementioned listing of substrates is merely meant to be illustrative, and it will be apparent that many other substrates may be used. Thus, by way of illustration, one may use any of the substrates mentioned in M. Sayer's “Ceramic Thin Films . . . ” article, supra. Thus, for example, in one embodiment it is preferred to use one or more of the substrates described on page 286 of “Superconducting Devices,” edited by S. T. Ruggiero et al. (Academic Press, Inc., Boston, 1990).

One advantage of this embodiment of applicants' process is that the substrate may be of substantially any size or shape, and it may be stationary or movable. Because of the speed of the coating process, the substrate 46 may be moved across the aperture 42 and have any or all of its surface be coated.

As will be apparent to those skilled in the art, in the embodiment depicted in FIG. 1, the substrate 46 and the coating 48 are not drawn to scale but have been enlarged to the sake of ease of representation.

Referring again to FIG. 1, the substrate 46 may be at ambient temperature. Alternatively, one may use additional heating means to heat the substrate prior to, during, or after deposition of the coating.

In one embodiment, illustrated in FIG. 1A, the substrate is cooled so that nanomagnetic particles are collected on such substrate. Referring to FIG. 1A, and in the preferred embodiment depicted therein, a precursor 1 that preferably contains moieties A, B, and C (which are described elsewhere in this specification) are charged to reactor 3; the reactor 3 may be the plasma reactor depicted in FIG. 1, and/or it may be the sputtering reactor described elsewhere in this specification.

Referring again to FIG. 1A, it will be seen that an energy source 5 is preferably used in order to cause reaction between moieties A, B, and C. The energy source 5 may be an electromagnetic energy source that supplies energy to the reactor 3.

Within reactor 3 moities A, B, and C are preferably combined into a metastable state. This metastable state is then caused to travel towards collector 7. Prior to the time it reaches the collector 7, the ABC moiety is formed, either in the reactor 3 and/or between the reactor 3 and the collector 7.

In one embodiment, collector 7 is preferably cooled with a chiller 9 so that its surface 11 is at a temperature below the temperature at which the ABC moiety interacts with surface 11; the goal is to prevent bonding between the ABC moiety and the surface 11. In one embodiment, the surface 11 is at a temperature of less than about 30 degrees Celsius. In another embodiment, the temperature of surface 11 is at the liquid nitrogen temperature, i.e., about 77 degrees Kelvin.

After the ABC moieties have been collected by collector 7, they are removed from surface 11.

Referring again to FIG. 1, and in one preferred embodiment, a heater (not shown) is used to heat the substrate to a temperature of from about 100 to about 800 degrees centigrade.

In one aspect of this embodiment, temperature sensing means (not shown) may be used to sense the temperature of the substrate and, by feedback means (not shown), adjust the output of the heater (not shown). In one embodiment, not shown, when the substrate 46 is relatively near flame region 40, optical pyrometry measurement means (not shown) may be used to measure the temperature near the substrate.

In one embodiment, a shutter (not shown) is used to selectively interrupt the flow of vapor 44 to substrate 46. This shutter, when used, should be used prior to the time the flame region has become stable; and the vapor should preferably not be allowed to impinge upon the substrate prior to such time.

The substrate 46 may be moved in a plane that is substantially parallel to the top of plasma chamber 24. Alternatively, or additionally, it may be moved in a plane that is substantially perpendicular to the top of plasma chamber 24. In one embodiment, the substrate 46 is moved stepwise along a predetermined path to coat the substrate only at certain predetermined areas.

In one embodiment, rotary substrate motion is utilized to expose as much of the surface of a complex-shaped article to the coating. This rotary substrate motion may be effectuated by conventional means. See, e.g., “Physical Vapor Deposition,” edited by Russell J. Hill (Temescal Division of The BOC Group, Inc., Berkeley, Calif., 1986).

The process of this embodiment of the invention allows one to coat an article at a deposition rate of from about 0.01 to about 10 microns per minute and, preferably, from about 0.1 to about 1.0 microns per minute, with a substrate with an exposed surface of 35 square centimeters. One may determine the thickness of the film coated upon said reference substrate material (with an exposed surface of 35 square centimeters) by means well known to those skilled in the art.

The film thickness can be monitored in situ, while the vapor is being deposited onto the substrate. Thus, by way of illustration, one may use an IC-6000 thin film thickness monitor (also referred to as “deposition controller”) manufactured by Leybold Inficon Inc. of East Syracuse, N.Y.

The deposit formed on the substrate may be measured after the deposition by standard profilometry techniques. Thus, e.g., one may use a DEKTAK Surface Profiler, model number 900051 (available from Sloan Technology Corporation, Santa Barbara, Calif.).

In general, at least about 80 volume percent of the particles in the as-deposited film are smaller than about 1 micron. It is preferred that at least about 90 percent of such particles are smaller than 1 micron. Because of this fine grain size, the surface of the film is relatively smooth.

In one preferred embodiment, the as-deposited film is post-annealed.

It is preferred that the generation of the vapor in plasma rector 24 be conducted under substantially atmospheric pressure conditions. As used in this specification, the term “substantially atmospheric” refers to a pressure of at least about 600 millimeters of mercury and, preferably, from about 600 to about 1,000 millimeters of mercury. It is preferred that the vapor generation occur at about atmospheric pressure. As is well known to those skilled in the art, atmospheric pressure at sea level is 760 millimeters of mercury.

The process of this invention may be used to produce coatings on a flexible substrate such as, e.g., stainless steel strips, silver strips, gold strips, copper strips, aluminum strips, and the like. One may deposit the coating directly onto such a strip. Alternatively, one may first deposit one or more buffer layers onto the strip(s). In other embodiments, the process of this invention may be used to produce coatings on a rigid or flexible cylindrical substrate, such as a tube, a rod, or a sleeve.

Referring again to FIG. 1, and in the embodiment depicted therein, as the coating 48 is being deposited onto the substrate 46, and as it is undergoing solidification thereon, it is preferably subjected to a magnetic field produced by magnetic field generator 50.

In this embodiment, it is preferred that the magnetic field produced by the magnetic field generator 50 have a field strength of from about 2 Gauss to about 40 Tesla.

It is preferred to expose the deposited material for at least 10 seconds and, more preferably, for at least 30 seconds, to the magnetic field, until the magnetic moments of the nano-sized particles being deposited have been substantially aligned.

As used herein, the term “substantially aligned” means that the inductance of the device being formed by the deposited nano-sized particles is at least 90 percent of its maximum inductance. One may determine when such particles have been aligned by, e.g., measuring the inductance, the permeability, and/or the hysteresis loop of the deposited material.

Thus, e.g., one may measure the degree of alignment of the deposited particles with an impedance meter, a inductance meter, or a SQUID.

In one embodiment, the degree of alignment of the deposited particles is measured with an inductance meter. One may use, e.g., a conventional conductance meter such as, e.g., the conductance meters disclosed in U.S. Pat. Nos. , 4,937,995, 5,728,814 (apparatus for determining and recording injection does in syringes using electrical inductance), U.S. Pat. Nos. 6,318,176, 5,014,012, 4,869,598, 4,258,315 (inductance meter), U.S. Pat. No. 4,045,728 (direct reading inductance meter), U.S. Pat. Nos. 6,252,923, 6,194,898, 6,006,023 (molecular sensing apparatus), U.S. Pat. No. 6,048,692 (sensors for electrically sensing binding events for supported molecular receptors), and the like. The entire disclosure of each of these United States patents is hereby incorporated by reference into this specification.

When measuring the inductance of the coated sample, the inductance is preferably measured using an applied wave with a specified frequency. As the magnetic moments of the coated samples align, the inductance increases until a specified value; and it rises in accordance with a specified time constant in the measurement circuitry.

In one embodiment, the deposited material is contacted with the magnetic field until the inductance of the deposited material is at least about 90 percent of its maximum value under the measurement circuitry. At this time, the magnetic particles in the deposited material have been aligned to at least about 90 percent of the maximum extent possible for maximizing the inductance of the sample.

By way of illustration and not limitation, a metal rod with a diameter of 1 micron and a length of 1 millimeter, when uncoated with magnetic nano-sized particles, might have an inductance of about 1 nanohenry. When this metal rod is coated with, e.g., nano-sized ferrites, then the inductance of the coated rod might be 5 nanohenries or more. When the magnetic moments of the coating are aligned, then the inductance might increase to 50 nanohenries, or more. As will be apparent to those skilled in the art, the inductance of the coated article will vary, e.g., with the shape of the article and also with the frequency of the applied electromagnetic field.

One may use any of the conventional magnetic field generators known to those skilled in the art to produce such as magnetic field. Thus, e.g., one may use one or more of the magnetic field generators disclosed in U.S. Pat. Nos. 6,503,364, 6,377,149 (magnetic field generator for magnetron plasma generation), U.S. Pat. No. 6,353,375 (magnetostatic wave device), U.S. Pat. No. 6,340,888 (magnetic field generator for MRI), U.S. Pat. Nos. 6,336,989, 6,335,617 (device for calibrating a magnetic field generator), U.S. Pat. Nos. 6,313,632, 6,297,634, 6,275,128, 6,246,066 (magnetic field generator and charged particle beam irradiator), U.S. Pat. No. 6,114,929 (magnetostatic wave device), U.S. Pat. No. 6,099,459 (magnetic field generating device and method of generating and applying a magnetic field), U.S. Pat. Nos. 5,795,212, 6,106,380 (deterministic magnetorheological finishing), U.S. Pat. No. 5,839,944 (apparatus for deterministic magnetorheological finishing), U.S. Pat. No. 5,971,835 (system for abrasive jet shaping and polishing of a surface using a magnetorheological fluid), U.S. Pat. Nos. 5,951,369, 6,506,102 (system for magnetorheological finishing of substrates), U.S. Pat. Nos. 6,267,651, 6,309,285 (magnetic wiper), and the like. The entire disclosure of each of these United States patents is hereby incorporated by reference into this specification.

In one embodiment, the magnetic field is 1.8 Tesla or less. In this embodiment, the magnetic field can be applied with, e.g., electromagnets disposed around a coated substrate.

For fields greater than about 2 Tesla, one may use superconducting magnets that produce fields as high as 40 Tesla. Reference may be had, e.g., to U.S. Pat. Nos. 5,319,333 (superconducting homogeneous high field magnetic coil), U.S. Pat. Nos. 4,689,563, 6,496,091 (superconducting magnet arrangement), U.S. Pat. No. 6,140,900 (asymmetric superconducting magnets for magnetic resonance imaging), U.S. Pat. No. 6,476,700 (superconducting magnet system), U.S. Pat. No. 4,763,404 (low current superconducting magnet), U.S. Pat. No. 6,172,587 (superconducting high field magnet), U.S. Pat. No. 5,406,204, and the like. The entire disclosure of each of these United States patents is hereby incorporated by reference into this specification.

In one embodiment, no magnetic field is applied to the deposited coating while it is being solidified. In this embodiment, as will be apparent to those skilled in the art, there still may be some alignment of the magnetic domains in a plane parallel to the surface of substrate as the deposited particles are locked into place in a matrix (binder) deposited onto the surface.

In one embodiment, depicted in FIG. 1, the magnetic field 52 is preferably delivered to the coating 48 in a direction that is substantially parallel to the surface 56 of the substrate 46. In another embodiment, depicted in FIG. 1, the magnetic field 58 is delivered in a direction that is substantially perpendicular to the surface 56. In yet another embodiment, the magnetic field 60 is delivered in a direction that is angularly disposed vis-à-vis surface 56 and may form, e.g., an obtuse angle (as in the case of field 62). As will be apparent, combinations of these magnetic fields may be used.

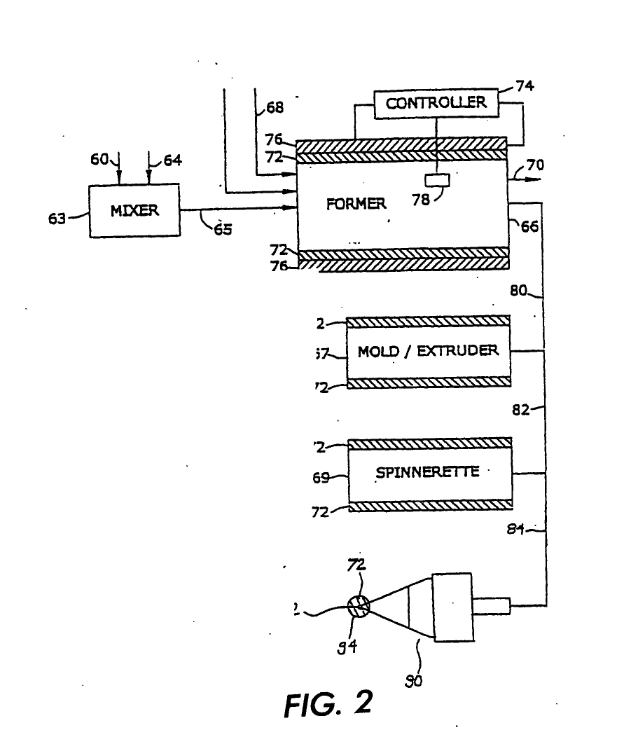

FIG. 2 is a flow diagram of another process that may be used to make the nanomagnetic compositions of this invention. Referring to FIG. 2, and to the preferred process depicted therein, it will be seen that nano-sized ferromagnetic material(s), with a particle size less than about 100 nanometers, is preferably charged via line 60 to mixer 62. It is preferred to charge a sufficient amount of such nano-sized material(s) so that at least about 10 weight percent of the mixture formed in mixer 62 is comprised of such nano-sized material. In one embodiment, at least about 40 weight percent of such mixture in mixer 62 is comprised of such nano-sized material. In another embodiment, at least about 50 weight percent of such mixture in mixer 62 is comprised of such nano-sized material.

In one embodiment, one or more binder materials are charged via line 64 to mixer 62. In one embodiment, the binder used is a ceramic binder. These ceramic binders are well known. Reference may be had, e.g., to pages 172-197 of James S. Reed's “Principles of Ceramic Processing,” Second Edition (John Wiley & Sons, Inc., New York, N.Y., 1995). As is disclosed in the Reed book, the binder may be a clay binder (such as fine kaolin, ball clay, and bentonite), an organic colloidal particle binder (such as microcrystalline cellulose), a molecular organic binder (such as natural gums, polyscaccharides, lignin extracts, refined alginate, cellulose ethers, polyvinyl alcohol, polyvinylbutyral, polymethyl methacrylate, polyethylene glycol, paraffin, and the like.). etc.

In one embodiment, the binder is a synthetic polymeric or inorganic composition. Thus, and referring to George S. Brady et al.'s “Materials Handbook,” (McGraw-Hill, Inc., New York, N.Y. 1991), the binder may be acrylonitrile-butadiene-styrene (see pages 5-6), an acetal resin (see pages 6-7), an acrylic resin (see pages 10-12), an adhesive composition (see pages 14-18), an alkyd resin (see page 27-28), an allyl plastic (see pages 31-32), an amorphous metal (see pages 53-54), a biocompatible material (see pages 95-98), boron carbide (see page 106), boron nitride (see page 107), camphor (see page 135), one or more carbohydrates (see pages 138-140), carbon steel (see pages 146-151), casein plastic (see page 157), cast iron (see pages 159-164), cast steel (see pages 166-168), cellulose (see pages 172-175), cellulose acetate (see pages 175-177), cellulose nitrate (see pages 177), cement (see page 178-180), ceramics (see pages 180-182), cermets (see pages 182-184), chlorinated polyethers (see pages 191-191), chlorinated rubber (see pages 191-193), cold-molded plastics (see pages 220-221), concrete (see pages 225-227), conductive polymers and elastomers (see pages 227-228), degradable plastics (see pages 261-262), dispersion-strengthened metals (see pages 273-274), elastomers (see pages 284-290), enamel (see pages 299-301), epoxy resins (see pages 301-302), expansive metal (see page 313), ferrosilicon (see page 327), fiber-reinforced plastics (see pages 334-335), fluoroplastics (see pages 345-347), foam materials (see pages 349-351), fusible alloys (see pages 362-364), glass (see pages 376-383), glass-ceramic materials (see pages 383-384), gypsum (see pages 406-407), impregnated wood (see pages 422-423), latex (see pages 456-457), liquid crystals (see page 479). lubricating grease (see pages 488-492), magnetic materials (see pages 505-509), melamine resin (see pages 5210-521), metallic materials (see pages 522-524), nylon (see pages 567-569), olefin copolymers (see pages 574-576), phenol-formaldehyde resin (see pages 615-617), plastics (see pages 637-639), polyarylates (see pages 647-648), polycarbonate resins (see pages 648), polyester thermoplastic resins (see pages 648-650), polyester thermosetting resins (see pages 650-651), polyethylenes (see pages 651-654), polyphenylene oxide (see pages 644-655), polypropylene plastics (see pages 655-656), polystyrenes (see pages 656-658), proteins (see pages 666-670), refractories (see pages 691-697), resins (see pages 697-698), rubber (see pages 706-708), silicones (see pages 747-749), starch (see pages 797-802), superalloys (see pages 819-822), superpolymers (see pages 823-825), thermoplastic elastomers (see pages 837-839), urethanes (see pages 874-875), vinyl resins (see pages 885-888), wood (see pages 912-916), mixtures thereof, and the like.

Referring again to FIG. 2, one may charge to line 64 either one or more of these “binder material(s)” and/or the precursor(s) of these materials that, when subjected to the appropriate conditions in former 66, will form the desired mixture of nanomagnetic material and binder.

Referring again to FIG. 2, and in the preferred process depicted therein, the mixture within mixer 62 is preferably stirred until a substantially homogeneous mixture is formed. Thereafter, it may be discharged via line 65 to former 66.

One process for making a fluid composition comprising nanomagnetic particles is disclosed in U.S. Pat. No. 5,804,095, “Magnetorheological Fluid Composition,”, of Jacobs et al; the disclosure of this patent is incorporated herein by reference. In this patent, there is disclosed a process comprising numerous material handling steps used to prepare a nanomagnetic fluid comprising iron carbonyl particles. One suitable source of iron carbonyl particles having a median particle size of 3.1 microns is the GAF Corporation.

The process of Jacobs et al, is applicable to the present invention, wherein such nanomagnetic fluid further comprises a polymer binder, thereby forming a nanomagnetic paint. In one embodiment, the nanomagnetic paint is formulated without abrasive particles of cerium dioxide. In another embodiment, the nanomagnetic fluid further comprises a polymer binder, and aluminum nitride is substituted for cerium dioxide.

There are many suitable mixing processes and apparatus for the milling, particle size reduction, and mixing of fluids comprising solid particles. For example, e.g., iron carbonyl particles or other ferromagnetic particles of the paint may be further reduced to a size on the order of 100 nanometers or less, and/or thoroughly mixed with a binder polymer and/or a liquid solvent by the use of a ball mill, a sand mill, a paint shaker holding a vessel containing the paint components and hard steel or ceramic beads; a homogenizer (such as the Model Ytron Z made by the Ytron Quadro Corporation of Chesham, United Kingdom, or the Microfluidics M700 made by the MFIC Corporation of Newton, Ma.), a powder dispersing mixer (such as the Ytron Zyclon mixer, or the Ytron Xyclon mixer, or the Ytron PID mixer by the Ytron Quadro Corporation); a grinding mill (such as the Model F10 Mill by the Ytron Quadro Corporation); high shear mixers (such as the Ytron Y mixer by the Ytron Quadro Corporation), the Silverson Laboratory Mixer sold by the Silverson Corporation of East Longmeadow, Ma., and the like. The use of one or more of these apparatus in series or in parallel may produce a suitably formulated nanomagnetic paint.

Referring again to FIG. 2, the former 66 is preferably equipped with an input line 68 and an exhaust line 70 so that the atmosphere within the former can be controlled. One may utilize an ambient atmosphere, an inert atmosphere, pure nitrogen, pure oxygen, mixtures of various gases, and the like. Alternatively, or additionally, one may use lines 68 and 70 to afford subatmospheric pressure, atmospheric pressure, or superatomspheric pressure within former 66.

In the embodiment depicted, former 66 is also preferably comprised of an electromagnetic coil 72 that, in response from signals from controller 74, can control the extent to which, if any, a magnetic field is applied to the mixture within the former 66 (and also within the mold 67 and/or the spinnerette 69).

The controller 74 is also adapted to control the temperature within the former 66 by means of heating/cooling assembly.

In the embodiment depicted in FIG. 2, a sensor 78 preferably determines the extent to which the desired nanomagnetic properties have been formed with the nano-sized material in the former 66; and, as appropriate, the sensor 78 imposes a magnetic field upon the mixture within the former 66 until the desired properties have been obtained.

In one embodiment, the sensor 78 is the inductance meter discussed elsewhere in this specification; and the magnetic field is applied until at least about 90 percent of the maximum inductance obtainable with the alignment of the magnetic moments has been obtained.

The magnetic field is preferably imposed until the nano-sized particles within former 78 (and the material with which it is admixed) have a mass density of at least about 0.001 grams per cubic centimeter (and preferably at least about 0.01 grams per cubic centimeter), a saturation magnetization of from about 1 to about 36,000 Gauss, a coercive force of from about 0.01 to about 5,000 Oersteds, and a relative magnetic permeability of from about 1 to about 500,000.

When the mixture within former 66 has the desired combination of properties (as reflected, e.g., by its substantially maximum inductance) and/or prior to that time, some or all of such mixture may be discharged via line 80 to a mold/extruder 67 wherein the mixture can be molded or extruded into a desired shape. A magnetic coil 72 also preferably may be used in mold/extruder 67 to help align the nano-sized particles.

Alternatively, or additionally, some or all of the mixture within former 66 may be discharged via line 82 to a spinnerette 69, wherein it may be formed into a fiber (not shown).

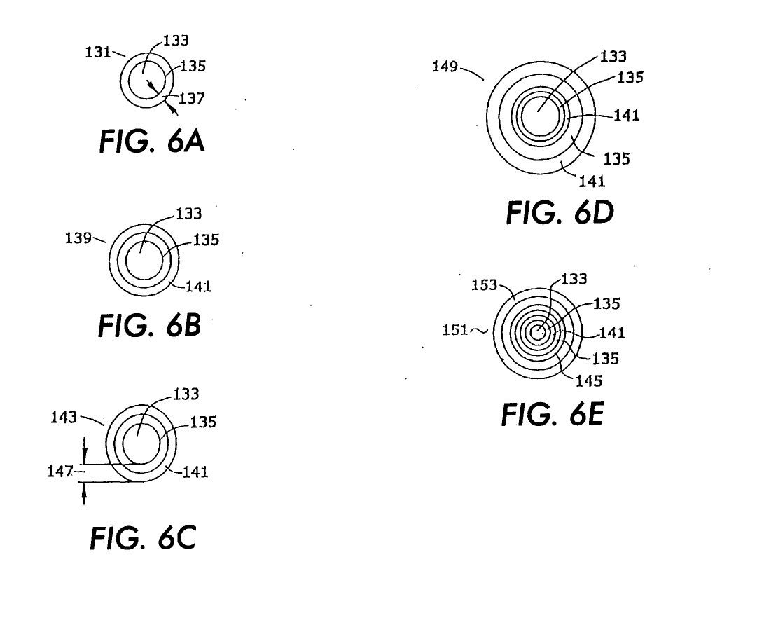

As will be apparent, one may make fibers by the process indicated that have properties analogous to the nanomagnetic properties of the coating 135 (see FIG. 6A), and/or nanoelectrical properties of the coating 141 (see FIG. 6B), and/or nanothermal properties of the coating 145 (see FIG. 6E). Such fiber or fibers may be made into fabric by conventional means. By the appropriate selection and placement of such fibers, one may produce a shielded fabric which provides protection against high magnetic voltages and/or high voltages and/or excessive heat.

Thus, in one embodiment, nanomagnetic and/or nanoelectrical and/or nanothermal fibers are woven together to produce a garment that will shield from the adverse effects of radiation such as, e.g., radiation experienced by astronauts in outer space.

Alternatively, or additionally, some or all of the mixture within former 66 may be discharged via line 84 to a direct writing applicator 90, such as a MicroPen applicator manufactured by OhmCraft Incorporated of Honeoye Falls, N.Y. Such an applicator is disclosed in U.S. Pat. No. 4,485,387, the disclosure of which is incorporated herein by reference. The use of this applicator to write circuits and other electrical structures is described in, e.g., U.S. Pat. No. 5,861,558 of Buhl et al, “Strain Gauge and Method of Manufacture”, the disclosure of which is incorporated herein by reference.

In one preferred embodiment, the nanomagnetic, nanoelectrical, and/or nanothermal compositions of the present invention, along with various conductor, resistor, capacitor, and inductor formulations, are dispensed by the MicroPen device, to fabricate the circuits and structures of the present invention on devices such as, e.g. catheters and other biomedical devices.

In one preferred embodiment, involving the writing of nanomagnetic circuit patterns and/or thin films, the direct writing applicator 90 (as disclosed in U.S. Pat. No. 4,485,387) comprises an applicator tip 92 and an annular magnet 94, which provides a magnetic field 72. The use of such an applicator 90 to apply nanomagnetic coatings is particularly beneficial because the presence of the magnetic field from magnet 94, through which the nanomagnetic fluid flows serves to orient the magnetic particles in situ as such nanomagnetic fluid is applied to a substrate. Such an orienting effect is described in U.S. Pat. No. 5,971,835, the disclosure of which is incorporated herein by reference. Once the nanomagnetic particles are properly oriented by such a field, or by another magnetic field source, the applied coating is cured by heating, by ultraviolet radiation, by an electron beam, or by other suitable means.

In one embodiment, not shown, one may form compositions comprised of nanomagentic particles and/or nanoelectrical particles and/or nanothermal particles and/or other nano-sized particles by a sol-gel process. Thus, by way of illustration and not limitation, one may use one or more of the processes described in U.S. Pat. Nos. 6,287,639 (nanocomposite material comprised of inorganic particles and silanes), U.S. Pat. No. 6,337,117 (optical memory device comprised of nano-sized luminous material), U.S. Pat. No. 6,527,972 (magnetorheological polymer gels), U.S. Pat. No. 6,589,457 (process for the deposition of ruthenium oxide thin films), U.S. Pat. No. 6,657,001 (polysiloxane compositions comprised of inorganic particles smaller than 100 nanometers), U.S. Pat. No. 6,666,935 (sol-gel manufactured energetic materials), and the like. The entire disclosure of each of these United States patents is hereby incorporated by reference into this specification.

Nanomagnetic Compositions Comprised of Moieties A, B, and C

The aforementioned process described in the preceding section of this specification, and the other processes described in this specification, may each be adapted to produce other, comparable nanomagnetic structures, as is illustrated in FIG. 3.

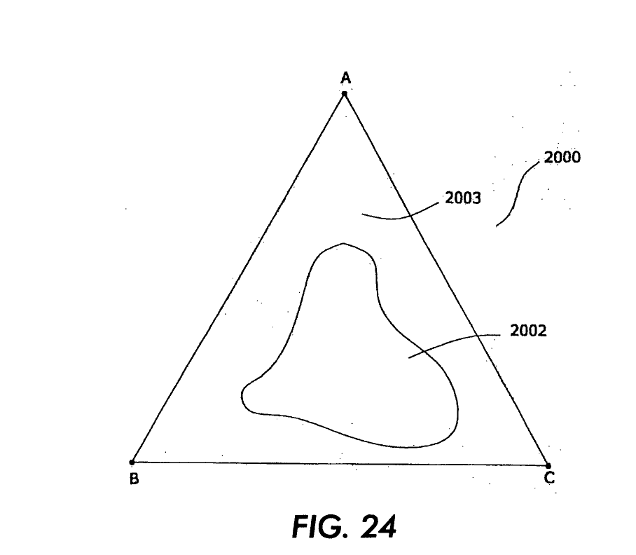

Referring to FIG. 3, and in the preferred embodiment depicted therein, a phase diagram 100 is presented. As is illustrated by this phase diagram 100, the nanomagnetic material used in this embodiment of the invention preferably is comprised of one or more of moieties A, B, and C. The moieties A, B, and C described in reference to phase 100 of FIG. 3 are not necessarily the same as the moieties A, B, and C described in reference to phase diagram 2000 of FIG. 24.

In the embodiment depicted, the moiety A depicted in phase diagram 100 is preferably comprised of a magnetic element selected from the group consisting of a transition series metal, a rare earth series metal, or actinide metal, a mixture thereof, and/or an alloy thereof. In one embodiment, the moiety A is iron. In another embodiment, moiety A is nickel. In yet another embodiment, moiety A is cobalt. In yet another embodiment, moiety A is gadolinium. In another embodiment, the A moiety is selected from the group consisting of samarium, holmium, neodymium, and one or more other members of the Lanthanide series of the periodic table of elements. In yet another embodiment, the moiety A is identical to the moiety A described in this specification by reference to FIG. 24.

As is known to those skilled in the art, the transition series metals include chromium, manganese, iron, cobalt, and nickel. One may use alloys of iron, cobalt and nickel such as, e.g., iron—aluminum, iron—carbon, iron—chromium, iron—cobalt, iron—nickel, iron nitride (Fe₃N), iron phosphide, iron-silicon, iron-vanadium, nickel-cobalt, nickel-copper, and the like. One may use alloys of manganese such as, e.g., manganese-aluminum, manganese-bismuth, MnAs, MnSb, MnTe, manganese-copper, manganese-gold, manganese-nickel, manganese-sulfur and related compounds, manganese-antimony, manganese-tin, manganese-zinc, Heusler alloy W, and the like. One may use compounds and alloys of the iron group, including oxides of the iron group, halides of the iron group, borides of the transition elements, sulfides of the iron group, platinum and palladium with the iron group, chromium compounds, and the like.

One may use a rare earth and/or actinide metal such as, e.g., Ce, Pr, Nd, Pm, Sm, Eu, Gd, Tb, Dy, Ho, Er, Tm, Yb, Lu, La, mixtures thereof, and alloys thereof. One may also use one or more of the actinides such as, e.g., the actinides of Th, Pa, U, Np, Pu, Am, Cm, Bk, Cf, Es, Fm, Md, No, Lr, Ac, and the like.

These moieties, compounds thereof, and alloys thereof are well known and are described, e.g., in the text of R. S. Tebble et al. entitled “Magnetic Materials.”

In one preferred embodiment, illustrated in FIG. 3, moiety A is selected from the group consisting of iron, nickel, cobalt, alloys thereof, and mixtures thereof. In this embodiment, the moiety A is magnetic, i.e., it has a relative magnetic permeability of from about 1 to about 500,000. As is known to those skilled in the art, relative magnetic permeability is a factor, being a characteristic of a material, which is proportional to the magnetic induction produced in a material divided by the magnetic field strength; it is a tensor when these quantities are not parallel. See, e.g., page 4-128 of E. U. Condon et al.'s “Handbook of Physics” (McGraw-Hill Book Company, Inc., New York, N.Y., 1958).

The moiety A of FIG. 3 also preferably has a saturation magnetization of from about 1 to about 36,000 Gauss, and a coercive force of from about 0.01 to about 5,000 Oersteds.

The moiety A of FIG. 3 may be present in the nanomagnetic material either in its elemental form, as an alloy, in a solid solution, or as a compound.

It is preferred at least about 1 mole percent of moiety A be present in the nanomagnetic material (by total moles of A, B, and C), and it is more preferred that at least 10 mole percent of such moiety A be present in the nanomagnetic material (by total moles of A, B, and C). In one embodiment, at least 60 mole percent of such moiety A is present in the nanomagnetic material, (by total moles of A, B, and C.)

In the embodiment depicted in FIG. 3, in addition to moiety A, it is preferred to have moiety B be present in the nanomagnetic material. In this embodiment, moieties A and B are admixed with each other. The mixture may be a physical mixture, it may be a solid solution, it may be comprised of an alloy of the A/B moieties, etc.

The Squareness of the Nanomagnetic Particles of the Invention

As is known to those skilled in the art, the squareness of a magnetic material is the ratio of the residual magnetic flux and the saturation magnetic flux density. Reference may be had, e.g., to U.S. Pat. Nos. 6,627,313, 6,517,934, 6,458,452, 6,391,450, 6,350,505, 6,248,437, 6,194,058, 6,042,937, 5,998,048, 5,645,652, and the like. The entire disclosure of such United States patents is hereby incorporated by reference into this specification. Reference may also be had to page 1802 of the McGraw-Hill Dictionary of Scientific and Techical Terms, Fourth Edition (McGraw-Hill Book Company, New York, N.Y., 1989). At such page 1802, the “squareness ratio” is defined as “The magnetic induction at zero magnetizing force divided by the maximum magnetic indication, in a symmetric cyclic magnetization of a material.”

In one embodiment, the squareness of applicants' nanomagnetic material is from about 0.05 to about 1.0. In one aspect of this embodiment, such squareness is from about 0.1 to about 0.9. In another aspect of this embodiment, the squareness is from about 0.2 to about 0.8. In applications where a large residual magnetic moment is desired, the squareness is preferably at least about 0.8.

Referring again to FIG. 3, and in the preferred embodiment depicted therein, the nanomagnetic material may be comprised of 100 percent of moiety A, provided that such moiety A has the required normalized magnetic interaction (M). Alternatively, the nanomagnetic material may be comprised of both moiety A and moiety B.

When moiety B is present in the nanomagnetic material, in whatever form or forms it is present, it is preferred that it be present at a mole ratio (by total moles of A and B) of from about 1 to about 99 percent and, preferably, from about 10 to about 90 percent.

The B moiety, in one ebodiment, in whatever form it is present, is preferably nonmagnetic, i.e., it has a relative magnetic permeability of about 1.0; without wishing to be bound to any particular theory, applicants believe that the B moiety acts as buffer between adjacent A moieties. One may use, e.g., such elements as silicon, aluminum, boron, platinum, tantalum, palladium, yttrium, zirconium, titanium, calcium, beryllium, barium, silver, gold, indium, lead, tin, antimony, germanium, gallium, tungsten, bismuth, strontium, magnesium, zinc, and the like.

In one embodiment, the B moiety has a relative magnetic permeability that is about equal to 1 plus the magnetic susceptilibity. The relative magnetic susceptilities of silicon, aluminum, boron, platinum, tantalum, palladium, yttrium, zirconium, titanium, calcium, beryllium, barium, silver, gold, indium, lead, tin, antimony, germanium, gallium, tungsten, bismuth, strontium, magnesium, zinc, copper, cesium, cerium, hafnium, iodine, iridium, lanthanum, lithium, lutetium, manganese, molybdenum, potassium, sodium, strontium, praseodymium, rhenium, rhodium, rubidium, ruthenium, scandium, selenium, tantalum, technetium, tellurium, chromium, thallium, thorium, thulium, titanium, vanadium, zinc, yttrium, ytterbium, zirconium, and the like. Reference may be had, e.g., to pages E-118 through E123 of the aforementioned CRC Handbook of Chemistry and Physics.

In one embodiment, the nanomagnetic particles may be represented by the formula AₓB_{y}C_{z}, wherein x+y+z is equal to 1. In this embodiment the ratio of x/y is at least 0.1 and preferably at least 0.2; and the ratio of z/x is from 0.001 to about 0.5.

In one embodiment, and without wishing to be bound to any particular theory, it is believed that B moiety provides plasticity to the nanomagnetic material that it would not have but for the presence of such B moiety. In one aspect of this embodiment, it is preferred that the bending radius of a substrate coated with both A and B moieties be no greater than 90 percent of the bending radius of a substrate coated with only the A moiety.

The use of the B material allows one, in one embodiment, to produce a coated substrate with a springback angle of less than about 45 degrees. As is known to those skilled in the art, all materials have a finite modulus of elasticity; thus, plastic deformation is followed by some elastic recovery when the load is removed. In bending, this recovery is called springback. See, e.g., page 462 of S. Kalparjian's “Manufacturing Engineering and Technology,” Third Edition (Addison Wesley Publishing Company, New York, N.Y., 1995).

In one preferred embodiment, the B material is aluminum and the C material is nitrogen, whereby an AlN moiety is formed. Without wishing to be bound to any particular theory, applicants believe that aluminum nitride (and comparable materials) are both electrically insulating and thermally conductive, thus providing a excellent combination of properties for certain end uses.

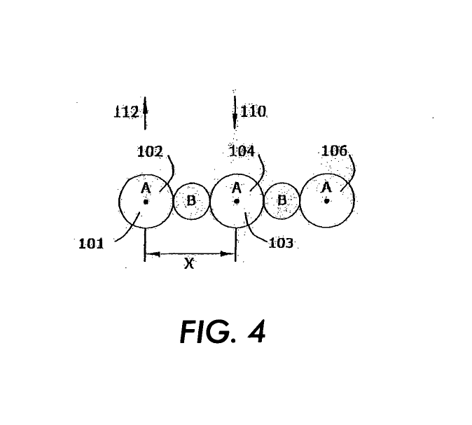

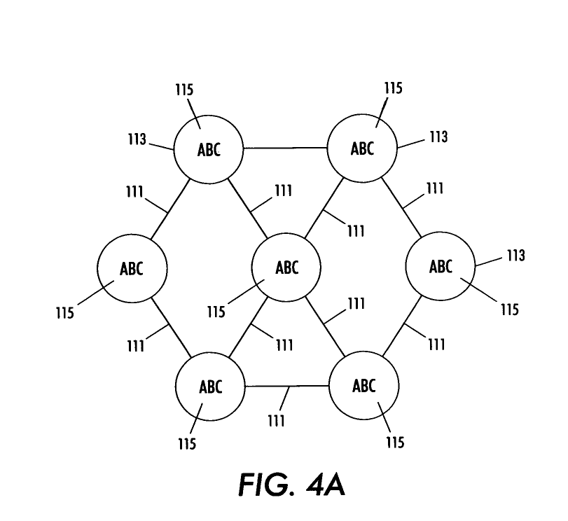

Referring again to FIGS. 3 and 4, when an electromagnetic field 110 is incident upon the nanomagnetic material comprised of A and B (see FIG. 3), such a field will be reflected to some degree depending upon the ratio of moiety A and moiety B. In one embodiment, it is preferred that at least 1 percent of such field is reflected in the direction of arrow 112 (see FIG. 4). In another embodiment, it is preferred that at least about 10 percent of such field is reflected. In yet another embodiment, at least about 90 percent of such field is reflected. Without wishing to be bound to any particular theory, applicants believe that the degree of reflection depends upon the concentration of A in the A/B mixture.

Referring again to FIG. 3, and in one embodiment, the nanomagnetic material is comprised of moiety A, moiety C, and optionally moiety B. The moiety C is preferably selected from the group consisting of elemental oxygen, elemental nitrogen, elemental carbon, elemental fluorine, elemental chlorine, elemental hydrogen, and elemental helium, elemental neon, elemental argon, elemental krypton, elemental xenon, elemental fluorine, elemental sulfur, elemental hydrogen, elemental helium, the elemental chlorine, elemental bromine, elemental iodine, elemental boron, elemental phosphorus, and the like. In one aspect of this embodiment, the C moiety is selected from the group consisting of elemental oxygen, elemental nitrogen, and mixtures thereof.

In one embodiment, the C moiety is chosen from the group of elements that, at room temperature, form gases by having two or more of the same elements combine. Such gases include, e.g., hydrogen, the halide gases (fluorine, chlorine, bromine, and iodine), inert gases (helium, neon, argon, krypton, xenon, etc.), etc.

It is preferred, when the C moiety is present, that it be present in a concentration of from about 1 to about 90 mole percent, based upon the total number of moles of the A moiety and/or the B moiety and the C moiety in the composition.

Referring again to FIG. 3, and in the embodiment depicted, the area 114 produces a composition which optimizes the degree to which magnetic flux are initially trapped and/or thereafter released by the composition when a magnetic field is withdrawing from the composition.

Without wishing to be bound to any particular theory, applicants believe that, when a composition as described by area 114 is subjected to an alternating magnetic field, at least a portion of the magnetic field is trapped by the composition when the field is strong, and then this portion tends to be released when the field lessens in intensity.

Thus, e.g., it is believed that, when the magnetic field 110 is applied to the nanomagnetic material, it starts to increase, in a typical sine wave fashion. After a specified period of time, a magnetic moment is created within the nanomagnetic material; but, because of the time delay, there is a phase shift.

The time delay will vary with the composition of the nanomagnetic material. By maximizing the amount of trapping, and by minimizing the amount of reflection and absorption, one may minimize the magnetic artifacts caused by the nanomagnetic shield.

Thus, and referring again to FIG. 3, one may optimize the A/B/C composition to preferably be within the area 114. In general, the A/B/C composition has molar ratios such that the ratio of A/(A and C) is from about 1 to about 99 mole percent and, preferably, from about 10 to about 90 mole percent. In one preferred embodiment, such ratio is from about 40 to about 60 molar percent.

The molar ratio of A/(A and B and C) generally is from about 1 to about 99 mole percent and, preferably, from about 10 to about 90 molar percent. In one embodiment, such molar ratio is from about 30 to about 60 molar percent.

The molar ratio of B/(A plus B plus C) generally is from about 1 to about 99 mole percent and, preferably, from about 10 to about 40 mole percent.

The molar ratio of C/(A plus B plus C) generally is from about 1 to about 99 mole percent and, preferably, from about 10 to about 50 mole percent.

In one embodiment, the composition of the nanomagnetic material is chosen so that the applied electromagnetic field 110 is absorbed by the nanomagnetic material by less than about 1 percent; thus, in this embodiment, the applied magnetic field 110 is substantially restored by correcting the time delay.

By utilizing nanomagnetic material that absorbs the electromagnetic field, one may selectively direct energy to various cells within a biological organism that are to treated. Thus, e.g., cancer cells can be injected with the nanomagnetic material and then destroyed by the application of externally applied electromagnetic fields. Because of the nano size of applicants' materials, they can readily and preferentially be directed to the malignant cells to be treated within a living organism. In this embodiment, the nanomagnetic material preferably has a particle size of from about 5 to about 10 nanometers.

Preferred Nanomagnetic Particles

In one embodiment of this invention, there is provided a multiplicity of nanomagnetic particles that may be in the form of a film, a powder, a solution, etc. This multiplicity of nanogmentic particles is hereinafter referred to as a collection of nanomagnetic particles.

The collection of nanomagnetic particles of this embodiment of the invention is generally comprised of at least about 0.05 weight percent of such nanomagentic particles and, preferably, at least about 5 weight percent of such nanomagnetic particles. In one embodiment, such collection is comprised of at least about 50 weight percent of such magnetic particles. In another embodiment, such collection consists essentially of such nanomagnetic particles.

When the collection of nanomagnetic particles consists essentially of nanomagnetic particles, the term “compact” will be used to refer to such collection of nanomagnetic particles.

The average size of the nanomagnetic particles is preferably less than about 100 nanometers. In one embodiment, the nanomagnetic particles have an average size of less than about 20 nanometers. In another embodiment, the nanomagnetic particles have an average size of less than about 15 nanometers. In yet another embodiment, such average size is less than about 11 nanometers. In yet another embodiment, such average size is less than about 3 nanometers.

In one embodiment of this invention, the nanomagnetic particles have a phase transition temperature of from about 0 degrees Celsius to about 1,200 degees Celsius. In one aspect of this embodiment, the phase transition temperature is from about 40 degrees Celsius to about 200 degrees Celsius.

As used herein, the term phase transition temperature refers to temperature in which the magnetic order of a magnetic particle transitions from one magnetic order to another. Thus, for example, when a magnetic particle transitions from the ferromagnetic order to the paramagnetic order, the phase transition temperature is the Curie temperature. Thus, e.g., when the magnetic particle transitions from the anti-ferromagnetic order to the paramagnetic order, the phase transition temperature is known as the Neel temperature.

As used herein, the term “Curie temperature” refers to the temperature marking the transition between ferromagnetism and paramagnetism, or between the ferroelectric phase and paraelectric phase. This term is also sometimes referred to as the “Curie point.” Reference may be had, e.g., to U.S. Pat. Nos. 5,429,583, 6,599,234, 6,565,887, 6,267,313, 4,138,998, 5,571,153, 6,635,009, and the like. The entire disclosure of each of these United States patents is hereby incorporated by reference into this specification.

As used herein, the term “Neel temperature” refers to a temperature, characteristic of certain metals, alloys, and salts, below which spontaneous magnetic ordering takes place so that they become antiferromagnetic, and above which they are paramagnetic; this is also known as the Neel point. Reference may be had, e.g., to U.S. Pat. Nos. 4,103,315, 3,791,843, 5,492,720, 6,181,533, 3,883,892, 5,264,980, 3,845,306, 6,083,632, 4,396,886, 6,020,060, and the like. The entire disclosure of each of these United States patents is hereby incorporated by refemec into this specification.

Neel temperature is also disussed at page F-92 of the “Handbook of Chemistry and Physics,” 63^{rd} Edition (CRC Press, Inc., Boca Raton, Fla., 1982-1983). As is disclosed on such page, ferromagnetic materials are “those in which the magnetic moments of atoms or ions tend to assume an ordered but nonparallel arrangement in zero applied field, below a characteristic temperature called the Neel point. In thie usual case, within a magnetic domain, a substantial net mangetization results form the antiparallel alignment of neighboring nonequivalent subslattices. The macroscopic behavior is similar to that in ferromagnetism. Above the Neel point, these materials become paramagnetic.”

As is disclosed in U.S. Pat. No. 5,412,182, the entire disclosure of which is hereby incorporated by reference into this specification, “The implants are accordingly heated by resistive loses from any induced current circulations and the tumor tissue is heated by thermal conduction. Implant temperatures are achieved in accordance with Curie temperature characteristics of the ferromagnetic material used. The ferromagnetic property of these implants changes as a function of temperature, heating is gradually reduced as the Curie temperature is approached and further reduced when the Curie temperature is exceeded. Thermal regulation is dependent on a sharp transition in the Curie temperature curve at the desired temperature. The availability of implants that can be thermally regulated at desirable temperatures is limited by practical metallurgy limitations. Further, coils used to generate required high intensity magnetic fields are extremely inefficient. In fact, 1500-3000 Watts can be required and the implants need to be aligned with the applied magnetic field. Due to the high power requirements, both very expensive radiofrequency shielded rooms and complex cooling systems are required.”

Without wishing to be bound to any particular theory, applicants believe that the phase temperature of their nanomagnetic particles can be varied by varying the ratio of the A, B, and C moieties described hereinabove as well as the particle sizes of the nanoparticles.

In one embodiment, the magnetic order of the nanomagnetic particles of this invention is destroyed at a temperature in excess of the phase transition temperature. This phenemon is illustrated in FIGS. 3A, 3B, and 3C.

Referring to FIG. 3A, it will be seen that a multiplicity of nano-sized particles 91 are disposed within a cell 93 which, in the embodiment depicted, is a cancer cell. The particles 91 are subjected to electromagnetic radiation 95 which causes them, in the embodiment depicted, to heat to a temperature sufficient to destroy the cancer cell but insufficient to destroy surrounding cells. The particles 91 are preferably delivered to the cancer cell 93 by one or more of the means described elsewhere in this specification and/or in the prior art.

In the embodiment depicted in FIG. 3A, the temperature of the particles 91 is less than the phase transition temperature of such particles, “Tₜᵣₐₙₛᵢₜᵢₒₙ” Thus, in this case, the particles 91 have a magnetic order, i.e., they are either ferromagnetic or superparamagnetic and, thus, are able to receive the external radiation 95 and transform at least a portion of the electromagnetic energy into heat.

When the temperature of the particles 91 exceeds the “Tₜᵣₐₙₛᵢₜᵢₒₙ” temperature (i.e., their phase transition temperature), the magnetic order of such particles is destroyed, and they are no longer able to transform electromagnetic energy into heat. This situation is depicted in FIG. 3B.

When the particles 91 cease transforming electromagnetic energy into heat, they tend to cool and then revert to a temperature below “Tₜᵣₐₙₛᵢₜᵢₒₙ”, as depicted in FIG. 3A. Thus, the particles 91 act as a heat switch, ceasing to transform electromagnetic energy into heat when they exceed their phase transition temperature and resuming such capability when they are cooled below their phase transition temperature. This capability is schematically illustrated in FIG. 3C.

In one embodiment, the phase transition temperature of the nanoparticles is higher than the temperature needed to kill cancer cells but lower than the temperature needed to kill normal cells. As is disclosed in, e.g., U.S. Pat. No. 4,776,086 (the entire disclosure of which is hereby incorporated by reference into this specification), “The use of elevated temperatures, i.e., hyperthermia, to repress tumors has been under continuous investigation for many years. When normal human cells are heated to 41°-43° C., DNA synthesis is reduced and respiration is depressed. At about 45° C., irreversible destruction of structure, and thus function of chromosome associated proteins, occurs. Autodigestion by the cell's digestive mechanism occurs at lower temperatures in tumor cells than in normal cells. In addition, hyperthermia induces an inflammatory response which may also lead to tumor destruction. Cancer cells are more likely to undergo these changes at a particular temperature. This may be due to intrinsic differences, between normal cells and cancerous cells. More likely, the difference is associated with the lop pH (acidity), low oxygen content and poor nutrition in tumors as a consequence of decreased blood flow. This is confirmed by the fact that recurrence of tumors in animals, after hyperthermia, is found in the tumor margins; probably as a consequence of better blood supply to those areas.”

In one embodiment of this invention, the phase transition temperature of the nanomagnetic material is less than about 50 degrees Celsius and, preferably, less than about 46 degrees Celsius. In one aspect of this embodiment, such phase transition temperature is less than about 45 degrees Celsius.

The nanomagnetic particles of this invention preferably have a saturation magnetization (“magnetic moment”) of from about 2 to about 2,000 electromagnetic units (emu) per cubic centimeter of material. This parameter may be measured by conventional means. Reference may be had, e.g., to U.S. Pat. Nos. 5,068,519 (magnetic document validator employing remanence and saturation measurements), U.S. Pat. Nos. 5,581,251, 6,666,930, 6,506,264 (ferromagnetic powder), U.S. Pat. Nos. 4,631,202, 4,610,911, 5,532,095, and the like. The entire disclosure of each of these United States patents is hereby incorporated by reference into this specification.

In one embodiment, the saturation magnetization of the nanomagnetic particles is measured by a SQUID (superconducting quantum interference device). Reference may be had, e.g., to U.S. Pat. Nos. 5,423,223 (fatigue detection in steel using squid mangetometry), U.S. Pat. No. 6,496,713 (ferromagnetic foreign body detection with background canceling), U.S. Pat. Nos. 6,418,335, 6,208,884 (noninvasive room temperature instrument to measure magnetic susceptibility variations in body tissue), U.S. Pat. No. 5,842,986 (ferromagnetic foreign body screening method), U.S. Pat. Nos. 5,471,139, 5,408,178, and the like. The entire disclosure of each of these United States patents is hereby incorporated by reference into this specification.

In one preferred embodiment, the saturation magnetization of the nanomagnetic particle of this invention is at least 100 electromagnetic units (emu) per cubic centimeter and, more preferably, at least about 200 electromagnetic units (emu) per cubic centimter. In one aspect of this embodiment, the saturation magnetization of such nanomagnetic particles is at least about 1,000 electromagnetic units per cubic centimeter.

Without wishing to be bound to any particular theory, applicants believe that the saturation magnetization of their nanomagnetic particles may be varied by varying the concentration of the “magnetic” moiety A in such particles, and/or the concentrations of moieties B and/or C.

Other Embodiments of the Invention

In this portion of the specification, certain other preferred embodiments of applicants' invention will be described.

In one embodiment, the composition of this invention is comprised of nanomagnetic particles with a specified magnetization. As is known to those skilled in the art, magnetization is the magnetic moment per unit volume of a substance. Reference may be had, e.g., to U.S. Pat. Nos. 4,169,998, 4,168,481, 4,166,263, 5,260,132, 4,778,714, and the like. The entire disclosure of each of these United States patents is hereby incorporated by reference into this specification.

In this embodiment, the nanomagnetic particles are present within a layer that preferably has a saturation magnetization, at 25 degrees Centigrade, of from about 1 to about 36,000 Gauss, or higher. In one embodiment, the saturation magnetization at room temperature of the nanomagentic particles is from about 500 to about 10,000 Gauss. For a discussion of the saturation magnetization of various materials, reference may be had, e.g., to U.S. Pat. Nos. 4,705,613, 4,631,613, 5,543,070, 3,901,741 (cobalt, samarium, and gadolinium alloys), and the like. The entire disclosure of each of these United States patents is hereby incorporated by reference into this specification. As will be apparent to those skilled in the art, especially upon studying the aforementioned patents, the saturation magnetization of thin films is often higher than the saturation magnetization of bulk objects.

In one embodiment, it is preferred to utilize a thin film with a thickness of less than about 2 microns and a saturation magnetization in excess of 20,000 Gauss. The thickness of the layer of nanomagentic material is measured from the bottom surface of the layer that contains such material to the top surface of such layer that contains such material; and such bottom surface and/or such top surface may be contiguous with other layers of material (such as insulating material) that do not contain nanomagnetic particles.

Thus, e.g., one may make a thin film in accordance with the procedure described at page 156 of Nature, Volume 407, Sep. 14, 2000, that describes a multilayer thin film that has a saturation magnetization of 24,000 Gauss.

By the appropriate selection of nanomagnetic particles, and the thickness of the films deposited, one may obtain saturation magnetizations of as high as at least about 36,000.

In one embodiment, the nanomagnetic materials used in the invention typically comprise one or more of iron, cobalt, nickel, gadolinium, and samarium atoms. Thus, e.g., typical nanomagnetic materials include alloys of iron and nickel (permalloy), cobalt, niobium, and zirconium (CNZ), iron, boron, and nitrogen, cobalt, iron, boron, and silica, iron, cobalt, boron, and fluoride, and the like. These and other materials are described in a book by J. Douglas Adam et al. entitled “Handbook of Thin Film Devices” (Academic Press, San Diego, Calif., 2000). Chapter 5 of this book, beginning at page 185, describes “magnetic films for planar inductive components and devices;” and Tables 5.1 and 5.2 in this chapter describe many magnetic materials.

In one embodiment, the nanomagnetic material has a saturation magnetization of from about 1 to about 36,000 Gauss. In one embodiment, the nanomagnetic material has a saturation magnetization of from about 200 to about 26,000 Gauss.

In one embodiment, the nanomagnetic material also has a coercive force of from about 0.01 to about 5,000 Oersteds. The term coercive force refers to the magnetic field, H, which must be applied to a magnetic material in a symmetrical, cyclically magnetized fashion, to make the magnetic induction, B, vanish; this term often is referred to as magnetic coercive force. Reference may be had, e.g., to U.S. Pat. Nos. 4,061,824, 6,257,512, 5,967,223, 4,939,610, 4,741,953, and the like. The entire disclosure of each of these United States patents is hereby incorporated by reference into this specification.

In one embodiment, the nanomagnetic material has a coercive force of from about 0.01 to about 3,000 Oersteds. In yet another embodiment, the nanomagnetic material 103 has a coercive force of from about 0.1 to about 10.

In one embodiment, the nanomagnetic material preferably has a relative magnetic permeability of from about 1 to about 500,000; in one embodiment, such material has a relative magnetic permeability of from about 1.5 to about 260,000. As used in this specification, the term relative magnetic permeability is equal to B/H, and is also equal to the slope of a section of the magnetization curve of the magnetic material. Reference may be had, e.g., to page 4-28 of E. U. Condon et al.'s “Handbook of Physics” (McGraw-Hill Book Company, Inc., New York, 1958).

Reference also may be had to page 1399 of Sybil P. Parker's “McGraw-Hill Dictionary of Scientific and Technical Terms,” Fourth Edition (McGraw Hill Book Company, New York, 1989). As is disclosed on this page 1399, permeability is “ . . . a factor, characteristic of a material, that is proportional to the magnetic induction produced in a material divided by the magnetic field strength; it is a tensor when these quantities are not parallel.

Reference also may be had, e.g., to U.S. Pat. Nos. 6,181,232, 5,581,224, 5,506,559, 4,246,586, 6,390,443, and the like. The entire disclosure of each of these United States patents is hereby incorporated by reference into this specification.

In one embodiment, the nanomagnetic material has a relative magnetic permeability of from about 1.5 to about 2,000.

In one embodiment, the nanomagnetic material preferably has a mass density of at least about 0.001 grams per cubic centimeter; in one aspect of this embodiment, such mass density is at least about 1 gram per cubic centimeter. As used in this specification, the term mass density refers to the mass of a give substance per unit volume. See, e.g., page 510 of the aforementioned “McGraw-Hill Dictionary of Scientific and Technical Terms.” In another embodiment, the material has a mass density of at least about 3 grams per cubic centimeter. In another embodiment, the nanomagnetic material has a mass density of at least about 4 grams per cubic centimeter.

In one embodiment, it is preferred that the nanomagnetic material, and/or the article into which the nanomagnetic material has been incorporated, be interposed between a source of radiation and a substrate to be protected therefrom.

In one embodiment, the nanomagnetic material is in the form of a layer that preferably has a saturation magnetization, at 25 degree Centigrade, of from about 1 to about 36,000 Gauss and, more preferably, from about 1 to about 26,000 Gauss. In one aspect of this embodiment, the saturation magnetization at room temperature of the nanomagnetic particles is from about 500 to about 10,000 Gauss.

In one embodiment, the nanomagnetic material is disposed within an insulating matrix so that any heat produced by such particles will be slowly dispersed within such matrix. Such matrix may be made from, e.g., ceria, calcium oxide, silica, alumina, and the like. In general, the insulating material preferably has a thermal conductivity of less than about 20 (calories centimeters/square centimeters-degree Kelvin second)×10,000. See, e.g., page E-6 of the 63^{rd} Edition of the “Handbook of Chemistry and Physics” (CRC Press, Inc. Boca Raton, Fla., 1982).

In one embodiment, there is provided a coating of nanomagnetic particles that consists of a mixture of aluminum oxide (Al₂O₃), iron, and other particles that have the ability to deflect electromagnetic fields while remaining electrically non-conductive. In one aspect of this embodiment, the particle size in such a coating is approximately 10 nanometers. Preferably the particle packing density is relatively low so as to minimize electrical conductivity. Such a coating, when placed on a fully or partially metallic object (such as a guide wire, catheter, stent, and the like) is capable of deflecting electromagnetic fields, thereby protecting sensitive internal components, while also preventing the formation of eddy currents in the metallic object or coating. The absence of eddy currents in a metallic medical device provides several advantages, to wit: (1) reduction or elimination of heating, (2) reduction or elimination of electrical voltages which can damage the device and/or inappropriately stimulate internal tissues and organs, and (3) reduction or elimination of disruption and distortion of a magnetic-resonance image.

Determination of the Heat Shielding Effect of the Magnetic Shield

In one preferred embodiment, the composition of this invention minimizes the extent to which a substrate increases its heat when subjected to a strong magnetic filed. This heat buildup can be determined in accordance with A.S.T.M. Standard Test F-2182-02, “Standard test method for measurement of radio-frequency induced heating near passive implant during magnetic resonance imaging.”

In this test, the radiation used is representative of the fields present during MRI procedures. As is known to those skilled in the art, such fields typically include a static field with a strength of from about 0.5 to about 2 Teslas, a radio frequency alternating magnetic field with a strength of from about 20 microTeslas to about 100 microTeslas, and a gradient magnetic field that has three components (x, y, and z), each of which has a field strength of from about 0.05 to 500 milliTeslas.

During this test, a temperature probe is used to measure the temperature of an unshielded conductor when subjected to the magnetic field in accordance with such A.S.T.M. F-2182-02 test.

The same test is then is then performed upon a shielded conductor assembly that is comprised of the conductor and a magnetic shield.

The magnetic shield used may comprise nanomagnetic particles, as described hereinabove. Alternatively, or additionally, it may comprise other shielding material, such as, e.g., oriented nanotubes (see, e.g., U.S. Pat. No. 6,265,466).

In one embodiment, the shield is in the form of a layer of shielding material with a thickness of from about 10 nanometers to about 1 millimeter. In another embodiment, the thickness is from about 10 nanometers to about 20 microns.

In one preferred embodiment the shielded conductor is an implantable device and is connected to a pacemaker assembly comprised of a power source, a pulse generator, and a controller. The pacemaker assembly and its associated shielded conductor are preferably disposed within a living biological organism.

In one preferred embodiment, when the shielded assembly is tested in accordance with A.S.T.M. 2182-02, it will have a specified temperature increase (“dTₛ”). The “dT_{c}” is the change in temperature of the unshielded conductor using precisely the same test conditions but omitting the shield. The ratio of dTₛ/dT_{c}, is the temperature increase ratio; and one minus the temperature increase ratio (1−dTₛ/dT_{z}) is defined as the heat shielding factor.

It is preferred that the shielded conductor assembly have a heat shielding factor of at least about 0.2. In one embodiment, the shielded conductor assembly has a heat shielding factor of at least 0.3.

In one embodiment, the nanomagnetic shield of this invention is comprised of an antithrombogenic material.

Antithrombogenic compositions and structures have been well known to those skilled in the art for many years. As is disclosed, e.g., in U.S. Pat. No. 5,783,570, the entire disclosure of which is hereby incorporated by reference into this specification, “Artificial materials superior in processability, elasticity and flexibility have been widely used as medical materials in recent years. It is expected that they will be increasingly used in a wider area as artificial organs such as artificial kidney, artificial lung, extracorporeal circulation devices and artificial blood vessels, as well as disposable products such as syringes, blood bags, cardiac catheters and the like. These medical materials are required to have, in addition to sufficient mechanical strength and durability, biological safety, which particularly means the absence of blood coagulation upon contact with blood, i.e., antithrombogenicity.”

“Conventionally employed methods for imparting antithrombogenicity to medical materials are generally classified into three groups of (1) immobilizing a mucopolysaccharide (e.g., heparin) or a plasminogen activator (e.g., urokinase) on the surface of a material, (2) modifying the surface of a material so that it carries negative charge or hydrophilicity, and (3) inactivating the surface of a material. Of these, the method of (1) (hereinafter to be referred to briefly as surface heparin method) is further subdivided into the methods of (A) blending of a polymer and an organic solvent-soluble heparin, (B) coating of the material surface with an organic solvent-soluble heparin, (C) ionical bonding of heparin to a cationic group in the material, and (D) covalent bonding of a material and heparin.”

“Of the above methods, the methods (2) and (3) are capable of affording a stable antithrombogenicity during a long-term contact with body fluids, since protein adsorbs onto the surface of a material to form a biomembrane-like surface. At the initial stage when the material has been introduced into the body (blood contact site) and when various coagulation factors etc. in the body have been activated, however, it is difficult to achieve sufficient antithrombogenicity without an anticoagulant therapy such as heparin administration.”

Other antithrombogenic methods and compositions are also well known. Thus, by way of further illustration, United States published patent application 20010016611 discloses an antithrombogenic composition comprising an ionic complex of ammonium salts and heparin or a heparin derivative, said ammonium salts each comprising four aliphatic alkyl groups bonded thereto, wherein an ammonium salt comprising four aliphatic alkyl groups having not less than 22 and not more than 26 carbon atoms in total is contained in an amount of not less than 5% and not more than 80% of the total ammonium salt by weight. The entire disclosure of this published patent application is hereby incorporated by reference into this specification.

Thus, e.g., U.S. Pat. No. 5,783,570 discloses an organic solvent-soluble mucopolysaccharide consisting of an ionic complex of at least one mucopolysaccharide (preferably heparin or heparin derivative) and a quaternary phosphonium, an antibacterial antithrombogenic composition comprising said organic solvent-soluble mucopolysaccharide and an antibacterial agent (preferably an inorganic antibacterial agent such as silver zeolite), and to a medical material comprising said organic solvent soluble mucopolysaccharide. The organic solvent-soluble mucopolysaccharide, and the antibacterial antithrombogenic composition and medical material containing same are said to easily impart antithrombogenicity and antibacterial property to a polymer to be a base material, which properties are maintained not only immediately after preparation of the material but also after long-term elution. The entire disclosure of this United States patent is hereby incorporated by reference into this specification.

By way of further illustration, U.S. Pat. No. 5,049,393 discloses anti-thrombogenic compositions, methods for their production and products made therefrom. The anti-thrombogenic compositions comprise a powderized anti-thrombogenic material homogeneously present in a solidifiable matrix material. The anti-thrombogenic material is preferably carbon and more preferably graphite particles. The matrix material is a silicon polymer, a urethane polymer or an acrylic polymer. The entire disclosure of this United States patent is hereby incorporated by reference into this specification.

By way of yet further illustration, U.S. Pat. No. 5,013,717 discloses a leach resistant composition that includes a quaternary ammonium complex of heparin and a silicone. A method for applying a coating of the composition to a surface of a medical article is also disclosed in the patent. Medical articles having surfaces that are both lubricious and antithrombogenic are produced in accordance with the method of the patent The entire disclosure of this United States patent is hereby incorporated by reference into this specification.

A Process for Preparation of an Iron-Containing Thin Film

In one preferred embodiment of the invention, a sputtering technique is used to prepare an AlFe thin film as well as comparable thin films containing other atomic moieties, such as, e.g., elemental nitrogen, and elemental oxygen. Conventional sputtering techniques may be used to prepare such films by sputtering. See, for example, R. Herrmann and G. Brauer, “D.C.- and R. F. Magnetron Sputtering,” in the “Handbook of Optical Properties: Volume I—Thin Films for Optical Coatings,” edited by R. E. Hummel and K. H. Guenther (CRC Press, Boca Raton, Fla., 1955). Reference also may be had, e.g., to M. Allendorf, “Report of Coatings on Glass Technology Roadmap Workshop,” Jan. 18-19, 2000, Livermore, Calif.; and also to U.S. Pat. No. 6,342,134, “Method for producing piezoelectric films with rotating magnetron sputtering system.” The entire disclosure of each of these prior art documents is hereby incorporated by reference into this specification.

Although the sputtering technique is advantageously used, the plasma technique described elsewhere in this specification also may be used. Alternatively, or additionally, one or more of the other forming techniques described elsewhere in this specification also may be used.

One may utilize conventional sputtering devices in this process. By way of illustration and not limitation, a typical sputtering system is described in U.S. Pat. No. 5,178,739, the entire disclosure of which is hereby incorporated by reference into this specification. As is disclosed in this patent, “ . . . a sputter system 10 includes a vacuum chamber 20, which contains a circular end sputter target 12, a hollow, cylindrical, thin, cathode magnetron target 14, a RF coil 16 and a chuck 18, which holds a semiconductor substrate 19. The atmosphere inside the vacuum chamber 20 is controlled through channel 22 by a pump (not shown). The vacuum chamber 20 is cylindrical and has a series of permanent, magnets 24 positioned around the chamber and in close proximity therewith to create a multiple field configuration near the interior surface 15 of target 12. Magnets 26, 28 are placed above end sputter target 12 to also create a multipole field in proximity to target 12. A singular magnet 26 is placed above the center of target 12 with a plurality of other magnets 28 disposed in a circular formation around magnet 26. For convenience, only two magnets 24 and 28 are shown. The configuration of target 12 with magnets 26, 28 comprises a magnetron sputter source 29 known in the prior art, such as the Torus-10E system manufactured by K. Lesker, Inc. A sputter power supply 30 (DC or RF) is connected by a line 32 to the sputter target 12. A RF supply 34 provides power to RF coil 16 by a line 36 and through a matching network 37. Variable impedance 38 is connected in series with the cold end 17 of coil 16. A second sputter power supply 39 is connected by a line 40 to cylindrical sputter target 14. A bias power supply 42 (DC or RF) is connected by a line 44 to chuck 18 in order to provide electrical bias to substrate 19 placed thereon, in a manner well known in the prior art.”

By way of yet further illustration, other conventional sputtering systems and processes are described in U.S. Pat. Nos. 5,569,506 (a modified Kurt Lesker sputtering system), U.S. Pat. No. 5,824,761 (a Lesker Torus 10 sputter cathode), U.S. Pat. Nos. 5,768,123, 5,645,910, 6,046,398 (sputter deposition with a Kurt J. Lesker Co. Torus 2 sputter gun), U.S. Pat. Nos. 5,736,488, 5,567,673, 6,454,910, and the like. The entire disclosure of each of these United States patents is hereby incorporated by reference into this specification.

By way of yet further illustration, one may use the techniques described in a paper by Xingwu Wang et al. entitled “Technique Devised for Sputtering AlN Thin Films,” published in “the Glass Researcher,” Volume 11, No. 2 (Dec. 12, 2002).

In one preferred embodiment, a magnetron sputtering technique is utilized, with a Lesker Super System III system The vacuum chamber of this system is preferably cylindrical, with a diameter of approximately one meter and a height of approximately 0.6 meters. The base pressure used is from about 0.001 to 0.0001 Pascals. In one aspect of this process, the target is a metallic FeAl disk, with a diameter of approximately 0.1 meter. The molar ratio between iron and aluminum used in this aspect is approximately 70/30. Thus, the starting composition in this aspect is almost non-magnetic. See, e.g., page 83 (FIG. 3.1aii) of R. S. Tebble et al.'s “Magnetic Materials” (Wiley-Interscience, New York, N.Y., 1969); this Figure discloses that a bulk composition containing iron and aluminum with at least 30 mole percent of aluminum (by total moles of iron and aluminum) is substantially non-magnetic.

In this aspect, to fabricate FeAl films, a DC power source is utilized, with a power level of from about 150 to about 550 watts (Advanced Energy Company of Colorado, model MDX Magnetron Drive). The sputtering gas used in this aspect is argon, with a flow rate of from about 0.0012 to about 0.0018 standard cubic meters per second. To fabricate FeAlN films in this aspect, in addition to the DC source, a pulse-forming device is utilized, with a frequency of from about 50 to about 250 MHz (Advanced Energy Company, model Sparc-le V). One may fabricate FeAl0 films in a similar manner but using oxygen rather than nitrogen.

In this aspect, a typical argon flow rate is from about (0.9 to about 1.5)×10⁻3 standard cubic meters per second; a typical nitrogen flow rate is from about (0.9 to about 1.8)×10⁻3 standard cubic meters per second; and a typical oxygen flow rate is from about. (0.5 to about 2)×10⁻³ standard cubic meters per second. During fabrication, the pressure typically is maintained at from about 0.2 to about 0.4 Pascals. Such a pressure range has been found to be suitable for nanomagnetic materials fabrications.

In this aspect, the substrate used may be either flat or curved. A typical flat substrate is a silicon wafer with or without a thermally grown silicon dioxide layer, and its diameter is preferably from about 0.1 to about 0.15 meters. A typical curved substrate is an aluminum rod or a stainless steel wire, with a length of from about 0.10 to about 0.56 meters and a diameter of from (about 0.8 to about 3.0)×10⁻³ meters The distance between the substrate and the target is preferably from about 0.05 to about 0.26 meters.

In this aspect, in order to deposit a film on a wafer, the wafer is fixed on a substrate holder. The substrate may or may not be rotated during deposition. In one embodiment, to deposit a film on a rod or wire, the rod or wire is rotated at a rotational speed of from about 0.01 to about 0.1 revolutions per second, and it is moved slowly back and forth along its symmetrical axis with a maximum speed of about 0.01 meters per second.

In this aspect, to achieve a film deposition rate on the flat wafer of 5×10⁻10 meters per second, the power required for the FeAl film is 200 watts, and the power required for the FeAlN film is 500 watts The resistivity of the FeAlN film is approximately one order of magnitude larger than that of the metallic FeAl film. Similarly, the resistivity of the FeAl0 film is about one order of magnitude larger than that of the metallic FeAl film.

Iron containing magnetic materials, such as FeAl, FeAlN and FeAl0, may be fabricated by sputtering. The magnetic properties of those materials vary with stoichiometric ratios, particle sizes, and fabrication conditions; see, e.g., R. S. Tebble and D. J. Craik, “Magnetic Materials”, pp. 81-88, Wiley-Interscience, New York, 1969 As is disclosed in this reference, when the iron molar ratio in bulk FeAl materials is less than 70 percent or so, the materials will no longer exhibit magnetic properties.

However, it has been discovered that, in contrast to bulk materials, a thin film material often exhibits different properties.

In one embodiment, the magnetic material A is dispersed within nonmagnetic material B. This embodiment is depicted schematically in FIG. 4.

Referring to FIG. 4, and in the preferred embodiment depicted therein, it will be seen that A moieties 102, 104, and 106 are preferably separated from each other either at the atomic level and/or at the nanometer level. The A moieties may be, e.g., A atoms, clusters of A atoms, A compounds, A solid solutions, etc. Regardless of the form of the A moiety, it preferably has the magnetic properties described hereinabove.

In the embodiment depicted in FIG. 4, each A moiety preferably produces an independent magnetic moment. The coherence length (L) between adjacent A moieties is, on average, preferably from about 0.1 to about 100 nanometers and, more preferably, from about 1 to about 50 nanometers.

Thus, referring again to FIG. 4, the normalized magnetic interaction between adjacent A moieties 102 and 104, and also between 104 and 106, is preferably described by the formula M=exp(−x/L), wherein M is the normalized magnetic interaction, exp is the base of the natural logarithm (and is approximately equal to 2.71828), x is the distance between adjacent A moieties, and L is the coherence length. M, the normalized magnetic interaction, preferably ranges from about 3×10⁻⁴⁴ to about 1.0. In one preferred embodiment, M is from about 0.01 to 0.99. In another preferred embodiment, M is from about 0.1 to about 0.9.

In one embodiment, and referring again to FIG. 4, x is preferably measured from the center 101 of A moiety 102 to the center 103 of A moiety 104; and x is preferably equal to from about 0.00001 times L to about 100 times L.

In one embodiment, the ratio of x/L is at least 0.5 and, preferably, at least 1.5.

In one embodiment, the “ABC particles” of nanomagentic material also have a specified coherence length. This embodiment is depicted in FIG. 4A.

As is used with regard to such “ABC particles,” the term “coherence length” refers to the smallest distance 111 between the surfaces 113 of any particles 115 that are adjacent to each other. It is preferred that such coherence length, with regard to such ABC particles, be less than about 100 nanometers and, preferably, less than about 50 nanometers. In one embodiment, such coherence length is less than about 20 nanometers.

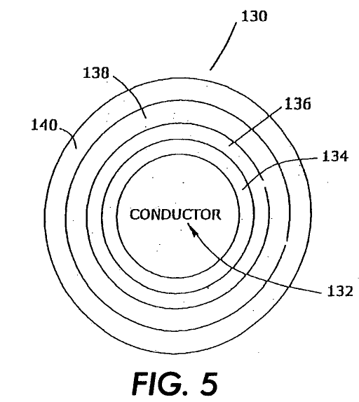

FIG. 5 is a schematic sectional view, not drawn to scale, of a shielded conductor assembly 130 that is comprised of a conductor 132 and, disposed around such conductor, a film 134 of nanomagnetic material. The conductor 132 preferably has a resistivity at 20 degrees Centigrade of from about 1 to about 100-microohom-centimeters.

The film 134 is comprised of nanomagnetic material that preferably has a maximum dimension of from about 10 to about 100 nanometers. The film 134 also preferably has a saturation magnetization of from about 200 to about 26,000 Gauss and a thickness of less than about 2 microns. In one embodiment, the magnetically shielded conductor assembly 130 is flexible, having a bend radius of less than 2 centimeters. Reference may be had, e.g., to U.S. Pat. No. 6,506,972, the entire disclosure of which is hereby incorporated by reference into this specification.

As used in this specification, the term flexible refers to an assembly that can be bent to form a circle with a radius of less than 2 centimeters without breaking. Put another way, the bend radius of the coated assembly is preferably less than 2 centimeters. Reference may be had, e.g., to U.S. Pat. Nos. 4,705,353, 5,946,439, 5,315,365, 4,641,917, 5,913,005, and the like. The entire disclosure of each of these United States patents is hereby incorporated by reference into this specification.

Without wishing to be bound to any particular theory, applicants believe that the use of nanomagnetic materials in their coatings and their articles of manufacture allows one to produce a flexible device that otherwise could not be produced were not the materials so used nano-sized (less than 100 nanometers).

Referring again to FIG. 5, and in the preferred embodiment depicted therein, one or more electrical filter circuit(s) 136 are preferably disposed around the nanomagnetic film 134. These circuit(s) may be deposited by conventional means.

In one embodiment, the electrical filter circuit(s) are deposited onto the film 134 by one or more of the techniques described in U.S. Pat. Nos. 5,498,289 (apparatus for applying narrow metal electrode), U.S. Pat. No. 5,389,573 (method for making narrow metal electrode), U.S. Pat. No. 5,973,573 (method of making narrow metal electrode), U.S. Pat. No. 5,973,259 (heated tool positioned in the X, Y, and 2-directions for depositing electrode), U.S. Pat. No. 5,741,557 (method for depositing fine lines onto a substrate), and the like. The entire disclosure of each of these United States patents is hereby incorporated by reference into this specification.

Referring again to FIG. 5, and in the preferred embodiment depicted therein, disposed around electrical filter circuit(s) 136 is a second film of nanomagnetic material 138, which may be identical to or different from film layer 134. In one embodiment, film layer 138 provides a different filtering response to electromagnetic waves than does film layer 134.

Disposed around nanomagnetic film layer 138 is a second layer of electrical filter circuit(s) 140. Each of circuit(s) 136 and circuit(s) 140 comprises at least one electrical circuit. It is preferred that the at least two circuits that comprise assembly 130 provide different electrical responses.

As is known to those skilled in the art, at high frequencies the inductive reactance of a coil is great. The inductive reactance (X_{L}) is equal to 2πFL, wherein F is the frequency (in hertz), and L is the inductance (in Henries).

At low-frequencies, by comparison, the capactitative reactance (X_{C}) is high, being equal to 1/2πFC, wherein C is the capacitance in Farads. The impedance of a circuit, Z, is equal to the square root of (R²+[X_{L}−X_{C}]²), wherein R is the resistance, in ohms, of the circuit, and X_{L} and X_{C} are the inductive reactance and the capacitative reactance, respectively, in ohms, of the circuit.

Thus, for any particular alternating frequency electromagnetic wave, one can, by the appropriate selection of values for R, L, and C, pick a circuit that is purely resistive (in which case the inductive reactance is equal to the capacitative reactance at that frequency), is primarily inductive, or is primarily capacitative.

Maximum power transfer occurs at resonance, when the inductance reactance is equal to the capactitative reactance and the difference between them is zero. Conversely, minimum power transfer occurs when the circuit has little resistance in it (all circuits have some finite resistance) but is predominantly inductive or predominantly capacitative.

An LC tank circuit is an example of a circuit in which minimum power is transmitted. A tank circuit is a circuit in which an inductor and capacitor are in parallel; such a circuit appears, e.g., in the output stage of a radio transmitter.

An LC tank circuit exhibits the well-known flywheel effect, in which the energy introduced into the circuit continues to oscillate between the capacitor and inductor after an input signal has been applied; the oscillation stops when the tank-circuit finally loses the energy absorbed, but it resumes when a new source of energy is applied. The lower the inherent resistance of the circuit, the longer the oscillation will continue before dying out.

A typical tank circuit is comprised of a parallel-resonant circuit; and it acts as a selective filter. As is known to those skilled in the art, and as is disclosed in Stan Gibilisco's “Handbook of Radio & Wireless Technology” (McGraw-Hill, New York, N.Y., 1999), a selective filter is a circuit designed to tailor the way an electronic circuit or system responds to signals at various frequencies (see page 62).

The selective filter may be a bandpass filter (see pages 62-63 of the Gibilisco book) that comprises a resonant circuit, or a combination of resonant circuits, designed to discriminate against all frequencies except a specified frequency, or a band of frequencies between two limiting frequencies. In a parallel LC circuit, a bandpass filter shows a high impedance at the desired frequency or frequencies and a low impedance at unwanted frequencies. In a series LC configuration, the filter has a low impedance at the desired frequency or frequencies, and a high impedance at unwanted frequencies.

The selective filter may be a band-rejection filter, also known as a band-stop filter (see pages 63-65 of the Gibilisco book). This band-rejection filter comprises a resonant circuit adapted to pass energy at all frequencies except within a certain range. The attenuation is greatest at the resonant frequency or within two limiting frequencies.

The selective filter may be a notch filter; see page 65 of the Gibilisco book. A notch filter is a narrowband-rejection filter. A properly designed notch filter can produce attenuation in excess of 40 decibels in the center of the notch.

The selective filter may be a high-pass filter; see pages 65-66 of the Gibilisco book. A high-pass filter is a combination of capacitance, inductance, and/or resistance intended to produce large amounts of attenuation below a certain frequency and little or no attenuation above that frequency. The frequency above which the transition occurs is called the cutoff frequency.

The selective filter may be a low-pass filter; see pages 67-68 of the Gibilisco book. A low-pass filter is a combination of capacitance, inductance, and/or resistance intended to produce large amounts of attenuation above a certain frequency and little or no attenuation below that frequency.

In the embodiment depicted in FIG. 5, the electrical circuit is preferably integrally formed with the coated conductor construct. In another embodiment, not shown in FIG. 5, one or more electrical circuits are separately formed from a coated substrate construct and then operatively connected to such construct.

FIG. 6A is a sectional schematic view of one preferred shielded assembly 131 that is comprised of a conductor 133 and, disposed around such conductor 133, a layer of nanomagnetic material 135.

In the embodiment depicted in FIG. 6A, the layer 135 of nanomagnetic material preferably has a thickness 137 of at least 150 nanometers and, more preferably, at least about 200 nanometers. In one embodiment, the thickness of layer 135 is from about 500 to about 1,000 nanometers.

The layer 135 of nanomagnetic material 137 preferably is comprised of nanomagnetic material that may be formed, e.g., by subjecting the material in layer 137 to a magnetic field of from about 10 Gauss to about 40 Tesla for from about 1 to about 20 minutes. The layer 135 preferably has a mass density of at least about 0.001 grams per cubic centimeter (and preferably at least about 0.01 grams per cubic centimeter), a saturation magnetization of from about 1 to about 36,000 Gauss, and a coercive force of from about 0.01 to about 5,000.

In one embodiment, the B moiety is added to the nanomagnetic A moiety, preferably with a B/A molar ratio of from about 5:95 to about 95:5 (see FIG. 3). In one aspect of this embodiment, the resistivity of the mixture of the B moiety and the A moiety is from about 1 micro-ohm-cm to about 10,000 micro-ohm-cm.

Without wishing to be bound to any particular theory, applicants believe that such a mixture of the A and B moieties provides two mechanisms for shielding the magnetic fields. One such mechanism/effect is the shielding provided by the nanomagnetic materials, described elsewhere in this specification. The other mechanism/effect is the shielding provided by the electrically conductive materials.

In one particularly preferred embodiment, the A moiety is iron, the B moiety is aluminum, and the molar ratio of A/B is about 70:30; the resistivity of this mixture is about 8 micro-ohms-cm.

FIG. 6B is a schematic sectional view of a magnetically shielded assembly 139 that is similar to assembly 131 but differs therefrom in that a layer 141 of nanoelectrical material is disposed around layer 135.

The layer of nanoelectrical material 141 preferably has a thickness of from about 0.5 to about 2 microns. In this embodiment, the nanoelectrical material comprising layer 141 has a resistivity of from about 1 to about 100 microohm-centimeters. As is known to those skilled in the art, when nanoelectrical material is exposed to electromagnetic radiation, and in particular to an electric field, it will shield the substrate over which it is disposed from such electrical field. Reference may be had, e.g., to International patent publication WO9820719 in which reference is made to U.S. Pat. No. 4,963,291; each of these patents and patent applications is hereby incorporated by reference into this specification.

As is disclosed in U.S. Pat. No. 4,963,291, one may produce electromagnetic shielding resins comprised of electroconductive particles, such as iron, aluminum, copper, silver and steel in sizes ranging from 0.5 to. 50 microns. The entire disclosure of this United States patent is hereby incorporated by reference into this specification.

The nanoelectrical particles used in this aspect of the invention preferably have a particle size within the range of from about 1 to about 100 microns, and a resistivity of from about 1.6 to about 100 microohm-centimeters. In one embodiment, such nanoelectrical particles comprise a mixture of iron and aluminum. In another embodiment, such nanoelectrical particles consist essentially of a mixture of iron and aluminum.

It is preferred that, in such nanoelectrical particles, and in one embodiment, at least 9 moles of aluminum are present for each mole of iron. In another embodiment, at least about 9.5 moles of aluminum are present for each mole of iron. In yet another embodiment, at least 9.9 moles of aluminum are present for each mole of iron.

In one embodiment, and referring again to FIG. 6D, the layer 141 of nanoelectrical material has a thermal conductivity of from about 1 to about 4 watts/centimeter-degree Kelvin.

In one embodiment, not shown, in either or both of layers 135 and 141 there is present both the nanoelectrical material and the nanomagnetic material One may produce such a layer 135 and/or 141 by simultaneously depositing the nanoelectrical particles and the nanomagnetic particles with, e.g., sputtering technology such as, e.g., the sputtering technology described elsewhere in this specification.

FIG. 6C is a sectional schematic view of a magnetically shielded assembly 143 that differs from assembly 131 in that it contains a layer 145 of nanothermal material disposed around the layer 135 of nanomagnetic material. The layer 145 of nanothermal material preferably has a thickness of less than 2 microns and a thermal conductivity of at least about 150 watts/meter-degree Kelvin and, more preferably, at least about 200 watts/meter-degree Kelvin. It is preferred that the resistivity of layer 145 be at least about 10¹⁰ microohm-centimeters and, more preferably, at least about 10¹² microohm-centimeters. In one embodiment, the resistivity of layer 145 is at least about 10¹³ microohm centimeters. In one embodiment, the nanothermal layer is comprised of AlN.

In one embodiment, depicted in FIG. 6C, the thickness 147 of all of the layers of material coated onto the conductor 133 is preferably less than about 20 microns.

In FIG. 6D, a sectional view of an assembly 149 is depicted that contains, disposed around conductor 133, layers of nanomagnetic material 135, nanoelectrical material 141, nanomagnetic material 135, and nanoelectrical material 141.

In FIG. 6E, a sectional view of an assembly 151 is depicted that contains, disposed around conductor 133, a layer 135 of nanomagnetic material, a layer 141 of nanoelectrical material, a layer 135 of nanomagnetic material, a layer 145 of nanotherrnal material, and a layer 135 of nanomagnetic material. Optionally disposed in layer 153 is antithrombogenic material that is biocompatible with the living organism in which the assembly 151 is preferably disposed.

In the embodiments depicted in FIGS. 6A through 6E, the coatings 135, and/or 141, and/or 145, and/or 153, are disposed around a conductor 133. In one embodiment, the conductor so coated is preferably part of medical device, preferably an implanted medical device (such as, e.g., a pacemaker). In another embodiment, in addition to coating the conductor 133, or instead of coating the conductor 133, the actual medical device itself is coated.

Filter Circuits that may be used with the Coating Constructs of the Invention.

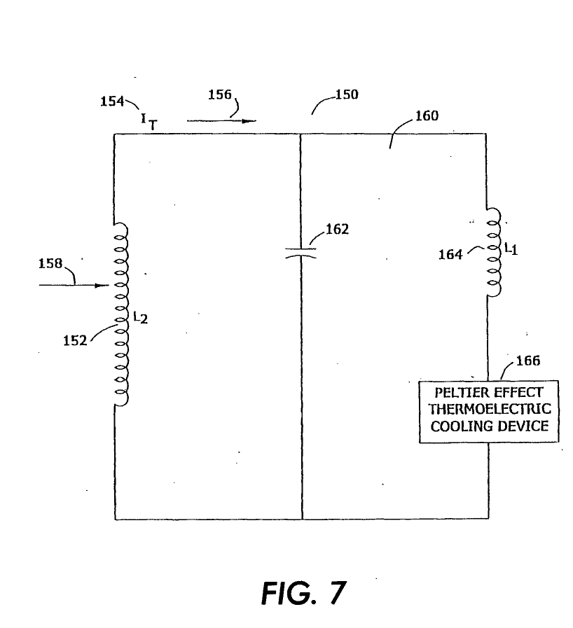

Many different electrical circuits, such as filter circuits, may be used in conjunction with the coating constructs of this invention. One such preferred filter circuit is illustrated in FIG. 7.

In the filter circuit 150 depicted in FIG. 7, a large coil 152 is chosen so that it generates a substantial amount of current 154 (I_{T}) when exposed to the high-frequency electromagnetic wave produced during, e.g., an MRI process. This current 154 flowing in the direction of arrow 156 supplies energy to the resonant circuit 160 defined by capacitor 162, inductor 164, and load 166.

In the embodiment depicted in FIG. 7, the load 166 is preferably a thermoelectric cooling device. As is known to those skilled in the art, thermoelectric cooling is cooling based upon the Peltier effect. An electric current is sent to a thermocouple whose cold junction is thermally coupled to a substrate to be cooled, while the hot junction dissipates heat to the surroundings. In the Peltier effect, heat is absorbed when current is sent through a junction of two dissimilar metals. See, e.g., page 1917 of the McGraw-Hill Dictionary of Scientific and Technical Terms, Fourth Edition (McGraw-Hill Book Company, New York, N.Y., 1989).

Thermoelectric coolers are often used to maintain a constant temperature; see, e.g., U.S. Pat. Nos. 5,313,333, 4,628,277, 5,347,869, 6,4445,487, 5,956,569, 5,930,430, 5,717,804, 5,596,228, 5,561,685, 6,240,113, 6,107,6390, and the like. The entire disclosure of each of these United States patents is hereby incorporated by reference into this specification.

By way of illustration and not limitation, U.S. Pat. No. 5,956,569 discloses an integrated thermoelectric cooler formed on the backside of a substrate. It appears that the device of this patent requires a direct current input; thus, one may utilize an appropriate D.C. power supply adapted to convert the alternating current to the required direct current.

From the foregoing, it will be apparent that, for each of the electromagnetic radiations produced during, e.g., a magnetic resonance imaging (MRI) process, one may utilize a series of energy-modifying devices to minimize the extent to which that particular electromagnetic radiation heats a particular substrate. Thus, e.g., one may convert much of the energy in the particular radiation into energy required to sustain a flywheel effect. Thus, e.g., one may absorb some of the energy (which will cause an increase in heat) and, with another portion of the energy, drive a thermoelectric cooler to cool the device, so that the neat heat change is zero.

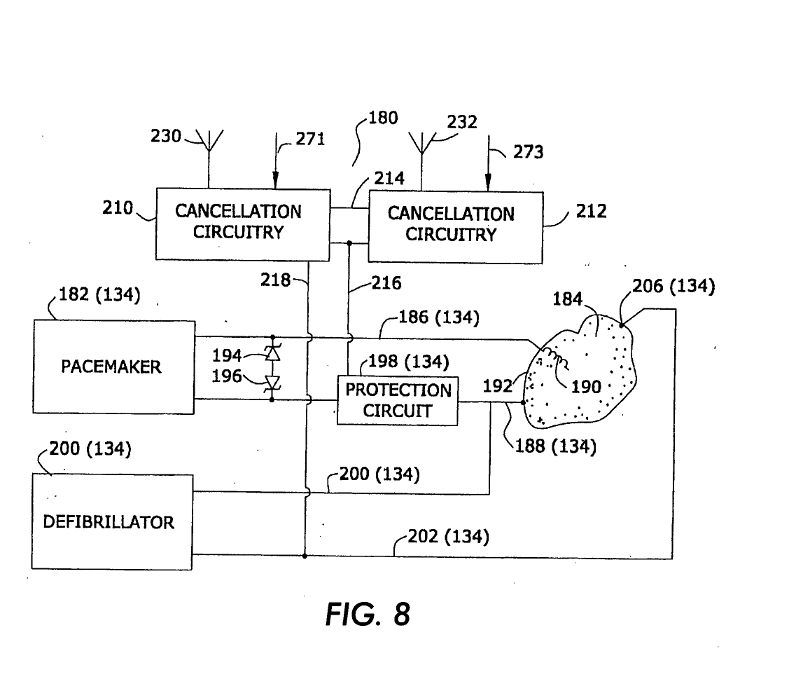

One may combine one or more selective filtering devices together with one or more of the nanomagnetic constructs of this invention to provide an assembly that is more effective in protecting against the adverse effects of high-frequency electromagnetic radiation that either device by itself. One such combined device is illustrated in FIG. 8.

FIG. 8 is a schematic of a magnetically shielded assembly 180 that is similar to the device depicted in FIG. 1 of U.S. Pat. No. 4,745,923. The entire disclosure of such U.S. Pat. No. 4,745,923 is hereby incorporated by reference into this specification. This patent describes and claims: “An apparatus for protecting an implantable electrical device having a plurality of electrically conductive terminals, including output and return terminals and electrically conductive leads connected to said terminals against excessive currents comprising: means connected to form an electrically conductive low-impedance path for connection in circuit with at least one of said leads; means connected to form an electrically conductive high-impedance path for connection in circuit with said at least one lead; means for generating a signal representative of the current flowing in said low-impedance path; switch means for opening and closing said low-impedance path; and means responsive to said signal representative of said current for controlling said switch means to open said low-impedance path when said current exceeds a predetermined level so that said current flows in said high-impedance path, whereby the current flowing into said electrical device is limited to a safe level.”

As is disclosed in U.S. Pat. No. 4,745,923, “The invention disclosed herein relates generally to protection devices used to protect other devices from damage or destruction resulting from voltage or current surges. In particular, the present invention relates to such a protection device which is implantable in the body of a patient with a heart pacemaker to protect the pacemaker against current surges, particularly those resulting from the operation of an external or implanted heart defibrillator.”

“It is well known that in many instances an implanted heart pacemaker can successfully regulate the otherwise faulty operation of a damaged or diseased heart. Generally, a typical pacemaker senses electrical activity or lack of such activity in the heart muscle, and supplies electrical stimulus pulses to the heart to stimulate contractions when necessary. The electrical stimulus pulses generated by a pacemaker, however, are ineffective to stop the lethal condition of fibrillation. However, it is well known that the application of a series of high-voltage pulses to the heart is often effective in arresting fibrillation. Of course it is desirable following defibrillation of the heart for the pacemaker to resume its normal regulatory role. A serious problem in this regard, however, is that without adequate protection against the large current flow induced by the application of high-voltage defibrillation pulses to the heart, a pacemaker can be damaged or destroyed. Obviously, from the standpoint of the patient's continued well being, this is a totally unacceptable consequence.”

“In the past, a number of attempts have been made to provide adequate protection against excessive currents and voltages for pacemakers and other medical devices such as electrocardiogram (ECG) amplifiers. For example, it is known to connect one or more zener diodes between the opposite leads of a pacemaker to limit the voltage differential therebetween.”

“However, as discussed in U.S. Pat. No. 4,320,763 to Money, this approach is not effective to limit the current flow between the heart tissue and the electrode at the distal end of the pacemaker lead. As a result, the heart tissue near the point of contact with the electrode can be severely damaged when high-voltage defibrillation pulses are applied to the heart. The U.S. Pat. No. 4,320,763 discloses that such tissue damage can be prevented by connecting a current limiting device such as a diode or a pair of field effect transistors (FETs) in series between a pacemaker output terminal and a distal electrode. However, it is apparent that the current limiting device thereby becomes a permanent part of the pacemaker circuit. When current limiting is not needed, for example during normal pacing operation, it is desirable to remove the current limiting device from the circuit to avoid unnecessary noise generation as well as loading effects.”

“An approach for protecting the pacemaker circuitry itself is disclosed in U.S. Pat. No. 4,440,172 to Langer. The U.S. Pat. No. 4,440,172 discloses an implantable pacemaker and defibrillator unit in which the pacemaker and defibrillator share common output and return lines. The pacemaker generates negative-going stimulus pulses and is protected against the positive-going high-voltage defibrillator pulses by a resistor and forward biased diode connected in series between the common output line and ground. This approach only provides limited protection to the pacemaker from unidirectional defibrillation pulses. Recent medical research has shown, however, that a number of benefits are obtained by using a bidirectional or “biphasic” pulse train to defibrillate the heart. Some of the benefits of “biphasic” defibrillation, which forms no part of the present invention, are discussed in Schuder, Defibrillation of 100 kg Calves With Asymmetrical, Bidirectional, Rectangular Pulses, Cardiovascular Research 419-426 (1984), and Jones, Decreased Defibrillator-Induced Dysfunction With Biphasic Rectangular Waveforms, Am. J. Physiol. 247 (Heart Circ. Physiol. 16): H792-H796 (1984).”

“ . . . the present invention has as an object to provide a protection device that protects both a pacemaker or other implantable device and the heart tissue near a lead thereof against damage from high current and voltage levels . . . ”

Referring again to FIG. 8, and in the preferred embodiment depicted therein, a heart pacemaker 182 implanted in the body of a patient is electrically connected in circuit with the patient's heart 184 via conventional electrically conductive pacing/sensing and return leads 186/188. Pacing/sensing lead 186 contains an electrically conductive barbed or screw-shaped pacing/sensing electrode 190 at its distal end for making firm electrical contact with the heart 184. Return lead 188 contains at its distal end a conductive patch 192 which may be sewn to the wall of the heart 184 to ensure a solid electrical connection. Electrically connected between the pacing/sensing and return leads 186,188 are oppositely polled first and second zener diodes 194, 196 to limit the voltage differential between the terminals of the pacemaker 182. First zener diode 194 preferably limits the positive voltage differential to approximately +3 volts. Second zener diode 196 preferably limits the negative differential to approximately −10 volts. A protection circuit 198 is implanted with the pacemaker 182 and is electrically connected in series with return lead 188 and patch 192 between the heart 184 and the pacemaker 182.

In addition, a defibrillator 200, which may be either an external or an implanted unit, is also electrically connected in circuit with the heart 184. If implanted, the defibrillator 200 is electrically connected to the heart 184 via conventional electrically conductive output and return leads 202,204. Output lead 202 has attached to its distal end a conductive patch 206 which may be sewn to the wall of the heart 184. In this embodiment, return lead 204 is electrically connected at its distal end by any suitable means to return lead 188 between the heart 184 and the protection circuit 198 so that the pacemaker 182 and the defibrillator 200 share a common return lead to some extent. Of course, if the defibrillator 200 is an external unit, then no direct connections to the heart 184 are present. Instead, electrically conductive paddles of a type well known to those skilled in the art are supplied externally to the chest of a patient in the vicinity of the heart 184 as output and return electrodes.

The pacemaker 182 and defibrillator 200 described above are exemplary devices only and that the protection circuit 198 comprising a presently preferred embodiment of the present invention will find use in many other applications where protection of a device against high voltages and currents is desirable.

As is illustrated in FIG. 2 of U.S. Pat. No. 4,745,923 (the entire disclosure of which is hereby incorporated by reference into this specification), the protection circuit 16 is electrically connected to conductive patch 15 via return lead 13. In series with return lead 13 are a first and a second field effect transistor (FET) 22, 23 and a 5 ohm sensing resistor 24. The drain of the second FET 23 connects to return lead 13 on the heart 11 side. The source of the second FET 23 connects to one end of the sensing resistor 24 and the source of the first FET 22 connects to the opposite end. The drain of the first FET 16 connects to the opposite end of return lead 13 on the pacemaker 10 side. The gates of the first and second FETs 22,23 are connected in parallel to one end of a 390K ohm current limiting resistor 29 and to the collectors of first and second parallel bipolar transistors 25,26. The other end of the 390K ohm current limiting resistor 29 connects to a DC voltage source 30.

In one preferred embodiment, illustrated in FIG. 8, one or more of the pacemaker 182, the defibrillator 200, the leads 186 and 188, the protection circuit l98, the leads 202 and 204, and the patches 192 and 206 are coated with film 134 of nanomagnetic material (see FIG. 5). This is indicated by the use of “(134)” after the element in question. Thus, e.g., “186(134)” indicates that lead 186 is coated with nanomagnetic film 134.

In another embodiment, not shown, one or more of the pacemaker 182, the defibrillator 200, the leads 186 and 188, the protection circuit 198, the leads 202 and 204, and the patches 192 and 206 are coated with film (not shown) that is comprised of nanomagnetic material and, optionally, one more more of dielectric material, insulative material, thermal material, etc. Thus, e.g., one or more of the one or more of the pacemaker 182, the defibrillator 200, the leads 186 and 188, the protection circuit 198, the leads 202 and 204, and the patches 192 and 206 may be coated with one or more of the constructs illustrated in FIGS. 5 and/or 6A through 6E.

Referring again to the preferred embodiment depicted in FIG. 8, the film 134 that is disposed about one or more of the components of the assembly 180 is preferably comprised of at least about 30 weight percent of nanomagnetic material with a mass density of at least about 0.01 grams per cubic centimeter, a saturation magnetization of from about 1 to about 36,000 Gauss, a coercive force of from about 0.01 to about 5,000 Oersteds, a relative magnetic permeability of from about 1 to about 500,000, and an average particle size of less than about 100 nanometers U.S. Pat. No. 4,745,923 discloses but one type of current-limiting protection circuit that may be used in the assembly 180 of FIG. 8. One may use other such protection circuits disclosed in the prior art.

Thus, by way of illustration and not limitation, U.S. Pat. No. 4,320,763 discloses a device for preventing tissue damage when high-currents flow through the tissue as a result of high voltage differentials. The patent claims: “In a pacemaker assembly comprising pulse-generator means for generating electrical pulses and electrode means having a proximal end coupled to said pulse-generating means and a distal end designed to be placed adjacent to body tissue for delivering said pulses to said tissue, the improvement comprising: current-limiting means coupled in series with said pulse-generating means and said electrode means for permitting passage of said electrical pulses to said tissue and for protecting said tissue against tissue damaging current flow between said distal end of said electrode means and said tissue as may occur with cardioversion.”

The object of the invention claimed in U.S. Pat. No. 4,320,763 was set forth in column 1 of the patent, wherein it was stated that: “It is therefore an object of the present invention to protect the heart tissue of a pacemaker implantee form damage upon application of high voltages to the users body.” The entire disclosure of this United States patent is hereby incorporated by reference into this specification.

By way of further illustration, U.S. Pat. No. 5,197,468 discloses a “device for protecting an electronic prosthesis from adverse effects of RF . . . energy.” This device includes “ . . . a Ferrite body electrically and thermally connected to the lead wire and to a ground element.”

In particular, U.S. Pat. No. 5,197,468 discloses and claims: “an electronic prosthesis that is implantable into a user's body including: A) an electronic device that is implantable into a user's body and includes a dc power source, electronic control elements, tissue stimulating elements and an electronic lead wire electrically connecting said power source, said electronic control elements and said tissue stimulating elements; and B) a protective device for protecting said electronic device from undesired RF energy induced operation and from undesired electrostatic energy induced operation, said protective device including (1) a ground element having a first impedance and electrically separated from said lead wire be said first impedance, and (2) an impedance element in said lead wire connected between said dc power source and said tissue stimulating elements having an impedance that is greater than said first impedance when exposed to RF energy.” The entire disclosure of this United States patent is hereby incorporated by reference into this specification.

As is disclosed in column 3 of U.S. Pat. No. 5,197,468, “ . . . such external influences as RF energy . . . have been identified as causing problems with artificial cardiac pacers . . . . The literature is replete with examples of cardiac pacer malfunctions traced to . . . MRI techniques . . . .”

By way of further illustration, U.S. Pat. No. 5,584,870 discloses a device for protecting a cochlear implant from external electrostatic charges. The entire disclosure of this United States patent is hereby incorporated by reference into this specification.

By way of further illustration, U.S. Pat. No. 5,833,710 provides a device for protecting cardiac tissue near low energy implanted electrodes; the entire disclosure of this United States patent is hereby incorporated by reference into this specification.

There is disclosed and claimed in this patent: “An implantable medical device comprising: an electronic circuit operable to provide low energy cardiac tissue stimulation and detection and at least two inputs to receive respectively, at least two low energy stimulation and detection electrodes, wherein the electronic circuitry has a reference potential as a system ground which is isolated from an earth ground; and an automatic, unidirectional current limiting circuit interposed in series between said electronic circuitry and each input and coupled to said reference potential, said automatic unidirectional current limiting circuitry having a protected output connected to said electronic circuitry and an unprotected input.”

As is disclosed in column 3 of U.S. Pat. No. 5,833,710, “ . . . the present invention pertains to protecting the circuitry connected to the low energy leads, and protecting the patient's tissue at the low energy lead sites, from the high energy pulses . . . and from high energy pulses from other medical electronic devices . . . .”

By way of yet further illustration, U.S. Pat. No. 5,591,218 describes a “current limiter for implantable electronic device lead” which, like the device of U.S. Pat. No. 5,833,710, “ . . . protects cardiac tissue near the low energy electrodes” (see the abstract); the entire disclosure of this U.S. Pat. No. 5,591,218 is hereby incorporated by reference into this specification. This patent discloses and claims: “A unidirectional current limiting circuit for use in series with the lead of an implanted medical device having low energy stimulation and detection electrodes, comprising: an unprotected input and a protected output; a current flow from the unprotected input to the protected output; a reference potential corresponding to a ground potential; a bias voltage; a first switch having an open circuit condition, a current limiting condition, and a closed circuit condition, the first switch having an input connected to the unprotected input and an output; a low value resistor connected to the output of the first switch producing a first voltage in response to said current flow through the first switch; a second switch having an open circuit condition and a closed circuit condition the second switch being operatively connected between the bias voltage and the protected output; a voltage divider connected to the unprotected input and the protected output, said voltage provider and producing a control voltage corresponding to a voltage across the unprotected input and the protected output; and a voltage clamp circuit connected between the reference potential and the protected output and operable to maintain the protected output voltage within a preset voltage range of the reference voltage; wherein the first switch is biased in the closed circuit condition when the voltage of the low value resistor is below the bias voltage by a first predetermined amount, the first switch is biased in the current limiting condition when the voltage of the low value resistor is not below the bias voltage by the first predetermined amount, and wherein the second switch is automatically biased in the open circuit condition when the control voltage is less than a second predetermined amount and in the closed circuit condition when the control voltage is greater than the second predetermined amount, the second switch closed circuit condition effectively lowering the bias voltage to place and maintain the first switch in the open circuit condition.”

By way of yet further illustration, one may use the “current limiter for an implantable cardiac device disclosed in U.S. Pat. No. 6,161,040, the entire disclosure of which is hereby incorporated by reference into this specification. This patent describes and claims: “A defibrillator for implantation into a patient to provide therapy to a patient's heart, comprising: a pulse generator generating selectively defibrillation pulses, said defibrillation pulses having positive and negative phases; defibrillator electrodes for delivering said defibrillation pulses to said heart; first and sensing electrodes extending to said heart; a sensing circuit sensing intrinsic activity within said heart; and a protection circuit arranged between sensing electrodes and said sensing circuit to protect said sensing circuit from an overvoltage resulting from said defibrillation pulses, said protection circuit including a first section and a second section; wherein said first section and a second section each include an electronic element arranged to limit current during said positive phase and said negative, respectively; and a biasing circuit disposed in said protection circuit and shared by said first and second sections for biasing said electronic elements.” As is disclosed in column 1 of U.S. Pat. No. 6,161,040, “ . . . because the impedance of the heart tissues through which the shocks are discharged are unknown, it is difficult to control the current delivered through the shocks. Abnormally high current levels are undesirable because a high current may damage the heart tissues.”

Referring again to FIG. 8, and in the preferred embodiment depicted therein, it will be seen that the assembly 180 is comprised of one or more cancellation circuits 210 and/or 212. These cancellation circuitries 210,212, in one embodiment, are not connected to any other circuitry or device. Alternatively, the circuits 210/212 may be connected to each other (via line 214) and/or to the protection circuit 198 (via line 216) and/or to lines 186, and/or 202 and/or 204 (via line 218), and/or to defibrillator 206 and/or to heart 184. Other possible circuit arrangements will be apparent to those skilled in the art.

The cancellation circuits 210 and 212 preferably minimize the effects of high frequency electromagnetic radiation by the mechanism of cancellation. Cancellation is the elimination of one quantity by another, as when a voltage is reduced to zero by another voltage of equal magnitude and opposite sign. See, e.g., page 91 of Stan Gibilisco's “The Illustrated Dictionary of Electronics,” Sixth Edition (Tab Books, Blue Ridge Summit, Pa., 1994).

One may use one or more of the cancellation circuits disclosed in the prior art, or variations thereof especially adapted to cancel the high-frequency electromagnetic waves present in a biological organism during MRI analyses. Some of these prior art cancellation circuits are discussed below.

U.S. Pat. No. 3,720,941 discloses a clutter cancellation circuit used in a monopulse radar system. This clutter cancellation circuit comprises: “ . . . a. means for deriving first and second signals respectively indicative of first and second reception lobe responses of a monopulse antenna; first and second channels respectively coupled to said first and second signals; signal combining means for algebraically combining the signals in said first and second channels, for providing a difference signal indicative of the algebraic difference of the signals in the said first and second channels, whereby a clutter cancelled output is provided when the phase and amplitude differences between the signals in said first and second channels are nulled; phase shifting means connected in series in said first channel for nulling the phase difference of the signals in said first and second channels; and amplitude adjusting means connected in said first channel for nulling the amplitude difference between the signals in said first and second channels.” The entire disclosure of this United States patent is hereby incorporated by reference into this specification.

U.S. Pat. No. 3,935,533 discloses a microwave transceiver comprised of a cancellation circuit. As is disclosed in claim 1 of this patent, the single oscillator microwave receiver comprises: “antenna means for transmitting and receiving microwave energy; means for coupling energy from said oscillator to said antenna means for transmission thereby, and for simultaneously coupling energy received at said antenna means and a small portion of the energy of said oscillator in mixed fashion to the input of said FM receiver; an AFC circuit connected to the output of said FM receiver; means for providing a substantially DC voltage suitable for controlling the carrier frequency of said microwave oscillator; summing means, the output of said summing means being connected to said frequency-controlling voltage input of said microwave oscillator; input means for applying transmitter input modulation to one input of said summing means; and first selectively operable means for connecting said AFC circuit or carrier voltage means to a second input of said summing means, alternatively, whereby said microwave oscillator provides a carrier frequency selectively determined by said AFC circuit or by said carrier voltage means, which is frequency modulated in accordance with said transmitter input modulation; wherein said input means includes a variable gain amplifier having a signal input and a gain control input, said signal input being connected to transmitter input modulation, the output of said variable gain amplifier being connected to the first input of said summing means; delay means responsive to transmitter input modulation for providing delayed transmitter input modulation which is delayed by a period of time substantially equal to the circuit signal propagation time from the input of said variable gain amplifier through said FM receiver; second selectively operable means responsive to the output of said FM receiver and to the output of said delay means for selectively combining said delayed transmitter input modulation with the output of said FM receiver in a voltage polarity relationship to provide a receiver output signal having transmitter input modulation substantially cancelled therefrom; and means responsive to said receiver output signal and to said delayed transmitter input modulation for providing a gain control signal to the gain control input of said variable gain amplifier, said gain control signal adjusting the gain of said variable gain amplifier so that the magnitude of transmitter input modulation included in the output of said FM receiver is adjusted with respect to the magnitude of delayed transmitter input modulation provided by said delay unit so that the transmitter input modulation in said receiver output signal is substantially nulled to zero.” The entire disclosure of this United States patent is hereby incorporated by reference into this specification.”

U.S. Pat. No. 4,535,476 discloses an offset geometry, interference canceling receiver that comprises: antenna means for receiving signals from a desired signal source and from an interference signal source located adjacent to the desired signal source, said antenna means comprising a main feedhorn which is focused on said desired signal source and an auxiliary feedhorn which is focused on said interference signal source, the antenna means being responsive to signals from the desired signal source for generating a composite signal including a desired message signal and a first interference signal, the antenna means also being responsive to signals from the interference signal source for generating a second interference signal comprising the first interference signal, combining means including a first feedback control circuit responsive to a representation of the desired message signal for generating appropriate control signals to cause variations of the phase and amplitude of the first interference signal, means responsive to the control signals for adjusting the phase and amplitude of the first interference signal, and a combiner for combining the adjusted first interference signal with the composite signal to generate said representation of the desired message signal, and signal translation means including a first duplexer coupled to the antenna means for interfacing the composite signal received therefrom, a first amplifier means for adjusting the amplitude of the composite signal to a predetermined level, a second duplexer coupled to the antenna means for interfacing the second interference signal received therefrom, and a second amplifier means for adjusting the amplitude of the second interference signal to a predetermined level. The entire disclosure of this United States patent is hereby incorporated by reference into this specification.

U.S. Pat. No. 4,698,634 discloses a subsurface insection radar signal comprised of a clutter cancellation circuit. As is disclosed in claim 1 of this patent, the clutter cancellation circuit is comprised of “ . . . clutter cancellation means operatively connected to said receiver means for eliminating internal reflections developed in said system to prevent interference by said internal reflections with the desired external reflections to enhance the system detection capability and reliability of evaluation of said external reflections, said internal reflections comprising signals generated within said system by said antenna means, said transmitter means and said receiver means.” The entire disclosure of this United States patent application is hereby incorporated by reference into this specification.

U.S. Pat. No. 5,280,290 discloses a self-oscillating mixer circuit that comprises “cancellation means for combining the IF signal with the modulating signal to cancel from the IF signal a modulation corresponding to that of the modulated RF signal, said cancellation means including a first input coupled to the output of the mixer, a second input for receiving the modulating signal, and an output for producing a demodulated signal.” The entire disclosure of this United States patent is hereby incorporated by reference into this specification.

U.S. Pat. No. 5,407,027 also discloses a “ . . . cancellation circuit for canceling offset voltage by storing, when said inverter is stopped while said current command generating circuit keeps generating the current command value, the output signal of said current detector, and by adding, when the inverter is in operation, the stored output signal to the present output signal of the current detector.” The entire disclosure of this United States patent is hereby incorporated by reference into this specification.

U.S. Pat. No. 6,008,760 discloses a cancellation system for frequency reuse in microwave communications. This patent discloses and claims: “A free-space electromagnetic wave communications system for canceling co-channel interference and transmit signal leakage, said communications system transmitting a plurality of signals from at least one transmit location to at least one receive location, said communications system utilizing spatial gain distribution processing of the transmitted signals for providing frequency-reuse of the transmitted signals, utilizing distributed frequency compensation for compensating for frequency dependent variations of transmitted and received antenna beam patterns, utilizing interferometric beam-shaping for controlling beamwidth of antenna beam patterns, and utilizing interference cancellation for reducing transmit signal leakage in received signals, said communications system comprising: a signal transmitter located at the transmit location for transmitting a plurality of transmission signals, each of said transmission signals having a predetermined spatial gain distribution at the receive location, an antenna array comprising a plurality of spatially-separated antenna elements located at the receive location, each of said antenna elements being responsive to at least one of said transmission signals for generating a desired receive communications signal and being responsive to one or more said transmission signals for generating a noise signal, a cancellation circuit coupled to each of said plurality of antenna elements for receiving said desired communications signals and said noise signals, said cancellation circuit providing weights to said desired communications signals and said noise signals wherein said weights are determined from said spatial gain distribution of said transmission signals, said cancellation circuit combining said weighted noise and desired communications signals for canceling said noise signals, thereby separating said communications signals from said noise signals, an excitation means coupled to said antenna elements for generating a predetermined distribution of excitation signals to electrically excite said antenna elements for producing a predetermined beam pattern, the excitation signals having distributed frequency characteristics, a transmit beam-shaping processor coupled to said excitation means for providing a frequency-dependent weight distribution to the excitation signals with respect to signal frequency such that a plurality of frequency-dependent beam patterns is generated by said array, each of the beam patterns corresponding to one of a plurality of different excitation signal frequencies, the beam patterns being substantially equal within a predetermined spatial region, a receiver coupled to said antenna elements for providing a predetermined weight distribution to the receive signals, the weighted receive signals being summed to provide a beam pattern that indicates responsiveness to the incident radiation with respect to an angle of incidence of the incident radiation, a receive beam-shaping processor coupled to said antenna elements for providing a frequency-dependent weight distribution to the receive signals with respect to receive signal frequency to produce a plurality of frequency-dependent beam patterns, each of the beam patterns corresponding to one of a plurality of different receive signal frequencies, the beam patterns being substantially equal within a predetermined spatial region, an interferometric receive beam-shaping processor coupled to said receiver for providing a plurality of weight distributions to the receive signals for providing a plurality of interfering receive beam patterns, the receive beam patterns being combined to produce a combined interferometric receive beam pattern, the combined interferometric receive beam pattern providing a predetermined receiver response in at least one direction, an interferometric transmit beam-shaping processor coupled to said excitation means for providing a plurality of weight distributions to the excitation signals for providing a plurality of interfering transmit beam patterns, the beam patterns being combined to produce a combined interferometric transmit beam pattern, the combined interferometric transmit beam pattern providing a predetermined transmit signal profile in at least one direction, and an isolator circuit coupled between said excitation means, said antenna array, and said receiver, for electrically isolating said receiver from said excitation means, said isolator circuit comprising: an active branch, said active branch comprising an active reference branch coupled to a splitting circuit for receiving a reference signal, and a transmit branch, said transmit branch comprising a transmit input port for receiving an input transmit signal, a splitting circuit coupled to the input port for splitting the input transmit signal into an output transmit signal and a reference signal, and an output transmit port coupled to an antenna for conducting the output transmit signal to the antenna, a reference sensing element coupled to said active reference branch, said reference sensing element being responsive to the reference signal in said active reference branch, a transmit sensing element coupled to said transmit branch, said transmit sensing element being responsive to the output transmit signal and a receive signal generated by said antenna in response to incident electromagnetic radiation, a combining circuit coupled to said reference sensing element and said transmit sensing element for combining the responses of said reference sensing element and said transmit sensing element for canceling the reference sensing element response to the reference signal and the transmit sensing element response to the output transmit signal, said combining circuit having an output port for coupling the response of said transmit sensing element to the receive signal to a receiver, a passive reference branch coupled to said splitting circuit for receiving the second reference signal, said passive reference branch comprising a reference splitting circuit coupled to a dummy reference branch and a dummy antenna branch, said reference splitting circuit splitting the second reference signal into a dummy reference branch signal and a dummy transmit signal, the dummy reference branch signal being coupled into said dummy reference branch, said dummy reference branch having a complex impedance that is proportional to the complex impedance of said active reference branch, and the dummy transmit signal being coupled into said dummy antenna branch, said dummy antenna branch comprising a variable impedance element, said dummy antenna branch having an impedance that is proportional to the impedance of said transmit branch, an injection circuit coupled between said combining circuit and said dummy antenna branch for injecting the receive signal at the output port of said combining circuit into said second reference branch, a control-signal generator coupled to said active signal branch and said passive reference branch, said control-signal generator being responsive to electrical signals in said active signal branch and said passive reference branch for generating a difference signal therefrom, the difference signal representing differences in the proportion of the complex impedance of said active signal branch to the complex impedance of said passive reference branch, and an impedance controller coupled between said control-signal generator and said variable impedance element for receiving the difference signal and adjusting the impedance of said variable impedance element in order to minimize the difference signal.” The entire disclosure of this United States patent is hereby incorporated by reference into this specification.

U.S. Pat. No. 6,211,671 discloses a cancellation circuit that removes interfering signals from desired signals in electrical systems having antennas or other electromagnetic pickup systems. This patent claims: “An electromagnetic receiver system adapted to receive and separate at least one desired electromagnetic transmission signal from at least one interfering electromagnetic transmission signal, the receiver system including: a plurality of electromagnetic receivers adapted to be responsive to the at least one transmitted desired electromagnetic signal and the at least one transmitted interfering electromagnetic signal, the receivers generating a plurality of receive signals, each of the receive signals including at least one desired signal component and at least one interfering signal component, the receivers being spatially separated to receive different proportions of the at least one transmitted desired electromagnetic signal and the at least one transmitted interfering electromagnetic signal and a canceller coupled to the receivers adapted to process the receive signals, the canceller including an amplitude-adjustment circuit adapted to provide amplitude adjustment to at least one of the receive signals to compensate for amplitude differences between the at least one interfering signal component in each of a plurality of the receive signals resulting from at least one of a) differences in propagation of the at least one transmitted interfering signal to the plurality of electromagnetic receivers, and b) differences in the responsiveness of the electromagnetic receivers to the at least one transmitted interfering signal, the canceller including a phase-adjustment circuit adapted to provide phase adjustment to at least one of the receive signals to compensate for phase differences between the at least one interfering signal component in each of a plurality of the receive signals resulting from at least one of: a) differences in propagation of the at least one transmitted interfering signal to the plurality of electromagnetic receivers, and b) differences in the responsiveness of the electromagnetic receivers to the at least one transmitted interfering signal, the canceller adapted to combine the receive signals to separate at least one of the desired signal components by canceling at least one of the interfering signal components.” The entire disclosure of this United States patent is hereby incorporated by reference into this specification.

U.S. Pat. No. 6,348,791 discloses an electromagnetic transceiver in which a cancellation circuit removes interfering signals. This patent claims: “An electromagnetic transceiver capable of simultaneously transmitting and receiving electromagnetic signals, the transceiver including: an antenna system capable of transmitting and receiving the electromagnetic signals, a signal transmitter coupled to the antenna system, the transmitter adapted to couple electromagnetic signals to the antenna system for transmission, a receiver coupled to the antenna system, the receiver adapted to be responsive to the transmitted electromagnetic signals and electromagnetic signals received by the antenna system, a cancellation circuit coupled to the transmitter and to the receiver, the cancellation circuit adapted to couple at least one cancellation signal to the receiver that reduces the responsiveness of the receiver to the transmitted signals, the cancellation circuit characterized by at least one of: an amplitude-adjustment circuit adapted to compensate for amplitude differences between the at least one cancellation signal and the receiver response to the transmitted signals resulting from at least one of: a) differences in propagation between the transmitted signals and the at least one cancellation signal to the receiver, and b) differences in the responsiveness of the receiver to the transmitted signals and the at least one cancellation signal, and a phase-adjustment circuit adapted to compensate for phase differences between the at least one cancellation signal and the receiver response to the transmitted signals resulting from at least one of: a) differences in propagation between the transmitted signals and the at least one cancellation signal to the receiver, and b) differences in the responsiveness of the receiver to the transmitted signals and the at least one cancellation signal.” The entire disclosure of this United States patent is hereby incorporated by reference into this specification.

It will be apparent that not every component, or every circuit, or every device of these patents will be suitable for use in the cancellation circuitry 210 and/or the cancellation circuitry 212. What will also be apparent is that many of these components, devices, and circuits, and the principles on which they operate, will be suitable for use in cancellation circuitry 210,212, taking into account the high-frequency MRI electromagnetic waves such circuitry is preferably designed to cancel and the goal of minimizing the amount of heat produced by such MRI electromagnetic waves. In particular, many of these components, devices, and/or circuits, and the principles on which they operate, will be suitable for modifying the current flow through biological tissue with which the medical device is contiguous or near to.

Thus, in one embodiment, the applicants provide a magnetically shielded assembly comprised of a medical device implanted in a biological organism, wherein said medical device is disposed near biological tissue, wherein said magnetically shielded assembly is comprised of a nanomagnetic coating (such as, e.g., coating 134) disposed on at least a portion of said medical device, wherein said magnetically shielded assembly is further comprised of means for limiting the flow of current through said biological tissue, and wherein said nanomagnetic coating has the properties described elsewhere in this specification.

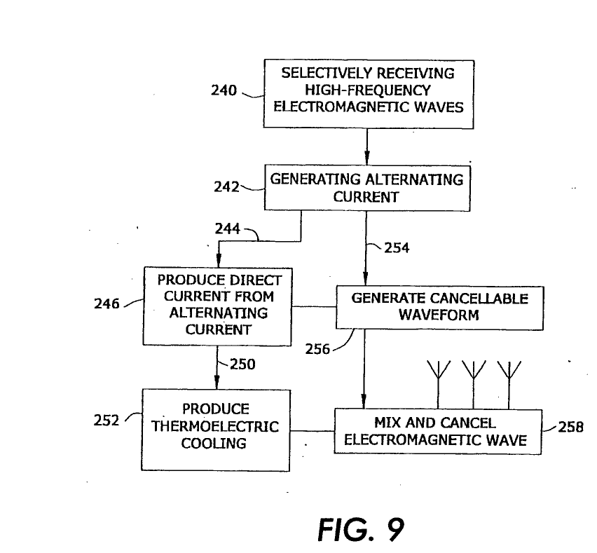

In general, the cancellation circuitry 210,212, and the rest of the devices depicted in FIG. 8, will enable one to follow the process depicted in FIG. 9.

Referring to FIG. 9, and in step 240 thereof, the high-frequency electromagnetic waves produced during the MRI analyses are selectively received by the cancellation circuitry assemblies 210 and/or 212 by means of antennas 230 and 232 (see FIG. 8). As is disclosed at page 110 of Stan Gibilisco's “Handbook of Radio and Wireless Technology,” Sixth Edition, supra, “ . . . an antenna is a . . . transducer . . . . A receiving antenna converts an electromagnetic field (EM) into an alternating current (AC).”

The antennas 232,232 are preferably tuned antennas that, with the appropriate combinations of antenna length, inductance, and/or capacitance, produce the maximum amount of AC current at the high frequencies produced during MRI analyses. Tuned antennas are well known to those skilled in the art. Reference may be had, e.g., to U.S. Pat. Nos. 6,310,346 (wavelength-tunable coupled antenna), U.S. Pat. No. 5,999,138 (switched diversity antenna system), U.S. Pat. No. 6,496,153 (magnetic-field sending antenna with RLC circuit), U.S. Pat. No. 5,614,917 (RF sail pumped tuned antenna), U.S. Pat. No. 5,528,251 (double tuned dipole antenna), U.S. Pat. Nos. 5,241,160, 5,231,355 (automatically tuned antenna), U.S. Pat. No. 4,984,296 (tuned radio apparatus), U.S. Pat. No. 4,739,516 (frequency tuned antenna assembly), U.S. Pat. Nos. 4,660,039, 4,450,588 4,280,129 (variable mutual inductance tuned antenna), U.S. Pat. Nos. 4,194,154, 3,571,716 (electronically tuned antenna), and the like. The entire disclosure of each of these United States patents is hereby incorporated by reference into this specification.

Referring again to FIG. 9, and in the preferred process depicted therein, in step 242, an alternating current is produced by the interaction of one or both of the antennas 230,232 with the high-frequency electromagnetic waves 273 (see FIG. 8). This alternating current is then distributed to several different locations.

A portion of the alternating current is fed via line 244 to a power supply (not shown), which converts the alternating current to direct current in step 246. Thereafter, the direct current so produced is preferably fed via line 250 to a thermoelectric cooling assembly (such as the Peltier device cooling assembly 166 depicted in FIG. 7), and in step 252 thermoelectric cooling is produced.

Referring again to FIG. 9, and in the preferred process depicted therein, another portion of the alternating current produced in step 242 is fed via 254 to a wave generator (not shown), and in step 256 a waveform is generated.

One may use, e.g., a conventional signal generator to produce the desired electromagnetic wave(s) in step 256. As is known to those skilled in the art, a signal generator is an instrument that delivers signals of precise frequency and amplitude, usually over a wide range. Reference may be had, e.g., to U.S. Pat. Nos. 6,256,157 (method for removing noise spikes), reissue 35,574 (method for acoustical echo cancellation), U.S. Pat. No. 5,126,681 (in-wire selective active cancellation system), U.S. Pat. No. 4,612,549 (interference canceller loop having automatic nulling of the loop phase shift for use in a reception system), U.S. Pat. No. 5,054,118 (balanced mixer using filters), U.S. Pat. No. 5,046,010 (fixed-echo canceling radio altimeter), U.S. Pat. No. 3,604,947 (variable filter device), U.S. Pat. No. 5,950,119 (image-reject mixers), U.S. Pat. No. 4,520,475 (duplex communication transceiver with modulation cancellation), U.S. Pat. No. 5,131,032 (echo canceller), U.S. Pat. No. 6,169,912 (RF front end with signal cancellation), U.S. Pat. No. 6,114,983 (electronic counter measures in radar), U.S. Pat. No. 5,023,620 (cross-polarization interference cancellation system), U.S. Pat. Nos. 5,924,024, 4,992,798 (interference canceller), U.S. Pat. No. 6,211,671 (interference-cancellation system for electromagnetic receivers), U.S. Pat. No. 6,208,135 (inductive noise cancellation for electromagnetic pickups), U.S. Pat. No. 6,269,165 (apparatus for reduction of unwanted feedback), U.S. Pat. No. 5,768,699 (amplifier with detuned test signal cancellation), U.S. Pat. No. 4,575,862 (cross-polarization distortion canceller), U.S. Pat. No. 6,147,576 (filter designs using parasitic and field effects), U.S. Pat. No. 4,438,530 (adaptive cross-polarization interference cancellation system), and the like. The entire disclosure of each of these United States patents is hereby incorporated by reference into this specification.

Referring again to FIG. 9, in step 256 one or more electromagnetic waves will be generated so that, when such wave(s) is mixed with the high-frequency electromagnetic waves produced by antennas 258, 260, and 262 in a mixer and mixed in step 258, some or all of such high-frequency electromagnetic waves will be cancelled.

Thus, as will be apparent, the process of FIG. 9 converts some of the high-frequency electromagnetic energy produced during MRI analyses to energy used for thermoelectric cooling (step 252), for conversion from alternating current to direct current (in step 246), for producing cancellable waveforms, and for mixing. All of this energy is energy that is not used to produce undesired heating of cardiac tissue.

A Preferred Sputtering Process

On Dec. 29, 2003, applicants filed U.S. patent application Ser. No. 10/747,472, for “Nanoelectrical Compositions.” The entire disclosure of this United States patent application is hereby incorporated by reference into this specification.

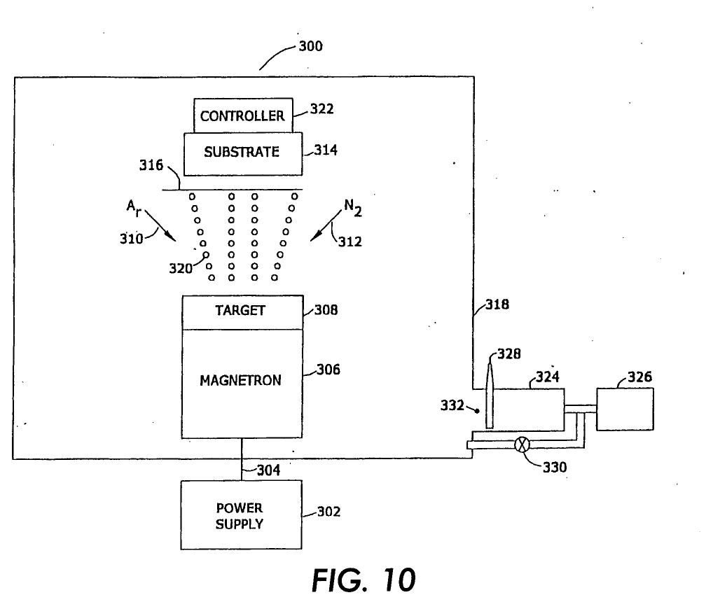

U.S. Ser. No. 10/747,472, at pages 10-15 thereof (and by reference to its FIG. 9), described the “ . . . preparation of a doped aluminum nitride assembly.” This portion of U.S. Ser. No. 10/747,472 is specifically incorporated by reference into this specification. It is also described below, by reference to FIG. 10, which is similar to the FIG. 9 of U.S. Ser. No. 10/747,472 but utilizes different reference numerals.

The system depicted in FIG. 10 may be used to prepare an assembly comprised of moieties A, B, and C (see FIG. 3). FIG. 10 will be described hereinafter with reference to one of the preferred ABC moieties, i.e., aluminum nitride doped with magnesium.

FIG. 10 is a schematic of a deposition system 300 comprised of a power supply 302 operatively connected via line 304 to a magnetron 306. Disposed on top of magnetron 306 is a target 308. The target 308 is contacted by gas 310 and gas 312, which cause sputtering of the target 308. The material so sputtered contacts substrate 314 when allowed to do so by the absence of shutter 316.

In one preferred embodiment, the target 308 is mixture of aluminum and magnesium atoms in a molar ratio of from about 0.05 to about 0.5 Mg/(Al+Mg). In one aspect of this embodiment, the ratio of Mg/(Al+Mg) is from about 0.08 to about 0.12. These targets are commercially available and are custom made by companies such as, e.g., Kurt Lasker and Company of Pittsburgh, Pa.

The power supply 302 preferably provides pulsed direct current. Generally, power supply 302 provides power in excess of 300 watts, preferably in excess of 500 watts, and more preferably in excess of 1,000 watts. In one embodiment, the power supplied by power supply 302 is from about 1800 to about 2500 watts.

The power supply preferably provides rectangular-shaped pulses with a duration (pulse width) of from about 10 nanoseconds to about 100 nanoseconds. In one embodiment, the pulse width is from about 20 to about 40 nanoseconds.

In between adjacent pulses, preferably substantially no power is delivered. The time between adjacent pulses is generally from about 1 microsecond to about 10 microseconds and is generally at least 100 times greater than the pulse width. In one embodiment, the repetition rate of the rectangular pulses is preferably about 150 kilohertz.

One may use a conventional pulsed direct current (d.c.) power supply. Thus, e.g., one may purchase such a power supply from Advanced Energy Company of Colorado, and/or from ENI Company of Rochester, N.Y.

The pulsed d.c. power from power supply 302 is delivered to a magnetron 306, that creates an electromagnetic field near target 308. In one embodiment, a magnetic field has a magnetic flux density of from about 0.01 Tesla to about 0.1 Tesla.

As will be apparent, because the energy provided to magnetron 306 preferably comprises intermittent pulses, the resulting magnetic fields produced by magnetron 306 will also be intermittent. Without wishing to be bound to any particular theory, applicants believe that the use of such intermittent electromagnetic energy yields better results than those produced by continuous radio-frequency energy.

Referring again to FIG. 10, it will be seen that the process depicted therein preferably is conducted within a vacuum chamber 118 in which the base pressure is from about 1×10⁻⁸ Torr to about 0.000005 Torr. In one embodiment, the base pressure is from about 0.000001 to about 0.000003 Torr.

The temperature in the vacuum chamber 318 generally is ambient temperature prior to the time sputtering occurs.

In one aspect of the embodiment illustrated in FIG. 10, argon gas is fed via line 310, and nitrogen gas is fed via line 312 so that both impact target 308, preferably in an ionized state.

The argon gas, and the nitrogen gas, are fed at flow rates such that the flow rate of the argon gas divided by the flow rate of the nitrogen gas preferably is from about 0.6 to about 1.2. In one aspect of this embodiment, such ratio of argon to nitrogen is from about 0.8 to about 0.95. Thus, for example, the flow rate of the argon may be 20 standard cubic centimeters per minute, and the flow rate of the nitrogen may be 23 standard cubic feet per minute.

The argon gas, and the nitrogen gas, contact a target 308 that is preferably immersed in an electromagnetic field. This field tends to ionize the argon and the nitrogen, providing ionized species of both gases. It is such ionized species that bombard target 308.

In one embodiment, target 308 may be, e.g., pure aluminum. In one preferred embodiment, however, target 308 is aluminum doped with minor amounts of one or more of the aforementioned moieties B.

In the latter embodiment, the moieties B are preferably present in a concentration of from about 1 to about 40 molar percent, by total moles of aluminum and moieties B. It is preferred to use from about 5 to about 30 molar percent of such moieties B.

The ionized argon gas, and the ionized nitrogen gas, after impacting the target 308, creates a multiplicity of sputtered particles 320. In the embodiment illustrated in FIG. 10, the shutter 316 prevents the sputtered particles from contacting substrate 314.

When the shutter 316 is removed, however, the sputtered particles 320 can contact and coat the substrate 314.

In one embodiment, illustrated in FIG. 10, the temperature of substrate 314 is controlled by controller 322 that can heat the substrate (by means such as a conduction heater or an infrared heater) and/or cool the substrate (by means such as liquid nitrogen or water).

The sputtering operation increases the pressure within the region of the sputtered particles 320. In general, the pressure within the area of the sputtered particles 320 is at least 100 times, and preferably 1000 times, greater than the base pressure.

Referring again to FIG. 10, a cryo pump 324 is preferably used to maintain the base pressure within vacuum chamber 318. In the embodiment depicted, a mechanical pump (dry pump) 326 is operatively connected to the cryo pump 324. Atmosphere from chamber 318 is removed by dry pump 326 at the beginning of the evacuation. At some point, shutter 328 is removed and allows cryo pump 324 to continue the evacuation. A valve 330 controls the flow of atmosphere to dry pump 326 so that it is only open at the beginning of the evacuation.

It is preferred to utilize a substantially constant pumping speed for cryo pump 324, i.e., to maintain a constant outflow of gases through the cryo pump 324. This may be accomplished by sensing the gas outflow via sensor 332 and, as appropriate, varying the extent to which the shutter 328 is open or partially closed.

Without wishing to be bound to any particular theory, applicants believe that the use of a substantially constant gas outflow rate insures a substantially constant deposition of sputtered nitrides.

Referring again to FIG. 10, and in one embodiment thereof, it is preferred to clean the substrate 314 prior to the time it is utilized in the process. Thus, e.g., one may use detergent to clean any grease or oil or fingerprints off the surface of the substrate. Thereafter, one may use an organic solvent such as acetone, isopropryl alcohol, toluene, etc.

In one embodiment, the cleaned substrate 314 is presputtered by suppressing sputtering of the target 308 and sputtering the surface of the substrate 314.

As will be apparent to those skilled in the art, the process depicted in FIG. 10 may be used to prepare coated substrates 314 comprised of moieties other than doped aluminum nitride.

A Preferred Coated Substrate

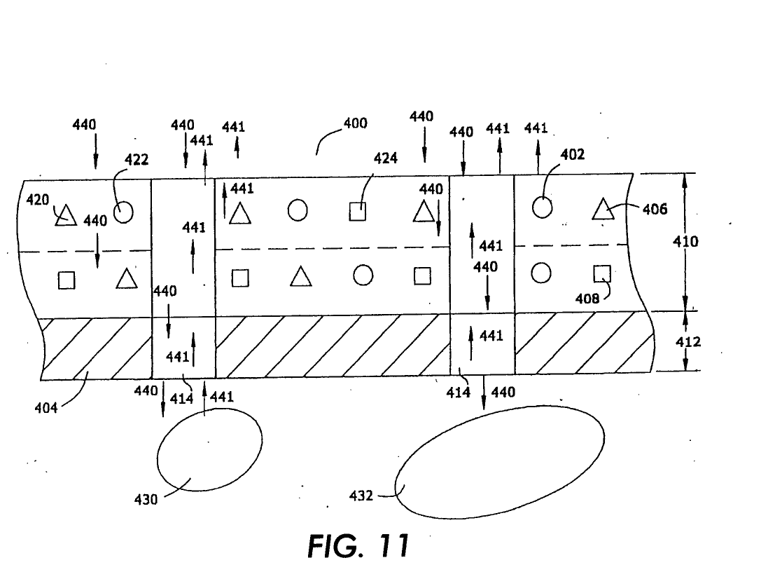

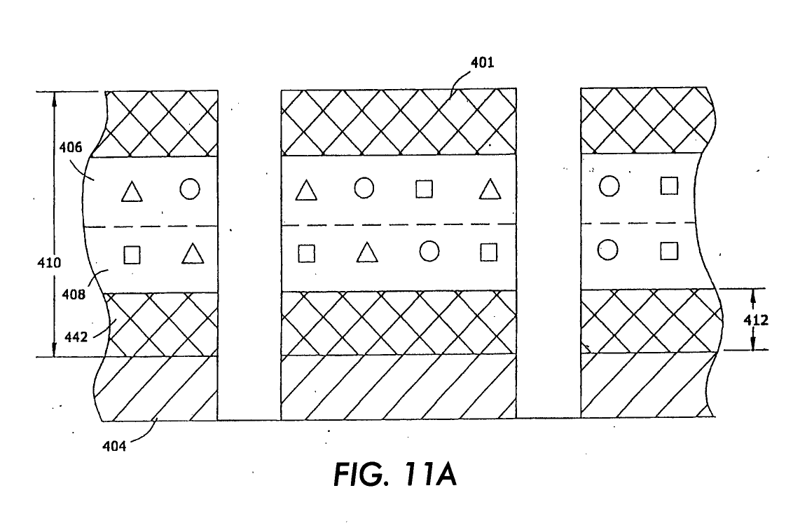

FIG. 11 is a schematic, partial sectional illustration of a coated substrate 400 that, in the preferred embodiment illustrated, is comprised of a coating 402 disposed upon a stent 404. As will be apparent, only one side of the coated stent 404 is depicted for simplicity of illustration.

In the preferred coated substrate depicted in FIG. 11, the coating 402 may be comprised of one layer of material, two layers of material, or three or more layers of material. In the embodiment depicted in FIG. 11, two coating layers, layers 406 and 408, are used.

Regardless of the number of coating layers used, it is preferred that the total thickness 410 of the coating 402 be at least about 400 nanometers and, preferably, be from about 400 to about 4,000 nanometers. In one embodiment, thickness 410 is from about 600 to about 1,000 nanometers. In another embodiment, thickness 410 is from about 750 to about 850 nanometers.

In the embodiment depicted, the substrate 404 has a thickness 412 that is substantially greater than the thickness 410. As will be apparent, the coated substrate 400 is not drawn to scale.

In general, the thickness 410 is less than about 5 percent of thickness 412 and, more preferably, less than about 2 percent. In one embodiment, the thickness of 410 is no greater than about 1.5 percent of the thickness 412.

The substrate 404, prior to the time it is coated with coating 402, has a certain flexural strength, and a certain spring constant.

The flexural strength is the strength of a material in bending, i.e., its resistance to fracture. As is disclosed in ASTM C-790, the flexural strength is a property of a solid material that indicates its ability to withstand a flexural or transverse load.

As is known to those skilled in the art, the spring constant is the constant of proportionality k which appears in Hooke's law for springs. Hooke's law states that: F=−kx, wherein F is the applied force and x is the displacement from equilibrium. The spring constant has units of force per unit length.

Means for measuring the spring constant of a material are well known to those skilled in the art. Reference may be had, e.g., to U.S. Pat. Nos. 6,360,589 (device and method for testing vehicle shock absorbers), U.S. Pat. No. 4,970,645 (suspension control method and apparatus for vehicle), U.S. Pat. Nos. 6,575,020, 4,157,060, 3,803,887, 4,429,574, 6,021,579, and the like. The entire disclosure of each of these United States patents is hereby incorporated by reference into this specification.

Referring again to FIG. 11, the flexural strength of the uncoated substrate 404 preferably differs from the flexural strength of the coated substrate 404 by no greater than about 5 percent. Similarly, the spring constant of the uncoated substrate 404 differs from the spring constant of the coated substrate 404 by no greater than about 5 percent.

Referring again to FIG. 11, and in the preferred embodiment depicted, the substrate 404 is comprised of a multiplicity of openings through which biological material is often free to pass. As will be apparent to those skilled in the art, when the substrate 404 is a stent, it will be realized that the stent has a mesh structure.

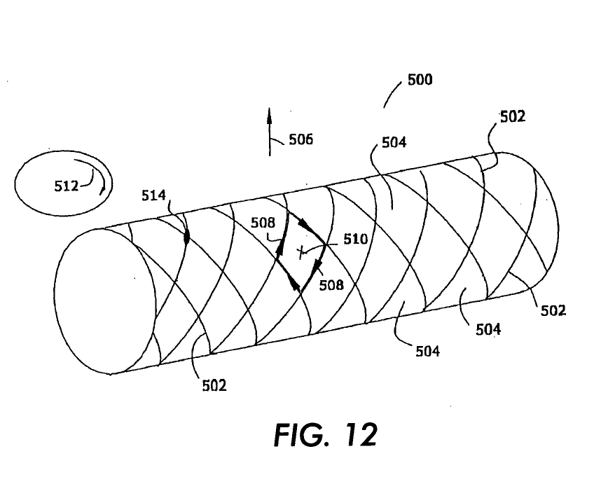

FIG. 12 is a schematic view of a typical stent 500 that is comprised of wire mesh 502 constructed in such a manner as to define a multiplicity of openings 504. The mesh material is typically a metal or metal alloy, such as, e.g., stainless steel, Nitinol (an alloy of nickel and titanium), niobium, copper, etc.

Typically the materials used in stents tend to cause current flow when exposed to a field 506. When the field 506 is a nuclear magnetic resonance field, it generally has a direct current component, and a radio-frequency component. For MRI (magnetic resonance imaging) purposes, a gradient component is added for spatial resolution.

The material or materials used to make the stent itself has certain magnetic properties such as, e.g., magnetic susceptibility. Thus, e.g., niobium has a magnetic susceptibility of 1.95×10⁻⁶ centimeter-gram-second units. Nitonol has a magnetic susceptibility of from about 2.5 to about 3.8×10⁻⁶ centimeter-gram-second units. Copper has a magnetic susceptibility of from −5.46 to about −6.16×10⁻⁶ centimeter-gram-second units.

When any particular material is used to make the stent, its response to an applied MRI field will vary depending upon, e.g., the relative orientation of the stent in relationship to the fields (including the d.c. field, the r.f. field, an the gradient field).

Any particular stent implanted in a human body will tend to have a different orientation than any other stent implanted in another human body due, in part, to the uniqueness of each human body. Thus, it cannot be predicated a priori what how any particular stent will respond to a particular MRI field.

The solution provided by one aspect of applicants' invention tends to cancel, or compensate for, the response of any particular stent in any particular body when exposed to an MRI field.

Referring again to FIG. 12, and to the uncoated stent 500 depicted therein, when an MRI field 506 is imposed upon the stent, it will tend to induce eddy currents. As used in this specification, the term eddy currents refers to loop currents and surface eddy currents.

Referring to FIG. 12, the MRI field 506 will induce a loop current 508. As is apparent to those skilled in the art, the MRI field 506 is an alternating current field that, as it alternates, induces an alternating eddy current 508. The radio-frequency field is also an alternating current field, as is the gradient field. By way of illustration, when the d.c. field is about 1.5 Tesla, the r.f. field has frequency of about 64 megahertz. With these conditions, the gradient field is in the kilohertz range, typically having a frequency of from about 2 to about 200 kilohertz.

Applying the well-known right hand rule, the loop current 508 will produce a magnetic field 510 extending into the plane of the paper and designated by an “x.” This magnetic field 510 will tend to oppose the direction of the applied field 506.

Referring again to FIG. 12, when the stent 500 is exposed to the MRI field 506, a surface eddy current will be produced where there is a relatively large surface area of conductive material such as, e.g., at junction 514.

The stent 500 must be constructed to have certain desirable mechanical properties. However, the materials that will provide the desired mechanical properties generally do not have desirable magnetic and/or electromagnetic properties. In an ideal situation, the stent 500 will produce no loop currents 508 and no surface eddy currents 512; in such situation, the stent 500 would have an effective zero magnetic susceptibility.

The prior art has heretofore been unable to provide such an ideal stent. Applicants' invention allows one to compensate for the deficiencies of the current stents by canceling the undesirable effects due to their magnetic susceptibilities, and/or by compensating for such undesirable effects.

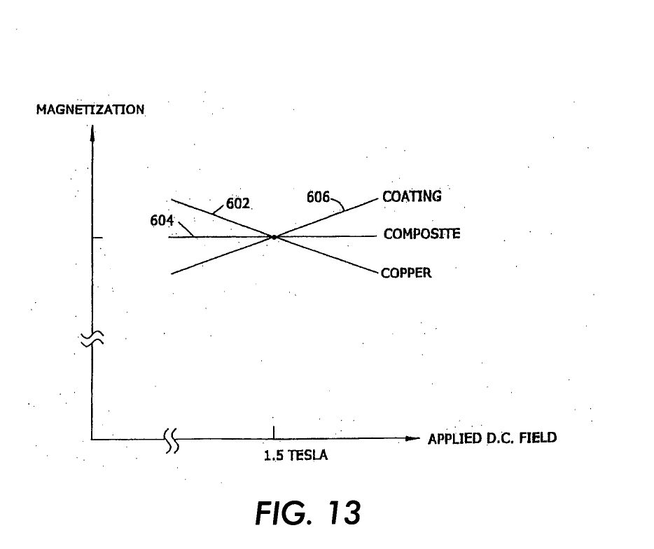

FIG. 13 is a graph of the magnetization of an object (such as an uncoated stent, or a coated stent) when subjected to an electromagnetic filed, such as an MRI field. It will be seen that, at different field strengths, different materials have different magnetic responses.

Thus, e.g., it will be seen that copper, at a d.c. field strength of 1.5 Tesla, is changing its magnetization as a function of the composite field strength (including the d.c. field strength, the r.f. field strength, and the gradient field strength) at a rate (defined by delta-magnetization/delta composite field strength) that is decreasing. With regard to the r.f. field and the gradient field, it should be understood that the order of magnitude of these fields is relatively small compared to the d.c. field, which is usually about 1.5 Tesla.

Referring again to FIG. 13, it will be seen that the slope of line 602 is negative. This negative slope indicates that copper, in response to the applied fields, is opposing the applied fields. Because the applied fields (including r.f. fields, and the gradient fields), are required for effective MRI imaging, the response of the copper to the applied fields tends to block the desired imaging, especially with the loop current and the surface eddy current described hereinabove.

Referring again to FIG. 13, the ideal magnetization response is illustrated by line 604, which is the response of the coated substrate of this invention, and wherein the slope is substantially zero. As used herein, the term substantially zero includes a slope will produce an effective magnetic susceptibility of from about 1×10⁻⁷ to about 1×10⁻⁸ centimeters-gram-second (cgs) units.

Referring again to FIG. 13, one means of correcting the negative slope of line 602 is by coating the copper with a coating which produces a response 606 with a positive slope so that the composite material produces the desired effective magnetic susceptibility of from about 1×10⁻⁷ to about 1×10⁻⁸ centimeters-gram-second (cgs) units.

FIG. 11 illustrates a coating that will produce the desired correction for the copper substrate 404. Referring to FIG. 11, it will be seen that, in the embodiment depicted, the coating 402 is comprised of at least nanomagnetic material 420 and nanodielectric material 422.

In one embodiment, the nanomagnetic material 402 preferably has an average particle size of less than about 20 nanometers and a saturation magnetization of from 10,000 to about 26,000 Gauss.

In one embodiment, the nanomagnetic material used is iron. In another embodiment, the nanomagentic material used is FeAlN. In yet another embodiment, the nanomagnetic material is FeAl. Other suitable materials will be apparent to those skilled in the art and include, e.g., nickel, cobalt, magnetic rare earth materials and alloys, thereof, and the like.

The nanodielectric material 422 preferably has a resistivity at 20 degrees Centigrade of from about 1×10⁻5 ohm-centimeters to about 1×10¹³ ohm-centimeters.

Referring again to FIG. 11, the nanomagnetic material 420 is preferably homogeneously dispersed within nanodielectric material 422, which acts as an insulating matrix. In general, the amount of nanodielectric material 422 in coating 402 exceeds the amount of nanomagnetic material 420 in such coating 402. In general, the coating 402 is comprised of at least about 70 mole percent of such nanodielectric material (by total moles of nanomagnetic material and nanodielectric material). In one embodiment, the coating 402 is comprised of less than about 20 mole percent of the nanomagnetic material, by total moles of nanomagnetic material and nanodielectric material. In one embodiment, the nanodielectric material used is aluminum nitride.

Referring again to FIG. 11, one may optionally include nanoconductive material 424 in the coating 402. This nanoconductive material generally has a resistivity at 20 degrees Centigrade of from about 1×10⁻⁶ ohm-centimeters to about 1×10⁻5 ohm-centimeters; and it generally has an average particle size of less than about 100 nanometers. In one embodiment, the nanoconductive material used is aluminum.

Referring again to FIG. 11, and in the embodiment depicted, it will be seen that two layers 406 and 408 are used to obtain the desired correction. In one embodiment, three or more such layers are used. This embodiment is depicted in FIG. 11A.

FIG. 11A is a schematic illustration of a coated substrate that is similar to coated substrate 400 but differs therefrom in that it contains two layers of dielectric material 440 and 442. In one embodiment, only one such layer of dielectric material 440 issued. Notwithstanding the use of additional layers 440 and 442, the coating 402 still preferably has a thickness 410 of from about 400 to about 4000 nanometers.—

As will be apparent, it may be difficult with only one layer of coating material to obtain the desired correction for the material comprising the stent (see FIG. 13). With a multiplicity of layers comprising the coating 402, which may have the same and/or different thicknesses, and/or the same and/or different compositions, more flexibility is provided in obtaining the desired correction.

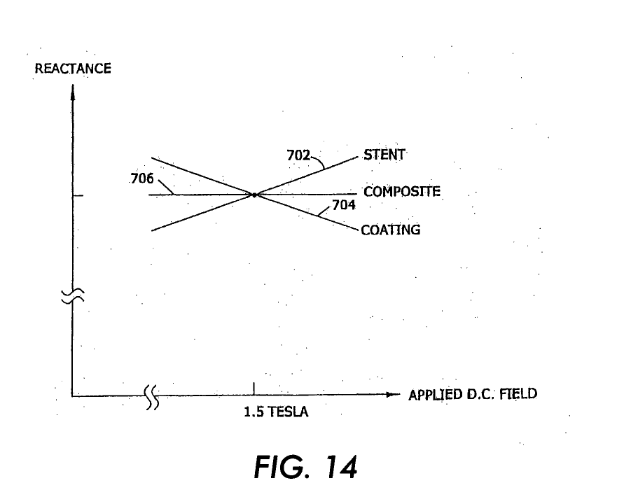

FIG. 13 illustrates the desired correction in terms of magnetization. FIG. 14 illustrates the desired correction in terms of reactance.

With regard to reactance, the r.f. field and the gradient field are treated as a radiation source which is applied to a living organism comprised of a stent in contact with biological material. The stent, with or without a coating, reacts to the radiation source by exhibiting a certain inductive reactance and a certain capacitative reactance. The net reactance is the difference between the inductive reactance and the capacitative reactance; and it desired that the net reactance be as close to zero as is possible. When the net reactance is greater than zero, it distorts some of the applied MRI fields and thus interferes with their imaging capabilities. Similarly, when the net reactance is less than zero, it also distorts some of the applied MRI fields.

Nullification of the Susceptibility Contribution due to the Substrate

As will be apparent by reference, e.g., to FIG. 13, the copper substrate depicted therein has a negative susceptibility, the coating depicted therein has a positive suceptibility, and the coated substrate thus has a substantially zero susceptibility. As will also be apparent, some substrates (such niobium, nitinol, stainless steel, etc.) have positive susceptibilities. In such cases, and in one preferred embodiment, the coatings should preferably be chosen to have a negative susceptibility so that, under the conditions of the MRI radiation (or of any other radiation source used), the net susceptibility of the coated object is still substantially zero.

The magnetic susceptibilities of various substrate materials are well known. Reference may be had, e.g., to pages E-118 to E-123 of the “Handbook of Chemistry and Physics,” 63rd edition (CRC Press, Inc., Boca Raton, Fla., 1974).

Once the susceptibility of the substrate material is determined, one can use the following equation: χ_{sub}+χ_{coat}=0, wherein χ_{sub} is the susceptibility of the substrate, and χ_{coat} is the susceptibility of the coating, when each of these is present in a 1/1 ratio. As will be apparent, the aforementioned equation is used when the coating and substrate are present in a 1/1 ratio. When other ratios are used other than a 1/1 ratio, the volume percent of each component must be taken into consideration in accordance with the equation: (volume percent of substrate×susceptibility of the substrate)+(volume percent of coating×susceptibility of the coating)=0. One may use a comparable formula in which the weight percent of each component is substituted for the volume percent, if the susceptibility is measured in terms of the weight percent.

By way of illustration, and in one embodiment, the uncoated substrate may either comprise or consist essentially of niobium, which has a susceptibility of +195.0×10⁻⁶ centimeter-gram seconds at 298 degrees Kelvin.

In another embodiment, the substrate may contain at least 98 molar percent of niobium and less than 2 molar percent of zirconium. Zirconium has a susceptibility of −122×0×10⁻6 centimeter-gram seconds at 293 degrees Kelvin. As will be apparent, because of the predominance of niobium, the net susceptibility of the uncoated substrate will be positive.

The substrate may comprise Nitinol. Nitinol is a paramagnetic alloy, an intermetallic compound of nickel and titanium; the alloy preferably contains from 50 to 60 percent of nickel, and it has a permeability value of about 1.002. The susceptibility of Nitinol is positive.

Nitinols with nickel content ranging from about 53 to 57 percent are known as “memory alloys” because of their ability to “remember” or return to a previous shape upon being heated. which is an alloy of nickel and titanium, in an approximate 1/1 ratio. The susceptibility of Nitinol is positive.

The substrate may comprise tantalum and/or titanium, each of which has a positive susceptibility. See, e.g., the CRC handbook cited above.

When the uncoated substrate has a positive susceptibility, the coating to be used for such a substrate should have a negative susceptibility. Referring again to said CRC handbook, it will be seen that the values of negative susceptibilities for various elements are −9.0 for beryllium, −280.1 for bismuth (s), −10.5 for bismuth (l), −6.7 for boron, −56.4 for bromine (l), −73.5 for bromine(g), −19.8 for cadmium(s), −18.0 for cadmium(l), −5.9 for carbon(dia), −6.0 for carbon (graph), −5.46 for copper(s), −6.16 for copper(l), −76.84 for germanium, −28.0 for gold(s), −34.0 for gold(l), −25.5 for indium, −88.7 for iodine(s), −23.0 for lead(s), −15.5 for lead(l), −19.5 for silver(s), −24.0 for silver(l), −15.5 for sulfur(alpha), −14.9 for sulfur(beta), −15.4 for sulfur(l), −39.5 for tellurium(s), −6.4 for tellurium(l), −37.0 for tin(gray), −31.7 for tin(gray), −4.5 for tin(l), −11.4 for zinc(s), −7.8 for zinc(l), and the like. As will be apparent, each of these values is expressed in units equal to the number in question x 10⁻⁶ centimeter-gram seconds at a temperature at or about 293 degrees Kelvin. As will also be apparent, those materials which have a negative susceptibility value are often referred to as being diamagnetic.

By way of further reference, a listing of organic compounds that are diamagnetic is presented on pages E123 to E134 of the aforementioned “Handbook of Chemistry and Physics,” 63rd edition (CRC Press, Inc., Boca Raton, Fla., 1974).

Preferred Magnetic Materials that may be used in the Process of the Invention

In one embodiment, and referring again to the aforementioned “Handbook of Chemistry and Physics,” 63rd edition (CRC Press, Inc., Boca Raton, Fla., 1974), one or more of the following magnetic materials described below are preferably incorporated into the coating.

The desired magnetic materials in this embodiment preferably have a positive susceptibility, with values ranging from +1×10⁻6 centimeter-gram seconds at a temperature at or about 293 degrees Kelvin, to about 1×10⁶ centimeter-gram seconds at a temperature at or about 293 degrees Kelvin.

Thus, by way of illustration and not limitation, one may use materials such as Alnicol (see page E-112 of the CRC handbook), which is an alloy containing nickel, aluminum, and other elements such as, e.g., cobalt and/or iron. Thus, e.g., one my use silicon iron (see page E113 of the CRC handbook), which is an acid resistant iron containing a high percentage of silicon. Thus, e.g., one may use steel (see page 117 of the CRC handbook). Thus, e.g., one may use elements such as dyprosium, erbium, europium, gadolinium, hafnium, holmium, manganese, molybdenum, neodymium, nickel-cobalt, alloys of the above, and compounds of the above such as, e.g., their oxides, nitrides, carbonates, and the like.

Referring to FIG. 14, and to the embodiment depicted therein, it will be seen that the uncoated stent has an effective inductive reactance at a d.c. field of 1.5 Tesla that exceeds its capacitative reactance, whereas the coating 704 has a capacitative reatance that exceeds its inductive reactance. The coated (composite) stent 706 has a net reactance that is substantially zero.

As will be apparent, the effective inductive reactance of the uncoated stent 702 may be due to a multiplicity of factors including, e.g., the positive magnetic susceptibility of the materials which it is comprised of it, the loop currents produced, the surface eddy produced, etc. Regardless of the source(s) of its effective inductive reactance, it can be “corrected” by the use of one or more coatings which provide, in combination, an effective capacitative reactance that is equal to the effective inductive reactance.

Referring again to FIG. 11, and in the embodiment depicted, plaque particles 430,432 are disposed on the inside of substrate 404. When the net reactance of the coated substrate 404 is essentially zero, the imaging field 440 can pass substantially unimpeded through the coating 402 and the sustrate 404 and interact with the plaque particles 430/432 to produce imaging signals 441.

The imaging signals 441 are able to pass back through the substrate 404 and the coating 402 because the net reactance is substantially zero. Thus, these imaging signals are able to be received and processed by the MRI apparatus.

Thus, by the use of applicant's technology, one may negate the negative substrate effect and, additionally, provide pathways for the image signals to interact with the desired object to be imaged (such as, e.g., the plaque particles) and to produce imaging signals that are capable of escaping the substrate assembly and being received by the MRI apparatus.

Incorporation by Reference of Certain Pending Patent Applications

In accordance with the Manual of Patent Examining Procedure (M.P.E.P.), section 60.8.01(p), applicants are hereby incorporating by reference certain disclosure from their copending patent applications into the instant case. In particular, applicants are incorporating the following disclosures into this case: (1) U.S. Ser. No. 60/533,200, Coated stent assembly, filed on Dec. 30, 2003, (2) U.S. Ser. No. 10/747,472, “Nanoelectrcial Compositions,” filed on Dec. 29, 2003, (3) U.S. Ser. No. 10/744,543, “Optical Fiber Assembly,” filed on Dec. 22, 2003, (4) U.S. S. No. 60/525,916, “MRI Contrast Agent Assembly,” filed on Dec. 1, 2003, (5) U.S. Ser. No. 10/477,120, “Novel Coating Process,” filed on Jun. 9, 2003, (6) U.S. Ser. No. 10/409,505, “Nanomagnetic Composition,” filed on Apr. 8, 2003, (7) U.S. Ser. No. 10/384,288, “Magnetic Resonance Imaging Coated Assembly,” filed on Mar. 7, 2003, (8) U.S. Ser. No. 10/373,377, “Protective Assembly,” filed on Feb. 24, 2003, (9) U.S. Ser. No. 10/366,082, “Magnetically Shielded Assembly,” filed on Feb. 12, 2003, (10) U.S. Ser. No. 10/336,088, “Optical Fiber Assembly,” filed on Jan. 3, 2003, (11) U.S. Ser. No. 10/324,773, “Nanomagnetically Shielded Substrate,” filed on Dec. 18, 2002, (12) U.S. Ser. No. 10/303,264, “Magnetically Shielded Assembly,” filed on Nov. 25, 2002, (13) U.S. Ser. No. 10/273,738, “Nanomagnetically Shielding Assembly,” filed on Oct. 18, 2002, (14) U.S. Ser. No. 10/260,247, “Magnetically Shielded Assembly,” filed on Sep. 30, 2002, (15) U.S. Ser. No. 10/242,969, “Magnetically Shielded Conductor,” filed on Sep. 13, 2002, (16) U.S. Ser. No. 10/090,553, “Mangetically Shielded Conductor,” filed on Mar. 4, 2002, and (17) U.S. Ser. No. 10/054,407, “Magnetically Shielded Conductor,” filed on Jan. 22, 2002. The entire disclosure of each of these United States patent applications is hereby incorporated by reference into this patent application.

Incorporation of Disclosure of U.S. Ser. No. 10/303/264, Filed on Nov. 25, 2002

Applicants' hereby incorporate by reference into this specification the entire disclosure of their copending United States patent application U.S. Ser. No. 10/303,264, filed on Nov. 25, 2002, and also the corresponding disclosure of their U.S. Pat. No. 6,713,671, issued on Mar. 30, 2004.

United States patent application U.S. Ser. No. 10/303,264 (and also U.S. Pat. No. 6,713,671) discloses a shielded assembly comprised of a substrate and, disposed above a substrate, a shield comprising from about 1 to about 99 weight percent of a first nanomagnetic material, and from about 99 to about 1 weight percent of a second material with a resistivity of from about 1 microohm-centimeter to about 1×1025 microohm centimeters; the nanomagnetic material comprises nanomagnetic particles, and these nanomagnetic particles respond to an externally applied magnetic field by realigning to the externally applied field. Such a shielded assembly and/or the substrte thereof and/or the shield thereof may be used in the processes, compositions, and/or constructs of this invention.

As is disclosed in U.S. Pat. No. 6,713,617, the entire disclosoure of which is hereby incorporated by reference into this specification, in one embodiment the substrate used may be, e.g, comprised of one or more conductive material(s) that have a resistivity at 20 degrees Centigrade of from about 1 to about 100 microohm-centimeters. Thus, e.g., the conductive material(s) may be silver, copper, aluminum, alloys thereof, mixtures thereof, and the like.

In one embodiment, the substrate consists consist essentially of such conductive material. Thus, e.g., it is preferred not to use, e.g., copper wire coated with enamel in this embodiment.

In the first step of the process preferably used to make this embodiment of the invention, (see step 40 of FIG. 1 of U.S. Pat. No. 6,713,671), conductive wires are coated with electrically insulative material. Suitable insulative materials include nano-sized silicon dioxide, aluminum oxide, cerium oxide, yttrium-stabilized zirconia, silicon carbide, silicon nitride, aluminum nitride, and the like. In general, these nano-sized particles will have a particle size distribution such that at least about 90 weight percent of the particles have a maximum dimension in the range of from about 10 to about 100 nanometers.

In such process, the coated conductors may be prepared by conventional means such as, e.g., the process described in U.S. Pat. No. 5,540,959, the entire disclosure of which is hereby incorporated by reference into this specification. Alternatively, one may coat the conductors by means of the processes disclosed in a text by D. Satas on “Coatings Technology Handbook” (Marcel Dekker, Inc., New York, N.Y., 1991). As is disclosed in such text, one may use cathodic arc plasma deposition (see pages 229 et seq.), chemical vapor deposition (see pages 257 et seq.), sol-gel coatings (see pages 655 et seq.), and the like.

FIG. 2 of U.S. Pat. No. 6,713,671 is a sectional view of the coated conductors 14/16. In the embodiment depicted in such FIG. 2, itt will be seen that conductors 14 and 16 are separated by insulating material 42. In order to obtain the structure depicted in such FIG. 2, one may simultaneously coat conductors 14 and 16 with the insulating material so that such insulators both coat the conductors 14 and 16 and fill in the distance between them with insulation.

Referring again to such FIG. 2 of U.S. Pat. No. 6,713,671, the insulating material 42 that is disposed between conductors 14/16, may be the same as the insulating material 44/46 that is disposed above conductor 14 and below conductor 16. Alternatively, and as dictated by the choice of processing steps and materials, the insulating material 42 may be different from the insulating material 44 and/or the insulating material 46. Thus, step 48 of the process of such FIG. 2 describes disposing insulating material between the coated conductors 14 and 16. This step may be done simultaneously with step 40; and it may be done thereafter.

Referring again to such FIG. 2, and to the preferred embodiment depicted therein, the insulating material 42, the insulating material 44, and the insulating material 46 each generally has a resistivity of from about 1,000,000,000 to about 10,000,000,000,000 ohm-centimeters.

Referring again to FIG. 2 of U.S. Pat. No. 6,713,671, after the insulating material 42/44/46 has been deposited, and in one embodiment, the coated conductor assembly is preferably heat treated in step 50. This heat treatment often is used in conjunction with coating processes in which the heat is required to bond the insulative material to the conductors 14/16.

The heat-treatment step may be conducted after the deposition of the insulating material 42/44/46, or it may be conducted simultaneously therewith. In either event, and when it is used, it is preferred to heat the coated conductors 14/16 to a temperature of from about 200 to about 600 degrees Centigrade for from about 1 minute to about 10 minutes.

Referring again to FIG. 1A of U.S. Pat. No. 6,713,67, and in step 52 of the process, after the coated conductors 14/16 have been subjected to heat treatment step 50, they are allowed to cool to a temperature of from about 30 to about 100 degrees Centigrade over a period of time of from about 3 to about 15 minutes.

One need not invariably heat treat and/or cool. Thus, referring to such FIG. 1A, one may immediately coat nanomagnetic particles onto to the coated conductors 14/16 in step 54 either after step 48 and/or after step 50 and/or after step 52.

Referring again to FIG. 1A of U.S. Pat. No. 6,713,67, in step 54, nanomagnetic materials are coated onto the previously coated conductors 14 and 16. This is best shown in FIG. 2 of such patent, wherein the nanomagnetic particles are identified as particles 24.

In general, and as is known to those skilled in the art, nanomagnetic material is magnetic material which has an average particle size less than 100 nanometers and, preferably, in the range of from about 2 to 50 nanometers. Reference may be had, e.g., to U.S. Pat. Nos. 5,889,091 (rotationally free nanomagnetic material), U.S. Pat. Nos. 5,714,136, 5,667,924, and the like. The entire disclosure of each of these United States patents is hereby incorporated by reference into this specification.

In general, the thickness of the layer of nanomagnetic material deposited onto the coated conductors 14/16 is less than about 5 microns and generally from about 0.1 to about 3 microns.

Referring again to FIG. 2 of U.S. Pat. No. 6,713,671, after the nanomagnetic material is coated in step 54, the coated assembly may be optionally heat-treated in step 56. In this optional step 56, it is preferred to subject the coated conductors 14/16 to a temperature of from about 200 to about 600 degrees Centigrade for from about 1 to about 10 minutes.

In one embodiment, illustrated in FIG. 3 of U.S. Pat. No. 6,713,671, one or more additional insulating layers 43 are coated onto the assembly depicted in FIG. 2 of such patent. This is conducted in optional step 58 (see FIG. 1A of such patent).

FIG. 4 of U.S. Pat. No. 6,713,671 is a partial schematic view of the assembly 11 of FIG. 2 of such patent, illustrating the current flow in such assembly. Referring again to FIG. 4 of U.S. Pat. No. 6,713,671, it will be seen that current flows into conductor 14 in the direction of arrow 60, and it flows out of conductor 16 in the direction of arrow 62. The net current flow through the assembly 11 is zero; and the net Lorentz force in the assembly 11 is thus zero. Consequently, even high current flows in the assembly 11 do not cause such assembly to move.

Referring again to FIG. 4 of U.S. Pat. No. 6,713,67. conductors 14 and 16 are substantially parallel to each other. As will be apparent, without such parallel orientation, there may be some net current and some net Lorentz effect.

In the embodiment depicted in such FIG. 4, and in one preferred aspect thereof, the conductors 14 and 16 preferably have the same diameters and/or the same compositions and/or the same length.

Referring again to FIG. 4 of U.S. Pat. No. 6,713,671, the nanomagnetic particles 24 are present in a density sufficient so as to provide shielding from magnetic flux lines 64. Without wishing to be bound to any particular theory, applicant believes that the nanomagnetic particles 24 trap and pin the magnetic lines of flux 64.

In order to function optimally, the nanomagnetic particles 24 preferably have a specified magnetization. As is known to those skilled in the art, magnetization is the magnetic moment per unit volume of a substance. Reference may be had, e.g., to U.S. Pat. Nos. 4,169,998, 4,168,481, 4,166,263, 5,260,132, 4,778,714, and the like. The entire disclosure of each of these United States patents is hereby incorporated by reference into this specification.

Referring again to FIG. 4 of U.S. Pat. No. 6,713,671, the layer of nanomagnetic particles 24 preferably has a saturation magnetization, at 25 degrees Centigrade, of from about 1 to about 36,000 Gauss, or higher. In one embodiment, the saturation magnetization at room temperature of the nanomagentic particles is from about 500 to about 10,000 Gauss. For a discussion of the saturation magnetization of various materials, reference may be had, e.g., to U.S. Pat. Nos. 4,705,613, 4,631,613, 5,543,070, 3,901,741 (cobalt, samarium, and gadolinium alloys), and the like. The entire disclosure of each of these United States patents is hereby incorporated by reference into this specification.

In one embodiment, it is preferred to utilize a thin film with a thickness of less than about 2 microns and a saturation magnetization in excess of 20,000 Gauss. The thickness of the layer of nanomagentic material is measured from the bottom surface of the layer that contains such material to the top surface of such layer that contains such material; and such bottom surface and/or such top surface may be contiguous with other layers of material (such as insulating material) that do not contain nanomagnetic particles.

Thus, e.g., one may make a thin film in accordance with the procedure described at page 156 of Nature, Volume 407, Sep. 14, 2000, that describes a multilayer thin film has a saturation magnetization of 24,000 Gauss.

Referring again to FIG. 4 of U.S. Pat. No. 6,713,671, the nanomagnetic particles 24 are disposed within an insulating matrix so that any heat produced by such particles will be slowly dispersed within such matrix. Such matrix, as indicated hereinabove, may be made from ceria, calcium oxide, silica, alumina. In general, the insulating material 42 preferably has a thermal conductivity of less than about 20 (caloriescentimeters/square centimeters−degree second)×10,000. See, e.g., page E-6 of the 63rd Edition of the “Handbook of Chemistry and Physics” (CRC Press, Inc., Boca Raton, Fla., 1982).

The nanomagnetic materials 24 typically comprise one or more of iron, cobalt, nickel, gadolinium, and samarium atoms. Thus, e.g., typical nanomagnetic materials include alloys of iron and nickel (permalloy), cobalt, niobium, and zirconium (CNZ), iron, boron, and nitrogen, cobalt, iron, boron, and silica, iron, cobalt, boron, and fluoride, and the like. These and other materials are described in a book by J. Douglas Adam et al. entitled “Handbook of Thin Film Devices” (Academic Press, San Diego, Calif., 2000). Chapter 5 of this book beginning at page 185, describes “magnetic films for planar inductive components and devices;” and Tables 5.1 and 5.2 in this chapter describe many magnetic materials.

FIG. 5 of U.S. Pat. No. 6,713,671 is a sectional view of the assembly 11 of FIG. 2 of such patent. The device of such FIG. 5 is preferably substantially flexible. As used in this specification, the term flexible refers to an assembly that can be bent to form a circle with a radius of less than 2 centimeters without breaking. Put another way, the bend radius of the coated assembly 11 can be less than 2 centimeters. Reference may be had, e.g., to U.S. Pat. Nos. 4,705,353, 5,946,439, 5,315,365, 4,641,917, 5,913,005, and the like. The entire disclosure of each of these United States patents is hereby incorporated by reference into this specification.

In another embodiment, not shown, the shield is not flexible. Thus, in one aspect of this embodiment, the shield is a rigid, removable sheath that can be placed over an endoscope or a biopsy probe used inter-operatively with magnetic resonance imaging.

In another embodiment of the invention of U.S. Pat. No. 6,713,671, there is provided a magnetically shielded conductor assembly comprised of a conductor and a film of nanomagnetic material disposed above said conductor. In this embodiment, the conductor has a resistivity at 20 degrees Centigrade of from about 1 to about 2,000 micro ohm-centimeters and is comprised of a first surface exposed to electromagnetic radiation. In this embodiment, the film of nanomagnetic material has a thickness of from about 100 nanometers to about 10 micrometers and a mass density of at least about about 1 gram per cubic centimeter, wherein the film of nanomagnetic material is disposed above at least about 50 percent of said first surface exposed to electromagnetic radiation, and the film of nanomagnetic material has a saturation magnetization of from about 1 to about 36,000 Gauss, a coercive force of from about 0.01 to about 5,000 Oersteds, a relative magnetic permeability of from about 1 to about 500,000, and a magnetic shielding factor of at least about 0.5. In this embodiment, the nanomagnetic material has an average particle size of less than about 100 nanometers.

In one preferred embodiment of this invention, and referring to FIG. 6 of U.S. Pat. No. 6,713,671, a film of nanomagnetic material is disposed above at least one surface of a conductor. Referring to such FIG. 6, and in the schematic diagram depicted therein, a source of electromagnetic radiation 100 emits radiation 102 in the direction of film 104. Film 104 is disposed above conductor 106, i.e., it is disposed between conductor 106 of the electromagnetic radiation 102.

Referring again to FIG. 6 of U.S. Pat. No. 6,713,671, the film 104 is adapted to reduce the magnetic field strength at point 108 (which is disposed less than 1 centimeter above film 104) by at least about 50 percent. Thus, if one were to measure the magnetic field strength at point 108, and thereafter measure the magnetic field strength at point 110 (which is disposed less than 1 centimeter below film 104), the latter magnetic field strength would be no more than about 50 percent of the former magnetic field strength. Put another way, the film 104 has a magnetic shielding factor of at least about 0.5.

Referring again to FIG. 6 of U.S. Pat. No. 6,713,671, in one embodiment, the film 104 has a magnetic shielding factor of at least about 0.9, i.e., the magnetic field strength at point 110 is no greater than about 10 percent of the magnetic field strength at point 108. Thus, e.g., the static magnetic field strength at point 108 can be, e.g., one Tesla, whereas the static magnetic field strength at point 110 can be, e.g., 0.1 Tesla. Furthermore, the time-varying magnetic field strength of a 100 milliTesla would be reduced to about 10 milliTesla of the time-varying field.

Referring again to FIG. 6 of U.S. Pat. No. 6,713,671, the nanomagnetic material 103 in film 104 has a saturation magnetization of form about 1 to about 36,000 Gauss. In one embodiment, the nanomagnetic material 103 a saturation magnetization of from about 200 to about 26,000 Gauss.

Referring again to FIG. 6 of U.S. Pat. No. 6,713,671, the nanomagnetic material 103 in film 104 also has a coercive force of from about 0.01 to about 5,000 Oersteds. The term coercive force refers to the magnetic field, H, which must be applied to a magnetic material in a symmetrical, cyclicly magnetized fashion, to make the magnetic induction, B, vanish; this term often is referred to as magnetic coercive force. Reference may be had, e.g., to U.S. Pat. Nos. 4,061,824, 6,257,512, 5,967,223, 4,939,610, 4,741,953, and the like. The entire disclosure of each of these United States patents is hereby incorporated by reference into this specification.

Referring again to FIG. 6 of U.S. Pat. No. 6,713,671, in one embodiment, the nanomagnetic material 103 has a coercive force of from about 0.01 to about 3,000 Oersteds. In yet another embodiment, the nanomagnetic material 103 has a coercive force of from about 0.1 to about 10.

Referring again to such FIG. 6, the nanomagnetic material 103 in film 104 preferably has a relative magnetic permeability of from about 1 to about 500,000; in one embodiment, such material 103 has a relative magnetic permeability of from about 1.5 to about 260,000. As used in this specification, the term relative magnetic permeability is equal to B/H, and is also equal to the slope of a section of the magnetization curve of the film. Reference may be had, e.g., to page 4-28 of E. U. Condon et al.'s “Handbook of Physics” (McGraw-Hill Book Company, Inc., New York, 1958).

Reference also may be had to page 1399 of Sybil P. Parker's “McGraw-Hill Dictionrary of Scientific and Technical Terms,” Fourth Edition (McGraw Hill Book Company, New York, 1989). As is disclosed on this page 1399, permeability is “ . . . a factor, characteristic of a material, that is proportional to the magnetic induction produced in a material divided by the magnetic field strength; it is a tensor when these quantities are not parallel.”

Reference also may be had, e.g., to U.S. Pat. Nos. 6,181,232, 5,581,224, 5,506,559, 4,246,586, 6,390,443, and the like. The entire disclosure of each of these United States patents is hereby incorporated by reference into this specification.

In one embodiment, the nanomagnetic material 103 in film 104 has a relative magnetic permeability of from about 1.5 to about 2,000.

Referring again to FIG. 6 of U.S. Pat. No. 6,713,671, the nanomagnetic material 103 in film 104 preferably has a mass density of at least about 0.001 grams per cubic centimeter; in one embodiment, such mass density is at least about 1 gram per cubic centimeter. As used in this specification, the term mass density refers to the mass of a give substance per unit volume. See, e.g., page 510 of the aforementioned “McGraw-Hill Dictionary of Scientific and Technical Terms.” In one embodiment, the film 104 has a mass density of at least about 3 grams per cubic centimeter. In another embodiment, the nanomagnetic material 103 has a mass density of at least about 4 grams per cubic centimeter.

Referring again to FIG. 6 of U.S. Pat. No. 6,713,671, and in the embodiment depicted in such FIG. 6, the film 104 is disposed above 100 percent of the surfaces 112, 114, 116, and 118 of the conductor 106. In the embodiment depicted in FIG. 2, by comparison, the nanomagnetic film is disposed around the conductor.

Yet another embodiment is depicted in FIG. 7 of U.S. Pat. No. 6,713,671 In the embodiment depicted in FIG. 7, the film 104 is not disposed in front of either surface 114, or 116, or 118 of the conductor 106. Inasmuch as radiation is not directed towards these surfaces, this is possible.

What is essential, however, is that the film 104 be interposed between the radiation 102 and surface 112. It is preferred that film 104 be disposed above at least about 50 percent of surface 112. In one embodiment, film 104 is disposed above at least about 90 percent of surface 112.

Referring again to FIG. 8A of U.S. Pat. No. 6,713,671, and in the preferred embodiment depicted in FIG. 8A, the nanomagnetic material 202 may be disposed within an insulating matrix (not shown) so that any heat produced by such particles will be slowly dispersed within such matrix. Such matrix, as indicated hereinabove, may be made from ceria, calcium oxide, silica, alumina, and the like. In general, the insulating material 202 preferably has a thermal conductivity of less than about 20 (calories centimeters/square centimeters-degree second)×10,000. See, e.g., page E-6 of the 63rd. Edition of the “Handbook of Chemistry and Physics” (CRC Press, Inc. Boca Raton, Fla., 1982).

Referring again to FIG. 8A of U.S. Pat. No. 6,713,67, and in the preferred embodiment depicted therein the nanomagnetic material 202 typically comprises one or more of iron, cobalt, nickel, gadolinium, and samarium atoms. Thus, e.g., typical nanomagnetic materials include alloys of iron, and nickel (permalloy), cobalt, niobium and zirconium (CNZ), iron, boron, and nitrogen, cobalt, iron, boron and silica, iron, cobalt, boron, and fluoride, and the like. These and other materials are described in a book by J. Douglass Adam et al. entitled “Handbook of Thin Film Devices” (Academic Press, San Diego, Calif., 2000). Chapter 5 of this book beginning at page 185 describes “magnetic films for planar inductive components and devices;” and Tables 5.1. and 5.2 in this chapter describes many magnetic materials.

FIG. 11 of U.S. Pat. No. 6,713,671 is a schematic sectional view of a substrate 401, which is part of an implantable medical device (not shown). Referring to such FIG. 11, and in the preferred embodiment depicted therein, it will be seen that substrate 401 is coated with a layer 404 of nanomagnetic material(s). The layer 404, in the embodiment depicted, is comprised of nanomagnetic particulate 405 and nanomagnetic particulate 406. Each of the nanomagnetic particulate 405 and nanomagnetic particulate 406 preferably has an elongated shape, with a length that is greater than its diameter. In one aspect of this embodiment, nanomagnetic particles 405 have a different size than nanomagnetic particles 406. In another aspect of this embodiment, nanomagnetic particles 405 have different magnetic properties than nanomagnetic particles 406. Referring again to such FIG. 11, and in the preferred embodiment depicted therein, nanomagnetic particulate material 405 and nanomagnetic particulate material 406 are designed to respond to an static or time-varying electromagnetic fields or effects in a manner similar to that of liquid crystal display (LCD) materials. More specifically, these nanomagnetic particulate materials 405 and nanomagnetic particulate materials 406 are designed to shift alignment and to effect switching from a magnetic shielding orientation to a non-magnetic shielding orientation. As will be apparent, the magnetic shield provided by layer 404, can be turned “ON” and “OFF” upon demand. In yet another embodiment (not shown), the magnetic shield is turned on when heating of the shielded object is detected.

In one embodiment of the invention, also described in U.S. Pat. No. 6,713,671, there is provided a coating of nanomagnetic particles that consists of a mixture of aluminum oxide (Al2O3), iron, and other particles that have the ability to deflect electromagnetic fields while remaining electrically non-conductive. Preferably the particle size in such a coating is approximately 10 nanometers. Preferably the particle packing density is relatively low so as to minimize electrical conductivity. Such a coating when placed on a fully or partially metallic object (such as a guide wire, catheter, stent, and the like) is capable of deflecting electromagnetic fields, thereby protecting sensitive internal components, while also preventing the formation of eddy currents in the metallic object or coating. The absence of eddy currents in a metallic medical device provides several advantages, to wit: (1) reduction or elimination of heating, (2) reduction or elimination of electrical voltages which can damage the device and/or inappropriately stimulate internal tissues and organs, and (3) reduction or elimination of disruption and distortion of a magnetic-resonance image.

In one portion of U.S. Pat. No. 6,713,671, the patentees described one embodiment of a composite shield. This embodiment involves a shielded assembly comprised of a substrate and, disposed above a substrate, a shield comprising from about 1 to about 99 weight percent of a first nanomagnetic material, and from about 99 to about 1 weight percent of a second material with a resistivity of from about 1 microohm-centimeter to about 1×1025 microohm centimeters.

FIG. 29 of U.S. Pat. No. 6,713,671 is a schematic of a preferred shielded assembly 3000 that is comprised of a substrate 3002. The substrate 3002 may be any one of the substrates illustrated hereinabove. Alternatively, or additionally, it may be any receiving surface which it is desired to shield from magnetic and/or electrical fields. Thus, e.g., the substrate can be substantially any size, any shape, any material, or any combination of materials. The shielding material(s) disposed on and/or in such substrate may be disposed on and/or in some or all of such substrate.

Referring again to FIG. 29 of U.S. Pat. No. 6,713,671, and by way of illustration and not limitation, the substrate 3002 may be, e.g., a foil comprised of metallic material and/or polymeric material. The substrate 3002 may, e.g., comprise ceramic material, glass material, composites, etc. The substrate 3002 may be in the shape of a cylinder, a sphere, a wire, a rectilinear shaped device (such as a box), an irregularly shaped device, etc.

Referring again to FIG. 29 of U.S. Pat. No. 6,713,67, and in one embodiment, the substrate 3002 preferably a thickness of from about 100 nanometers to about 2 centimeters. In one aspect of this embodiment, the substrate 3002 preferably is flexible.

Referring again to FIG. 29 of U.S. Pat. No. 6,713,671, and in the preferred embodiment depicted therein, it will be seen that a shield 3004 is disposed above the substrate 3002. As used herein, the term “above” refers to a shield that is disposed between a source 3006 of electromagnetic radiation and the substrate 3002.

The shield 3004 is comprised of from about 1 to about 99 weight percent of nanomagnetic material 3008; such nanomagnetic material, and its properties, are described elsewhere in this specification. In one embodiment, the shield 3004 is comprised of at least about 40 weight percent of such nanomagnetic material 3008. In another embodiment, the shield 3004 is comprised of at least about 50 weight percent of such nanomagnetic material 3008.

Referring again to FIG. 29 of such U.S. Pat. No. 6,713,671, and in the preferred embodiment depicted therein, it will be seen that the shield 3004 is also comprised of another material 3010 that preferably has an electrical resistivity of from about about 1 microohm-centimeter to about 1×1025 microohm-centimeters. This material 3010 is preferably present in the shield at a concentration of from about 1 to about 1 to about 99 weight percent and, more preferably, from about 40 to about 60 weight percent.

In one embodiment, the material 3010 has a dielectric constant of from about 1 to about 50 and, more preferably, from about 1.1 to about 10. In another embodiment, the material 3010 has resistivity of from about 3 to about 20 microohm-centimeters.

In one embodiment, the material 3010 preferably is a nanoelectrical material with a particle size of from about 5 nanometers to about 100 nanometers.

In another embodiment, the material 3010 has an elongated shape with an aspect ratio (its length divided by its width) of at least about 10. In one aspect of this embodiment, the material 3010 is comprised of a multiplicity of aligned filaments.

In one embodiment, the material 3010 is comprised of one or more of the compositions of U.S. Pat. Nos. 5,827,997 and 5,643,670.

Thus, e.g., the material 3010 may comprise filaments, wherein each filament comprises a metal and an essentially coaxial core, each filament having a diameter less than about 6 microns, each core comprising essentially carbon, such that the incorporation of 7 percent volume of this material in a matrix that is incapable of electromagnetic interference shielding results in a composite that is substantially equal to copper in electromagnetic interference shielding effectives at 1-2 gigahertz. Reference may be had, e.g., to U.S. Pat. No. 5,827,997, the entire disclosure of which is hereby incorporated by reference into this specification.

In another embodiment, the material 3010 is a particulate carbon complex comprising: a carbon black substrate, and a plurality of carbon filaments each having a first end attached to said carbon black substrate and a second end distal from said carbon black substrate, wherein said particulate carbon complex transfers electrical current at a density of 7000 to 8000 milliamperes per square centimeter for a Fe+2/Fe+3 oxidation/reduction electrochemical reaction couple carried out in an aqueous electrolyte solution containing 6 millmoles of potassium ferrocyanide and one mole of aqueous potassium nitrate.

In another embodiment, the material 3010 may be a diamond-like carbon material. As is known to those skilled in the art, this diamond-like carbon material has a Mohs hardness of from about 2 to about 15 and, preferably, from about 5 to about 15. Reference may be had, e.g., to U.S. Pat. Nos. 5,098,737 (amorphic diamond material), U.S. Pat. No. 5,658,470 (diamond-like carbon for ion milling magnetic material), U.S. Pat. No. 5,731,045 (application of diamond-like carbon coatings to tungsten carbide components), U.S. Pat. No. 6,037,016 (capacitively coupled radio frequency diamond-like carbon reactor), U.S. Pat. No. 6,087,025 (application of diamond like material to cutting surfaces), and the like. The entire disclosure of each of these United States patents is hereby incorporated by reference into this specification.

In another embodiment, material 3010 is a carbon nanotube material. These carbon nanotubes generally have a cylindrical shape with a diameter of from about 2 nanometers to about 100 nanometers, and length of from about 1 micron to about 100 microns.

These carbon nanotubes are well known to those skilled in the art. Reference may be had, e.g., to U.S. Pat. No. 6,203,864 (heterojunction comprised of a carbon nanotube), U.S. Pat. No. 6,361,861 (carbon nanotubes on a substrate), U.S. Pat. No. 6,445,006 (microelectronic device comprising carbon nanotube components), U.S. Pat. No. 6,457,350 (carbon nanotube probe tip), and the like. The entire disclosure of each of these United States patents is hereby incorporated by reference into this specification.

In one embodiment, material 3010 is silicon dioxide particulate matter with a particle size of from about 10 nanometers to about 100 nanometers.

In another embodiment, the material 3010 is particulate alumina, with a particle size of from about 10 to about 100 nanometers. Alternatively, or additionally, one may use aluminum nitride particles, cerium oxide particles, yttrium oxide particles, combinations thereof, and the like; regardless of the particle(s) used, it is preferred that its particle size be from about 10 to about 100 nanometers.

Referring again to FIG. 29 of U.S. Pat. No. 6,713,671, and in the embodiment depicted in such FIG. 29, the shield 3004 is in the form of a layer of material that has a thickness of from about 100 nanometers to about 10 microns. In this embodiment, both the nanomagnentic particles 3008 and the electrical particles 3010 are present in the same layer.

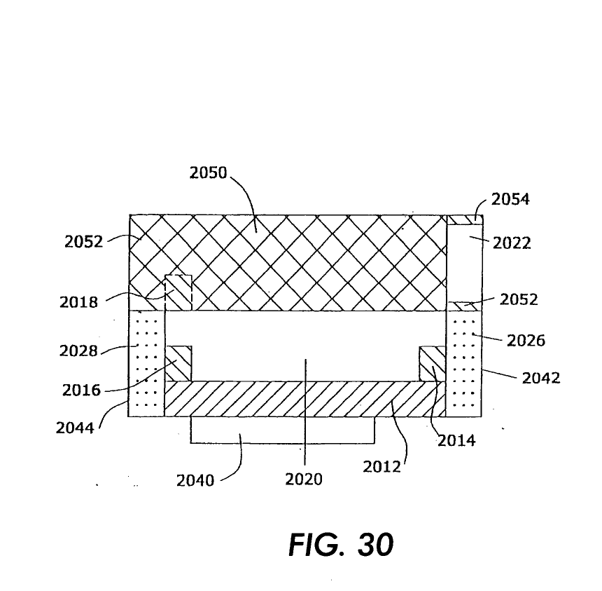

In the embodiment depicted in FIG. 30 of U.S. Pat. No. 6,713,671, by comparison, the shield 3012 is comprised of layers 3014 and 3016. The layer 3014 is comprised of at least about 50 weight percent of nanomagnetic material 3008 and, preferably, at least about 90 weight percent of such nanomagnetic material 3008. The layer 3016 is comprised of at least about 50 weight percent of electrical material 3010 and, preferably, at least about 90 weight percent of such electrical material 3010.

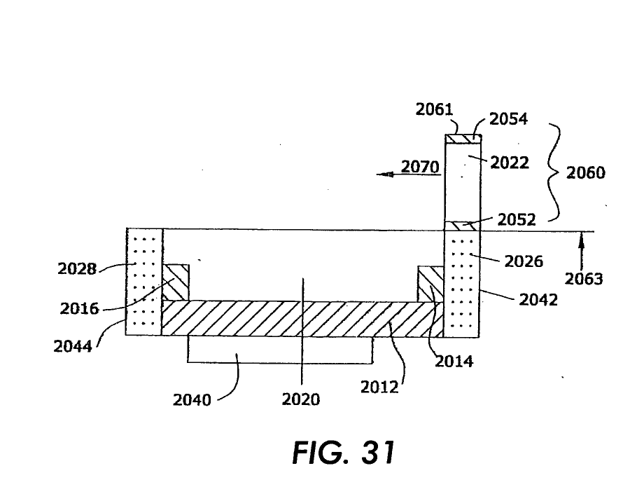

Referring to FIG. 30 of U.S. Pat. No. 6,713,671, and in the embodiment depicted therein, the layer 3014 is disposed between the substrate 3002 and the layer 3016. In the embodiment depicted in FIG. 31, the layer 3016 is disposed between the substrate 3002 and the layer 3014. Each of the layers 3014 and 3016 preferably has a thickness of from about 10 nanometers to about 5 microns.

Referring again to FIG. 30 of U.S. Pat. No. 6,713,671, and in one embodiment, the shield 3012 has an electromagnetic shielding factor of at least about 0.9., i.e., the electromagnetic field strength at point 3020 is no greater than about 10 percent of the electromagnetic field strength at point 3022.

Referring again to FIG. 31 of U.S. Pat. No. 6,713,671, and in one preferred embodiment, the nanomagnetic material preferably has a mass density of at least about 0.01 grams per cubic centimeter, a saturation magnetization of from about 1 to about 36,000 Gauss, a coercive force of from about 0.01 to about 5000 Oersteds, a relative magnetic permeability of from about 1 to about 500,000, and an average particle size of less than about 100 nanometers.

Preparation of a Coated Stent

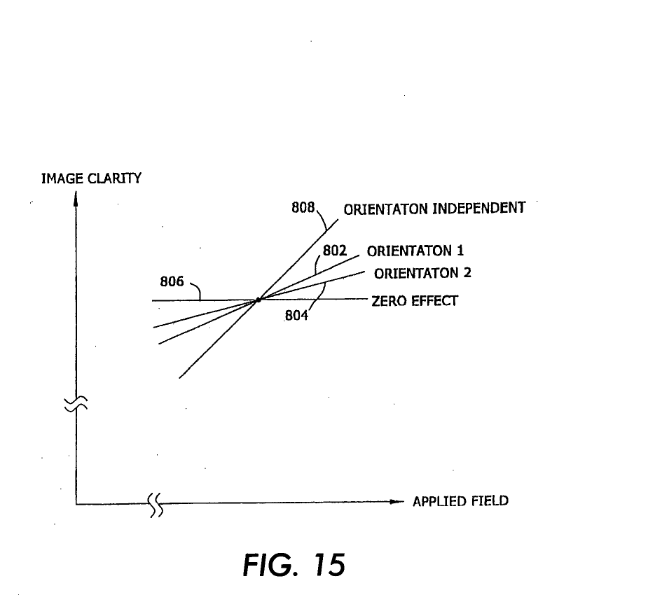

In one embodiment, the stent described elsewhere in this specification is coated with a coating that provides specified “signature” when subjected to the MRI field, regardless of the orientation of the stent. This effect is illustrated in FIG. 15.

FIG. 15 is a plot of the image response of the MRI apparatus (image clarity) as a function of the applied MRI fields. The image clarity is generally related to the net reactance.

Referring to FIG. 15, plot 802 illustrates the response of a particular uncoated stent in a first orientation in a patient's body. As will be seen from plot 802, this stent in this first orientation has an effective net inductive response.

FIG. 15, and in particular plot 804, illustrates the response of the same uncoated stent in a second orientation in a patient's body. As has been discussed elsewhere in this specification, the response of an uncoated stent is orientation specific. Thus, plot 804 shows a smaller inductive response than plot 802.

When the uncoated stent is coated with the appropriate coating, as described elsewhere in this specification, the net reactive effect is zero, as is illustrated in plot 806. In this plot 806, the magnetic response of the substrate is nullified regardless of the orientation of such substrate within a patient's body.

In one embodiment, illustrated as plot 808, a stent is coated in such a manner that its net reactance is substantially larger than zero, to provide a unique imaging signature for such stent. Because the imaging response of such coated stent is also orientation independent, one may determine its precise location in a human body with the use of conventional MRI imaging techniques. In effect, the coating on the stent 808 acts like a tracer, enabling one to locate the position of the stent 808 at will.

In one embodiment, if one knows the MRI signature of a stent in a certain condition, one may be able to determine changes in such stent. Thus, for example, if one knows the signature of such stent with plaque deposited on it, and the signature of such stent without plaque deposited on it, one may be able to determine a human body's response to such stent.

Devices Incorporating the Shielded Conductor Assembly

In this section of the specification, various devices that incorporate the shielded conductor assemblies disclosed in, e.g., FIGS. 6A through 6E are described. The devices described in this section of the specification may also utilize other coating constructs disclosed in this specification.

The inventions described in this section of the specification relates generally to an implantable device that is immune or hardened to electromagnetic insult or interference. More particularly, and in one preferred embodiment, the invention is directed to implantable medical leads that utilize shielding to harden or make these systems immune from electromagnetic insult, namely magnetic-resonance imaging insult.

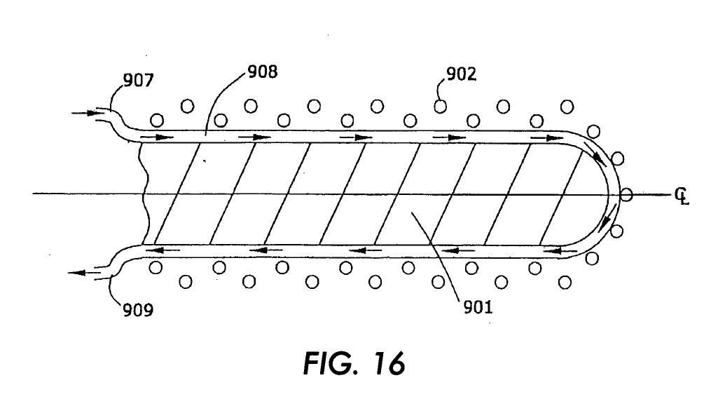

Reference may be had to an article by Neil Mathur et al. entitled “Mesoscopic Texture in Magnanites” (January, 2003, Physics Today” for a discussion of the fact that “ . . . in cetain oxides of manganese, a spectacularly diverse range of exotic electronic and magnetic phases can coexist at different locations within a single crystal. This striking behavior arises in FIG. 12, which is a schematic sectional view of substrate 901, which is part of an implantable medical device (not shown). Referring to FIG. 16, and to the embodiment depicted therein, it will be seen that substrate 901 is coated with nanomagnetic particulate material 902.

In the embodiment depicted in FIG. 16, the substrate 901 may be a cylinder, such as an enclosure for a catheter, medical stent, guide wire, and the like. The assembly depicted in FIG. 16 preferably includes a channel 508 located on the periphery of the medical device. An actively circulating, heat-dissipating fluid (not shown) can be pumped into channel 908 through port 907, and exit channel 908 through port 909. The heat-dissipation fluid (not shown) will draw heat to another region of the device, including regions located outside of the body where the heat can be dissipated at a faster rate. In the embodiment depicted, the heat-dissipating flow flows internally to the layer of nanomagnetic particles 902

In another embodiment, not shown, the heat dissipating fluid flows externally to the layer of nanomagnetic particulate material 902.

In another embodiment (not shown), one or more additional polymer layers (not shown) are coated on top of the layer of nanomagnetic particulate 902. In one aspect of this embodiment, a high thermal conductivity polymer layer is coated immediately over the layer of nanomagnetic particulate 902; and a low thermal conductivity polymer layer is coated over the high thermal conductivity polymer layer, It is preferred that neither the high thermal conductivity polymer layer nor the low thennal conductivity polymer layer be electrically or magnetically conductive. In the event of the occurrence of “hot spots” on the surface of the medical device, heat from the localized “hot spots” will be conducted along the entire length of the device before moving radially outward through the insulating outer layer. Thus, heat is distributed more uniformly.

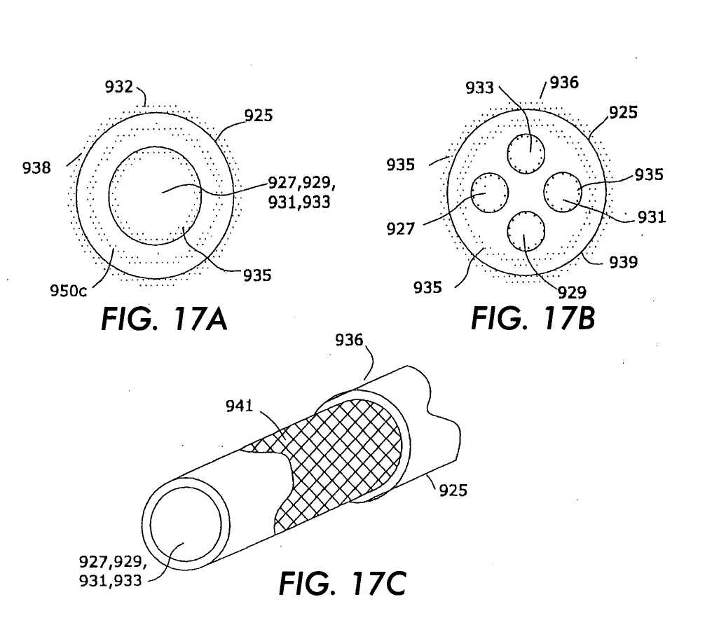

FIGS. 17A, 17B, and 17C are schematic views of a catheter assembly similar to the assembly depicted in FIG. 2 of U.S. Pat. No. 3,995,623; the entire disclosure of such patent is hereby incorporated by reference into this specification. Referring to FIG. 6 of such patent, and also to FIGS. 17A, 17B, and 17C, it will be seen that catheter tube 625 contains multiple lumens 927, 929, 931, and 933, which can be used for various functions such as inflating balloons, enabling electrical conductors to communicate with the distal end of the catheter, etc. While such four lumens are shown, it is to be understood that this invention applies to a catheter with any number of lumens.

The similar catheter disclosed and claimed in U.S. Pat. No. 3,995,623 may be shielded by coating it in whole or in part with a coating of nanomagnetic particulate.

In the embodiment depicted in FIG. 17B, a nanomagnetic material 935 is applied to the interior walls of multiple lumens 927, 929, 931, 933 within a single catheter 934 or the common exterior wall 939 or imbibed into the common wall 939.

In the embodiment depicted in FIG. 17C, a nanomagnetic material 925 is applied to the mesh-like material 941 used within the wall of catheter 936 to give it desired mechanical, electrical, and magnetic properties.

In another embodiment (not shown) a sheath coated with nanomagnetic material on its internal surface, exterior surface, or imbibed into the wall of such sheath, is placed over a catheter to shield it from electromagnetic interference. In this manner, existing catheters can be made MRI safe and compatible, The modified catheter assembly thus produced is resistant to electromagnetic radiation.

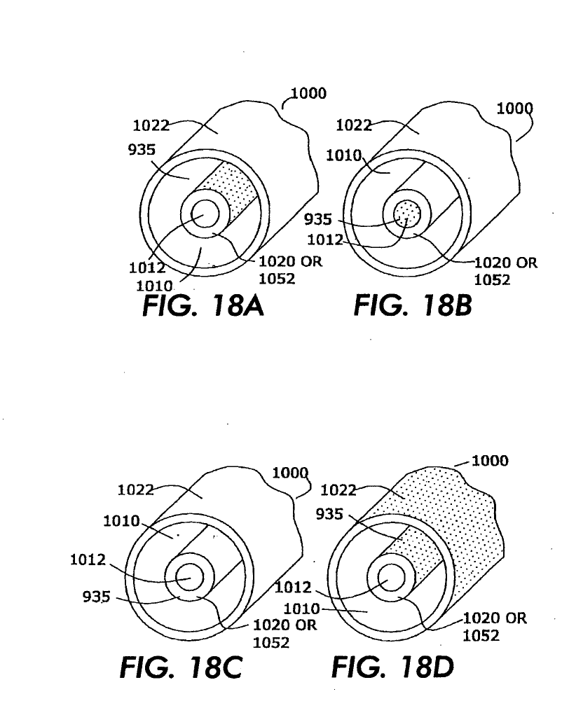

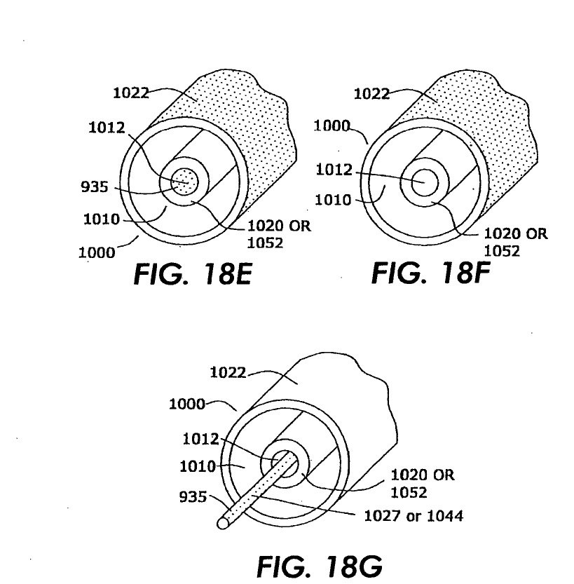

FIGS. 18A through 18G are schematic views of a catheter assembly 1000 consisting of multiple concentric elements. While two elements are shown; 1020 and 1022 are shown, it is to be understood that any number of overlapping elements may be used, either concentrically or planarly positioned with respect to each other.

Referring to FIGS. 18A through 18G, and in the preferred embodiment depicted therein, it will be seen that catheter assembly 1000 comprises an elongated tubular construction having a single, central or axial lumen 1010. The exterior catheter body 1022 and concentrically positioned internal catheter body 1020 with internal lumen 1012 are preferably flexible, i.e., bendable, but substantially non-compressible along its length. The catheter bodies 1020 and 1022 may be made of any suitable material. A presently preferred construction comprises an outer wall 1022 and inner wall 1020 made of a polyurethane, silicone, or nylon.

The outer wall 1022 preferably comprises an imbedded braided mesh of stainless steel or the like to increase torsional stiffness of the catheter assembly 1000 so that, when a control handle, not shown, is rotated, the tip sectionally of the catheter will rotate in corresponding manner.

The catheter assembly 1000 may be shielded by coating it in whole or in part with a coating of nanomagnetic particulate 935, in any one or more of the manners described in this specification.

Referring to FIG. 18A, a nanomagnetic material 935 may be coated on the outside surface of the inner concentrically positioned catheter body 1020.

Referring to FIG. 18C, a nanomagnetic material 935 may be imbibed into the walls of the inner concentrically positioned catheter body 1020 and externally positioned catheter body 1022. Although not shown, a nanomagnetic material may be imbibed solely into either inner concentrically positioned catheter body 1020 or externally positioned catheter body 1022.

Referring to FIG. 18D, a nanomagnetic material 935 may be coated onto the exterior wall of the inner concentrically positioned catheter body 1020 and external catheter body 1022.

Referring to FIG. 18E, a nanomagnetic material 935 may be coated onto the interior wall of the inner concentrically positioned catheter body 1020 and externally wall of externally positioned catheter body 1022.

Referring to FIG. 18F, a nanomagnetic material 935 may be coated on the outside surface of the externally positioned catheter body 1022.

Referring to FIG. 18G, a nanomagnetic material 935 may be coated onto the exterior surface of an internally positioned solid element 1027.

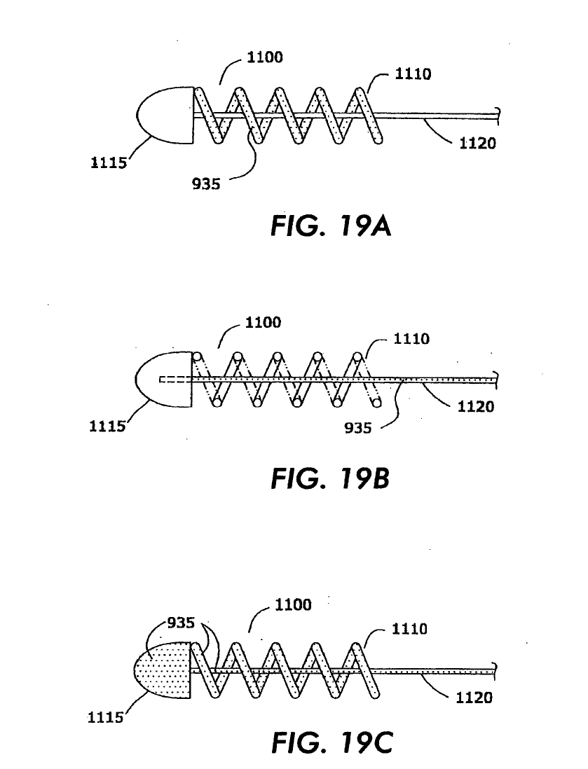

By way of further illustration, one may apply nanomagnetic particulate material to one or more of the catheter assemblies disclosed and claimed in U.S. Pat. Nos. 5,178,803, 5,041,083, 6,283,959, 6,270,477, 6,258,080, 6,248,092, 6,238,408, 6,208,881, 6,190,379, 6,171,295, 6,117,064, 6,019,736, 5,964,757, 5,853,394, and 6,235,024, the entire disclosure of each of which is hereby incorporated by reference into this specification. The catheters assemblies disclosed and claimed in the above-mentioned United States patents may be shielded by coating them in whole or in part with a coating of nanomagnietic particulate 935 FIGS. 19A, 19B. and 19C are schematic views of a guide wire assembly 1100 for insertion into a vascular vessel (not shown), and it is similar to the assembly depicted in U.S. Pat. No. 5,460,187, the entire disclosure of such patent is incorporated by reference into this specification. Referring to FIG. 19A, a coiled guide wire 1110 is formed of a proximal section (not shown) and central support wire 120 that terminates in hemispherical shaped tip 115. The proximal end has a retaining device (not shown) that enables the person operating the guide wire to turn an orient the guide wire within the vascular conduit.

The guide wire assembly may be shielded by coating it in whole or in part with a coating of nanomagnetic particulate 935.

By way of further illustration, one may coat with nanomagnetic particulate matter the guide wire assemblies disclosed and claimed in U.S. Pat. Nos. 5,211,183, 6,168,604, 6,093,157, 6,019,737, 6,001,068, 5,938,623, 5,797,857, 5,588,443, 5,452,726, and the like; the entire disclosure of each of these United States patents is hereby incorporated by reference into this specification.

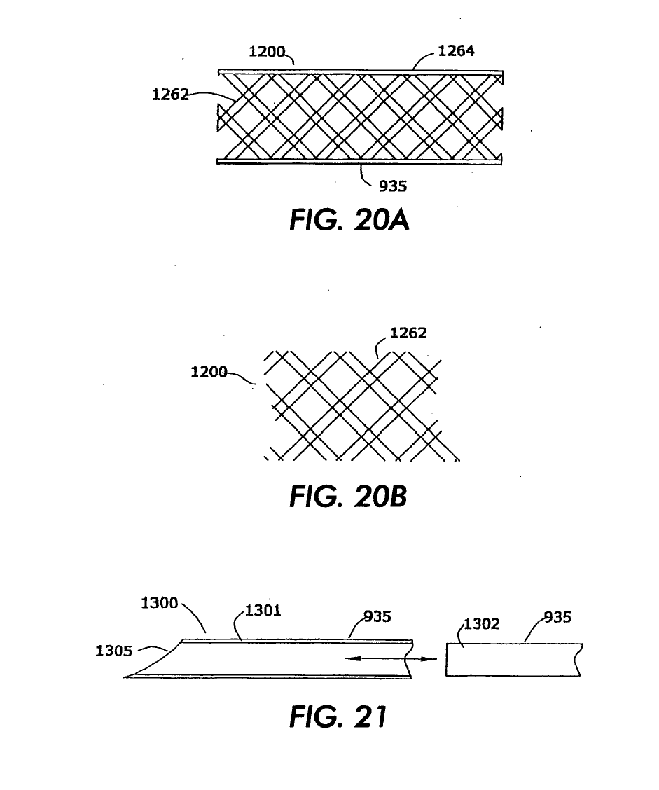

FIGS. 20A and 20B are schematic views of a medical stent assembly 1200 similar to the assembly depicted in FIG. 15 of U.S. Pat. No. 5,443,496; the entire disclosure of such patent is hereby incorporated by reference into this specification.

Referring to FIG. 20, a self-expanding stent 1200 comprising joined metal stent elements 1262 is shown. The stent 1200 also comprises a flexible film 1264. The flexible film 1264 can be applied as a sheath to the metal stent elements 1262 after which the stent 1200 can be compressed, attached to a catheter, and delivered through a body lumen to a desired location. Once in the desired location, the stent 1200 can be released from the catheter and expanded into contact with the body lumen, where it can conform to the curvature of the body lumen. The flexible film 1264 is able to form folds, which allow the stent elements to readily adapt to the curvature of the body lumen. The medical stent assembly disclosed and claimed in U.S. Pat. No. 5,443,496 may be shielded by coating it in whole or in part with a nanomagnetic coating 935 (not shown).

In the embodiment depicted in FIG. 20A, flexible film 1264 is coated with a nanomagentic coating 935 on its inside or outside surfaces, or within the film itself.

It is to be understood that any one of the above embodiments may be used independently or in conjunction with one another within a single device.

In yet another embodiment (not shown), a sheath (not shown), coated or imbibed with a nanomagnetic material 935 is placed over the stent 1200, particularly the flexible film 1264, to shield it from electromagnetic interference. In this manner, existing stents can be made MRI safe and i le.

By way of further illustration, one may coat one or more of the medical stent assemblies disclosed and claimed in U.S. Pat. Nos. 6,315,794, 6,190,404, 5,968,091, 4,969,458, 6,342,068, 6,312,460, 6,309,412, and 6,305,436, the entire disclosure of each of which is hereby incorporated by reference into this specification. The medical stent assemblies disclosed and claimed in the above-mentioned United States patents may be shielded by coating them in whole or in part with a coating of nanomagmetic particulate, as described above.

FIG. 21 is a schematic view of a biopsy probe assembly 1300 similar to the assembly depicted in FIG. 1 of U.S. Pat. No. 5,005,585 the entire disclosure of such patent is hereby incorporated by reference into this specification. Such biopsy probe assembly 1300 is composed of three separate components, a hollow tubular cannula or needle 1301, a solid intraluminar rod-like stylus 1302, and a clearing rod or probe (not shown).

The components of the assembly 1300 are preferably formed of an alloy, such as stainless steel, which is corrosion resistant and non-toxic. Cannula 1301 has a proximal end (not shown) and a distal end 1305 that is cut at an acute angle with respect to the longitudinal axis of the cannula and provides an annular cutting edge.

By way of further illustration, biopsy probe assemblies are disclosed and claimed in U.S. Pat. Nos. 4,671,292, 5,437,283, 5,494,039, 5,398,690, and 5,335,663, the entire disclosure of each of which is hereby incorporated by reference into this specification. The biopsy probe assemblies disclosed and claimed in the above-mentioned United States patents may be shielded by coating them in whole or in part with a coating of nanomagnetic particulate. Thus, e.g., cannula 1301 may be coated, intralurninar stylus 1302 may be coated, and/or the clearing rod may be coated.

In one variation on this design (not shown), a biocompatible sheath is placed over the coated cannula 1301 to protect the nanomagnetic coating from abrasion and from contacting body fluids.

In another embodiment, the biocompatible sheath has on its interior surface or within its walls a nanomagnetic coating.

In yet another embodiment (not shown), a sheath coated or imbibed with a nanomagnetic material is placed over the biopsy probe, to shield it from electromagnetic MRI is increasingly being used interoperatively to guide the placement of medical devices such as endoscopes which are very good at treating or examining tissues close up, but generally cannot accurately determine where the tissues being examined are located within the body.

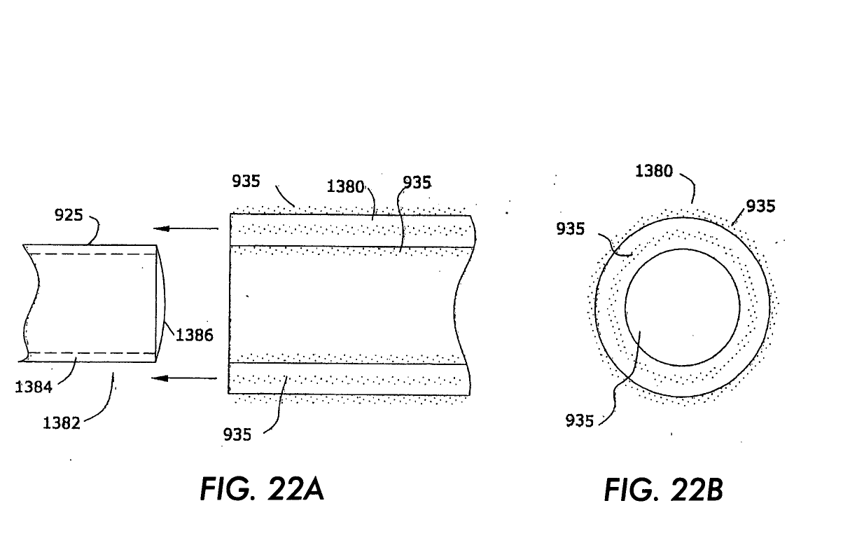

FIGS. 22A and 22B are schematic views of a flexible tube endoscope assembly 1380. Referring to FIG. 22A, the endoscope 1382 employs a flexible tube 1384 with a distally positioned objective lens 1386. Flexible tube 1384 is preferably formed in such manner that the outer side of a spiral tube is closely covered with a braided-wire tube (not shown) formed by weaving fine metal wires into a braid. The spiral tube is formed using a precipitation hardening alloy material, for example, beryllium bronze (copper-beryllium alloy).

By way of further illustration, endoscope tube assemblies are disclosed and claimed in U.S. Pat. Nos. 4,868,015, 4,646,723, 3,739,770, 4,327,711, and 3,946,727, the entire disclosure of each of which is hereby incorporated by reference into this specification. The endoscope tube assemblies disclosed and claimed in the above-mentioned United States patents may be shielded by coating them in whole or in part with a coating of nanomagnetic particulates.

Referring again to FIG. 22A; sheath 1380 is a sheath coated with nanomagnetic material 935 on its inside surface and its exterior surface, or imbibed into its structure; and such sheath 1380 is placed over the endoscope 1382, particularly the flexible tube 1384, to shield it from electromagnetic interference.

In yet another embodiment (not shown), flexible tube 1384 is coated with nanomagnetic materials on its internal surface, or imbibed with nanomagnetic materials within its wall.

In another embodiment (not shown), the braided-wire element within flexible tube 1384 is coated with a nanomagnetic material.

In this manner, existing endoscopes can be made MRI safe and compatible. The modified endoscope tube assemblies thus produced are resistant to electromagnetic radiation.

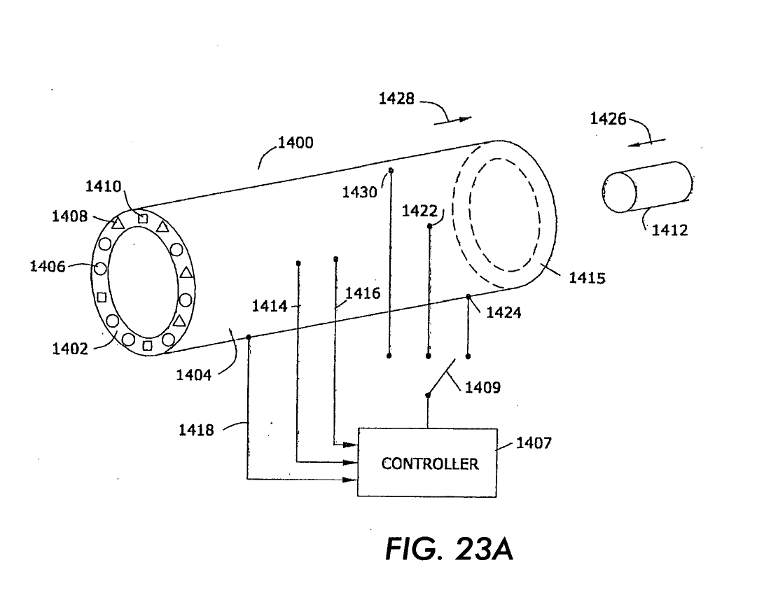

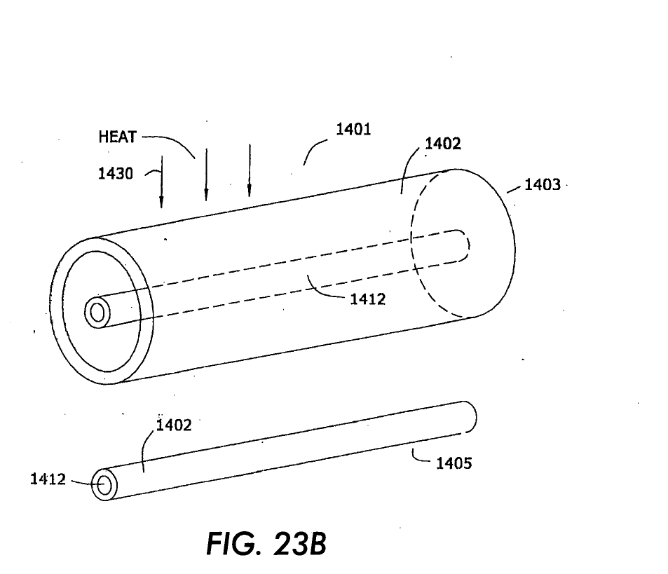

FIG. 23A is a schematic illustration of a sheath assembly 1400 comprised of a sheath 1402 whose surface 1404 is comprised of a multiplicity of nanomagentic materials 1406, 1408, and 1410.

The sheath 1402 may be formed from electrically conductive materials that include metals, carbon composites, carbon nanotubes, metal-coated carbon filaments (wherein the metal may be either a ferromagnetic material such as nickel, cobalt, or magnetic or nonmagnetic stainless steel; a paramagnetic material such as titanium, aluminum, magnesium, copper, silver, gold, tin, or zinc; a diamagnetic material such as bismuth, or well known superconductor materials), metal-coated ceramic filaments (wherein the metal may be one of the following metals: nickel, cobalt, magnetic or non-magnetic stainless steel, titanium, aluminum, magnesium, copper, silver, gold, tin, zinc, bismuth, or well known superconductor materials, a composite of metal-coated carbon filaments and a polymer (wherein the polymer may be one of the following: polyether sulfone, silicone, polymide, polyvinylidene fluoride, epoxy, or urethane), a composite of metal-coated ceramic filaments and a polymer (wherein the polymer may be one of the following: polyether sulfane, silicone, polymide, polyvinylidene fluoride, epoxy, or urethane), a composite of metal-coated carbon filaments and a ceramic (wherein the ceramic may be one of the following: cement, silicates, phosphates, silicon carbide, silicon nitride, aluminum nitride, or titanium diboride), a composite of metal-coated ceramic filaments and a ceramic (wherein the ceramic may be one of the following: cement, silicates, phosphates, silicon carbide, silicon nitride, aluminum nitride, or titanium diboride), or a composite of metal-coated (carbon or ceramic) filaments (wherein the metal may be one of the following metals: nickel, cobalt, magnetic or nonmagnetic stainless steel, titanium, aluminum, magnesium, copper, silver, gold, tin, zinc, bismuth, or well known superconductor materials), and a polymer/ceramic combination (wherein the polymer may be one of the following: polyether sulfone, silicone, polymide, polyvinylidene fluoride, or epoxy and the ceramic may be one of the following: cement, silicates, phosphates, silicon carbide, silicon nitride, aluminum nitride, or titanium diboride).

In one preferred embodiment, the sheath 1402 is comprised of at least about 50 volume percent of the nanomagnetic material 935 described elsewhere in this specification.

As is known to those skilled in the art, liquid crystals are anonisotrpic materials (that are neither crystalline nor liquid) composed of long molecules that, when aligned, are parallel to each other in long crystals. Ferromagnetic liquid crystals are known to those in the art, and they are often referred to as FMLC. Reference may be had, e.g., to U.S. Pat. Nos. 4,241,521, 6,451,207, 5,161,030, 6375,330, 6,130,220, and the like. The entire disclosure of each of these United States patents is hereby incorporated by reference into this specification.

Reference also may be had to U.S. Pat. No. 5,825,448, which describes a reflective liquid crystalline diffractive light valve. The figures of this patent illustrate how the orientations of the magnetic liquid crystal particles align in response to an applied magnetic field. The entire disclosure of this United States patent is hereby incorporated by reference into this specifiction.

Referring again to FIG. 23A, and to the embodiment depicted therein, it will be seen that sheath 1402 may be disposed in whole or in part over medical device 1412. In the embodiment depicted, the sheath 1402 is shown as being bigger than the medical device 1412. It will be apparent that such sheath 1402 may be smaller than the medical device 1412, may be the same size as the medical device 1412, may have a different cross-sectional shape than the medical 1412, and the like.

In one preferred embodiment, the sheath 1402 is disposed over the medical device 1412 and caused to adhere closely thereto. One may create this adhesion either by use of adhesive(s) and/or by mechanical shrinkage.

In one embodiment, shrinkage of the sheath 1412 is caused by heat, utilizing well known shrink tube technology. Reference may be had, e.g., to U.S. Pat. Nos. 6,438,229, 6,245,053, 6,082,760, 6,055,714, 5,903,693. and the like. The entire disclosure of each of these United States patents is hereby incorporated by reference into this specification.

In another embodiment of the invention, the sheath 1402 is a rigid or flexible tube formed from polytetrafluoroethylene that is heat shrunk into resilient engagement with the implantable medical device. The sheath can also be formed from heat shrinkable polymer materials e.g., low density polyethylene (LDPE), linear low-density polyethylene (LLDPE), ethylene vinyl acrylate (EVA), ethylene methacrylate (EMA), ethylene methacrylate acid (EMAA) and ethyl glycol methacrylic acid (EGMA). The polymer material of the heat shrinkable sheath should have a Vicat softening point less than 50 degrees Centigrade and a melt index less than 25. A particularly suitable polymer material for the sheath of the invention is a copolymer of ethylene and methyl acrylate.

In another embodiment of the invention, the sheath 1402 is a collapsible tube that can be extended over the implantable medical device such as by unrolling or stretching.

In yet another embodiment of the invention, the sheath 1402 contains a tearable seam along its axial length, to enable the sheath to be withdrawn and removed from the implantable device without explanting the device or disconnecting the device from any attachments to its proximal end, thereby enabling the electromagnetic shield to be removed after the device is implanted in a patient. This is a preferred feature of the sheath, since it eliminates the need to disconnect any devices connected to the proximal (external) end of the device, which could interrupt the function of the implanted medical device. This feature is particularly critical if the shield is being applied to a life-sustaining device, such as a temporary implantable cardiac pacemaker.

The ability of the sheath 1402 to be easily removed, and therefore easily disposed of, without disposing of the typically much more expensive medical device being shielded, is a preferred feature since it prevents cross-contamination between patients using the same medical device.

In still another embodiment of the invention, an actively circulating, heat-dissipating fluid is pumped into one or more internal channels within the sheath. The heat-dissipation fluid will draw heat to another region of the device, including regions located outside of the body where the heat can be dissipated at a faster rate. The heat-dissipating flow may preferably flow internally to the layer of nanomagnetic particles 935, or external to the layer of nanomagnetic particulate material 935.

FIG. 23B illustrates a process 1401 in which heat 1430 is applied to a shrink tube assembly 1403 to produce the final product 1405. For the sake of simplicity of representation, the controller 1407 has been omitted from FIG. 23B.

Referring again to FIG. 23A, and in the preferred embodiment depicted therein, it will be seen that a controller 1407 is connected by switch 1409 to the sheath 1402. A multiplicity of sensors 1414 and 1416, e.g., can detect the effectiveness of sheath 1402 by measuring, e.g., the temperature and/or the electromagnetic field strength within the shield 1412. One or more other sensors 1418 are adapted to measure the properties of sheath 1412 at its exterior surface 1404.

For the particular sheath embodiment utilizing a liquid crystal nanomagnetic particle construction, and depending upon the data received by controller 1407, the controller 1407 may change the shielding properties of shield 1412 by delivering electrical and/or magnetic energy to locations 1420, 1422, 1424, etc. The choice of the energy to be delivered, and its location and duration, will vary depending upon the status of the sheath 1412.

In the embodiment depicted in FIG. 23A, the medical device may be moved in the direction of arrow 1426, while the sheath 1402 may be moved in the direction of arrow 1428, to produce the assembly 1401 depicted in FIG. 23B. Thereafter, heat may be applied to this assembly to produce the assembly 1405 depicted in FIG. 23B.

In one embodiment, not shown, the sheath 1402 is comprised of an elongated element consisting of a proximal end and a distal end, containing one or more internal hollow lumens, whereby the lumens at said distal end may be open or closed; this device is used to temporarily or permanently encase an implantable medical device.

In this embodiment, the elongated hollow element is similar to the sheath disclosed and claimed in U.S. Pat. No. 5,964,730; the entire disclosure of which is hereby incorporated by reference into this specification.

Referring again to FIG. 23A, and in the embodiment depicted therein, the sheath 1402 is preferably coated and/or impregnated with nanomagnetic shielding material 1406/1408/1410 that comprises at least 50 percent of its external surface, and/or comprises at least 50 percent of one or more lumen internal surfaces, or imbibed within the wall 1415 of sheath 1402, thereby protecting at least fifty percent of the surface area of one or more of its lumens, or any combination of these surfaces or areas, thus forming a shield against electromagnetic interference for the encased medical device.

The coatings of this invention may be used to coat a single conductor 133. Alternatively, or additionally, one may coat a multiple strand conductor. Thus, e.g., multiple strand conductors may be shielded by coating each strand separately, or by coating the multiple strand bundle. Thus, e.g., the multiple conductors within a single lead may be positioned concentrically to one another, or positioned spaced apart. Thus, e.g., the internally positioned conductors may be free to move, for example to rotate or translate, to for example control the motion of an active fixation electrode. By way of illustration, the shielded conductors may be used in the lead designs shown in U.S. Pat. Nos. 6,289,251, 6,285,910, 6,192,280, 6,185,463, 6,178,355, 6,144,882, 6,119,042, 6,096,069, 6,066,166, 6,061,598, 6,040,369, 6,038,463, 6,026,567, 6,018,683, 6,016,436, 6,006,122, 5,999,858, 5,991,668, 5,968,087, 5,968,086, 5,967,977, 5,964,795, 5,957,970, 5,957,967, 5,957,965, 5,954,759, 5,948,015, 5,935,159, 5,897,585, 5,871,530, 5,871,528, 5,853,652, 5,796,044, 5,760,341, 5,702,437, 5,676,694, 5,584,873, 5,522,875, 5,423,881, 5,411,545, 5,354,327, 5,336,254, 5,336,253, 5,324,321, 5,303,704, 5,238,006, 5,217,027, 5,007,435, and the like. The entire disclosure of each of these United States patents is hereby incorporated by reference into this specification.

In one embodiment, a conductor assembly comprised of a multifilar coiled conductor with a spiral configuration; is coated with one or more of the coating constructs of this invention. Reference to such a multifilar conductor is made, e.g., in U.S. Pat. No. 5,954,759, the entire disclosure of which is hereby incorporated by reference into this specification.

In one embodiment, one or more of such coating constructs are applied to a monofilar coiled conductor such as, e.g., the monofilar coiled conductor disclosed in U.S. Pat. No. 5,954,759. The entire disclosure of such United States patent is hereby incorporated by reference into this specification.

By way of further illustration, the one or more of the coating constructs may be used to coat one or more of the lead designs shown in U.S. Pat. Nos. 6,289,251, 6,285,910, 6,192,280, 6,185,463, 6,178,355, 6,144,882, 6,119,042, 6,096,069, 6,066,166, 6,061,598, 6,040,369, 6,038,463, 6,026,567, 6,018,683, 6,016,436, 6,006,122, 5,999,858, 5,991,668, 5,968,087, 5,968,086, 5,967,977, 5,964,795, 5,957,970, 5,957,967, 5,957,965, 5,954,759, 5,948,015, 5,935,159, 5,897,585, 5,871,530, 5,871,528, 5,853,652, 5,796,044, 5,760,341, 5,702,437, 5,676,694, 5,584,873, 5,522,875, 5,423,881, 5,411,545, 5,354,327, 5,336,254, 5,336,253, 5,324,321, 5,303,704, 5,238,006, 5,217,027, and 5,007,435; the entire disclosure of each of these United States patents is hereby incorporated by reference into this specification. When so used, the modified assemblies thus produced are resistant to electromagnetic radiation.

In one embodiment, the coating constructs are used to coat a conductor assembly comprised of a multifilar conductor disposed inside a monofilar conductor. In another embodiment, the coating constructs are used to coat a conductor assembly wherein the multifiar conductor is disposed outside the monofilar conductor. In one aspect of this embodiment, only portions of the conductors are shielded.

By way of further illustration, a discontinuous shield is produced by a discontinuous coating of nanomagnetic particles and/or other coating constructs. This coating, e.g., may be may be intermittingly discontinuous along its axial dimension, to provide for example, reduced exposure to an externally applied electromagnetic field. This coating may be, e.g., discontinuous at its proximal end, to provide for example, an electrically conductive surface for attachment to a medical device, such as an implantable pulse generator, a cardioversion-defibrilator pacemaker, an insulin pump, or other tissue or organ stimulating or sensing device. This coating, e.g., may be discontinuous along its distal end, to provide for example, an electrically conductive surface for contacting tissues or organs.

A discontinuous shield may be applied to non-wire conductors, such as for example a solid rod or other geometry conductor, used for example as an electrode for transmitting and/or receiving electrical signals to/from tissues or organs. The discontinuous shield may be applied to any of the conductor or lead configurations described above and/or in U.S. Pat. Nos. 6,289,251, 6,285,910, 6,192,280, 6,185,463, 6,178,355, 6,144,882, 6,119,042, 6,096,069, 6,066,166, 6,061,598, 6,040,369, 6,038,463, 6,026,567, 6,018,683, 6,016,436, 6,006,122, 5,999,858, 5,991,668, 5,968,087, 5,968,086, 5,967,977, 5,964,795, 5,957,970, 5,957,967, 5,957,965, 5,954,759, 5,948,015, 5,935,159, 5,897,585, 5,871,530, 5,871,528, 5,853,652, 5,796,044, 5,760,341, 5,702,437, 5,676,694, 5,584,873, 5,522,875, 5,423,881, 5,411,545, 5,354,327, 5,336,254, 5,336,253, 5,324,321, 5,303,704, 5,238,006, 5,217,027, and 5,007,435; the entire disclosure of each of these patents is hereby incorporated by reference into this specification. Because these devices are coated with nanomagnetic particles, they are resistant to electromagnetic radiation.

In one embodiment, one or more of the coating constructs are used to coat a multiple discontinuously shielded conductor assembly that is comprised of a multiplicity of shielded conductors each of which is coated discontinuously or continuously with nanomagnetic shielding. The centrally disposed conductor is preferably a pacing lead, and the other shielded conductors are preferably cardioversion defibrillation leads. In the embodiment depicted, the entire assembly is shielded with a layer of nanomagnetic material. As will be apparent, the use of discontinuous coating enables the multiple conductors to make electrical contact at one or more points along their axial dimension, to provide redundant electrical channels, in the event one channel should break. The discontinuous coating provides reduced exposure to externally applied electromagnetic fields. The discontinuous shield may be; intermittingly discontinuous along its axial dimension, discontinuous at its proximal end, or discontinuous along its distal end. It is to be understood that the discontinuous shield may be applied to any of the conductor or lead configurations described above.

By way of further illustration, one may use one or more of the coating constructs of this invention to coat a multiconductor lead connected to a catheter and a sheath. This assembly is similar to the assembly depicted in U.S. Pat. No. 6,178,355 (the entire disclosure of which is hereby incorporated by reference into this specification) but differs therefrom in that the use of nanomagnetic particle shielding provides resistance to electromagnetic radiation.

Thus, by way of further illustration, one or more of the nanomagnetic coating constructs of this invention may be used in the lead designs shown in U.S. Pat. Nos. 6,285,910, 6,178,355, 6,119,042, 6,061,598, 6,018,683, 5,968,086, 5,957,967, 5,954,759, 5,871,530, 5,676,694; the entire disclosure of each of which is hereby incorporated by reference into this specification.

In one embodiment, the coating constructs are used to prepare a discontinuously shielded conductor similar to the assembly depicted in FIG. 1 of U.S. Pat. No. 6,016,436. The entire disclosure of this patent is hereby incorporated by reference into this specification In one embodiment, the coated substrate is a lead body that carries at its distal end an insulative electrode head which may be fabricated of a relatively rigid biocompatible plastic, such as a polyurethane; the electrode head carries an advanceable helical electrode. At its proximal end, the lead carries a trifurcated connector assembly provided with two connector pins each coupled to one of two elongated defibrillation electrode coils.

In one embodiment, a coated substrate is produced in which the coating is intermittingly discontinuous along its axial dimension, to enable, for example, direct stimulation and sensing of tissues and organs, while providing, for example, reduced exposure to an externally applied electromagnetic field. Reference may be had, e.g., to the lead designs shown in U.S. Pat. Nos. 6,289,251, 6,285,910, 6,119,042, 6,066,166, 6,061,598, 6,038,463, 6,018,683, 5,957,970, 5,957,967, 5,935,159, 5,871,530, 5,702,437, 5,676,694, 5,584,873, 5,336,254, 5,336,253, 5,238,006, 5,217,027, the entire disclosure of each of which is hereby incorporated by reference into this specification.

In one embodiment, the layer of nanomagnetic material is disposed on or within such medical device(s) and is comprised of electrical circuitry.

One may use the nanomagnetic coating(s) used to shield electronic components located within leads. One may use these coatings to shield medical leads with stranded conductors similar to those depicted in U.S. Pat. No. 6,026,567, the entire disclosure of which is hereby incorporated by reference into this specification. In the embodiment depicted therein, the assembly is comprised of a ring electrode a core 254, a distal insulative sleeve a conductor, a lumen, cross bores, a distal portion and a point adjacent to a shoulder (but see FIGS. 2, 3, and 4 of U.S. Pat. No. 6,026,567).

One may use the coatings constructs to coat a guidewire placed implantable lead with tip seal, such as that disclosed in U.S. Pat. No. 6,192,280 (the entire disclosure of which is hereby incorporated by reference into this specification). Such a lead is preferably comprised of an elongated insulative lead body, a laterally extending ridge, an internal conductive sleeve, a bore, a cup-shaped seal member, a plastic band, a controlled release device, an electrode, a distal tip, and a coiled conductor.

One may use the coating constructs to coat a catheter assembly that is similar to the catheter assembly disclosed in U.S. Pat. No. 6,144,882, the entire disclosure of which is hereby incorporated by reference into this specification.

By way of further illustration, one may use the coating constructs to coat conductor assemblies similar to those depicted in U.S. Pat. No. 5,935,159, the entire disclosure of which is hereby incorporated by reference into this specification. Thus, e.g., one may use the coatings to coat a medical electrical lead system having a torque transfer stylet assembly similar to the assembly depicted in U.S. Pat. No. 5,522,875, the entire disclosure of which is hereby incorporated by reference into this specification.

By way of yet further illustration, the coating constructs may be used to coat a stylet, similar to the stylet depicted in FIG. 7A of U.S. Pat. No. 5,522,875, supra.

In one embodiment, the coating constructs form a film with a thickness of about 100 nanometers or larger, and they produce an article with a specified modulus of elasticity (Young's Modulus). As is known to those skilled in the art, the modulus of elasticity is the ratio of the stress acting on a substance to the strain produced. In general, and in this embodiment, the nanomagnetic particle coatings and films produced by the process of this invention have a tensile modulus of elasticity of at least about 15×10⁶ pounds per square inch.

The coating constructs may be used to coat a steerable wire. Steerable guide wires can be created, for example, by producing differential strain through tension wires electrically exciting piezoelectric elements. Each of these configurations is electrically conductive and susceptible to externally applied electromagnetic fields. The present invention preferably coats these elements with a nanomagnetic coating shield to protect these elements during magnetic resonance imaging-guided installation.

The coating constructs of this invention may be used to coat a transesophageal medical lead similar to the device depicted in U.S. Pat. No. 5,967,977 (see FIG. 1), the entire disclosure of which is hereby incorporated by reference into this specification.

The coating constructs of this invention may be used to coat a torque stylet used to activate a helix in a bent lead; see, e.g., U.S. Pat. No. 5,522,875. The entire disclosure of this United States patent is hereby incorporated by reference into this specification.

The coating constructs of this invention may be used to coat a sheath, in order to shield uncoated conductors positioned within the sheath. Multiple concentrically positioned sheaths are also used to provide additional protection of uncoated conductors positioned within the sheaths. In one embodiment, this sheath is constructed of a tube impregnated with nanomagnetic particles, or a braided wire mesh coated with nanomagnetic particles. In one embodiment, an internally positioned conductor is free to move, e.g., free to rotate or translate. In another embodiment, the motion of the active fixation electrode is controlled. By way of illustration, the shielded conductors described in this specification may be used in the lead designs illustrated in U.S. Pat. Nos. 6,289,251, 6,285,910, 6,192,280, 6,185,463, 6,178,355, 6,144,882, 6,119,042, 6,096,069, 6,066,166, 6,061,598, 6,040,369, 6,038,463, 6,026,567, 6,018,683, 6,016,436, 6,006,122, 5,999,858, 5,991,668, 5,968,087, 5,968,086, 5,967,977, 5,964,795, 5,957,970, 5,957,967, 5,957,965, 5,954,759, 5,948,015, 5,935,159, 5,897,585, 5,871,530, 5,871,528, 5,853,652, 5,796,044, 5,760,341, 5,702,437, 5,676,694, 5,584,873, 5,522,875, 5,423,881, 5,411,545, 5,354,327, 5,336,254, 5,336,253, 5,324,321, 5,303,704, 5,238,006, 5,217,027, and 5,007,435. The entire disclosure of each of these United States patent is hereby incorporated by reference into this specification.

Preparation of Coatings Comprised of Nanoelectrical Material

In this portion of the specification, coatings comprised of nanoelectrical material will be described. In accordance with one aspect of this invention, there is provided a nanoelectrical material with an average particle size of less than 100 nanometers, a surface area to volume ratio of from about 0.1 to about 0.05 l /nanometer, and a relative dielectric constant of less than about 1.5.

The nanoelectrical particles of aspect of the invention have an average particle size of less than about 100 nanometers. In one embodiment, such particles have an average particle size of less than about 50 nanometers. In yet another embodiment, such particles have an average particle size of less than about 10 nanometers.

The nanoelectrical particles of this invention have surface area to volume ratio of from about 0.1 to about 0.05 l /nanometer.

When the nanoelectrical particles of this invention are agglomerated into a cluster, or when they are deposited onto a substrate, the collection of particles preferably has a relative dielectric constant of less than about 1.5. In one embodiment, such relative dielectric constant is less than about 1.2.

In one embodiment, the nanoelectrical particles of this invention are preferably comprised of aluminum, magnesium, and nitrogen atoms. This embodiment is illustrated in FIG. 24.

FIG. 24 illustrates a phase diagram 2000 comprised of moieties A, B, and C. Moiety A is preferably selected from the group consisting of aluminum, copper, gold, silver, and mixtures thereof. It is preferred that the moiety A have a resistivity of from about 2 to about 100 microohm-centimeters. In one preferred embodiment, A is aluminum with a resistivity of about 2.824 microohm-centimeters. As will apparent, other materials with resistivities within the desired range also may be used.

Referring again to FIG. 24, C is selected from the group consisting of nitrogen and oxygen. It is preferred that C be nitrogen, and A is aluminum; and aluminum nitride is present as a phase in system.

Referring again to FIG. 24, B is preferably a dopant that is present in a minor amount in the preferred aluminum nitride. In general, less than about 50 percent (by weight) of the B moiety is present, by total weight of the doped aluminum nitride. In one aspect of this embodiment, less than about 10 weight percent of the B moiety is present, by total weight of the doped aluminum nitride.

The B moiety may be, e.g., magnesium, zinc, tin, indium, gallium, niobium, zirconium, strontium, lanthanum, tungsten, mixtures thereof, and the like. In one embodiment, B is selected from the group consisting of magnesium, zinc, tin, and indium. In another especially preferred embodiment, the B moiety is magnesium.

Referring again to FIG. 24, and when A is aluminum, B is magnesium, and C is nitrogen, it will be seen that regions 2002 and 2003 correspond to materials which have a low relative dielectric constant (less than about 1.5), and a high relative dielectric constant (greater than about 1.5), respectively.

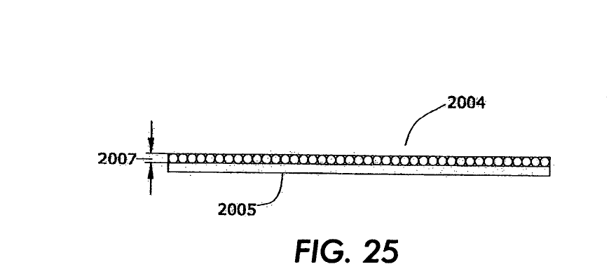

FIG. 25 is a schematic view of a coated substrate 2004 comprised of a substrate 2005 and a multiplicity of nanoelectrical particles 2006. In this embodiment, it is preferred that the nanoelectrical particles 2006 form a film with a thickness 2007 of from about 10 nanometers to about 2 micrometers and, more preferably, from about 100 nanometers to about 1 micrometer.

The description of some of the remaining Figures in this section of the specfication is related to technology that is disclosed in U.S. Pat. No. 6,329,305, the entire disclosure of which is hereby incorporated by reference in to this specification.

Such U.S. Pat. No. 6,329,305, in its Column 1, refers to a patent application U.S. Ser. No. 09/503,225, for a “Method for Producing Piezoelectric Films . . . ;” this patent application issued as U.S. Pat. No. 6,342,134 on Jan. 29, 2003. The entire disclosure of such patent application and such patent is hereby incorporated by reference into this application.

Such U.S. Pat. No. 6,329,305, in its Column 1, also refers to pending patent application U.S. Ser. No. 09/145,323, filed on Sep. 1, 1998, for a “Pulsed DC Reactive Sputtering Method . . . ;” the entire disclosure of such pending application is also hereby incorporated by reference into this application.

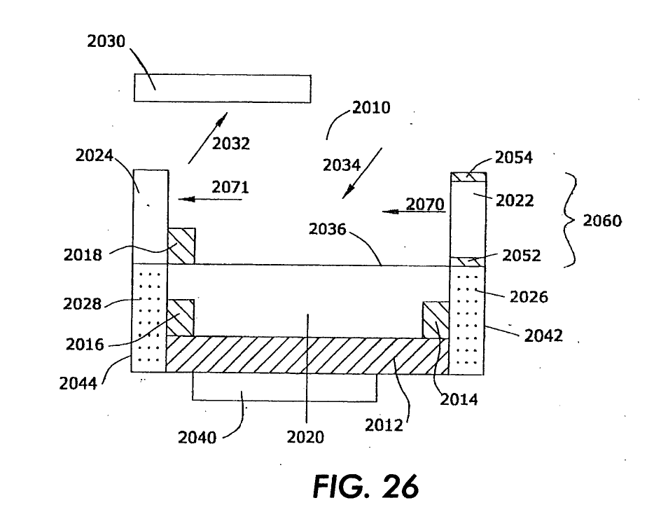

FIG. 26 is a sectional view of a sensor assembly 2010 comprised of a substrate 2012, a conductor 2014, a conductor 2016, a conductor 2018, a piezoelectric element 2020, a source of laser light 2060, a photodetector 2024, and heat conductors 2026 and 2028.

The substrate 2012, in one embodiment, is preferably pure silicon, which, in one embodiment, is single crystal silicon. Processes for making and using single crystal silicon structures are well known. Reference may be had, e.g., to U.S. Pat. Nos. 6,284,309 (epitaxial silicon waver), U.S. Pat. No. 6,136,630 (single crystal silicon), U.S. Pat. No. 5,912,068 (single crystal silicon), U.S. Pat. No. 5,818,100 (single crystal silicon), U.S. Pat. No. 5,646,073 (single crystal silicon), and the like. The entire disclosure of each of these United States patents is hereby incorporated by reference into this specification. The entire disclosure of this United States patent is hereby incorporated by reference into this specification.

Referring again to FIG. 26, and in the preferred embodiment depicted therein, the substrate 2012 generally has a thickness of from about 1 to about 2 millimeters.

In one embodiment, the single-crystal silicon substrate 2012 preferably has a <100> orientation. As is known to those skilled in the art, <100> refers to the lattice orientation of the silicon (see, e.g., Column 5 of U.S. Pat. No. 6,329,305). Reference also may be had to a text by S.M. Sze entitled “Physics of Semiconductor Devices,” 2d Edition (Wiley-Interscience, New York, N.Y., 1981). At page 386 of this text, Table 1 indicates that there are three silicon crystal plane orientations, <111>, <110>, and <100>. The <100> orientation is preferred for one embodiment, the <110> orientation is preferred for a second embodiment, and the <111> orientation is preferred for a third embodiment. In any case, the single crystal silicon substrate 12 has only one of such orientations.

Referring again to FIG. 26, aluminum conductors 2014 and 2016 are grown near the periphery of substrate 2012. The structure depicted in FIG. 26 may be produced by growing an entire layer of aluminum and then etching away a portion thereof.

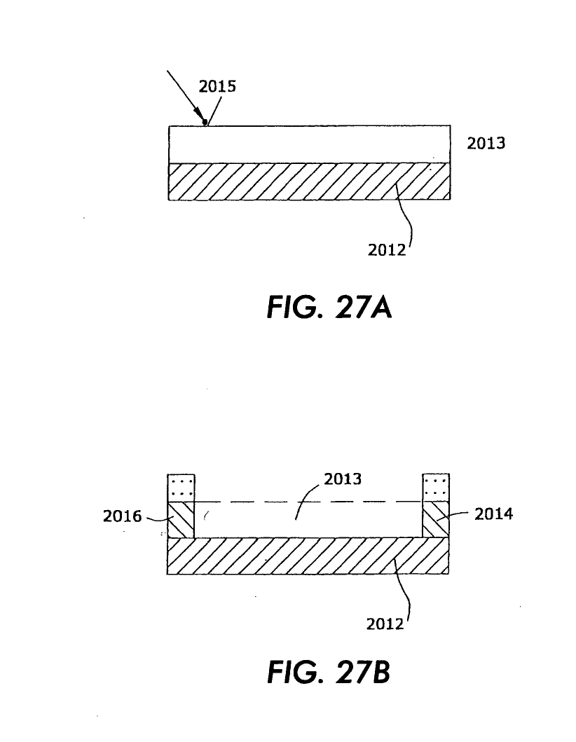

Referring to FIG. 27A, an aluminum layer 2013 may be grown on substrate 2012, preferably by conventional sputtering techniques. Reference may be had, e.g., to U.S. Pat. Nos. 5,835,273 (deposition of an aluminum mirror), U.S. Pat. No. 5,711,858 (deposition of aluminum alloy film), U.S. Pat. No. 4,976,839 (aluminum electrode), and the like. The entire disclosure of each of these United States patents is hereby incorporated by reference into this specification.

One may deposit either aluminum or an aluminum alloy, provided that such aluminum material preferably has a certain conductivity. It is preferred that the aluminum conductor 2014 have a resistivity of less than about 3 microohms-centimeter. Conductor 2016 should have a resistivity of at least 1.5 times as great as the resistivity of conductor 2014, and such resistivity is generally less than about 5 microohms-centimters.

One can vary the resistivity of elements 2014 and 2016 during deposition thereof by preferentially providing a high oxygen content near point 2015 so that conductor 2016, after it has been formed, will contain more oxide material and have a higher resistivity.

Referring again to FIG. 27A, a layer 2013 of aluminum may be deposited onto substrate 2012 by reactive sputtering, as described hereinabove; and, during such deposition, selective reaction with oxygen (or other gases) may be caused to occur at specified points (such as point 2015) of the aluminum layer being deposited. Thereafter, after the solid layer 2013 has been deposited, it can be preferentially etched away.

In one embodiment, and referring again to FIG. 27B, a mask (indicated in dotted line outline) may be deposited onto the layer 2013, and thereafter the unmasked deposited aluminum may be etched away with conventional aluminum etching techniques.

Thus, e.g., one may etch the unmasked area with sputtered with argon or hydrogen or oxygen gas, using conventional sputtering technology; as is known to those skilled in the art, etching is the opposite of deposition. Reference may be had, e.g., to U.S. Pat. Nos. 5,851,364, 5,685,960, 6,222,271, 6,194,783, and the like. The entire disclosure of each of these United States patents is hereby incorporated by reference into this specification.

After the conductors 2014 and 2016 have been integrally formed with substrate 2012, a piezoelectric material 2020 is deposited onto the substrate 2012/conductors/2014-2016 assembly by sputtering. In one preferred embodiment, the piezoelectric material 2020 is piezoelectric aluminum nitride.

In one aspect of this embodiment, after conductors 2014 and 2016 have been formed by sputtering/etching, aluminum nitride is preferably formed by sputtering an aluminum target 2030 with nitrogen gas directed in the direction of arrows 2032 and/or 2034.

In one embodiment, the aluminum nitride layer 2020 (see FIG. 26) has a preferred <002> orientation. Means for producing aluminum nitride with such <002> orientation are well known to those skilled in the art. Reference may be had, e.g., to U.S. Pat. No. 6,329,305, which, at Column 1, refers to “An example of an advantageous film orientation is <002> of AIN perpendicular to the substrate.” This patent claims: “A method for fabricating an electronic device having a piezoelectric material deposited on at least one metal layer, the method comprising depositing the at least one metal layer on a substrate and depositing the piezoelectric material on the metal layer, wherein the texture of the piezoelectric material is determined by controlling the surface roughness of the metal layer.” The entire disclosure of this United States patent is hereby incorporated by reference into this specification.

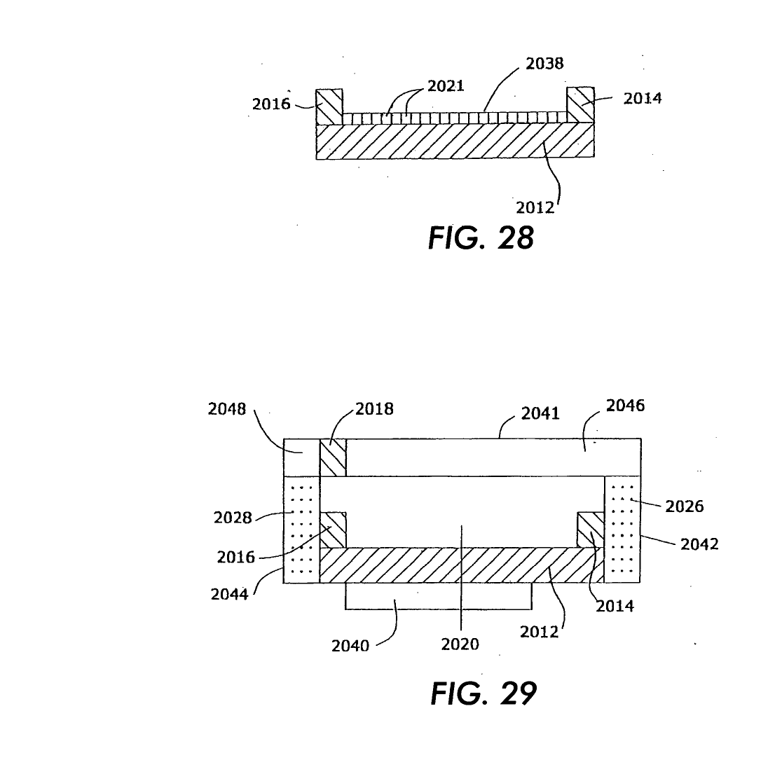

FIG. 28 is a schematic representation of a film orientation <002> of aluminum nitride, with respect to substrate 2012 and/or film plane 2038. Referring to FIG. 26, and in the preferred embodiment depicted therein, it will be seen that columnized growths 2021 preferably form such aluminum nitride 2020. These columnar growths 2021 are substantially perpendicular to the substrate 2012. Reference may be had, e.g., to R. F. Bunshah's “Deposition Technologies for Films and Coatings” (Noyes Publications, Park Ridge, N.J., 1982). At page 131 of such text, columnar grains in a condensate are shown in FIG. 4.36.

Referring again to FIG. 26, the <002> aluminum nitride is deposited up to level 2036 so that layer 2020 has a thickness of about 1 micron. Thereafter, layers 2026 and 2028 are deposited onto the assembly by sputtering.

These layers 2026 and 2028 also preferably consist essentially of aluminum nitride, but they preferably are not piezoelectric. One may obtain such non-piezoelectric properties (or lack thereof) by conventional sputtering techniques in which the aluminum nitride is deposited but no alignment thereof is inducted.

Thus, e.g., in the embodiment depicted in FIG. 26 one may dispose a heater 2040 beneath the substrate 2012 and operate such heater when one is depositing the aluminum nitride material with the <002> orientation (with respect to substrate 2012 and/or film plane 2038) and the piezoelectric properties. Thereafter, one may turn the heater 2040 off while depositing the aluminum nitride layers 2026/2028, neither of which has piezoelectric properties or the <002> orientation with respect to film planes 2042/2044.

However, although the layers 2026 and 2028 do not have piezoelectric properties, they do have certain heat conductivity properties. It is preferred that each of layers 2026 and 2028 have a heat conductance of about 2 Watt/degrees Centigrade/centimeter and a resistivity of about 1×10¹⁶ ohm-centimeter. As will be apparent, each of layers 2026 and 2028 are heat conductors.

FIG. 29 is a schematic of a preferred process similar to that depicted in FIG. 26. Referring to FIG. 26, in the manner described elsewhere in this specification, a layer 2041 of aluminum material is deposited by sputtering (also see FIG. 27A). Thereafter, in the manner depicted in FIG. 27B, portions 2046 and 2048 are etched away by reactive sputtering to leave the integrally formed conductive layer 2018. Thereafter, another layer of aluminum nitride is deposited, as is illustrated in FIG. 30.

Referring to FIG. 30, a layer of aluminum nitride 2050 is deposited by sputtering. This is preferably done only after conductor 2052 is deposited in the manner described hereinabove; and, after it has been done, conductor 2054 is formed in the manner described hereinabove.

The aluminum nitride material that forms layer 2050 preferably has a direct energy band gap of 6.2 electron volts, a heat conductance of about 2 Watt/degrees-Centigrade/centimeter and a resistivity of about 1×10¹⁶ ohm-centimeter. This material also is substantially pure aluminum nitride; and, consequently, it functions as a laser material after it has been formed into the structure depicted in FIG. 30, wherein the section that is shown as being crossed-out is etched away in the manner described elsewhere.

In this embodiment, the final desired structure is depicted in FIG. 31. In another embodiment, shown in FIG. 26, a photodetector layer 2024 is deposited with material which, in one aspect, is substantially the same as material 2022. In this aspect, both structure 2022 and 2024 are preferably simultaneously formed by etching. In this aspect, two aluminum conductors (not shown) are formed in the same manner as conductors 2052 and 2054 (see FIG. 31), but are integrally connected to device 2024.

Referring to FIG. 31, when the laser device 2060 receives electrical current via lines 2061 and 2063, laser light is emitted in the direction of arrow 2070.

Referring to FIG. 26, when photonic energy 2071 impacts photodetector 2024, the electrical properties of photodetector 2024 are changed, whereby a signal is produced from such sensor.

A Coated Substrate with a Dense Coating

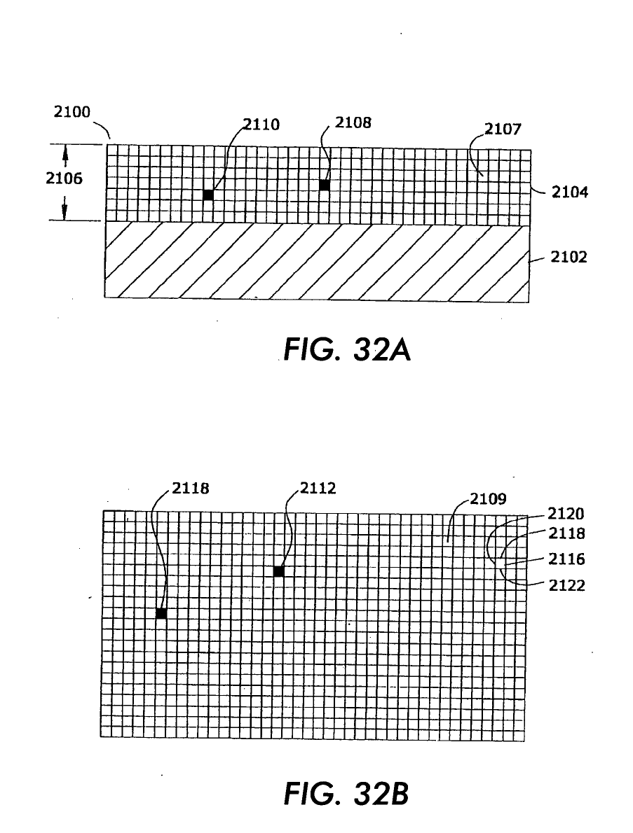

FIGS. 32A and 32B are sectional and top views, respectively, of a coated substrate 2100 assembly comprised of a substrate 2102 and, disposed therein, a coating 2104.

In the embodiment depicted, the coating 2104 has a thickness 2106 of from about 400 to about 2,000 nanometers and, in one embodiment, has a thickness of from about 600 to about 1200 nanometers.

Referring again to FIGS. 32A and 32B, it will be seen that coating 2104 has a morphological density of at least about 98 percent. As is known to those skilled in the art, the morphological density of a coating is a function of the ratio of the dense coating material on its surface to the pores on its surface; and it is usually measured by scanning electron microscopy.

By way of illustration, published United States patent application US 2003/0102222A1 contains a FIG. 3A that is a scanning electron microscope (SEM) image of a coating of “long” single-walled carbon nanotubes on a substrate. Referring to this SEM image, it will be seen that the white areas are the areas of the coating where pores occur.

The technique of making morphological density measurements also is described, e.g., in a M.S. thesis by Raymond Lewis entitled “Process study of the atmospheric RF plasma deposition system for oxide coatings” that was deposited in the Scholes Library of Alfred University, Alfred, N.Y. in 1999 (call Number TP2 a75 1999 vol 1., no. 1.).

FIGS. 32A and 32B schematically illustrate the porosity of the side 2107 of coating 2104, and the top 2109 of the coating 2104. The SEM image depicted shows two pores 2108 and 2110 in the cross-sectional area 2107, and it also shows two pores 2212 and 2114 in the top 2109. As will be apparent, the SEM image can be divided into a matrix whose adjacent lines 2116/2120, and adjacent lines 2118/2122 define square portion with a surface area of 100 square nanometers (10 nanometers×10 nanometers). Each such square portion that contains a porous area is counted, as is each such square portion that contains a dense area. The ratio of dense areas/porous areas,×100, is preferably at least 98. Put another way, the morphological density of the coating 2104 is at least 98 percent. In one embodiment, the morphological density of the coating 2104 is at least about 99 percent. In another embodiment, the morphological density of the coating 2104 is at least about 99.5 percent.

One may obtain such high morphological densities by atomic size deposition, i.e., the particles sizes deposited on the substrate are atomic scale. The atomic scale particles thus deposited often interact with each other to form nano-sized moieties that are less than 100 nanometers in size.

In one embodiment, the coating 2104 (see FIGS. 32A and 32B) has an average surface roughness of less than about 100 nanometers and, more preferably, less than about 10 nanometers. As is known to those skilled in the art, the average surface roughness of a thin film is preferably measured by an atomic force microscope (AFM). Reference may be had, e.g., to U.S. Pat. Nos. 5,420,796 (method of inspecting planarity of wafer surface), U.S. Pat. Nos. 6,610,004, 6,140,014, 6,548,139, 6,383,404, 6,586,322, 5,832,834, and U.S. Pat. No. 6,342,277. The entire disclosure of each of these United States patents is hereby incorporated by reference into this specification.

Alternatively, or additionally, one may measure surface roughness by a laser interference technique. This technique is well known. Reference may be had, e.g., to U.S. Pat. Nos. 6,285,456 (dimension measurement using both coherent and white light interferometers), U.S. Pat. Nos. 6,136,410, 5,843,232 (measuring deposit thickness), U.S. Pat. No. 4,151,654 (device for measuring axially symmetric aspherics), and the like. The entire disclosure of these United States patents are hereby incorporated by reference into this specification.

In one embodiment, the coated substrate of this invention has durable magnetic properties that do not vary upon extended exposure to a saline solution. If the magnetic moment of a coated substrate is measured at “time zero” (i.e., prior to the time it has been exposed to a saline solution), and then the coated substrate is then immersed in a saline solution comprised of 7.0 mole percent of sodium chloride and 93 mole percent of water, and if the substrate/saline solution is maintained at atmospheric pressure and at temperature of 98.6 degrees Fahrenheit for 6 months, the coated substrate, upon removal from the saline solution and drying, will be found to have a magnetic moment that is within plus or minus 5 percent of its magnetic moment at time zero.

In another embodiment, the coated substrate of this invention has durable mechanical properties when tested by the saline immersion test described above.

In one embodiment, the coating 2104 is biocompatible with biological organisms. As used herein, the term biocompatible refers to a coating whose chemical composition does not change substantially upon exposure to biological fluids. Thus, when the coating 2104 is immersed in a 7.0 mole percent saline solution for 6 months maintained at a temperature of 98.6 degrees Fahrenheit, its chemical composition (as measured by, e.g., energy dispersive X-ray analysis [EDS, or EDAX]) is substantially identical to its chemical composition at “time zero.”

A Preferred Process of the Invention

In one embodiment of the invention, best illustrated in FIG. 11, a coated stent is imaged by an MRI imaging process.

In the first step of this process, the coated stent described by reference to FIG. 11 is contacted with the radio-frequency, direct current, and gradient fields normally associated with MRI imaging processes; these fields are discussed elsewhere in this specification. They are depicted as an MRI imaging signal 440 in FIG. 11.

In the second step of this process, the MRI imaging signal 440 penetrates the coated stent 400 and interacts with material disposed on the inside of such stent, such as, e.g., plaque particles 430 and 432. This interaction produces a signal best depicted as arrow 441 in FIG. 11.

In one embodiment, the signal 440 is substantially unaffected by its passage through the coated stent 400. Thus, in this embodiment, the radio-frequency field that is disposed on the outside of the coated stent 400 is substantially the same as the radio-frequency field that passes through and is disposed on the inside of the coated stent 400.

By comparison, when the stent (not shown) is not coated with the coatings of this invention, the characteristics of the signal 440 are substantially varied by its passage through the uncoated stent. Thus, with such uncoated stent, the radio-frequency signal that is disposed on the outside of the stent (not shown) differs substantially from the radio-frequency field inside of the uncoated stent (not shown). In some cases, because of substrate effects, substantially none of such radio-frequency signal passes through the uncoated stent (not shown).

In the third step of this process, and in one embodiment thereof, the MRI field(s) interact with material disposed on the inside of coated stent 400 such as, e.g., plaque particles 430 and 432. This interaction produces a signal 441 by means well known to those in the MRI imaging art.

In the fourth step of the preferred process of this invention, the signal 441 passes back through the coated stent 400 in a manner such that it is substantially unaffected by the coated stent 400. Thus, in this embodiment, the radio-frequency field that is disposed on the inside of the coated stent 400 is substantially the same as the radio-frequency field that passes through and is disposed on the outside of the coated stent 400.

By comparison, when the stent (not shown) is not coated with the coatings of this invention, the characteristics of the signal 441 are substantially varied by its passage through the uncoated stent. Thus, with such uncoated stent, the radio-frequency signal that is disposed on the inside of the stent (not shown) differs substantially from the radio-frequency field outside of the uncoated stent (not shown). In some cases, because of substrate effects, substantially none of such signal 441 passes through the uncoated stent (not shown).

Another Preferred Process of the Invention

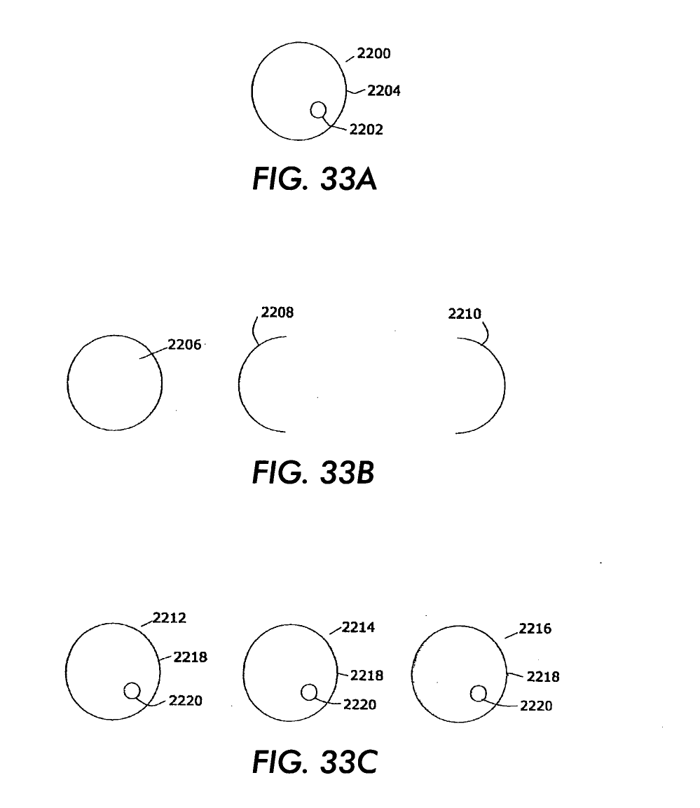

FIGS. 33A, 33B, and 33C illustrate another preferred process of the invention in which a stent 2200 may be imaged with an MRI imaging process. In the embodiment depicted in FIG. 33A, the stent 2200 is comprised of plaque 2202 disposed inside the inside wall 2204 of the stent 2200.

FIG. 33B illustrates three images produced from the imaging of stent 2200, depending upon the orientation of such stent 2200 in relation to the MRI imaging apparatus reference line (not shown). With a first orientation, an image 2206 is produced. With a second orientation, an image 2208 is produced. With a third orientation, an image 2210 is produced.

By comparison, FIG. 33C illustrates the images obtained when the stent 2200 has the nanomagnetic coating of this invention disposed about it. Thus, when the coated stent 400 of FIG. 11 is imaged, the images 2212, 2214, and 2216 are obtained.

The images 2212, 2214, and 2216 are obtained when the coated stent 400 is at the orientations of the uncoated stent 2200 the produced images 2206, 2208, and 2210, respectively. However, as will be noted, despite the variation in orientations, one obtains the same image with the coated stent 400.

Thus, e.g., the image 2218 of the coated stent will be identical regardless of how such coated stent is oriented vis-a-vis the MRI imaging apparatus reference line (not shown). Thus, e.g., the image 2220 of the plaque particles will be the same regardless of how such coated stent is oriented vis-a-vis the MRI imaging apparatus reference line (not shown).

Consequently, in this embodiment of the invention, one may utilize a nanomagnetic coating that, when imaged with the MRI imaging apparatus, will provide a distinctive and reproducible imaging response regardless of the orientation of the stent.

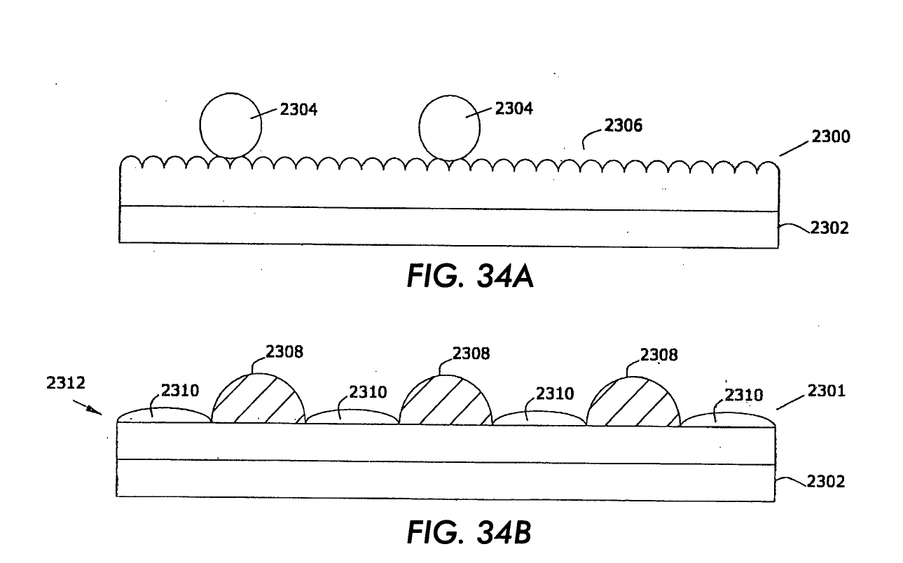

FIGS. 34A and 34B illustrate a hydrophobic coating 2300 and a hydrophilic coating 2301 that may be produced by the process of this invention.

As is known to those skilled in the art, a hydrophobic material is antagonistic to water and incapable of dissolving in water. A hydrophobic surface is illustrated in FIG. 34A.

Referring to FIG. 34A, it will be seen that a coating 2300 is deposited onto substrate 2302. In the embodiment depicted, the coating 2300 an average surface roughness of less than about 1 nanometer. Inasmuch as the average water droplet has a minimum cross-sectional dimension of at least about 3 nanometers, the water droplets 2304 will tend not to bond to the coated surface 2306 which, thus, is hydrophobic with regard to such water droplets.

One may vary the average surface roughness of coated surface 2306 by varying the pressure used in the sputtering process described elsewhere in this specification. In general, the higher the gas pressure used, the rougher the surface.

FIG. 34B illustrates water droplets 2308 between surface features 2310 of coated surface 2312. In this embodiment, because the surface features 2310 are spaced from each other by a distance of at least about 10 nanometers, the water droplets 2308 have an opportunity to bond to the surface 2312 which, in this embodiment, is hydrophilic.

The Bond Formed Between the Substrate and the Coating

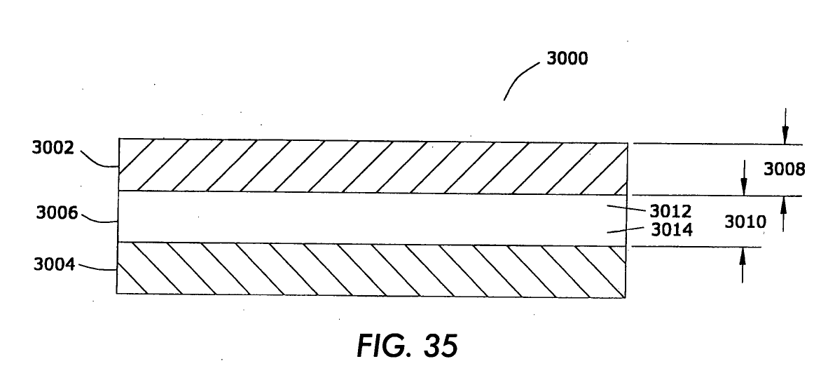

Applicants believe that, in at least one preferred embodiment of the process of their invention, the particles in their coating diffuse into the substrate being coated to form a interfacial diffusion layer. This structure is best illustrated in FIG. 35 which, as will be apparent, is not drawn to scale.

Referring to FIG. 35, the coated assembly 3000 is preferably comprised of a coating 3002 disposed on a substrate 3004. The coating 3002 preferably has at thickness 3008 of at least about 150 nanometers.

The interlayer 3006, by comparison, has a thickness of 3010 of less than about 10 nanometers and, preferably, less than about 5 nanometers. In one embodiment, the thickness of interlayer 3010 is less than about 2 nanometers.

The interlayer 3006 is preferably comprised of a heterogeneous mixture of atoms from the substrate 3004 and the coating 3002. It is preferred that at least 10 mole percent of the atoms from the coating 3002 are present in the interlayer 3006, and that at least 10 mole percent of the atoms from the substrate 3004 are in the interlayer 3006. It is more preferred that from about 40 to about 60 mole percent of the atoms from each of the coating and the substrate be present in the interlayer 3006, it being apparent that more atoms from the coating will be present in that portion 3012 of the interlayer closest to the coating, and more atoms from the substrate will be present in that portion 3014 closest to the substrate.

In one embodiment, the substrate 3004 will consist essentially of niobium atoms with from about 0 to about 2 molar percent of zirconium atoms present. In another embodiment, the substrate 3004 will comprise nickel atoms and titanium atoms. In yet another embodiment, the substrate will comprise tantalum atoms, or titanium atoms.

The coating may comprise any of the A, B, and/or C atoms described hereinabove. By way of way of illustration, the coating may comprise aluminum atoms and oxygen atoms (in the form of aluminum oxide), iridium atoms and oxygen atoms (in the form of irdium oxide), etc.

A Coated Substrate with a Specified Surface Morphology

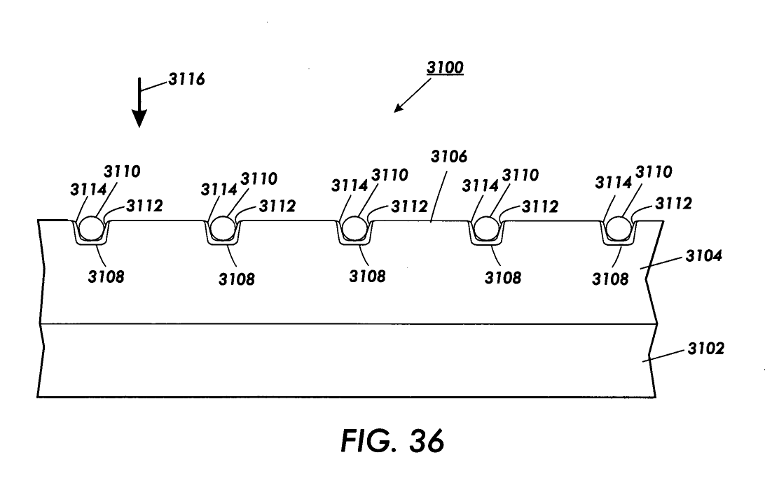

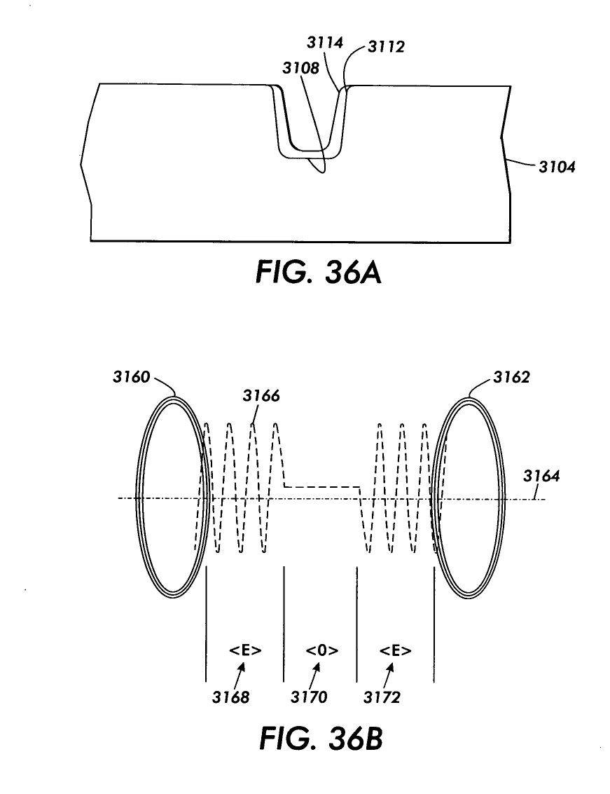

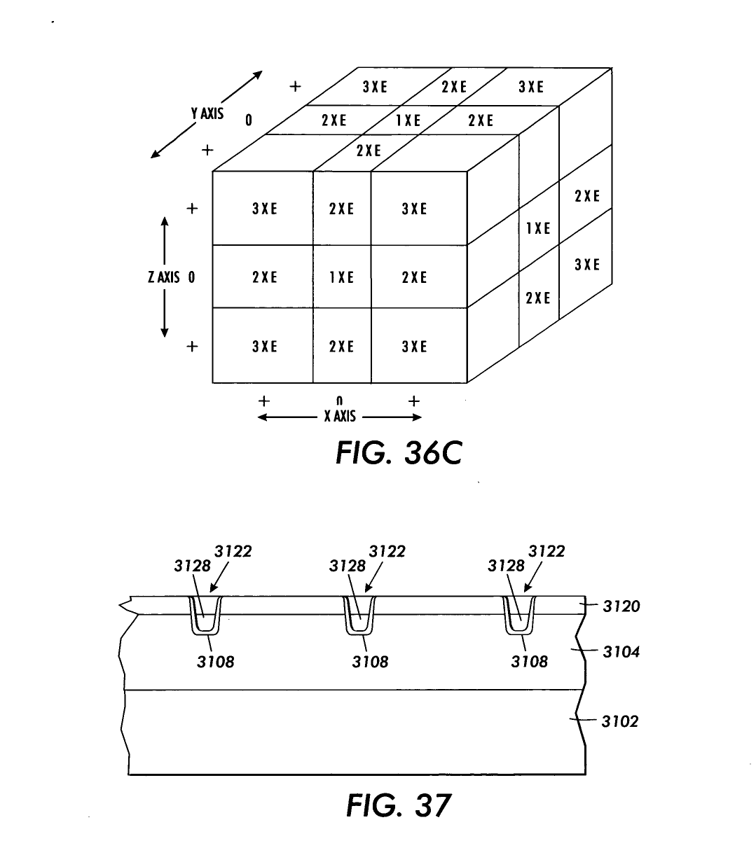

FIG. 36 is a sectional schematic view of a coated substrate 3100 comprised of a substrate 3102 and, bonded thereto, a layer 3104 of nano-sized particles that may comprise nanomagnetic particles, nanoelectrical particles, nanoinsulative particles, nanothermal particles. These particles, the mixtures thereof, and the matrices in which they are disposed have all been described elsewhere in this specification. Depending upon the properties desired from the coated substrate 3100 and/or the layer 3104, one may use one or more of the coating constructs described elsewhere in this specification. Thus, e.g., depending upon the type of particle(s) used and its properties, one may produce a desired set of electrical and magnetic properties for either the coated substrate 3100, the substrate 3200, and/or the coating 3104.

In one embodiment, the coating 3104 is comprised of at least about 5 weight percent of nanomagnetic material with the properties described elsewhere in this specification. In another embodiment, the coating 3104 is comprised of at least 10 weight percent of nanomagnetic material. In yet another embodiment, the coating 3104 is comprised of at least about 40 weight percent of nanomagnetic material.

Referring again to FIG. 36, and to the preferred embodiment depicted therein, the surface 3106 of the coating 3104 is comprised of a multiplicity of morphological indentations 3108 sized to receive drug particles 3110.

In one embodiment, the drug particles are particles of an anti-microtubule agent, as that term is described and defined in U.S. Pat. No. 6,333,347. The entire disclosure of this United States patent is hereby incorporated by reference into this specification.

As is known to those skilled in the art, paclitaxel is an anti-microtubule agent. As that term is used in this specification (and as it also is used in the specification of U.S. Pat. No. 6,333,347), the term “anti-microtubule agent” includes any protein, peptide, chemical, or other molecule which impairs the function of microtubules, for example, through the prevention or stabilization of polymerization. As is known to those in the art, a wide variety of methods may be utilized to determine the anti-microtubule activity of a particular compound, including for example, assays described by Smith et al. (Cancer Lett 79(2):213-219, 1994) and Mooberry et al., (Cancer Lett. 96(2):261-266, 1995).

As is disclosed at columns 3-5 of U.S. Pat. No. 6,333,347, “ . . . a wide variety of anti-microtubule agents may be delivered, either with or without a carrier (e.g., a polymer or ointment), in order to treat or prevent disease. Representative examples of such agents include taxanes (e.g., paclitaxel (discussed in more detail below) and docetaxel) (Schiff et al., Nature 277: 665-667, 1979; Long and Fairchild, Cancer Research 54: 4355-4361, 1994; Ringel and Horwitz, J. Natl. Cancer Inst. 83(4): 288-291, 1991; Pazdur et al., Cancer Treat. Rev. 19(4): 351-386, 1993), campothecin, eleutherobin (e.g., U.S. Pat. No. 5,473,057), sarcodictyins (including sarcodictyin A), epothilones A and B (Bollag et al., Cancer Research 55: 2325-2333, 1995), discodermolide (ter Haar et al., Biochemistry 35: 243-250, 1996), deuterium oxide (D2 O) (James and Lefebvre, Genetics 130(2): 305-314, 1992; Sollott et al., J. Clin. Invest. 95: 1869-1876, 1995), hexylene glycol (2-methyl-2,4-pentanediol) (Oka et al., Cell Struct. Funct. 16(2): 125-134, 1991), tubercidin (7-deazaadenosine) (Mooberry et al., Cancer Lett. 96(2): 261-266, 1995), LY290181 (2-amino-4-(3-pyridyl)-4H-naphtho(1,2-b)pyran-3-cardonitrile) (Panda et al., J. Biol. Chem. 272(12): 7681-7687, 1997; Wood et al., Mol. Pharmacol. 52(3): 437-444, 1997), aluminum fluoride (Song et al., J. Cell. Sci. Suppl. 14: 147-150, 1991), ethylene glycol bis-(succinimidylsuccinate) (Caplow and Shanks, J. Biol. Chem. 265(15): 8935-8941, 1990), glycine ethyl ester (Mejillano et al., Biochemistry 31(13): 3478-3483, 1992), nocodazole (Ding et al., J. Exp. Med. 171(3): 715-727, 1990; Dotti et al., J. Cell Sci. Suppl. 15: 75-84, 1991; Oka et al., Cell Struct. Funct. 16(2): 125-134, 1991; Weimer et al., J. Cell. Biol. 136(1), 71-80, 1997), cytochalasin B (Illinger et al., Biol. Cell 73(2-3): 131-138, 1991), colchicine and CI 980 (Allen et al., Am. J. Physiol. 261(4 Pt. 1) L315-L321, 1991; Ding et al., J. Exp. Med. 171(3): 715-727, 1990; Gonzalez et al., Exp. Cell. Res. 192(1): 10-15, 1991; Stargell et al., Mol. Cell. Biol. 12(4): 1443-1450, 1992; Garcia et al., Antican. Drugs 6(4): 533-544, 1995), colcemid (Barlow et al., Cell. Motil. Cytoskeleton 19(1): 9-17, 1991; Meschini et al., J Microsc. 176(Pt. 3): 204-210, 1994; Oka et al., Cell Struct. Funct. 16(2): 125-134, 1991), podophyllotoxin (Ding et al., J. Exp. Med 171(3): 715-727, 1990), benomyl (Hardwick et al., J. Cell. Biol. 131(3): 709-720, 1995; Shero et al., Genes Dev. 5(4): 549-560, 1991), oryzalin (Stargell et al., Mol. Cell. Biol. 12(4): 1443-1450, 1992), majusculamide C (Moore, J. Ind. Microbiol. 16(2): 134-143, 1996), demecolcine (Van Dolah and Ramsdell, J. Cell. Physiol. 166(1): 49-56, 1996; Wiemer et al., J. Cell. Biol. 136(1): 71-80, 1997), methyl-2-benzimidazolecarbamate (MBC) (Brown et al., J. Cell. Biol. 123(2): 387-403, 1993), LY195448 (Barlow & Cabral, Cell Motil. Cytoskel. 19: 9-17, 1991), subtilisin (Saoudi et al., J. Cell Sci. 108: 357-367, 1995), 1069C85 (Raynaud et al., Cancer Chemother. Pharmacol. 35: 169-173, 1994), steganacin (Hamel, Med Res. Rev. 16(2): 207-231, 1996), combretastatins (Hamel, Med Res. Rev. 16(2): 207-231, 1996), curacins (Hamel, Med Res. Rev. 16(2): 207-231, 1996), estradiol (Aizu-Yokata et al., Carcinogen. 15(9): 1875-1879, 1994), 2-methoxyestradiol (Hamel, Med Res. Rev. 16(2): 207-231, 1996), flavanols (Hamel, Med Res. Rev. 16(2): 207-231, 1996), rotenone (Hamel, Med Res. Rev. 16(2): 207-231, 1996), griseofulvin (Hamel, Med Res. Rev. 16(2): 207-231, 1996), vinca alkaloids, including vinblastine and vincristine (Ding et al., J. Exp. Med 171(3): 715-727, 1990; Dirk et al., Neurochem. Res. 15(11): 1135-1139, 1990; Hamel, Med Res. Rev. 16(2): 207-231, 1996; Illinger et al., Biol. Cell 73(2-3): 131-138, 1991; Wiemer et al., J. Cell. Biol. 136(1): 71-80, 1997), maytansinoids and ansamitocins (Hamel, Med Res. Rev. 16(2): 207-231, 1996), rhizoxin (Hamel, Med Res. Rev. 16(2): 207-231, 1996), phomopsin A (Hamel, Med. Res. Rev. 16(2): 207-231, 1996), ustiloxins (Hamel, Med Res. Rev. 16(2): 207-231, 1996), dolastatin 10 (Hamel, Med. Res. Rev. 16(2): 207-231, 1996), dolastatin 15 (Hamel, Med. Res. Rev. 16(2): 207-231, 1996), halichondrins and halistatins (Hamel, Med. Res. Rev. 16(2): 207-231, 1996), spongistatins (Hamel, Med Res. Rev. 16(2): 207-231, 1996), cryptophycins (Hamel, Med. Res. Rev. 16(2): 207-231, 1996), rhazinilam (Hamel, Med. Res. Rev. 16(2): 207-231, 1996), betaine (Hashimoto et al., Zool. Sci. 1: 195-204, 1984), taurine (Hashimoto et al., Zool. Sci. 1: 195-204, 1984), isethionate (Hashimoto et al., Zool. Sci. 1: 195-204, 1984), HO-221 (Ando et al., Cancer Chemother. Pharmacol. 37: 63-69, 1995), adociasulfate-2 (Sakowicz et al., Science 280: 292-295, 1998), estramustine (Panda et al., Proc. Natl. Acad. Sci. USA 94: 10560-10564, 1997), monoclonal anti-idiotypic antibodies (Leu et al., Proc. Natl. Acad. Sci. USA 91(22): 10690-10694, 1994), microtubule assembly promoting protein (paclitaxel-like protein, TALP) (Hwang et al., Biochem. Biophys. Res. Commun. 208(3): 1174-1180, 1995), cell swelling induced by hypotonic (190 mosmol/L) conditions, insulin (100 nmol/L) or glutamine (10 mmol/L) (Haussinger et al., Biochem. Cell. Biol. 72(1-2): 12-19, 1994), dynein binding (Ohba et al., Biochim. Biophys. Acta 1158(3): 323-332, 1993), gibberelin (Mita and Shibaoka, Protoplasma 119(1/2): 100-109, 1984), XCHO1 (kinesin-like protein) (Yonetani et al., Mol. Biol. Cell 7(suppl): 211A, 1996), lysophosphatidic acid (Cook et al., Mol. Biol Cell 6(suppl): 260A, 1995), lithium ion (Bhattacharyya and Wolff, Biochem. Biophys. Res. Commun. 73(2): 383-390, 1976), plant cell wall components (e.g., poly-L-lysine and extensin) (Akashi et al., Planta 182(3): 363-369, 1990), glycerol buffers (Schilstra et al., Biochem. J. 277(Pt. 3): 839-847, 1991; Farrell and Keates, Biochem. Cell. Biol. 68(11): 1256-1261, 1990; Lopez et al., J. Cell. Biochem. 43(3): 281-291, 1990), Triton X-100 microtubule stabilizing buffer (Brown et al., J. Cell Sci. 104(Pt. 2): 339-352, 1993; Safiejko-Mroczka and Bell, J. Histochem. Cytochem. 44(6): 641-656, 1996), microtubule associated proteins (e.g, MAP2, MAP4, tau, big tau, ensconsin, elongation factor-1-alpha (EF-1.alpha.) and E-MAP-115) (Burgess et al., Cell Motil. Cytoskeleton 20(4): 289-300, 1991; Saoudi et al., J. Cell. Sci. 108(Pt. 1): 357-367, 1995; Bulinski and Bossler, J. Cell. Sci. 107(Pt. 10): 2839-2849, 1994; Ookata et al., J. Cell Biol. 128(5): 849-862, 1995; Boyne et al., J. Comp. Neurol. 358(2): 279-293, 1995; Ferreira and Caceres, J. Neurosci. 11(2): 392-400, 1991; Thurston et al., Chromosoma 105(1): 20-30, 1996; Wang et al., Brain Res. Mol. Brain Res. 38(2): 200-208, 1996; Moore and Cyr, Mol. Biol. Cell 7(suppl): 221-A, 1996; Masson and Kreis, J. Cell Biol. 123(2), 357-3.71, 1993), cellular entities (e.g., histone H1, myelin basic protein and kinetochores) (Saoudi et al., J. Cell. Sci. 108(Pt. 1): 357-367, 1995; Simerly et al., J. Cell Biol. 111(4): 1491-1504, 1990), endogenous microtubular structures (e.g., axonemal structures, plugs and GTP caps) (Dye et al., Cell Motil. Cytoskeleton 21(3): 171-186, 1992; Azhar and Murphy, Cell Motil. Cytoskeleton 15(3): 156-161, 1990; Walker et al., J. Cell Biol. 114(1): 73-81, 1991; Drechsel and Kirschner, Curr. Biol. 4(12): 1053-1061, 1994), stable tubule only polypeptide (e.g., STOP145 and STOP220) (Pirollet et al., Biochim. Biophys. Acta 1160(1): 113-119, 1992; Pirollet et al., Biochemistry 31(37): 8849-8855, 1992; Bosc et al., Proc. Natl. Acad. Sci. USA 93(5): 2125-2130, 1996; Margolis et al., EMBO J. 9(12): 4095-4102, 1990) and tension from mitotic forces (Nicklas and Ward, J. Cell Biol. 126(5): 1241-1253, 1994), as well as any analogues and derivatives of any of the above. Such compounds can act by either depolymerizing microtubules (e.g., colchicine and vinblastine), or by stabilizing microtubule formation (e.g., paclitaxel).”

One preferred anti-microtuble agent is paclitaxel, a compound which disrupts microtubule formation by binding to tubulin to form abnormal mitotic spindles. As is disclosed at columns 5-6 of such U.S. Pat. No. 6,333,347 (the entire disclosure of which is hereby incorporated by reference into this specification), “ . . . paclitaxel is a highly derivatized diterpenoid (Wani et al., J. Am. Chem. Soc. 93:2325, 1971) which has been obtained from the harvested and dried bark of Taxus brevifolia (Pacific Yew) and Taxomyces Andreanae and Endophytic Fungus of the Pacific Yew (Stierle et al., Science 60:214-216, 1993). ‘Paclitaxel’ (which should be understood herein to include prodrugs, analogues and derivatives such as, for example, PACLITAXEL®, TAXOTERE®, Docetaxel, 10-desacetyl analogues of paclitaxel and 3′N-desbenzoyl-3′N-t-butoxy carbonyl analogues of paclitaxel) may be readily prepared utilizing techniques known to those skilled in the art (see e.g., Schiff et al., Nature 277:665-667, 1979; Long and Fairchild, Cancer Research 54:4355-4361, 1994; Ringel and Horwitz, J. Natl. Cancer Inst. 83(4):288-291, 1991; Pazdur et al., Cancer Treat. Rev. 19(4):351-386, 1993; WO 94/07882; WO 94/07881; WO 94/07880; WO 94/07876; WO 93/23555; WO 93/10076; WO 94/00156; WO. 93/24476; EP 590267; WO 94/20089; U.S. Pat. Nos. 5,294,637; 5,283,253; 5,279,949; 5,274,137; 5,202,448; 5,200,534; 5,229,529; 5,254,580; 5,412,092; 5,395,850; 5,380,751; 5,350,866; 4,857,653; 5,272,171; 5,411,984; 5,248,796; 5,248,796; 5,422,364; 5,300,638; 5,294,637; 5,362,831; 5,440,056; 4,814,470; 5,278,324; 5,352,805; 5,411,984; 5,059,699; 4,942,184; Tetrahedron Letters 35(52):9709-9712, 1994; J. Med Chem. 35:4230-4237, 1992; J. Med. Chem. 34:992-998, 1991; J. Natural Prod. 57(10):1404-1410, 1994; J. Natural Prod. 57(11):1580-1583, 1994; J. Am. Chem. Soc. 110:6558-6560, 1988), or obtained from a variety of commercial sources, including for example, Sigma Chemical Co., St. Louis, Mo. (T7402-from Taxus brevifolia).” The entire disclosure of each of the United States patents described in this paragraph of the specification is hereby incorporated by reference into this specification.

Paclitaxel derivatives and/or analogues are also drugs which may be used in the process of this invention. As is disclosed at columns 5-6 of such U.S. Pat. No. 6,333,347, “Representative examples of such paclitaxel derivatives or analogues include 7-deoxy-docepaclitaxel, 7,8-cyclopropataxanes, N-substituted 2-azetidones, 6,7-epoxy paclitaxels, 6,7-modified paclitaxels, 10-desacetoxypaclitaxel, 10-deacetylpaclitaxel (from 10-deacetylbaccatin III), phosphonooxy and carbonate derivatives of paclitaxel, paclitaxel 2′,7-di(sodium 1,2-benzenedicarboxylate, 10-desacetoxy-11,12-dihydropaclitaxel-10,12(18)-diene derivatives, 10-desacetoxypaclitaxel, Propaclitaxel (2′-and/or 7-O-ester derivatives), (2′-and/or 7-O-carbonate derivatives), asymmetric synthesis of paclitaxel side chain, fluoro paclitaxels, 9-deoxotaxane, (13-acetyl-9-deoxobaccatine III, 9-deoxopaclitaxel, 7-deoxy-9-deoxopaclitaxel, 10-desacetoxy-7-deoxy-9-deoxopaclitaxel, Derivatives containing hydrogen or acetyl group and a hydroxy and tert-butoxycarbonylamino, sulfonated 2′-acryloylpaclitaxel and sulfonated 2′-O-acyl acid paclitaxel derivatives, succinylpaclitaxel, 2′-.gamma.-aminobutyrylpaclitaxel formate, 2′-acetyl paclitaxel, 7-acetyl paclitaxel, 7-glycine carbamate paclitaxel, 2′-OH-7-PEG(5000) carbarnate paclitaxel, 2′-benzoyl and 2′,7-dibenzoyl paclitaxel derivatives, other prodrugs (2′-acetylpaclitaxel; 2′,7-diacetylpaclitaxel; 2′succinylpaclitaxel; 2′-(beta-alanyl)-paclitaxel); 2′gamrnma-aminobutyrylpaclitaxel formate; ethylene glycol derivatives of 2′-succinylpaclitaxel; 2′-glutarylpaclitaxel; 2′-(N,N-dimethylglycyl) paclitaxel; 2′-(2-(N,N-dimethylamino)propionyl)paclitaxel; 2′orthocarboxybenzoyl paclitaxel; 2′aliphatic carboxylic acid derivatives of paclitaxel, Prodrugs {2′(N,N-diethylaminopropionyl)paclitaxel, 2′(N,N-dimethylglycyl)paclitaxel, 7(N,N-dimethylglycyl)paclitaxel, 2′,7-di-(N,N-dimethylglycyl)paclitaxel, 7(N,N-diethylaminopropionyl)paclitaxel, 2′,7-di(N,N-diethylaminopropionyl)paclitaxel, 2′-(L-glycyl)paclitaxel, 7-(L-glycyl)paclitaxel, 2′,7-di(L-glycyl)paclitaxel, 2′-(L-alanyl)paclitaxel, 7-(L-alanyl)paclitaxel, 2′,7-di(L-alanyl)paclitaxel, 2′-(L-leucyl)paclitaxel, 7-(L-leucyl)paclitaxel, 2′,7-di(L-leucyl)paclitaxel, 2′-(L-isoleucyl)paclitaxel, 7-(L-isoleucyl)paclitaxel, 2′,7-di(L-isoleucyl)paclitaxel, 2′-(L-valyl)paclitaxel, 7-(L-valyl)paclitaxel, 2′7-di(L-valyl)paclitaxel, 2′-(L-phenylalanyl)paclitaxel, 7-(L-phenylalanyl)paclitaxel, 2′,7-di(L-phenylalanyl)paclitaxel, 2′-(L-prolyl)paclitaxel, 7-(L-prolyl)paclitaxel, 2′,7-di(L-prolyl)paclitaxel, 2′-(L-lysyl)paclitaxel, 7-(L-lysyl)paclitaxel, 2′,7-di(L-lysyl)paclitaxel, 2′-(L-glutamyl)paclitaxel, 7-(L-glutamyl)paclitaxel, 2′,7-di(L-glutamyl)paclitaxel, 2′-(L-arginyl)paclitaxel, 7-(L-arginyl)paclitaxel, 2′,7-di(L-arginyl)paclitaxel}, Paclitaxel analogs with modified phenylisoserine side chains, taxotere, (N-debenzoyl-N-tert-(butoxycaronyl)-10-deacetylpaclitaxel, and taxanes (e.g., baccatin III, cephalomannine, 10-deacetylbaccatin III, brevifoliol, yunantaxusin and taxusin).”

In the process of this invention, the anti-microtubule agent may be utilized by itself, and/or it may be utilized in a formulation that comprises such agent and a carrier. The carrier may be either of polymeric or non-polymeric origin. May suitable carriers for anti-microtubule agents are disclosed at columns 6-9 of such U.S. Pat. No. 6,333,347.

Thus, e.g., and as is disclosed in U.S. Pat. No. 6,333,347, “ . . . a wide variety of polymeric carriers may be utilized to contain and/or deliver one or more of the therapeutic agents discussed above, including for example both biodegradable and non-biodegradable compositions. Representative examples of biodegradable compositions include albumin, collagen, gelatin, hyaluronic acid, starch, cellulose (methylcellulose, hydroxypropylcellulose, hydroxypropylmethylcellulose, hydroxyethylcellulose, carboxymethylcellulose, cellulose acetate phthalate, cellulose acetate succinate, hydroxypropylmethylcellulose phthalate), casein, dextrans, polysaccharides, fibrinogen, poly(D,L lactide), poly(D,L-lactide-coglycolide), poly(glycolide), poly(hydroxybutyrate), poly(alkylcarbonate) and poly(orthoesters), polyesters, poly(hydroxyvaleric acid), polydioxanone, poly(ethylene terephthalate), poly(malic acid), poly(tartronic acid), polyanhydrides, polyphosphazenes, poly(amino acids) and their copolymers (see generally, Illum, L., Davids, S. S. (eds.) “Polymers in Controlled Drug Delivery” Wright, Bristol, 1987; Arshady, J. Controlled Release 17:1-22, 1991; Pitt, Int. J. Phar. 59:173-196, 1990; Holland et al., J. Controlled Release 4:155-0180, 1986). Representative examples of nondegradable polymers include poly(ethylene-vinyl acetate) (“EVA”) copolymers, silicone rubber, acrylic polymers (polyacrylic acid, polymethylacrylic acid, polymethylmethacrylate, polyalkylcynoacrylate), polyethylene, polyproplene, polyamides (nylon 6,6), polyurethane, poly(ester urethanes), poly(ether urethanes), poly(ester-urea), polyethers (poly(ethylene oxide), poly(propylene oxide), Pluronics and poly(tetramethylene glycol)), silicone rubbers and vinyl polymers (polyvinylpyrrolidone, poly(vinyl alcohol), poly(vinyl acetate phthalate). Polymers may also be developed which are either anionic (e.g., alginate, carrageenin, carboxymethyl cellulose and poly(acrylic acid), or cationic (e.g, chitosan, poly-L-lysine, polyethylenimine, and poly (allyl amine)) (see generally, Dunn et al., J. Applied Polymer Sci. 50:353-365, 1993; Cascone et al., J. Materials Sci. Materials in Medicine 5:770-774, 1994; Shiraishi et al., Biol. Pharm. Bull. 16(11):1164-1168, 1993; Thacharodi and Rao, Int'l J. Pharm. 120:115-118, 1995; Miyazaki et al., Int'l J. Pharm. 118:257-263, 1995). Particularly preferred polymeric carriers include poly(ethylene-vinyl acetate), poly (D,L-lactic acid) oligomers and polymers, poly (L-lactic acid) oligomers and polymers, poly (glycolic acid), copolymers of lactic acid and glycolic acid, poly (caprolactone), poly (valerolactone), polyanhydrides, copolymers of poly (caprolactone) or poly (lactic acid) with a polyethylene glycol (e.g., MePEG), and blends thereof.”

“Polymeric carriers can be fashioned in a variety of forms, with desired release characteristics and/or with specific desired properties. For example, polymeric carriers may be fashioned to release a therapeutic agent upon exposure to a specific triggering event such as pH (see e.g., Heller et al., “Chemically Self-Regulated Drug Delivery Systems,” in Polymers in Medicine III, Elsevier Science Publishers B. V., Amsterdam, 1988, pp. 175-188; Kang et al., J. Applied Polymer Sci. 48:343-354, 1993; Dong et al., J. Controlled Release 19.171-178, 1992; Dong and Hoffman, J. Controlled Release 15:141-152, 1991; Kim et al., J. Controlled Release 28:143-152, 1994; Cornejo-Bravo et al., J. Controlled Release 33:223-229, 1995; Wu and Lee, Pharm. Res. 10(10):1544-1547, 1993; Serres et al., Pharm. Res. 13(2):196-201, 1996; Peppas, “Fundamentals of pH— and Temperature-Sensitive Delivery Systems,” in Gurny et al. (eds.), Pulsatile Drug Delivery, Wissenschaftliche Verlagsgesellschaft mbH, Stuttgart, 1993, pp. 41-55; Doelker, “Cellulose Derivatives,” 1993, in Peppas and Langer (eds.), Biopolymers I, Springer-Verlag, Berlin). Representative examples of pH-sensitive polymers include poly(acrylic acid) and its derivatives (including for example, homopolymers such as poly(aminocarboxylic acid); poly(acrylic acid); poly(methyl acrylic acid), copolymers of such homopolymers, and copolymers of poly(acrylic acid) and acrylmonomers such as those discussed above. Other pH sensitive polymers include polysaccharides such as cellulose acetate phthalate; hydroxypropylmethylcellulose phthalate; hydroxypropylmethylcellulose acetate succinate; cellulose acetate trimellilate; and chitosan. Yet other pH sensitive polymers include any mixture of a pH sensitive polymer and a water soluble polymer.”

“Likewise, polymeric carriers can be fashioned which are temperature sensitive (see e.g., Chen et al., “Novel Hydrogels of a Temperature-Sensitive Pluronic Grafted to a Bioadhesive Polyacrylic Acid Backbone for Vaginal Drug Delivery,” in Proceed Intern. Symp. Control. Rel. Bioact. Mater. 22:167-168, Controlled Release Society, Inc., 1995; Okano, “Molecular Design of Stimuli-Responsive Hydrogels for Temporal Controlled Drug Delivery,” in Proceed Intern. Symp. Control. Rel. Bioact. Mater. 22:111-112, Controlled Release Society, Inc., 1995; Johnston et al., Pharm. Res. 9(3):425-433, 1992; Tung, Int'l J. Pharm. 107:85-90, 1994; Harsh and Gehrke, J. Controlled Release 17:175-186, 1991; Bae et al., Pharm. Res. 8(4):531-537, 1991; Dinarvand and D'Emanuele, J. Controlled Release 36:221-227, 1995; Yu and Grainger, “Novel Thermo-sensitive Amphiphilic Gels: Poly N-isopropylacrylamide-co-sodium acrylate-co-n-N-alkylacrylamide Network Synthesis and Physicochemical Characterization,” Dept. of Chemical & Biological Sci., Oregon Graduate Institute of Science & Technology, Beaverton, Oreg., pp. 820-821; Zhou and Smid, “Physical Hydrogels of Associative Star Polymers,” Polymer Research Institute, Dept. of Chemistry, College of Environmental Science and Forestry, State Univ. of New York, Syracuse, N.Y., pp. 822-823; Hoffman et al., “Characterizing Pore Sizes and Water ‘Structure’ in Stimuli-Responsive Hydrogels,” Center for Bioengineering, Univ. of Washington, Seattle, Wash., p. 828; Yu and Grainger, “Thermo-sensitive Swelling Behavior in Crosslinked N-isopropylacrylamide Networks: Cationic, Anionic and Ampholytic Hydrogels,” Dept. of Chemical & Biological Sci., Oregon Graduate Institute of Science & Technology, Beaverton, Oreg., pp. 829-830; Kim et al., Pharm. Res. 9(3):283-290, 1992; Bae et al., Pharm. Res. 8(5):624-628, 1991; Kono et al., J. Controlled Release 30:69-75, 1994; Yoshida et al., J. Controlled Release 32:97-102, 1994; Okano et al., J. Controlled Release 36:125-133, 1995; Chun and Kim, J. Controlled Release 38:39-47, 1996; D'Emanuele and Dinarvand, Int'l J. Pharm. 1.18:237-242, 1995; Katono et al., J. Controlled Release 16:215-228, 1991; Hoffman, “Thermally Reversible Hydrogels Containing Biologically Active Species,” in Migliaresi et al. (eds.), Polymers in Medicine III, Elsevier Science Publishers B. V., Amsterdam, 1988, pp. 161-167; Hoffman, “Applications of Thermally Reversible Polymers and Hydrogels in Therapeutics and Diagnostics,” in Third International Symposium on Recent Advances in Drug Delivery Systems, Salt Lake City, Utah, Feb. 24-27, 1987, pp. 297-305; Gutowska et al., J. Controlled Release 22:95-104, 1992; Palasis and Gehrke, J. Controlled Release 18:1-12, 1992; Paavola et al., Pharm. Res. 12(12):1997-2002, 1995).”

“Representative examples of thermogelling polymers, and their gelatin temperature (LCST (° C.)) include homopolymers such as poly(N-methyl-N-n-propylacrylamide), 19.8; poly(N-n-propylacrylamide), 21.5; poly(N-methyl-N-isopropylacrylamide), 22.3; poly(N-n-propylmethacrylamide), 28.0; poly(N-isopropylacrylamide), 30.9; poly(N, n-diethylacrylamide), 32.0; poly(N-isopropylmethacrylamide), 44.0; poly(N-cyclopropylacrylamide), 45.5; poly(N-ethylmethyacrylamide), 50.0; poly(N-methyl-N-ethylacrylamide), 56.0; poly(N-cyclopropylmethacrylamide), 59.0; poly(N-ethylacrylamide), 72.0. Moreover thermogelling polymers may be made by preparing copolymers between (among) monomers of the above, or by combining such homopolymers with other water soluble polymers such as acrylmonomers (e.g. acrylic acid and derivatives thereof such as methylacrylic acid, acrylate and derivatives thereof such as butyl methacrylate, acrylamide, and N-n-butyl acrylamide).”

“Other representative examples of thermogelling polymers include cellulose ether derivatives such as hydroxypropyl cellulose, 41° C.; methyl cellulose, 55° C.; hydroxypropylmethyl cellulose, 66° C.; and ethylhydroxyethyl cellulose, and Pluronics such as F-127, 10-15° C.; L-122, 19° C.; L-92, 26° C.; L-81, 20° C.; and L-61, 24° C.”

“A wide variety of forms may be fashioned by the polymeric carriers of the present invention, including for example, rod-shaped devices, pellets, slabs, or capsules (see e.g., Goodell et al., Am. J. Hosp. Pharm. 43:1454-1461, 1986; Langer et al., ‘Controlled release of macromolecules from polymers’, in Biomedical Polymers, Polymeric Materials and Pharmaceuticals for Biomedical Use, Goldberg, E. P., Nakagim, A. (eds.) Academic Press, pp. 113-137, 1980; Rhine et al., J. Pharm. Sci. 69:265-270, 1980; Brown et al., J. Pharm. Sci. 72:1181-1185, 1983; and Bawa et al., J. Controlled Release 1:259-267, 1985). Therapeutic agents may be linked by occlusion in the matrices of the polymer, bound by covalent linkages, or encapsulated in microcapsules. Within certain preferred embodiments of the invention, therapeutic compositions are provided in non-capsular formulations such as microspheres (ranging from nanometers to micrometers in size), pastes, threads of various size, films and sprays.”

“Preferably, therapeutic compositions of the present invention are fashioned in a manner appropriate to the intended use. Within certain aspects of the present invention, the therapeutic composition should be biocompatible, and release one or more therapeutic agents over a period of several days to months. For example, “quick release” or “burst” therapeutic compositions are provided that release greater than 10%, 20%, or 25% (w/v) of a therapeutic agent (e.g., paclitaxel) over a period of 7 to 10 days. Such “quick release” compositions should, within certain embodiments, be capable of releasing chemotherapeutic levels (where applicable) of a desired agent. Within other embodiments, “low release” therapeutic compositions are provided that release less than 1% (w/v) of a therapeutic agent over a period of 7 to 10 days. Further, therapeutic compositions of the present invention should preferably be stable for several months and capable of being produced and maintained under sterile conditions.”

“Within certain aspects of the present invention, therapeutic compositions may be fashioned in any size ranging from 50 nm to 500 μm, depending upon the particular use. Alternatively, such compositions may also be readily applied as a “spray”, which solidifies into a film or coating. Such sprays may be prepared from microspheres of a wide array of sizes, including for example, from 0.1 μm to 3 μm, from 10 μm to 30 μm, and from 30 μm to 100 μm.”

“Therapeutic compositions of the present invention may also be prepared in a variety of “paste” or gel forms. For example, within one embodiment of the invention, therapeutic compositions are provided which are liquid at one temperature (e.g., temperature greater than 37° C., such as 40° C., 45° C., 50° C., 55° C. or 60° C.), and solid or semi-solid at another temperature (e.g., ambient.body temperature, or any temperature lower than 37° C.). Such “thermopastes” may be readily made given the disclosure provided herein.” The nanomagnetic particles of this invention may be disposed in a medium so that they are either in a liquid form, a semi-solid form, or a solid form.

The anti-microtuble agents used in one embodiment of the process of this invention may be formulated in a variety of forms suitable for administration; and they may be formulated to contain more than one anti-microtubule agents, to contain a variety of additional compounds, to have certain physical properties such as, e.g., elasticity, a particular melting point, or a specified release rate.

As is disclosed at columns 6-9 of U.S. Pat. No. 6,333,347, the anti-microtubule agents “ . . . may be administered either alone, or in combination with pharmaceutically or physiologically acceptable carrier, excipients or diluents. Generally, such carriers should be nontoxic to recipients at the dosages and concentrations employed. Ordinarily, the preparation of such compositions entails combining the therapeutic agent with buffers, antioxidants such as ascorbic acid, low molecular weight (less than about 10 residues) polypeptides, proteins, amino acids, carbohydrates including glucose, sucrose or dextrins, chelating agents such as EDTA, glutathione and other stabilizers and excipients. Neutral buffered saline or saline mixed with nonspecific serum albumin are exemplary appropriate diluents.”

“The anti-microtubule agent can be administered in a dosage which achieves a statistically significant result. In one embodiment, an antimicrotubule agent such as paclitaxel is administered at a dosage ranging from 100 ug to 50 mg, depending on the mode of administration and the type of carrier, if any for delivery. For treatment of restenosis, a single treatment may be provided before, during or after balloon angioplasty or stenting. For the treatment of instent restenosis, the anti-microtubule agent may be administered directly to prevent closure of the stented vessel. For the treatment of atherosclerosis, an anti-microtubule agent such as paclitaxel may be administered periodically, e.g., once every few months. In the case of cardiac transplantation, the anti-microtubule agent may be delivered in a slow release form that delivers from 1 to 75 mg/m2 (preferably 10 to 50 mg/m2) over a selected period of time. With any of these embodiments, the anti-microtubule agent (e.g., paclitaxel) may be administered along with other therapeutics.”

“Pericardial administration may be accomplished by a variety of manners including, for example, direct injection (preferably with ultrasound, CT, fluoroscopic, MRI or endoscopic guidance). (See e.g., U.S. Pat. Nos. 5,840,059 and 5,797,870). Within certain embodiments, a Saphenous Vein Harvester such as GSI's ENDOsaph, or Comedicus Inc.,’ PerDUCER (Pericardial Access Device) may be utilized to administer the desired anti-microtubule agent (e.g., paclitaxel).” In one embodiment, an anti-microtubule agent is bonded to the nanomagnetic particles of this invention, and the construct thus made is administered to a patient in one or more of the manners described above.

“Within one embodiment, the antimicrotubule agent or composition (e.g., paclitaxel and a polymer) may be delivered trans-myocardially through the right or left ventricle.”

“Within other embodiments, the antimicrotubule agent or composition (e.g., paclitaxel and a polymer) may be administered trans-myocardially through the right atrium. (See, e.g., U.S. Pat. Nos. 5,797,870 and 5,269,326). Briefly, the right atrium lies between the pericardium and the epicardium. An appropriate catheter is guided into the right atrium and positioned parallel with the wall of the pericardium. This positioning allows piercing of the right atrium (either by the catheter, or by an instrument that is passed within the catheter), without risk of damage to either the pericardium or the epicardium. The catheter can then be passed into the pericardial space, or an instrument passed through the lumen of the catheter into the pericardial space.”

“Alternatively, access to the pericardium, heart, or coronary vasculature may be gained operatively, by, for example, sub-xiphoid entry, a thoracotomy, or, open heart surgery. Preferably, the thoracotomy should be minimal, through an intercostal space for example. Fluoroscopy, or ultrasonic visualization may be utilized to assist in any of these procedures.”

Anti-Microtubule Agents with a Magnetic Moment

In one embodiment of the process of this invention, the drug particles 3110 used (see FIG. 36) are particles of an anti-microtubule agent with a magnetic moment.

Illustrative “magnetic moment anti-microtubule agents” are disclosed in applicants' copending U.S. patent application U.S. S. No. 60/516,134, filed on Oct. 31, 2003, the entire disclosure of which is hereby incorporated by reference into this specification.

By way of further illustration, means for producing a composition comprised of magnetic carrier particles having therapeutic quantities of absorbed paclitaxel are known to those skilled in the art. Thus, by way of illustration and not limitation, U.S. Pat. No. 6,200,547 describes: “magnetically controllable, or guided, carrier composition and methods of use and production are disclosed, the composition for carrying biologically active substances to a treatment zone in a body under control of a magnetic field. The composition comprises composite, volume-compounded paclitaxel-adsorbed particles of 0.2 to 5.0 μm in size, and preferably between 0.5 and 5.0 μm, containing 1.0 to 95.0% by mass of carbon, and preferably from about 20% to about 60%. The particles are produced by mechanical milling of a mixture of iron and carbon powders. The obtained particles are placed in a solution of a biologically active substance to adsorb the substance onto the particles. The composition is generally administered in suspension. Magnetic carrier particles having therapeutic quantities of adsorbed paclitaxel, doxorubicin, Tc99, and antisense-C Myc oligonucleotide, an hematoporphyrin derivative, 6-mercaptopurine, Amphotericin B, and Camptothecin have been produced using this invention . . . ”. The entire disclosure of this United States patent is hereby incorporated by reference into this specification.

In one embodiment, paclitaxel is bonded to the nanomagnetic particles of this invention in the manner described in U.S. Pat. No. 6,200,547.

By way of yet further illustration, one may use the process of U.S. Pat. No. 6,483,536. This patent describes: “A magnetically controllable, or guided, carrier composition and methods of use and production are disclosed, the composition for carrying biologically active substances to a treatment zone in a body under control of a magnetic field. The composition comprises composite, volume-compounded paclitaxel-, adsorbed particles of 0.2 to 5.0 μm in size, and preferably between 0.5 and 5.0 μm, containing 1.0 to 95.0% by mass of carbon, and preferably from about 20% to about 60%. The particles are produced by mechanical milling of a mixture of iron and carbon powders. The obtained particles are placed in a solution of a biologically active substance to adsorb the substance onto the particles. The composition is generally administered in suspension. Magnetic carrier particles having therapeutic quantities of adsorbed paclitaxel, doxorubicin, Tc99, and antisense-C Myc oligonucleotide, an hematoporphyrin derivative, 6-mercaptopurine, Amphotericin B, and Camptothecin have been produced using this invention. Magnetic carrier particles having diagnostic quantities of adsorbed Re186 and Re188 have also been produced using this invention.” The entire disclosure of this United States patent is hereby incorporated by reference into this specification. As will be apparent, the process of this patent may be used to adsorb paclitaxel onto the nanomagentic particles of this invention.

By way of yet further illustration, one may enhance the an anti-microtubule agent by using magnetotactic bacteria as a drug carrier that can be directed to the desired site of drug action by guiding the bacteria through the body of a patient via an applied magnetic field whose intensity increases in the vicinity of the desired site.

The preparation and use of magnetotactic bacteria assemblies is well known to those skilled in the art. Thus, and by way of illustration, in U.S. Pat. No. 4,394,451 of Blakemore (the entire disclosure of which is hereby incorporated by reference into this specification), there is described and claimed: “An aqueous culture medium for the growth of a biologically pure culture of magnetic bacteria, comprising, per 100 ml, about 2-30 μM of ferric quinate, about 10-1000 mg of an organic compound selected from the group consisting of fumaric acid, tartaric acid, malic acid, succinic acid, lactic acid, pyruvic acid, oxaloacetic acid, malonic acid, β-hydroxybutyric acid, maleic acid, galactose, rhanmose, melibiose, acetic acid, adipic acid, and glutaric acid, a vitamin source, a mineral source, a nitrogen source, an acetate source, and a pH buffer, said pH buffer resulting in a pH of said aqueous culture medium of about 5.2-7.5.” In the specification of this patent (starting at line 49 of Column 2 thereof), it was disclosed that: “A magnetotactic bacterium was isolated from fresh water swamps and was cultured in the laboratory on the special growth medium of the present invention. Frankel, Blakemore, and Wolfe, Science, 203, 1355 (1979). The organism is a magnetotactic Aquaspirillum and appears to be a new bacterial species by criteria separate from its magnetic properties. It has been designated strain MS-1. A culture of this microorganism has been deposited in the permanent collection of the American Type Culture Collection, Rockville, Md. A subculture of the microorganism may be obtained upon request. Its accession number in this repository is ATCC 31632”

U.S. Pat. No. 4,452,896 of Richard P. Blakemore et al. is another United States patent relating to magnetic bacteria; the entire disclosure of this United States patent is also incorporated by reference into this specification. This United States patent describes and claims: “A method for growing a biologically pure culture of magnetic bacteria, comprising mixing, per 100 ml, about 2-30 μM of ferric quinate, about 10-1000 mg. of an organic compound selected from the group consisting of fumaric acid, tartaric acid, malic acid, succinic acid, lactic acid, pyruvic acid, oxaloacetic acid, malonic acid, β-hydroxybutyric acid, maleic acid, galactose, rhamnose, melibiose, acetic acid, adipic acid, and glutaric acid, a vitamin source, a mineral source, a nitrogen source, an acetate source, and a pH buffer within the range of about 5.2-7.5, inoculating the mixture with said magnetic bacteria, providing said magnetic bacteria with an atmosphere having an initial oxygen concentration of about 0.2-6% by volume, and maintaining the ambient temperature in the range of about 18°-35° C.”

In one embodiment of this invention, magnetotactic bacteria comprised of one or more anti-microtubule agents are caused to migrate to the coated substrate assembly 3100 (see FIG. 36) by the application of an external magnetic field.

Magnetotactic bacteria migrate along the direction of a magnetic field. In one embodiment, of this invention, one or more anti-microtubule agents, such as paclitaxel (or other similar cancer drugs) are incorporated into such bacteria. One may, e.g., coat the paclitaxel with an organic material that the specific type of bacteria used will be attracted to as a nutrient and hence ingest drug molecules in the process. Subsequently, the paclitaxel-containing bacteria are directed towards the desired site in a patient's body through an application of a magnetic field as guidance for their migration to such site. In one aspect of this embodiment, paclitaxel-containing bacteria are injected into, onto, or near the desired site. In another aspect of this embodiment, the paclitaxel-containing bacteria are fed to the patient, who is then subjected to electromagnetic radiation in accordance with the procedure described elsewhere in this specification.

Thus, e.g., the electromagnetic radiation or an inhomogeneous magnetic field can be focused onto the desired site(s), in which case the magnetotactic bacterial would drift towards the tumor site and excrete the Paclitaxel at such site executing a drug delivery mechanism to the site in the process. This process would continue as long as the electromagnetic radiation continued to be applied.

It should be noted that bacteria are prokaryotic organisms that are not as adversely affected by anti-microtubule agents as are human beings in that the bacteria do not express tubulin.

Referring again to FIGS. 36 and 37 of the instant specification, and to the preferred embodiment depicted therein, the morphologically indented surface 3106 may be made by conventional means.

Referring again to FIG. 36, and in one preferred embodiment thereof, the size of the indentations 3108 is preferably chosen such that it matches the size of the drug particles 3110. In one embodiment, depicted in FIG. 36A, the surface 3112 of the indentations 3108 is coated with receptor material 3114 adapted to bind to the drug particles 3110.

Receptor material 3114 is comprised of a “recognition molecule”. As is known to those skilled in the art, recognition is a specific binding interaction occurring between macromolecules.

Many recognition molecules and recognition systems are described in, e.g., United States patents.

Thus, by way of illustration, U.S. Pat. No. 5,482,836 (the entire disclosure of which is hereby incorporated by reference into this specification) discloses a process which utilizes both a “first recognition molecule of a specific molecular recognition system” and a “second recognition molecule specifically binding to the first recognition molecule.” As is disclosed in column 3 of this patent, “ . . . a molecular recognition sytem is a system of at least two molecules which have a high capacity of molecular recognition for each other.” This term is also dicussed at column 6 of U.S. Pat. No. 5,482,836, wherein it is stated that: “A ‘molecular recognition system’ is a system of at least two molecules which have a high capacity of molecular recognition for each other and a high capacity to specifically bind to each other. Molecular recognition systems for use in the invention are conventional and are not described here in detail. Techniques for preparing and utilizing such systems are well-known in the literature and are exemplified in the publication Tijssen, P., Laboratory Techniques in Biochemistry and Molecular Biology Practice and Theories of Enzyme Immunoassays, (1988), eds. Burdon and Knippenberg, N.Y.:Elsevier.”

The terms “bind” or “bound”, etc. include both covalent and non-covalent associations, but can also include other molecular associations where appropriate such as Hoogsteen hydrogen bonding and Watson-Crick hydrogen bonding.”

At column 7 of U.S. Pat. No. 5,482,836, a description of some typical molecular recognition systems is presented. These systems include “ . . . an antigen/antibody, an avidin/biotin, a streptavidin/biotin, a protein A/Ig and a lectin/carbohydrate system. The preferred embodiment of the invention uses the streptavidin/biotin molecular recognition system and the preferred oligonucleotide is a 5′-biotinylated homopyrimidine oligonucleotide.”

Thus, by way of further illustration, U.S. Pat. No. 5,705,163 describes “A method for killing a target cell, said method comprising contacting said target cell with a cytotoxic amount of a composition comprising a recombinant Pseudomonas exotoxin (PE) having a first recognition molecule for binding said target cell and a carboxyl terminal sequence of 4 to 16 amino acids which permits translocation of the PE molecule into a cytosol of said target cell, the first recognition molecule being inserted in domain III after and no acid 600 and before amino acid 613 of the PE” (see claim 1). The entire disclosure of this United States patent is hereby incorporated by reference into this specification.

Thus, by way of yet further illustration, U.S. Pat. No. 5,922,537 describes a “binding agent bound through specific recognition sites to an immobilized analyte” (see claim 1). The entire disclosure of this United States patent is hereby incorporated by reference into this specification.

Thus, by way of further illustration, U.S. Pat. No. 6,297,059 describes “An optical biosensor for detection of a multivalent target biomolecule comprising: a substrate having a fluid membrane thereon; recognition molecules situated at a surface of said fluid membrane, said recognition molecule capable of binding with said multivalent target biomolecule and said recognition molecule linked to a single fluorescence molecule and as being movable upon said surface of said fluid membrane; and, a means for measuring a change in fluorescent properties in response to binding between multiple recognition molecules and said multivalent target biomolecule” (see claim 1.). As is disclosed in column 1 of this patent, “Biological sensors are based upon the immobilization of a recognition molecule at the surface of a transducer (a device that transforms the binding event between the target molecule and the recognition molecule into a measurable signal). In one prior approach, the transducer has been sensitive to any binding, specific or non-specific, that occurred at the transducer surface. Thus, for surface plasmon resonance or any other transduction that depended on a change in the index of refraction, such sensors have been sensitive to both specific and non-specific binding. Another prior approach has relied on a sandwich assay where, for example, the binding of an antigen by an antibody has been followed by the secondary binding of a fluorescently tagged antibody that is also in the solution along with the protein to be sensed. In this approach, any binding of the fluorescently tagged antibody will give rise to a change in the signal and, therefore, sandwich assay approaches have also been sensitive to specific as well as non-specific binding events. Thus, selectivity of many prior sensors has been a problem.

Another previous approach where signal transduction and amplification have been directly coupled to the recognition event is the gated ion channel sensor as described by Cornell et al., “A Biosensor That Uses Ion-Channel Switches”, Nature, vol. 387, Jun. 5, 1997. In that approach an electrical signal was generated for measurement. Besides electrical signals, optical biosensors have been described in U.S. Pat. No. 5,194,393 by Hugl et al. and U.S. Pat. No. 5,711,915 by Siegmund et al. In the later patent, fluorescent dyes were used in the detection of molecules.” In one embodiment of the process of this invention, the binding of a specific binding pair that is facilitated by the process of this invention is sensed and reported by a biological sensor.

Thus, by way of further illustration, U.S. Pat. No. 6,337,215 (the entire disclosure of which is hereby incorporated by reference into this specification) discloses “an affinity recognition molecule attached to the coating of the magnetic particle for selectively binding with a target molecule” (see claim 1 of the patent). In particular, claim 1 of U.S. Pat. No. 6,337,215 describes: “A composition of matter comprising: a magnetic particle comprising a first ferromagnetic layer having a moment oriented in a first direction, a second ferromagnetic layer having a moment oriented in a second direction generally antiparallel to said first direction, and a nonmagnetic spacer layer located between and in contact with the first and second ferromagnetic layers, and wherein the magnitude of the moment of the first ferromagnetic layer is substantially equal to the magnitude of the moment of the second ferromagnetic layer so that the magnetic particle has substantially zero net magnetic moment in the absence of an applied magnetic field, and wherein the thickness of the magnetic particle is substantially the same as the total thickness of said layers making up the particle; a coating on the surface of the magnetic particle; and an affinity recognition molecule attached to the coating of the magnetic particle for selectively binding with a target molecule.”

The “affinity recognition molecules” of U.S. Pat. No. 6,337,215, and means for attaching them to magnetic particles, are described in columns 16-18 of such patent, wherein it is disclosed that: “The following sections discuss the use of the above identified magnetic particles as nuclei for affinity molecules that are bound to the magnetic particles of the present invention. As indicated above, magnetic particles according to the present invention are attached to at least one affinity recognition molecule. As used herein, the term ‘affinity recognition molecule’ refers to a molecule that recognizes and binds another molecule by specific three-dimensional interactions that yield an affinity and specificity of binding comparable to the binding of an antibody with its corresponding antigen or an enzyme with its substrate. Typically, the binding is noncovalent, but the binding can also be covalent or become covalent during the course of the interaction. The noncovalent binding typically occurs by means of hydrophobic interactions, hydrogen bonds, or ionic bonds. The combination of the affinity recognition molecule and the molecule to which it binds is referred to generically as a ‘specific binding pair.’ Either member of the specific binding pair can be designated the affinity recognition molecule; the designation is for convenience according to the use made of the interaction. One or both members of the specific binding pair can be part of a larger structure such as a virion, an intact cell, a cell membrane, or a subcellular organelle such as a mitochondrion or a chloroplast.” As will be apparent, one or more of such recognition molecules may be attached to the surface(s) of the nanomagnetic particles of this invention.

“Examples of affinity recognition molecules in biology include antibodies, enzymes, specific binding proteins, nucleic acid molecules, and receptors. Examples of receptors include viral receptors and hormone receptors. Examples of specific binding pairs include antibody-antigen, antibodyhapten, nucleic acid molecule-complementary nucleic acid molecule, receptor-hormone, lectin-carbohydrate moiety, enzyme substrate, enzyme-inhibitor, biotin-avidin, and viruscellular receptor. One particularly important class of antigens is the Cluster of Differentiation (CD) antigens found on cells of hematopoietic origin, particularly on leukocytes, as well as on other cells. These antigens are significant in the activity and regulation of the immune system. One particularly significant CD antigen is CD34, found on stem cells. These are totipotent cells that can regenerate all of the cells of hematopoietic origin, including leukocytes, erythrocytes, and platelets.”

“As used herein, the term “antibody” includes both intact antibody molecules of the appropriate specificity and antibody fragments (including Fab, F(ab′), Fv, and F(ab′)2 fragments), as well as chemically modified intact antibody molecules and antibody fragments such as Fv fragments, including hybrid antibodies assembled by in vitro reassociation of subunits. The term also encompasses both polyclonal and monoclonal antibodies. Also included are genetically engineered antibody molecules such as single chain antibody molecules, generally referred to as sFv. The term “antibody” also includes modified antibodies or antibodies conjugated to labels or other molecules that do not block or alter the binding capacity of the antibody.”

“As used herein, the terms ‘nucleic acid molecule,’ ‘nucleic acid segment’ or ‘nucleic acid sequence’ include both DNA and RNA unless otherwise specified, and, unless otherwise specified, include both double-stranded and single stranded nucleic acids. Also included are hybrids such as DNA-RNA hybrids. In particular, a reference to DNA includes RNA that has either the equivalent base sequence except for the substitution of uracil and RNA for thymine in DNA, or has a complementary base sequence except for the substitution of uracil for thymine, complementarity being determined according to the Watson-Crick base pairing rules. Reference to nucleic acid sequences can also include modified bases or backbones as long as the modifications do not significantly interfere either with binding of a ligand such as a protein by the nucleic acid or with Watson-Crick base pairing.”

“Methods for the covalent attachment of biological recognition molecules to solid phase surfaces, including the magnetic particles of the present invention, are well known in the art and can be chosen according to the functional groups available on the biological recognition molecule and the solid phase surface.”

“Many reactive groups on both protein and non-protein compounds are available for conjugation. For example, organic moieties containing carboxyl groups or that can be carboxylated can be conjugated to proteins via the mixed anhydride method, the carbodiimide method, using dicyclohexylcarbodiimide, and the N hydroxysuccinimide ester method.”

“If the organic moiety contains amino groups or reducible nitro groups or can be substituted with such groups, conjugation can be achieved by one of several techniques. Aromatic amines can be converted to diazonium salts by the slow addition of nitrous acid and then reacted with proteins at a pH of about 9. If the organic moiety contains aliphatic amines, such groups can be conjugated to proteins by various methods, including carbodiimide, tolylene-2,4-diisocyanate, or malemide compounds, particularly the N-hydroxysuccinimide esters of malemide derivatives. An example of such a compound is 4(Nmaleimidomethyl)-cyclohexane-1-carboxylic acid. Another example is m-male imidobenzoyl-N-hydroxysuccinimide ester. Still another reagent that can be used is N-succinimidyl-3 (2-pyridyldithio) propionate. Also, bifunctional esters, such as dimethylpimelimidate, dimethyladipimidate, or dimethylsuberimidate, can be used to couple amino-group containing moieties to proteins.”

“Additionally, aliphatic amines can also be converted to aromatic amines by reaction with p-nitrobenzoylchloride and subsequent reduction to a p-aminobenzoylamide, which can then be coupled to proteins after diazotization.”

“Organic moieties containing hydroxyl groups can be cross-linked by a number of indirect procedures. For example, the conversion of an alcohol moiety to the half ester of succinic acid (hemisuccinate) introduces a carboxyl group available for conjugation. The bifunctional reagent sebacoyldichloride converts alcohol to acid chloride which, at pH 8.5, reacts readily with proteins. Hydroxyl containing organic moieties can also be conjugated through the highly reactive chlorocarbonates, prepared with an equal molar amount of phosgene.”

“For organic moieties containing ketones or aldehydes, such carbonyl-containing groups can be derivatized into carboxyl groups through the formation of O-(carboxymethyl) oximes. Ketone groups can also be derivatized with p-hydrazinobenzoic acid to produce carboxyl groups that can be conjugated to the specific binding partner as described above. Organic moieties containing aldehyde groups can be directly conjugated through the formation of Schiff bases which are then stabilized by a reduction with sodium borohydride.”

“One particularly useful cross-linking agent for hydroxyl-containing organic moieties is a photosensitive noncleavable heterobifunctional cross-linking reagent, sulfosuccinimidyl 6-[4¢-azido-2¢-nitrophenylamino] hexanoate. Other similar reagents are described in S. S. Wong, “Chemistry of Protein Conjugation and CrossLinking,” (CRC Press, Inc., Boca Raton, Fla. 1993). Other methods of crosslinking are also described in P. Tijssen, “Practice and Theory of Enzyme Immunoassays” (Elsevier, Amsterdam, 1985), pp. 221-295.”

“Other cross-linking reagents can be used that introduce spacers between the organic moiety and the biological recognition molecule. The length of the spacer can be chosen to preserve or enhance reactivity between the members of the specific binding pair, or, conversely, to limit the reactivity, as may be desired to enhance specificity and inhibit the existence of cross-reactivity.”

“Although, typically, the biological recognition molecules are covalently attached to the magnetic particles, alternatively, noncovalent attachment can be used. Methods for noncovalent attachment of biological recognition molecules to magnetic particles are well known in the art and need not be described further here.”

“Conjugation of biological recognition molecules to magnetic particles is described in U.S. Pat. No. 4,935,147 to Ullman et al., and in U.S. Pat. No. 5,145,784 to Cox et al., both of which are incorporated herein by this reference.”

Thus, by way of further illustration, U.S. Pat. No. 6,682,648 describes “a recognition molecule capable of specifically binding an analyte in a structure restricted manner” (see claim 1); the entire disclosure of this United States patent is hereby incorporated by reference into this specification. The “analyte” disclosed in such patent is preferably an antigen or antibody. Thus, as is disclosed at column 7 of this patent, “The term “antibody” refers to immunoglobulins of any isotype or subclass as well as any fab or fe fragment of the aforementioned. Antibodies of any source are applicable including polyclonal materials obtained from any animal species; monoclonal antibodies from any hybridoma source; and all immunoglobulins (or fragments) generated using viral, prokaryotic or eukaryotic expression systems. Biologic recognition molecules other than antibodies, are equally applicable for use with the current invention. These include, but are not limited to: cell adhesion molecules, cell surface receptor molecules, and solubilized binding proteins. Non-biologic binding molecules, such as ‘molecular imprints’ (synthetic polymers with pre-determined specifically for binding/complex formation), are also applicable to the invention. The terms ‘antigens,’ ‘immunogens’ or ‘haptens’ refer to substances which can be recognized by in vivo or in vitro immune elements, and are capable of eliciting a cellular or humoral immunologic response.” Although the electrochemically active reporter utilized in the embodiment is specified as para-aminophenol (generated by the action of a beta-galactosidase conjugate in conjunction with a specific substrate), it should be noted that the invention is generally applicable to molecules capable of redox recycling, and enzyme systems capable of generating such reporters.

Thus, by way of illustration, U.S. Pat. No. 6,686,209 discloses a recognition molecule having a binding site that is capable of binding to tetrahydrocannabinoids. The entire disclosure of this United Slates patent is hereby incorporated by reference into this specification.

By way of further illustration, “recognition molecules” and/or “recognition systems” and/or “affinity molecules” and/or “specific binding pairs” are disclosed, e.g., in U.S. Pat. Nos. 5,268,306 (preparation of a solid phase matrix containing a bound specific pair), U.S. Pat. No. 6,103,537 (separation of free and bound species), U.S. Pat. Nos. 5,972,630, 6,399,299, 6,261,554 (compositions for targeted gene delivery), U.S. Pat. No. 6,054,281 (binding assays), U.S. Pat. No. 6,004,745 (hybridization protection assay), U.S. Pat. Nos. 5,998,192, 5,851,770 (detection of mismatches by resolvase cleavage using a magentic bead support), U.S. Pat. No. 5,716,778 (concentrating immunochemical test device), U.S. Pat. No. 5,639,604 (homogeneous protection assay), U.S. Pat. No. 4,629,690 (homogeneous enzyme specific binding assay on non porous surface), U.S. Pat. Nos. 4,435,504, 6,489,123 (labelling and selection of molecules), U.S. Pat. Nos. 6,342,588, 6,180,336, 6,1543,442 (reagents and methods for specific binding assays), U.S. Pat. No. 6,068,981 (marking of orally ingested products), U.S. Pat. No. 5,8538,983 (inhibition of cell adhesion protein-carbohydrate interactions), U.S. Pat. No. 5,801,000 (detection and isolation of receptors), U.S. Pat. No. 5,766,934 (sensors with immobilized indicator molecules), U.S. Pat. No. 5,554,499 (detection and isolation of ligands), U.S. Pat. No. 4,713,350 (hydrophilic assay containing one member of a specific binding pair), U.S. Pat. No. 4,650,751 (protected binding assay), U.S. Pat. No. 4,575,485 (ultasonic ehanced immuno-reactions), and the like. The entire disclosure of each of these United States patents is hereby incorporated by reference into this specification.

Referring again to FIG. 36, and in the embodiment depicted, an external attachment electromagnetic field 3116 is shown being applied near the surface 3106 of the coated substrate 3100. This applied field 3116 is adapted to facilitate the bonding of the drug particles 3110 to the indentations 3108. As long as such indentations are not totally filled, and as long as the appropriate electromagentic field is applied, then the drug molecules 3110 will continue to bond to such indentations 3108.In one embodiment, not depicted in FIG. 36, instead of drug particles 3110 or in addition thereto, one or more of the nanomagnetic particles of this invention may be caused to bind to a specific site within a biological organism.

The external attachment electromagnetic field may, e.g., be ultrasound. It is known that ultrasound can be used to greatly enhance the rate of binding between members of a specific binding pair. Reference may be had, e.g., to U.S. Pat. No. 4,575,485, which claims: “In a method for measuring the binding of members of a specific binding pair in an aqueous medium, the improvement which comprises ultrasonicating the medium containing the members of the specific binding pair for a sufficient time to enhance the rate of binding of said members” (see claim 1). As is disclosed in this patent, improved “ . . . rates are obtained in the binding between members of a specific binding pair, particularly where one of the members of the specific binding pair is bound to a solid support . . . .” The entire disclosure of this United States patent is hereby incorporated by reference into this specification.

As is further disclosed in U.S. Pat. No. 4,575,485, “As mentioned above, of particular interest for the subject invention is where one of the members of the specific binding pair is conjugated to a solid support, usually non-diffusibly conjugated to a non-dispersible solid support . . . . The specific binding member may be conjugated to the support either covalently or non-covalently, normally depending upon the specific member, as well as the nature of the support.”

“To enhance the rate of reaction of the ligand and receptor to form the complex in an assay such as one described above, the assay medium may be subjected to ultrasonication such as by introduction into a bath in an ultrasonic device. Generally, the medium is subjected to ultrasonic sound for a time sufficient to allow for at least about 25% of the binding between the members of the specific binding pair to occur. The frequency of ultrasonication will vary from about 5 to 103 kHz, preferably from about 15 to 500 kHz, depending upon the size of the bath, the time for the ultrasonication, and the available equipment. The power will generally be from about 10 to 100 watts, more usually from about 25 to 75 watts, and preferably from about 45 to 60 watts. The temperature will generally be maintained in the range of about 15° to 40° C. The assay medium will generally be a volume in the range of about 0.1 ml to 10 ml, usually from about 0.1 ml to 5 ml. The time may vary, depending on the frequency and power, from about 30 seconds to 2 hours, more usually from about 1 minute to 30 minutes. The power, frequency, and time will be chosen so as not to have a deleterious effect on the binding members and to assure accuracy of the assay.”

As is known to those skilled in the art, paclitaxel, and paclitaxel-type compounds, stabilize microtubules, preventing them from shortening and dividing the cell as a result of their shortening as they segregate the genetic material in chromosomes. Furthermore, paclitaxel increases the rigidity of microtubules making them susceptible to breaking given the right physical stimuli.

Ultrasound induces mechanical vibrations of microtubules. At the right frequency, and at the right power level, the application of ultrasound will cause the microtubules to first buckle and then break up.

The ultrasound used in one embodiment of the process of this invention preferably has a frequency of from about 50 megahertz to about 2 Gigahertz, and more preferably has a frequency of from about 100 megahertz to about 1 Gigahertz. The power of such ultrasound is preferably at least about 0.01 watts per square meter and, more preferably, at least about 0.1 watts per square meter. The ultrasound is preferably focused on the site to be treated, such as, e.g., a tumor. One may use any conventional means for focusing the ultrasound. Thus, e.g., one may use one or more of the devices disclosed in U.S. Pat. Nos. 6,613,0055 (systems and methods for steering a focused ultrasound array), U.S. Pat. Nos. 6,613,004, 6,595,934 (skin rejuvenation using high intensity focused ultrasound), U.S. Pat. No. 6,543,272 (calibrating a focused ultrasound array), U.S. Pat. No. 6,506,154 (phased array focused ultrasound system), U.S. Pat. No. 6,488,639 (high intensity focused ultrasound treatment apparatus), U.S. Pat. No. 6,451,013 (tonsil reduction using high intensity focused ultrasound to form an ablated tissue area), U.S. Pat. No. 6,432,067 (medical procedures using high-intensity focused ultrasound), U.S. Pat. No. 6,425,867 (noise-free real time ultrasonic imaging of a treatment site undergoing high intensity focused ultrasound therapy), and the like. The entire disclosure of each of these patent applications is hereby incorporated by reference into this specification.

In one embodiment, paclitaxel (or a similar composition) is delivered to the patient and, as is its wont, makes the microtubules more rigid. Thereafter, when the microtubules are polymerized in a dividing cell and substantially immobilized, the ultrasound is selectively delivered to the microtubules in delivery site, thereby breaking such microtubules and halting the process of cell growth.

In one aspect of this embodiment, after the paclitaxel (or similar material) has been delivered to the patient, the high intensity magnetic field is applied to the delivery site in order to selectively cause the paclitaxel to bind the microtubules in the site. Thereafter, the ultrasound is applied to break the microtubules so bound to the Paclitaxel enhancing the efficacy of the drug due to a combined effect of the magnetic field, ultrasound and chemotherapeutic action of Paclitaxel itself.

When microtubules have been broken, they tend to reform. Therefore, in one embodiment, the ultrasound is periodically or continuously delivered to the delivery site synchronized to the typical time elapsed between subsequent cell division processes during which microtubules are polymerized.

In one embodiment, a portable device is worn by the patient; and this device periodically and/or continuously delivers ultrasound and/or magnetic energy to the patient. In one aspect of this embodiment, the device first delivers high intensity magnetic energy, and then it delivers the ultrasound energy.

As is known to those skilled in the art, ultrasound is by one of the many forms of electromagnetic radiation that affect biological processes in general and, in particular, may affect the rate of binding or disassociation between two members of a specific binding pair. Some of these forms of electromagnetic radiation are disclosed in columns 2-4 of U.S. Pat. No. 5,566,685, the entire disclosure of which is hereby incorporated by reference into this specification. As is disclosed in this patent, at columns 1-2 thereof, “The prevalence of ELF EMFs at home, in educational establishments and in the work place, where people spend a great deal of their time, has for the past 10 years fueled considerable interest in scientific research to examine the possibility of adverse health effects from exposure to these fields. At the present time overwhelming evidence exists which shows that a wide range of biological effects are possible even at very low levels of exposure (<5 milligauss-mG). These effects include changes in transcription of specific genes, changes in enzyme activities, production of morphological abnormalities and biochemical modifications in developing chick embryos, stimulation of bone cell growth, suppression of nocturnal melatonin in humans, and alterations in cellular Ca2+ pools [Goodman, R., L.-X. Wei, J.-C. Xu, and A. Henderson, ‘Exposure of human cells to low-frequency electromagnetic fields results in quantitative changes in transcripts’, Biochim. Biophys. Acta, 1009:216-220, 1989; Battini, R., M. G. Monti, M. S. Moruzzi, S. Ferrari, P. Zaniol, and B. Barbiroli, ‘ELF electromagnetic fields affect gene expression of regenerating rat liver following partial hepatectomy’, J. Bioelec. 10:131-139, 1991; Krause, D., W. J. Skowronski, J. M. Mullins, R. M. Nardone, and J. J. Greene ‘Selective enhancement of gene expression by 60 Hz electromagnetic radiation’, in C. T. Brighton and S. R. Pollack, Eds. ‘Electromagnetics in Biology and Medicine’ (San Francisco Press, Inc., San Francisco, Calif.) pp. 133-138, 1991; Phillips, J. L., W. Haggren, W. J. Thomas, T. Ishida-Jones, and W. R. Adey, ‘Magnetic field-induced changes in specific gene transcription’, Biochim. Biophys. Acta 1132:140-144, 1992; Greene, J. J., S. L. Pearson, W. J. Skowronski, R. M. Nardone, J. M. Mullins, and D. Krause, ‘Gene-specific modulation of RNA synthesis and degradation by extremely low frequency electromagnetic fields’, Cell. Mol. Biol. 39:261-268, 1993; Byus, C. V., R. L. Lundak, R. M. Fletcher, and W. R. Adey, ‘Alterations in protein kinase activity following exposure of cultured human lymphocytes to modulated microwave fields’, Bioelectromag. 5:341-351, 1984; Byus, C. V., S. E. Pieper, and W. R. Adey, ‘The effects of low-energy 60-Hz environmental electromagnetic fields upon the growth-related enzyme ornithine decarboxylase’, Carcinogenesis 8:1385-1389, 1987; Litovitz, T. A., D. Krause, and J. M. Mullins, ‘Effects of coherence time of the applied magnetic field on omithine decarboxylase activity’, Biochem. Biophys. Res. Commun. 178:862-865, 1991; Litovitz, T. A., D. Krause, M. Penafiel, E. C. Elson, and J. M. Mullins, ‘The role of coherence time in the effect of microwaves on ornithine decarboxylase’, Bioelectromagnetics 14:395-403, 1993; Monti, M. G., L. Pernecco, M. S. Moruzzi, R. Battini, P. Zaniol, and B. Barbiroli, ‘Effect of ELF pulsed electromagnetic fields on protein kinase C activation process in HL-60 leukemia cells’, J. Bioelec. 10:119-130, 1991; Blank, M., ‘Na K-ATPase function in alternating electric fields’, FASEB J. 6:2434-2438, 1992; Delgado, J. M. R., J. Leal, J. L. Monteagudo, and M. G. Garcia, ‘Embryological changes induced by weak, extremely low frequency electromagnetic fields’, J. Anat. 134:533—551, 1992; Juutilainen, J., E. Laara, and K. Saali, ‘Relationship between field strength and abnormal development in chick embryos exposed to 50 Hz magnetic fields’, Int. J. Radiat. Biol. 52:787-793, 1987; Martin, A. H., ‘Magnetic fields and time dependent effects on development’, Bioelectromagnetics 9:393-396, 1988; Aaron, R., D. Ciombor, and G. Jolly, ‘Stimulation of experimental endochondral ossification by low-energy pulsing electromagnetic fields’, J. Bone Mineral Res. 4:227-233, 1989; Bassett, C. A. L., ‘Beneficial effects of electromagnetic fields’, J. Cell. Biochem. 51:387-393, 1993; Ciombor, D. M., and R. K. Aaron, ‘Influence of electromagnetic fields on endochondral bone formation’, J. Cell. Biochem. 52:37-41, 1993; Graham, C., M. R. Cook, H. D. Cohen, D. W. Riffle, S. J. Hoffman, F. J. McClemon, D. Smith, and M. M. Gerkovich, ‘EMF suppression of nocturnal melatonin in human volunteers, Abstract in the Proceedings of the Department of Energy Contractors Review Meeting October 1993; Wilson B. W., Wright C. W., Morris J. E., Buschbom R. L., and others ‘Evidence for an effect of ELF electromagnetic fields on human pineal gland function’, J. Pineal Res. 9:259-69, 1990; Reiter R. J., Anderson L. E., Busschbom R. L., Wilson B. W., ‘Reduction of the nocturnal melatonin rise in rats exposed to 60 Hz electric fields in utero and for 23 days after birth’, Life Sci. 42:2203-2206, 1988; Bawin, S. M., and W. R. Adey, ‘Sensitivity of calcium binding in cerebral tissue to weak environmental electric fields oscillating at low frequency’, Proc. Natl. Acad. Sci. USA 73:1999-2003, 1976; Bawin, S. M., W. R. Adey, and I. M. Sabbot, ‘Ionic factors in release of Ca2+ from chicken cerebral tissue by electromagnetic fields’, Proc. Natl. Acad. Sci. USA 75:6314-6318, 1978; Blackman, C. F., S. G. Benane, L. S. Kinney, D. E. House, and W. T. Joines, ‘Effects of ELF fields on calcium-ion efflux from brain tissue, in vitro’, Radiat. Res. 92:510-520, 1982; Lindstrom, E., P. Linstrom, A. Berglund, K. H. Mild, and E. Lundgren, ‘Intracellular calcium oscillations induced in a T-cell line by a weak 50 Hz magnetic field’, J. Cell. Physiol. 156:395-398 1993].”

A recent article by J. Ratoff appeared in “Science News” (published by Science Service, 1719 N. Street, N.W., Washington, D.C. 20036. This article, entitled “Magnetic Fields can diminish drug action,” disclosed that “The low-level electromagnetic fields present in some North American homes today can diminish or wipe out a wide prescribed drug's actions . . . . Researcher's have found that, when exposed to such fields, the drug tamoxifen lost its ability to halt the proliferation of cancer cells . . . . Gamoxifen is a synthetic hormone used to prevent the recurrence of breast cancer.”

A Jul. 3, 1993 article in “Science News” (see page 10 thereof) reported research that showed that while melatonin, a natural antioxidant hormone, would inhibit the growth of breast cance4r cells exposed to 2 milligauss magnetic fields, its activity was essentially reased when the cells were based in a 12 milliGauss field.

Articles on similar subjects have been published by: Blackman, C. F., et al., 1996, “Independent replication of the 12-mg magnetic field effect on melatonin and mcf-7 cells in vitro,” Eighteenth annual meeting of the Bioelectromagnetic Society, Victoria, British, Columbia; Harland, J. D. and R. P. Liburdy, 1997, “Environmental magnetic fields inhibit the antiproliferative action of tamoxifen and melatonin in a human breast cancer cell line,” Bioelectromagnetics 18; and Liburdy, R. P., et al., 1997, “A 12 mG . . . magnetic field inhibits tamoxifen's oncostatic action in a second human breast cancer cell line, T47D, Second World Congress for Electricity and Magnetism in Biology and Medicine, Bologna, Italy.

Related articles appearing in “Science News” include, e.g., “EMFs on the brain?,” Science News 147 (Jan. 21, 1995):44; “Study reaffirms tamoxifen's dark side,” Science News 145(Jun. 4, 1994): 356; “Cells haywire in electromagnetic field?,” Science News 133 (Apr. 2, 2988):216, “Power-line static,” Science News 140 (Sep. 28, 1991): 202; and “Do EMFs pose breast cancer risk?,” Science News 145 (Jun. 18, 1994): 388.

In one embodiment, the electromagnetic radiation used in the process of this invention is a magnetic field with a field strength of at least about 6 Tesla. It is known, e.g., that microtubules move linearly in magnetic fields of at least about 6 Tesla.

In this embodiment, the focusing of the magnetic field onto an in vivo site within a patient may be done by conventional magnetic focusing means. Thus, and referring to U.S. Pat. No. 5,929,732 (the entire disclosure of which is hereby incorporated by reference into this specification), one may utilize: “An apparatus and method for creating a magnetic beam wherein a focusing magnet assembly (45) is comprised of a first opposing magnet pair (20) and a second opposing magnet pair (30) disposed in a focusing plane, each magnet of the respective opposing magnet pairs having a like pole directed towards the geometric center of the focusing magnet assembly (45) to form an alignment path, two like magnetic beams extending from the alignment path on each side of the focusing magnet assembly (45), each beam being generally perpendicular to the focusing plane. A like pole of an unopposed magnet (10) can be directed down the alignment path from one side of the focusing magnet assembly (45) to produce a single magnetic beam extending generally perpendicular from the focusing magnet assembly opposite unopposed magnet (10). This beam is a magnetic monopole which emits pulses, levitates, degausses, stops electronics and separates materials.”

By way of further illustration, one may use the “Permanent Magnetic Keeper-Shield Assembly” disclosed in U.S. Pat. No. 6,488,615; the entire disclosure of this United States patent application is hereby incorporated by reference into this specification. This patent discloses: “A magnet keeper-shield assembly adapted to hold and store a permanent magnet used to generate a high gradient magnetic field. Such a field may penetrate into deep targeted tumor sites in order to attract magnetically responsive micro-carriers. The magnet keeper-shield assembly includes a magnetically permeable keeper-shield with a bore dimensioned to hold the magnet. A screw driven actuator is used to push the magnet partially out of the keeper-shield. The actuator is assisted by several springs extending through the base of the keeper-shield.”

Without wishing to be bound to any particular theory, applicants believe that the use of the high intensity magnetic field(s) focused onto or into a desired site will attract paclitaxel molecules to the site of the tumor. Paclitaxel is comprised of a 6-member aromatic ring and, thus, will have an induced magnetic moment when subjected to an external field as a result of the magnetically induced electron currents in the ring. Without wishing to be bound to any particular theory, applicants believe that, in the presence of a magnetic field, a magnetic moment is induced in the paclitaxel molecule. This effect will enhance the docking and binding of the paclitaxel molecule to the nearest tubulin molecule in a microtubule.

In one embodiment, after a patient has taken paclitaxel, he is exposed to the focused magnetic radiation for at least about 30 minutes, and this process is repeated at least once a week.

It is known that paclitaxel has an inherent magnetic moment. It is also known that paclitaxel may be chemically fixed to magnetic particles that are relatively large with respect to paclitaxel molecules, that is, equivalent to or larger than individual paclitaxel molecules. Nanomagnetic particles that are substantially smaller than paclitaxel molecules, such as the nanomagnetic particles of this invention, may be chemically bound to the drug. For all of the above described methods of binding, the result is a chemical agent that will bind to tubulin and thus effect a cellular therapy for, e.g., cancer, wherein the chemical agent may also be manipulated in a magnetic field. While this disclosure will relate largely to the use of paclitaxel as a chemotoxin, the approach may be extended to any other drug or chemical therapy wherein a large contrast in uptake between tissues and/or body regions is preferred.

FIG. 36B is a schematic of an electromagnetic coil set 3160 and 3162, aligned to an axis 3164, and which in combination create a magnetic standing wave 3166. The excitation energy delivered to the two coils 3160 and 3162 comprises a set of high frequency sinusoidal signals that are determined via well known Fourier techniques, to create a first zone 3168 having a positive standing wave magnetic field ‘E’, a second zone 3170 having a zero or near-zero magnetic field, and a third zone 3172 having a positive magnetic field ‘E’. It should be noted that the two zones 3168 and 3172 need not have exactly matched waveforms, in frequency, phase, or amplitude; it is sufficient that the magnetic fields in both are large with respect to the near-zero magnetic field in zone 3170. The fields in zones 3168 and 3172 may be static standing wave fields or time-varying standing waves. It should be noted that in order to create a zone 3170 of useful size (1 to 5 cm at the lower limit) and having reasonably sharp ‘edges’, the frequencies of the Fourier waveforms used to create standing wave 3166 may be in the gigahertz range. These fields may be switched on and off at some secondary frequency that is substantially lower; the resulting switched-standing-wave fields in zones 3168 and 3172 will impart vibrational energy to any magnetic materials within them, while the near-zero switched field in zone 3170 will not impart substantial energy into magnetic materials within its boundaries. This secondary switching frequency may be adjusted in concert with the amplitude of the standing wave field to tune the vibrational energy to impart an optimal level of thermal energy to a specific molecule (e.g. paclitaxel) by virtue of the natural resonant frequency of that molecule. The energy imparted to an individual molecule will follow the relationship E_{T}=C×M×A×F², where ET is the thermal energy imparted to an individual moledule, C is a constant, M is the magnetic moment of the molecule and any bound magnetic particles, A is the amplitude of the time-varying magnetic field, and F is the frequency of field switching.

FIG. 36C is a three-dimensional schematic showing the use of three sets of magnetic coils arranged orthogonally. Each of the axes, ‘X’, ‘Y’, and ‘Z’ will impart either positive thermal energy (E) in its outer zones that correspond to zones 3168 and 3172 (from FIG. 36B), or zero thermal energy, in its central zone which corresponds to zone 3170 (from FIG. 36B). It may be seen from FIG. 36C that there will be a small volume at the centroid of the overall 3-D volume that will have overall zero magnetically-induced thermal energy. The notations ‘1×E’, ‘2×E’, and ‘3×E’ denote the relative magnetically-induced thermal energy in other regions. Since the overall volume is made up of three zones in each of three dimensions, the overall volume will have 27 sectors. Of these sectors one (the centroid) will have near-zero magnetically-induced thermal energy, (6) sectors will have a ‘1×E’ energy level, (12) sectors will have a ‘2×E’ energy level, and (8) sectors will have a ‘3×E’ energy level.

If the energy imported to any individual molecule (e.g. paclitaxel bound to one or more nanomagnetic particles) is sufficiently larger than the binding energy of that molecule to its target (e.g. tubulin in the case of paclitaxel) to account for thermal losses in coupling magnetically-induced energy into the molecule, then binding between the paclitaxel molecule and the tubulin target will not occur. Thus if we define the binding energy between the two (e.g. paclitaxel to tubulin) as E_{B}, and D as a constant that compensates for damping losses due to a molecule that is not purely elastic, then the equation E_{T}>D×E_{B} will have been satisfied, and chemical binding (in this case between paclitaxel and tubulin) will not occur.

In one embodiment, a device having matched coil sets as shown in FIG. 36B, but in three orthogonal axes, creates an overall operational volume that imparts an relatively low energy in the above-described centroid (E_{T}<D×E_{B}), and imparts a relatively higher energy in the other surrounding (26) segments (E_{T}>D×E_{B}); and if the centroid volume corresponds to the site under treatment, then a high degree of binding will occur in the centroid and no binding will occur in the exterior regions. The size of the non-binding centroid region may be adjusted via alterations to the Fourier waveforms, relative energy levels may be adjusted via amplitude and frequency of field switching, and the region may be aligned to correspond to the volume of the tumor under treatment. One preferred method for use is to place the patient in the device as disclosed herein, administer either native paclitaxel (or other drug having an innate magnetic characteristic) or magnetically-enhanced Paclitaxel (nanomagnetic or other magnetic particles either chemically or magnetically bound), maintain the patient in the controlled fields for a period of time necessary for the drug to pass out of the patient's excretory system, and then remove the patient from the device.

In another embodiment, the three fields in the X, Y, and Z directions are selectively activated and deactivated in a predetermined pattern. For example, one may activate the field in the X axis, thus causing the therapeutic agent to align with the X axis. A certain time later the field along the X axis is deactivated and the field corresponding to the Y axis is activated for a predetermined period of time. The agent then aligns with the new axis. This may be repeated along any axis. By rapidly activating and deactivating the respective fields in a predetermined pattern, one imparts thermal and/or rotational energy to the molecule. When the energy imparted to the therapeutic agent is greater than the binding energy necessary to bring about a biological effect, such binding is drastically reduced.

In another embodiment, the Fourier techniques are selected so as to create a near-zero magnetic field zone external to the tissue to be treated, while a time-varying standing wave is generated within the centroid region. A therapeutic agent that is weakly attached to a magnetic carrier particle (a carrier-agent complex) is introduced into the body. In one embodiment, the carrier particle acts to inhibit the biological activity of the therapeutic agent. When the carrier-agent complex enters the region of variable magnetic field located at the centroid, the thermal energy imparted to the carrier-agent complex the agent is liberated from its carrier and is no longer inhibited by the presence of that carrier. The region external to the centroid is a near-zero magnetic field, thus minimizing any premature dissociation of the carrier-agent complex.

In one embodiment the carrier particles are organic moieties that are covalently attached to the therapeutic agent. By way of illustration and not limitation, one may covalently attach a nitroxide spin label to a therapeutic agent. As is know to those skilled in the art, a nitroxide spin label is a persistent paramagnetic free radical. Biomolecules are routinely modified by the attachment of such labeling compounds, thus generating paramagnetic biomolecules. Reference may be had to U.S. Pat. No. 6,271,382, the entire disclosure of which is hereby incorporated by reference into this specification.

In another embodiment the carrier particles are magnetic encapsulating agents that surround the therapeutic agent. By way of illustration and not limitation, one may encapsulate a therapeutic agent within magnetosomes or magnetoliposomes described elsewhere in this specification. The agent exhibits minimal biological activity when in a near-zero magnetic field as the agent is at least partially encapsulated. When the carrier-agent complex is exposed to a variable magnetic field of sufficient intensity, the carrier particle releases the agent at or near the desired location.

Referring again to FIGS. 36 and 36A, it will be seen that FIG. 36A is a partial sectional view of an indentation 3108 coated with a multiplicity of receptors 3114 for the drug molecules.

FIG. 37 is a schematic illustration of one process for preparing a coating with morphological indentations 3108. In this process, a mask 3120 is disposed over the film 3014. The mask 3120 is comprised of a multiplicity of holes 3122 through which etchant 3124 is applied for a time sufficient to create the desired indentations 3108

One may use conventional etching technology to prepare the desired indentations 3108.

By way of illustration and not limitation, one may use the process described in claim 23 of U.S. Pat. No. 4,252,865 to prepare a surface with indentations 3108; the entire disclosure of this United States patent is hereby incorporated by reference into this specification. Claim 23 of this patent describes “The method of making a highly solar-energy absorbing surface on a substrate body, which comprises the controlled sputtering application of a layer of amorphous semiconductor material to an exposed-surface area of said body, and then altering the exposed-surface morphology of said layer by etching the same to form an array of outwardly projecting structural elements, the etchant being selected for the particular semiconductor material and applied in such strength and for such exposure time and ambient conditions of temperature as to form said structural elements with an aspect ratio in the range 2:1 to 10:1 and at lateral spacings which are in the order of magnitude of a wavelength within the solar-energy spectrum.”

By way of further illustration, one may prepare a surface with the “unique surface morphology” described in claim 1 of U.S. Pat. No. 4,233,107, the entire disclosure of which is hereby incorporated by reference into this specification. This claim 1 describes “A method of producing an ultra-black coating, having an extremely high light absorption capacity, on a substrate, the blackness being associated with a unique surface morphology consisting of a dense array of microscopic pores etched into the surface, said method comprising: (a) preparing a substrate for plating with a nickel-phosphorus alloy; (b) immersing the thus-prepared substrate in an electroless plating bath containing nickel and hypophosphite ions in solution until an electroless nickel-phosphorus alloy coating has been deposited on said substrate; (c) removing the resulting substrate with the electroless nickel-phosphorus alloy coated thereon from the plating both and washing and drying it; (d) immersing the dried substrate with the electroless nickel-phosphorus alloy coated thereon obtained in step (c) in an etchant bath consisting of an aqueous solution of nitric acid wherein the nitric acid concentration ranges from a 1:5 ratio with distilled or de-ionized water to concentrated, until the substrate surface develops ultra-blackness, said ultra-blackness being associated with said uniqud morphology; and (e) washing and drying the resulting substrate covered with the nickel-phosphorus alloy coating having said ultra-black surface.”

By way of yet further illustration, one may use the texturing process described in U.S. Pat. No. 5,830,793 and claimed in, e.g., claim 1 thereof. As is described in such claim 1, such texturing process comprises the steps of “ . . . seeding a semiconductor surface adjacent a substrate surface; annealing the seeded surface; and removing seeding formations from the substrate surface, wherein seeding comprises inducing nucleation sites in a greater amount on the semiconductor surface than on the substrate surface, and removing seeding formations from the substrate surface comprises selectively etching the substrate surface relative to the semiconductor surface.”

Referring again to FIG. 37, and to the process depicted therein, after the indentations 3108 have been formed, the etchant is removed from the holes 3122 and the indentations 3108 by conventional means, such as, e.g., by risning, and then receptor material 3114 is used to form the receptor surface. The receptor material 3114 may be deposited within the indentations by one or more of the techniques described elsewhere in this specification.

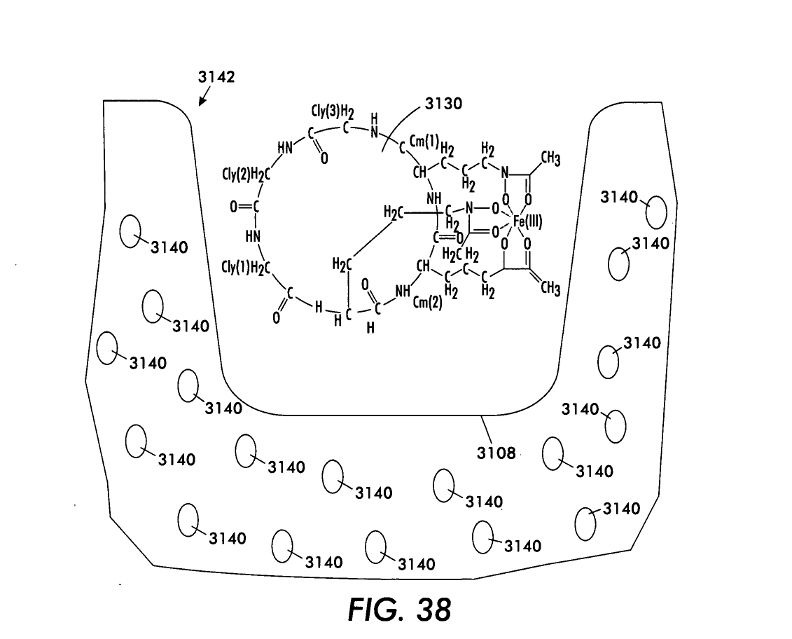

FIG. 38 is a schematic illustration of a drug molecule 3130 disposed inside of a indentation 3108. Referring to FIG. 38, and to the preferred embodiment depicted therein, it will be seen that a multiplicity of nanomagnetic particles 3140 are disposed around the drug molecule 3130. In the embodiment depicted, the forces between particles 3140 and 3130 may be altered by the application of an external field 3142. In one case, the characteristics of the field are chosen to facilitate the attachment of the particles 3130 to the particles 3140. In another case, the characteristics of the field are chosen to cause detachment of the particles 3130 from the particles 3140.

In one embodiment, the drug molecule 3130 is an anti-microtubule agent. Thus, and referring to U.S. Pat. No. 6,333,347 (the entire disclosure of which is hereby incorporated by reference into this specification), the anti-microtubule agent is preferably administered to the pericardium, heart, or coronary vasculature.

As is known to those skilled in the art, most physical and chemical interactions are facilitated by certain energy patterns, and discouraged by other energy patterns. Thus, e.g., electromagnetic attractive force may be enhanced by one applied electromagnetic filed, and electromagnetic repulsive force may be enhanced by another applied electromagnetic field. One, thus, by choosing the appropriate field(s), can determine the degree to which the one recognition molecule will bind to another, or to which a drug will bind to a implantable device, such as, e.g., a stent.

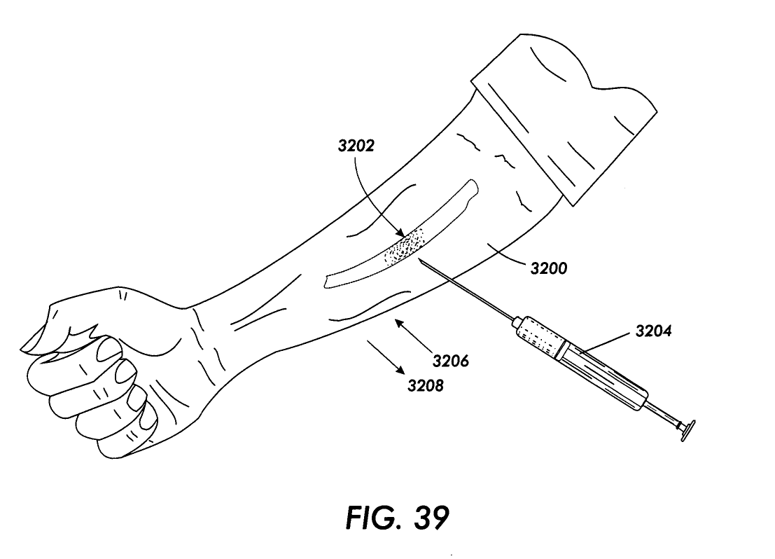

In one process, illustrated in FIG. 39, paclitaxel is administered into the arm 3200 of a patient near a stent 3202, via an injector 3204. During this administration, a first electromagnetic field 3206 is directed towards the stent 3202 in order to facilitate the binding of the paclitaxel to the stent. When it has been determined that a sufficient amount of paclitaxel has bound to the stent, a second electromagnetic field 3208 is directed towards the stent 3202 to discourage the binding of paclitaxel to the stent. The strength of the second electromagentic field 3208 is sufficient to discourage such binding but not necessarily sufficient to dislodge paclitaxel particles already bound to the stent and disposed within indentations 3208.

A Preferred Binding Process

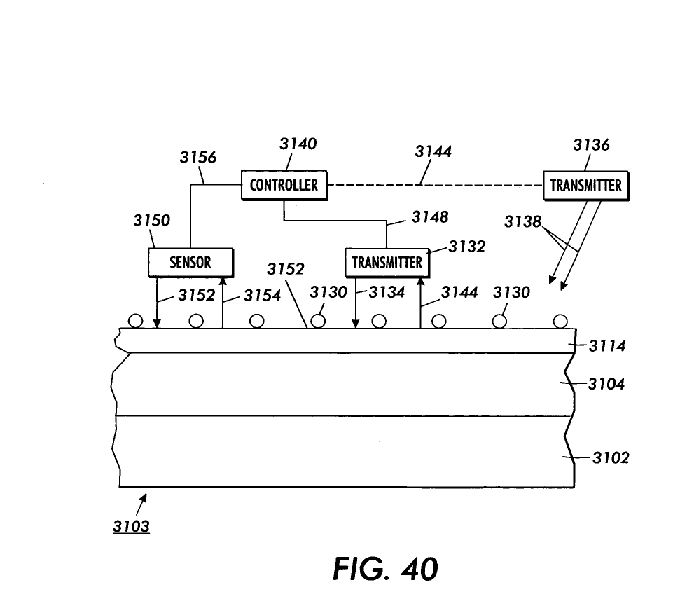

FIG. 40 is a schematic illustration of a preferred binding process of the invention. As will be apparent, FIG. 40 is not drawn to scale, and unnecessary detail has been omitted for the sake of simplicity of representation.

In the first step of the process of FIG. 40, a multiplicity of drug particles, such as drug particles 3130, are brought close to or contiguous with a coated substrate 3103 comprised of receptor material 3114 disposed on its top surface. The drug particles 3130 are near and/or contiguous with the receptor material 3114. They may be delivered to such receptor material 3114 by one or more of the drug delivery processes discussed elsewhere in this specification.

In the second step of the process depicted in FIG. 40, the substrate 3102/coating 3104/receptor material 3114/drug particles 3130 assembly is contacted with electromagnetic radiation to affect, e.g., the binding of the drug particles 3130 to the receptor material 3114. This may be done by, e.g., the transmission of ultrasonic radiation, as is discussed elsewhere in this specification. Alternatively, or additionally, it may be done by the use of other electromagnetic radiation that is known to affect the rate of binding between two recognition moieties and/or other biological processes.

The electromagnetic radiation may be conveyed by transmitter 3132 in the direction of arrow 3134. Alternatively, or additionally, the electromagnetic radiation may be conveyed by transmitter 3136 in the direction of arrows 3138. In the embodiment depicted in FIG. 40, both transmitter 3132 and/or transmitter 3136 are operatively connected to a controller 3140. The connection may be by direct means (such as, e.g., line 3142), and/or by indirect means (such as, e.g., telemetry link 3144).

Referring again to FIG. 40, and in the preferred embodiment depicted therein, transmitter 3132 is comprised of a sensor (not shown) that can monitor the radiation 3144 retransmitted from the surface 3114 of assembly 3103.

One may use many forms of electromagnetic radiation to affect the binding of the drug moieties 3130 to the receptor surface 3114. By way of illustration, and referring to agent U.S. Pat. No. 6,095,148 (the entire disclosure of which is hereby incorporated by reference into this specification), the growth and differentiation of nerve cells may be affected by electrical stimulation of such cells. As is disclosed in column 1 of such patent, “Electrical charges have been found to play a role in enhancement of neurite extension in vitro and nerve regeneration in vivo. Examples of conditions that stimulate nerve regeneration include piezoelectric materials and electrets, exogenous DC electric fields, pulsed electromagnetic fields, and direct application of current across the regenerating nerve. Neurite outgrowth has been shown to be enhanced on piezoelectric materials such as poled polyvinylidinedifluoride (PVDF) (Aebischer et al., Brain Res., 436;165 (1987); and R. F. Valentini et al., Biomaterials, 13:183 (1992)) and electrets such as poled polytetrafluoroethylene (PTFE) (R. F. Valentini et al., Brain. Res. 480:300 (1989)). This effect has been attributed to the presence of transient surface charges in the material which appear when the material is subjected to minute mechanical stresses. Electromagnetic fields also have been shown to be important in neurite extension and regeneration of transected nerve ends. R. F. Valentini et al., Brain. Res., 480:300 (1989); J. M. Kerns et al., Neuroscience 40:93 (1991); M. J. Politis et al., J. Trauma, 28:1548 (1988); and B. F. Sisken et al., Brain. Res., 485:309 (1989). Surface charge density and substrate wettability have also been shown to affect nerve regeneration. Valentini et al., Brain Res., 480:300-304 (1989).”

By way of further illustration, and again referring to U.S. Pat. No. 5,566,685, extremely low frequency electromagnetic fields may be used to cause, e.g., “ . . . changes in enzyme activities . . . ” “ . . . stimulation of bone cell growth . . . ,” “ . . . suppression of nocturnal melatonin . . . ,” “ . . . quantative changes in transcripts . . . ,” changes in “ . . . gene expression of regenerating rate liver . . . ,” changes in “ . . . gene expression . . . ,” changes in “ . . . gene transcription . . . ,” changes in “ . . . modulation of RNA synthesis and degradation . . . ,” . . . alterations in protein kinase activity . . . ,” changes in “ . . . growth-related enzyme ornithine decarboxylase . . . ,” changes in embryological activity, “ . . . stimulation of experimental endochondral ossification . . . ,” “ . . . suppression of nocturnal melatonin . . . ,” changes in “ . . . human pineal gland function . . . ,” changes in “ . . . calcium binding . . . ,” etc. Reference may be had, in particular, to columns 2 and 3 of U.S. Pat. No. 5,566,685.

Referring again to FIG. 40, and to the preferred embodiment depicted therein, the transmitter 3132 preferably has a sensor to determine the extent to which radiation incident upon, e.g., surface 3146 is reflected. Information from transmitter 3132 may be conveyed to and from controller 3140 via line 3148.

In the embodiment depicted in FIG. 40, a sensor 3150 is adapted to sense the degree of binding on surface 3146 between the drug molecules 3130 and the receptor molecules 3114. This sensor 3150 preferably transmits radiation in the direction of arrow 3152 and senses reflected radiation traveling in the direction of arrow 3154. Information from and to controller 3140 is fed to and from sensor 3150 via line 3156.

There are many sensors known to those skilled in the art which can determine the extent to which two recognition molecules have bound to each other.

Thus, e.g., one may use the process and apparatus described in U.S. Pat. No. 5,376,556, in which an analyte-mediated ligand binding event is monitored; the entire disclosure of this United States patent is hereby incorporated by reference into this specification. Claim 1 of this patent describes “A method for determining the presence or amount of an analyte, if any, in a test sample by monitoring an analyte-mediated ligand binding event in a test mixture the method comprising: forming a test mixture comprising the test sample and a particulate capture reagent, said particulate capture reagent comprising a specific binding member attached to a particulate having a surface capable of inducing surface-enhanced Raman light scattering and also having attached thereto a Raman-active label wherein said specific binding member attached to the particulate is specific for the analyte, an analyte-analog or an ancillary binding member; providing a chromatographic material having a proximal end and a distal end, wherein the distal end of said chromatographic material comprises a capture reagent immobilized in a capture situs and capable of binding to the analyte; applying the test mixture onto the proximal end of said chromatographic material; allowing the test mixture to travel from the proximal end toward the distal end by capillary action; illuminating the capture situs with a radiation sufficient to cause a detectable Raman spectrum; and monitoring differences in spectral characteristics of the detected surface-enhanced Raman scattering spectra, the differences being dependent upon the amount of analyte present in the test mixture.”

By way of further illustration, one may use the “triggered optical sensor” described and claimed in U.S. Pat. No. 6,297,059, the entire disclosure of which is hereby incorporated by reference into this specification. This patent claims (in claim 1) thereof”. An optical biosensor for detection of a multivalent target biomolecule comprising: a substrate having a fluid membrane thereon; recognition molecules situated at a surface of said fluid membrane, said recognition molecule capable of binding with said multivalent target biomolecule and said recognition molecule linked to a single fluorescence molecule and as being movable upon said surface of said fluid membrane; and, a means for measuring a change in fluorescent properties in response to binding between multiple recognition molecules and said multivalent target biomolecule.” In column 1 of this patent, other biological sensors are discussed, it being stated that: “Biological sensors are based on the immobilization of a recognition molecule at the surface of a transducer (a device that transforms the binding event between the target molecule and the recognition molecule into a measurable signal). In one prior approach, the transducer has been sensitive to any binding, specific or non-specific, that occurred at the transducer surface. Thus, for surface plasmon resonance or any other transduction that depended on a change in the index of refraction, such sensors have been sensitive to both specific and non-specific binding. Another prior approach has relied on a sandwich assay where, for example, the binding of an antigen by an antibody has been followed by the secondary binding of a fluorescently tagged antibody that is also in the solution along with the protein to be sensed. In this approach, any binding of the fluorescently tagged antibody will give rise to a change in the signal and, therefore, sandwich assay approaches have also been sensitive to specific as well as non-specific binding events. Thus, selectivity of many prior sensors has been a problem. Another previous approach where signal transduction and amplification have been directly coupled to the recognition event is the gated ion channel sensor as described by Cornell et al., ‘A Biosensor That Uses Ion-Channel Switches’, Nature, vol. 387, Jun. 5, 1997. In that approach an electrical signal was generated for measurement. Besides electrical signals, optical biosensors have been described in U.S. Pat. No. 5,194,393 by Hugl et al. and U.S. Pat. No. 5,711,915 by Siegmund et al. In the later patent, fluorescent dyes were used in the detection of molecules.”

By way of yet further illustration, one may use the sensor element disclosed in U.S. Pat. No. 6,589,731, the entire dislcosure of which is hereby incorporated by reference into this specification. This patent, at column 1 thereof, also discusses biosensors, stating that: “Biosensors are sensors that detect chemical species with high selectivity on the basis of molecular recognition rather than the physical properties of analytes. See, e.g., Advances in Biosensors, A. P. F. Turner, Ed. JAI Press, London, (1991). Many types of biosensing devices have been developed in recent years, including enzyme electrodes, optical immunosensors, ligand-receptor amperometers, and evanescent-wave probes. The detection mechanism in such sensors can involve changes in properties such as conductivity, absorbance, luminescence, fluorescence and the like. Various sensors have relied upon a binding event directly between a target agent and a signaling agent to essentially turn off a property such as fluorescence and the like. The difficulties with present sensors often include the size of the signal event which can make actual detection of the signal difficult or affect the selectivity or make the sensor subject to false positive readings. Amplification of fluorescence quenching has been reported in conjugated polymers. For example, Swager, Accounts Chem. Res., 1998, v. 31, pp. 201-207, describes an amplified quenching in a conjugated polymer compared to a small molecule repeat unit by methylviologen of 65; Zheng et al., J. Appl. Polymer Sci., 1998, v. 70, pp. 599-603, describe a Stem-Volmer quenching constant of about 1000 for poly(2-methoxy,5-(2′-ethylhexloxy)-p-phenylene-vinylene (MEH-PPV) by fullerenes; and, Russell et al., J. Am. Chem. Soc., 1982, v. 103, pp. 3219-3220, describe a Stem-Volmer quenching constant for a small molecule (stilbene) in micelles of about 2000 by methylviologen. Despite these successes, continued improvements in amplification of fluorescence quenching have been sought. Surprisingly, a KSV of greater than 105 has now been achieved.”

Similarly, and by way of further illustration, one may use the light-based sensors discussed at column 1 of U.S. Pat. No. 6,594,011, the entire disclosure of which is hereby incorporated by reference into this specification. As is disclosed in such column 1, “It is well known that the presence or the properties of substances on a material's surface can be determined by light-based sensors. Polarization-based techniques are particularly sensitive; ellipsometry, for example, is a widely used technique for surface analysis and has successfully been employed for detecting attachment of proteins and smaller molecules to a surface. In U.S. Pat. No. 4,508,832 to Carter, et al. (1985), an ellipsometer is employed to measure antibody-antigen attachment in an immunoassay on a test surface. Recently, imaging ellipsometry has been demonstrated, using a light source to illuminate an entire surface and employing a two-dimensional array for detection, thus measuring the surface properties for each point of the entire surface in parallel(G. Jin, R. Janson and H. Arwin, “Imaging Ellipsometry Revisited: Developments for Visualization of Thin Transparent Layers on Silicon Substrates,” Review of Scientific Instruments, 67(8), 2930-2936, 1996). Imaging methods are advantageous in contrast to methods performing multiple single-point measurements using a scanning method, because the status of each point of the surface is acquired simultaneously, whereas the scanning process takes a considerable amount of time (for example, some minutes), and creates a time lag between individual point measurements. For performing measurements where dynamic changes of the surface properties occur in different locations, a time lag between measurements makes it difficult or impossible to acquire the status of the entire surface at any given time. Reported applications of imaging ellipsometry were performed on a silicon surface, with the light employed for the measurement passing through +the surrounding medium, either air or a liquid contained in a cuvette. For applications where the optical properties of the surrounding medium can change during the measurement process, passing light through the medium is disadvantageous because it introduces a disturbance of the measurement.”

U.S. Pat. No. 6,594,011 goes on to disclose (at columns 1-2) that: “By using an optically transparent substrate, this problem can be overcome using the principle of total internal reflection (TIR), where both the illuminating light and the reflected light pass through the substrate. In TIR, the light interacting with the substance on the surface is confined to a very thin region above the surface, the so-called evanescent field. This provides a very high contrast readout, because influences of the surrounding medium are considerably reduced. In U.S. Pat. No. 5,483,346 to Butzer, (1996) the use of polarization for detecting and analyzing substances on a transparent material's surface using TIR is described. In the system described by Butzer, however, the light undergoes multiple internal reflections before being analyzed, making it difficult or impossible to perform an imaging technique, because it cannot distinguish which of the multiple reflections caused the local polarization change detected in the respective parts of the emerging light beam. U.S. Pat. No. 5,633,724 to King, et al. (1997) describes the readout of a biochemical array using the evanescent field. This patent focuses on fluorescent assays, using the evanescent field to excite fluorescent markers attached to the substances to be detected and analyzed. The attachment of fluorescent markers or other molecular tags to the substances to be detected on the surface requires an additional step in performing the measurement, which is not required in the current invention. The patent further describes use of a resonant cavity to provide on an evanescent field for exciting analytes.”

By way of yet further illustration, one may use one or more of the biological sensors disclosed in U.S. Pat. Nos. 6,546,267 (biological sensor), U.S. Pat. No. 5,972,638 (biosensor), U.S. Pat. Nos. 5,854,863, 6,411,834 (biological sensor), U.S. Pat. No. 4,513,280 (device for detecting toxicants), U.S. Pat. Nos. 6,666,905, 5,205,292, 4,926,875, 4,947,854 (epicardial multifunctional probe), U.S. Pat. Nos. 6,523,392, 6,169,494 (biotelemetry locator), U.S. Pat. No. 5,284,146 (removable implanted device), U.S. Pat. Nos. 6,624,940, 6,571,125, 5,971,282, 5,766,934 (chemical and biological sensosrs having electroactive polymer thin films attached to microfabricated device and possessing immobilized indicator molecules), U.S. Pat. No. 6,607,480 (evaluation system for obtaining diagnostic information from the signals and data of medical sensor systems), U.S. Pat. Nos. 6,493,591, 6,445,861, 6,280,586, 5,327,225 (surface plasmon resonance sensor), and the like. The entire disclosure of each of these United States patents is hereby incorporated by reference into this specification.

In one embodiment, the biological sensor is an implantable biological sensor. One may use one or more of the implantable sensors known to those skilled in the art.) By way of illustration, one may use the implantable extractable probe described in U.S. Pat. No. 5,205,292, the entire disclosure of which is hereby incorporated by reference into this specification. This probe comprises a biological sensor attached to the body of the probe such as, e.g., a doppler transducer for measuring blood flow.

In one embodiment, the nanowire sensor described in published U.S. patent application US20020117659 is used; the entire disclosure of this United States patent application is hereby incorporated by reference into this specification. As is disclosed in this published patent aplication, “The invention provides a nanowire or nanowires preferably forming part of a system constructed and arranged to determine an analyte in a sample to which the nanowire(s) is exposed. ‘Determine’, in this context, means to determine the quantity and/or presence of the analyte in the sample. Presence of the analyte can be determined by determining a change in a characteristic in the nanowire, typically an electrical characteristic or an optical characteristic. E.g. an analyte causes a detectable change in electrical conductivity of the nanowire or optical properties. In one embodiment, the nanowire includes, inherently, the ability to determine the analyte. The nanowire may be functionalized, i.e. comprising surface functional moieties, to which the analytes binds and induces a measurable property change to the nanowire. The binding events can be specific or non-specific. The functional moieties may include simple groups, selected from the groups including, but not limited to, —OH, —CHO, —COOH, —SO3H, —CN, —NH2, SH, —COSH, COOR, halide; biomolecular entities including, but not limited to, amino acids, proteins, sugars, DNA, antibodies, antigens, and enzymes; grafted polymer chains with chain length less than the diameter of the nanowire core, selected from a group of polymers including, but not limited to, polyamide, polyester, polyimide, polyacrylic; a thin coating covering the surface of the nanowire core, including, but not limited to, the following groups of materials: metals, semiconductors, and insulators, which may be a metallic element, an oxide, an sulfide, a nitride, a selenide, a polymer and a polymer gel. In another embodiment, the invention provides a nanowire and a reaction entity with which the analyte interacts, positioned in relation to the nanowire such that the analyte can be determined by determining a change in a characteristic of the nanowire.”

A drug delivery device that is comprised of a biological sensor is disclosed in published United States patent application US 2002/011601. As is disclosed in the “Abstract” of this published patent application, “An Implantable Medical Device (IMD) for controllably releasing a biologically-active agent such as a drug to a body is disclosed. The IMD includes a catheter having one or more ports, each of which is individually controlled by a respective pair of conductive members located in proximity to the port. According to the invention, a voltage potential difference generated across a respective pair of conductive members is used to control drug delivery via the respective port. In one embodiment of the current invention, each port includes a cap member formed of a conductive material. This cap member is electrically coupled to one of the conductive members associated with the port to form an anode. The second one of the conductive members is located in proximity to the port and serves as a cathode. When the cap member is exposed to a conductive fluid such as blood, a potential difference generated between the conductors causes current to flow from the anode to the catheter, dissolving the cap so that a biologically-active agent is released to the body. In another embodiment of the invention, each port is in proximity to a reservoir or other expandable member containing a cross-linked polymer gel of the type that expands when placed within an electrical field. Creation of an electric field between respective conductive members across the cross-linked polymer gel causes the gel to expand. In one embodiment, this expansion causes the expandable member to assume a state that blocks the exit of the drug from the respective port. Alternatively, the expansion may be utilized to assert a force on a bolus of the drug so that it is delivered via the respective port. Drug delivery is controlled by a control circuit that selectively activates one or more of the predetermined ports.”

At column 1 of published U.S. patent application US 2002/0111601, reference is made to other implantable drug delivery systems. It is disclosed that (in paragraph 0004) that “While implantable drug delivery systems are known, such systems are generally not capable of accurately controlling the dosage of drugs delivered to the patient. This is particularly essential when dealing with drugs that can be toxic in higher concentrations. One manner of controlling drug delivery involves using electro-release techniques for controlling the delivery of a biologically-active agent or drug. The delivery process can be controlled by selectively activating the electro-release system, or by adjusting the rate of release. Several systems of this nature are described in U.S. Pat. Nos. 5,876,741 and 5,651,979 which describe a system for delivering active substances into an environment using polymer gel networks. Another drug delivery system is described in U.S. Pat. No. 5,797,898 to Santini, Jr. which discusses the use of switches provided on a microchip to control the delivery of drugs. Yet another delivery device is discussed in U.S. Pat. No. 5,368,704 which describes the use of an array of valves formed on a monolithic substrate that can be selectively activated to control the flow rate of a substance through the substrate.” The disclosures of each of U.S. Pat. Nos. 5,368,704, 5,797,898, and 5,876,741 are hereby incorporated by reference into this specification.

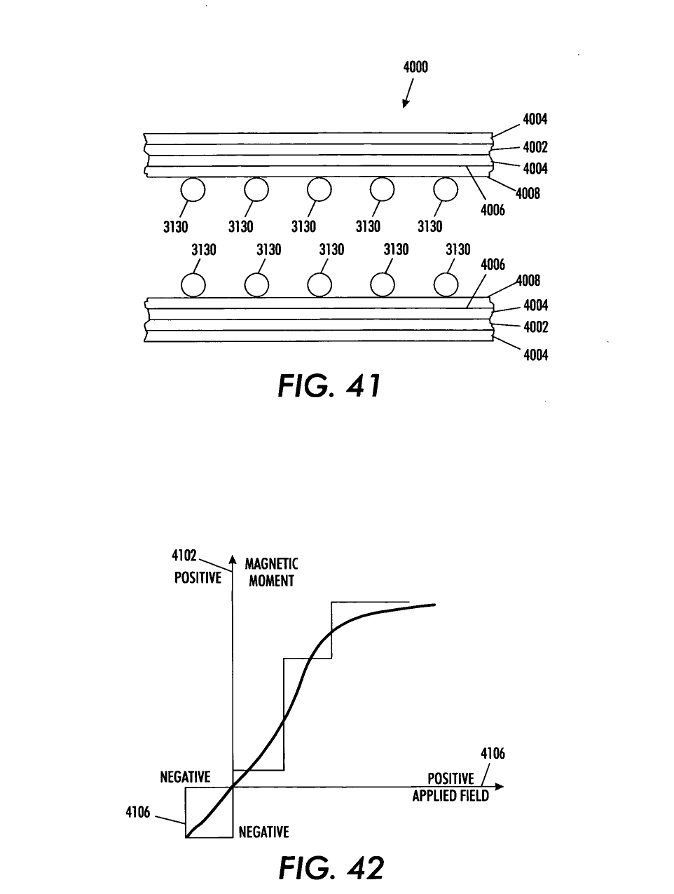

FIG. 41 is a schematic view of a preferred coated stent 4000 of the invention. Referring to FIG. 41, and to the preferred embodiment depicted therein, it will be seen that coated stent 4000 is comprised of a stent 4002 onto which is deposited one or more of the nanomagnetic coatings 4004 described elsewhere in this specification. Disposed above the nanomagnetic coatings 4004 is a coating of drug-eluting polymer 4006.

One may use any of the drug eluting polymers known to those skilled in the art to produce coated stent 4000.

By way of illustration, one may use the drug eluting polymeric material discribed in U.S. Pat. No. 5,716,981, the entire disclosure of this United States patent is hereby incorporated by reference into this specification. This patent describes and claims “A stent for expanding the lumen of a body passageway, comprising a generally6 tubular strucutre coated with a composition comprising paclitaxel, an analogue or derivative thereof, and a polymeric carrier” (see claim 1). The “polymeric carrier” may comprise poly(caprolactone), as is described in claim 2. The polymeric carirer may comprise poly (lactic) acid, as is described in claim 3. The polymeric carrier may comprise poly (ethyelne-vinyl acetate), as is described in claim 4. The polymeric carrier may comprise a copolymer of poly carprolactone and polylactic acid, as is described in claim 5.

The polymeric carrier described in U.S. Pat. No. 5,716,981 preferably is comprised of a moiety which utilize anti-angiogenic factors, i.e., factors (such as a protein, peptide, chemical, or other molecule) that acts to inhibit vascular growth. As is disclosed in this patent, “As noted above, the present invention provides compositions comprising an anti-angiogenic factor, and a polymeric carrier. Briefly, a wide variety of anti-angiogenic factors may be readily utilized within the context of the present invention. Representative examples include Anti-Invasive Factor, retinoic acid and derivatives thereof, paclitaxel, Suramin, Tissue Inhibitor of Metalloproteinase-1, Tissue Inhibitor of Metalloproteinase-2, Plasminogen Activator Inhibitor-1, Plasminogen Activator Inhibitor-2, and various forms of the lighter “d group” transition metals. These and other anti-angiogenic factors will be discussed in more detail below.”

“Briefly, Anti-Invasive Factor, or ‘AIF’ which is prepared from extracts of cartilage, contains constituents which are responsible for inhibiting the growth of new blood vessels. These constituents comprise a family of 7 low molecular weight proteins (<50,000 daltons) (Kuettner and Pauli, ‘Inhibition of neovascularization by a cartilage factor” in Development of the Vascular System, Pitman Books (CIBA Foundation Symposium 100), pp. 163-173, 1983), including a variety of proteins which have inhibitory effects against a variety of proteases (Eisentein et al, Am. J. Pathol. 81:337-346, 1975; Langer et al., Science 193:70-72, 1976: and Horton et al., Science 199:1342-1345, 1978). AIF suitable for use within the present invention may be readily prepared utilizing techniques known in the art (e.g., Eisentein et al, supra; Kuettner and Pauli, supra; and Langer et al., supra). Purified constituents of AIF such as Cartilage-Derived Inhibitor (‘CDI’) (see Moses et at., Science 248:1408-1410, 1990) may also be readily prepared and utilized within the context of the present invention.”

“Retinoic acids alter the metabolism of extracellular matrix components, resulting in the inhibition of angiogenesis. Addition of proline analogs, angiostatic steroids, or heparin may be utilized in order to synergistically increase the anti-angiogenic effect of transretinoic acid. Retinoic acid, as well as derivatives thereof which may also be utilized in the context of the present invention, may be readily obtained from commercial sources, including for example, Sigma Chemical Co. (#R2625).”

“Paclitaxel is a highly derivatized diterpenoid (Wani et al., J. Am. Chem. Soc. 93:2325, 1971) which has been obtained from the harvested and dried bark of Taxus brevifolia (Pacific Yew.) and Taxomyces Andreanae and Endophytic Fungus of the Pacific Yew (Stierle et al., Science 60:214-216, 1993). Generally, paclitaxel acts to stabilize microtubular structures by binding tubulin to form abnormal mitotic spindles. ‘Paclitaxel’ (which should be understood herein to include analogues and derivatives such as, for example, TAXOL®, TAXOTERE®, 10-desacetyl analogues of paclitaxel and 3′N-desbenzoyl-3′N-t-butoxy carbonyl analogues of paclitaxel) may be readily prepared utilizing techniques known to those skilled in the art (see also WO 94/07882, WO 94/07881, WO 94/07880, WO 94/07876, WO 93/23555, WO 93/10076, U.S. Pat. Nos. 5,294,637, 5,283,253, 5,279,949, 5,274,137, 5,202,448, 5,200,534, 5,229,529, and EP 590267), or obtained from a variety of commercial sources, including for example, Sigma Chemical Co., St. Louis, Miss. (T7402—from Taxus brevifolia).”

“Suramin is a polysulfonated naphthylurea compound that is typically used as a trypanocidal agent. Briefly, Suramin blocks the specific cell surface binding of various growth factors such as platelet derived growth factor (‘PDGF’), epidermal growth factor (‘EGF’), transforming growth factor (‘TGF-β’), insulin-like growth factor (‘IGF-I’), and fibroblast growth factor (‘βFGF’). Suramin may be prepared in accordance with known techniques, or readily obtained from a variety of commercial sources, including for example Mobay Chemical Co., New York. (see Gagliardi et al., Cancer Res. 52:5073-5075, 1992; and Coffey, Jr., et al., J. of Cell. Phys. 132:143-148, 1987).”

“A wide variety of other anti-angiogenic factors may also be utilized within the context of the present invention. Representative examples include Platelet Factor 4 (Sigma Chemical Co., #F1385); Protamine Sulphate (Clupeine) (Sigma Chemical Co., #P4505); Sulphated Chitin Derivatives (prepared from queen crab shells), (Sigma Chemical Co., #C3641; Murata et al., Cancer Res. 51:22-26, 1991); Sulphated Polysaccharide Peptidoglycan Complex (SP-PG) (the function of this compound may be enhanced by the presence of steroids such as estrogen, and tamoxifen citrate); Staurosporine (Sigma Chemical Co., #S4400); Modulators of Matrix Metabolism, including for example, proline analogs {[(L-azetidine-2-carboxylic acid (LACA) (Sigma Chemical Co., #A0760)), cishydroxyproIine, d,L-3,4-dehydroproline (Sigma Chemical Co., #D0265), Thiaproline (Sigma Chemical Co., #T0631)], .alpha.,.alpha.-dipyridyl (Sigma Chemical Co., #D7505), β-aminopropionitrile fumarate (Sigma Chemical Co., #A3134)]}; MDL 27032 (4-propyl-5-(4-pyridinyl)-2(3H)-oxazolone; Merion Merrel Dow Research Institute); Methotrexate (Sigma Chemical Co., #A6770; Hirata et al., Arthritis and Rheumatism 32:1065-1073, 1989); Mitoxantrone (Polyerini and Novak, Biochem. Biophys. Res. Comm. 140:901-907); Heparin (Folkman, Bio. Phar. 34:905-909, 1985; Sigma Chemical Co., #P8754); Interferons (e.g., Sigma Chemical Co., #13265); 2 Macroglobulin-serum (Sigma Chemical Co., #M7151); ChIMP-3 (Pavloffet al., J. Bio. Chem. 267:17321-17326, 1992); Chymostatin (Sigma Chemical Co., #C7268; Tomkinson et al., Biochem J. 286:475-480, 1992); 13-Cyclodextrin Tetradecasulfate (Sigma Chemical Co., #C4767); Eponemycin; Camptothecin; Fumagillin (Sigma Chemical Co., #F6771; Canadian Patent No. 2,024,306; Ingber et al., Nature 348:555-557, 1990); Gold Sodium Thiomalate (“GST”; Sigma:G4022; Matsubara and Ziff, J. Clin. Invest. 79:1440-1446, 1987); (D-Penicillamine (“CDPT”; Sigma Chemical Co., #P4875 or P5000(HCl)); B-1-anticollagenase-serum; .alpha.2-antiplasmin (Sigma Chem. Co.:A0914; Holmes et al., J. Biol. Chem. 262(4):1659-1664, 1987); Bisantrene (National Cancer Institute); Lobenzarit disodium (N-(2)-carboxyphenyl-4-chloroanthronilic acid disodium or “CCA”; Takeuchi et al., Agents Actions 36:312-316, 1992); Thalidomide; Angostatic steroid; AGM-1470; carboxynaminolmidazole; metalloproteinase inhibitors such as BB94 . . . . ”

The polymeric carrier may be, e.g., a polyvinyl aromatic polymer, as is disclosed in U.S. Pat. No. 6,306,166, the entire disclsoure of which is hereby incorporated by reference into this specification. As is disclosed in this patent, some suitable polyvinyl aromatic polymers include a polymter that is “ . . . hydrophilic or hydrophobic, and is selected from the group consisting of polycarboxylic acids, cellulosic polymers, including cellulose acetate and cellulose nitrate, gelatin, polyvinylpyrrolidone, cross-linked polyvinylpyrrolidone, polyanhydrides including maleic anhydride polymers, polyamides, polyvinyl alcohols, copolymers of vinyl monomers such as EVA, polyvinyl ethers, polyvinyl aromatics, polyethylene oxides, glycosaminoglycans, polysaccharides, polyesters including polyethylene terephthalate, polyacrylamides, polyethers, polyether sulfone, polycarbonate, polyalkylenes including polypropylene, polyethylene and high molecular weight polyethylene, halogenated polyalkylenes including polytetrafluoroethylene, polyurethanes, polyorthoesters, proteins, polypeptides, silicones, siloxane polymers, polylactic acid, polyglycolic acid, polycaprolactone, polyhydroxybutyrate valerate and blends and copolymers thereof as well as other biodegradable, bioabsorbable and biostable polymers and copolymers. Coatings from polymer dispersions such as polyurethane dispersions . . . and acrylic latex dispersions are also within the scope of the present invention. The polymer may be a protein polymer, fibrin, collage and derivatives thereof, polysaccharides such as celluloses, starches, dextrans, alginates and derivatives of these polysaccharides, an extracellular matrix component, hyaluronic acid, or another biologic agent or a suitable mixture of any of these, for example. In one embodiment of the invention, the preferred polymer is polyacrylic acid, available as HYDROPLUS® (Boston Scientific Corporation, Natick, Mass.), and described in U.S. Pat. No. 5,091,205, the disclosure of which is hereby incorporated herein by reference. U.S. Pat. No. 5,091,205 describes medical devices coated with one or more polyisocyanates such that the devices become instantly lubricious when exposed to body fluids. In a most preferred embodiment of the invention, the polymer is a copolymer of polylactic acid and polycaprolactone.”

In one embodiment, the polymeric carrier is a water souble polymer, such as the water soluble polymers disclose in U.S. Pat. No. 6,441,025, the entire dislcosure of which is hereby incorporated by reference into this specification. These polymers include, e.g., “ . . . a water soluble-polymer having a molecular weight of at least about 5,000 D and dispersed in a pharmaceutically acceptable solution . . . ” (claim 1), “ . . . poly-glutamic acids, poly-aspartic acids or poly-lysines . . . ” (claim 13), etc.

In one embodiment, the polymeric carrier is a biocompatible, pharmaceutically active, bioerodible polymer, as that term is used and defined in published United States patent application US 2002/0042645. The entire disclosure of this published U.S. patent application is hereby incorporated by reference into this specificaiton. As is disclosed in this published patent application: “This invention generally embraces drug eluting stented grafts wherein the drug eluting capability is provided by a composite of drug material and a bioerodible polymer. A feature of the invention is the discovery of a particularly useful group of bioerodible polymers for this purpose. These polymers are fully described In U.S. Pat. No. 4,131,648 by Nam S. Choi and Jorge Heller, issued Dec. 26, 1978, assigned to Alza Corporation, and entitled “Structured Orthoester and Orthocarbonate Drug Delivery Devices”, which is incorporated herein in its entirety by reference. The patent discloses a class of polymers comprising a polymeric backbone having a repeating unit comprising hydrocarbon radicals and a symmetrical dioxycarbon unit with a multiplicity of organic groups bonded thereto. The polymers prepared by the invention have a controlled degree of hydrophobicity with a corresponding controlled degree of erosion in an aqueous or like environment to innocuous products. The polymers can be fabricated into coatings for releasing a beneficial agent, as the polymers erode at a controlled rate, and thus can be used as carriers for drugs for releasing drug at a controlled rate to a drug receptor, especially where bioerosion is desired.”

Some of the polymers specifically described in the claims of published United States patent application US 2002/0042645 include, e.g., “ . . . a biocompatible, pharmaceutically acceptable, bioerodible polymer . . . ,” “ . . . a polyester . . . ,” “ . . . a hydrophobic, bioerodible, copolymer comprising mers I and II according to the following formula: . . . ” (see claim 6), a polymer in which “ . . . . . a multiplicity of microcapsules is dispersed within said at least one polymer, wherein said microcapsules have a wall formed of a drug release rate controlling material; said at least one therapeutic substance is contained within said multiplicity of microcapsules . . . ,” “ . . . . . a pharmaceutically acceptable biocompatible non-bioerodible polymer that sequesters an agent for brachytherapy . . . ,”

Referring again to FIG. 41, and to the preferred embodiment depicted therein, disposed on the surface 4008 of the drug eluting polymer are a multiplicity of magnetic drug particles, such the magnetic drug particle 3130 (see FIG. 38).

FIG. 42 is a graph of a typical response of a magnetic drug particle, such as magnetic drug particles 3130 (see, e.g., FIG. 38) to an applied electromagnetic field. As will be seen by reference to FIG. 42, as the magnetic field strength 4100 of an applied mangetic field is increased along the positive axis, the magnetic moment 4102 of the magnetic drug particle(s) also continuously increases along the positive axis. As will be apparent, a decrease in the magnetic field strength also causes a decrease in magnetic moment. Thus, when the polarity of the applied magnetic field changes (see section 4106 of the graph), the magnetic moment also decreases. Thus, one may affect the magnetic moment of the magnetic drug particles by varying either the intensity of the applied electromagnetic field and/or its polarity.

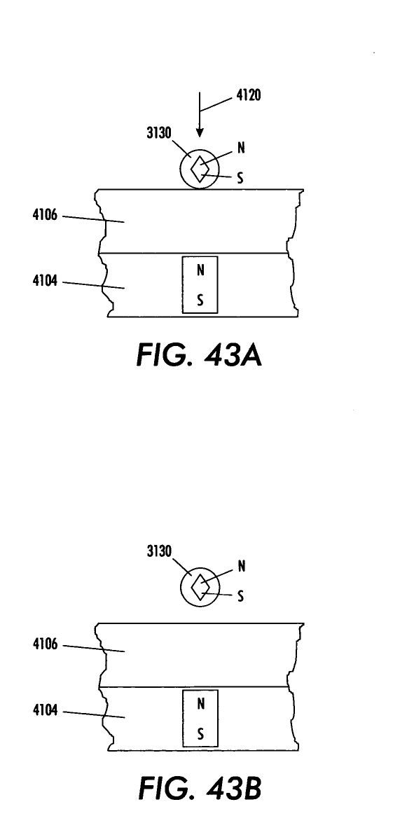

FIGS. 43A and 43B illustrate the effect of applied fileds upon the nanomagnetic coating 4004 (see FIG. 41) and the magnetic drug particles 3130. Referring to FIG. 43A, when the applied magnetic field 4120 is sufficient to align the drug particle 3130 in a north(up)/south(down) orientation (see FIG. 43A), it will also tend to align the nanomagnetic material is such an orientation. However, because the magnetic hardness of the nanomagentic material will be chosen to substantially exceed the magnetic hardness of the drug particles 3130, then the applied magnetic field will not be able to realign the nanomagnetic material.

In the ensuing discussion relating to the effects of an applied electromagnetic field, certain terms (such as, e.g., “magnetization saturation”) will be used. These terms (and others) have the meaning set forth in several of applicants' published patent applications and patents, including (without limitation) published patent application US 20030107463, U.S. Pat. Nos. 6,700,472, 6,673,999, 6,506,972, 5,540,959, and the like. The entire disclosure of each of these documents is hereby incorporated by reference into this specification.

Thus, by way of illustration, reference is made to the term “magnetization.” As is disclsoed in applicants' publications, magnetization is the magnetic moment per unit volume of a substance. Reference may be had, e.g., to U.S. Pat. Nos. 4,169,998, 4,168,481, 4,166,263, 5,260,132, 4,778,714, and the like. The entire disclosure of each of these United States patents is hereby incorporated by reference into this specification.

Thus, by way of further illustration, reference is made to the term “saturation magnetization.” As is disclosed in applicants' publications, for a discussion of the saturation magnetization of various materials, reference may be had, e.g., to U.S. Pat. Nos. 4,705,613, 4,631,613, 5,543,070, 3,901,741 (cobalt, samarium, and gadolinium alloys), and the like. The entire disclosure of each of these United States patents is hereby incorporated by reference into this specification. As will be apparent to those skilled in the art, especially upon studying the aforementioned patents, the saturation magnetization of thin films is often higher than the saturation magnetization of bulk objects.

By way of further illustration, reference is made to the term “coercive force.” As is disclosed in applicants' publications, the term coercive force refers to the magnetic field, H, which must be applied to a magnetic material in a symmetrical, cyclicly magnetized fashion, to make the magnetic induction, B, vanish; this term often is referred to as magnetic coercive force. Reference may be had, e.g., to U.S. Pat. Nos. 4,061,824, 6,257,512, 5,967,223, 4,939,610, 4,741,953, and the like. The entire disclosure of each of these United States patents is hereby incorporated by reference into this specification.

In one embodiment, the nanomagnetic material 103 has a coercive force of from about 0.01 to about 3,000 Oersteds. In yet another embodiment, the nanomagnetic material 103 has a coercive force of from about 0.1 to about 10.

By way of yet further illustration, reference is made to the term relative magnetic permeability. As is disclosed in applicants' publications, the term relative magnetic permeability is equal to B/H, and is also equal to the slope of a section of the magnetization curve of the film. Reference may be had, e.g., to page 4-28 of E. U. Condon et al.'s “Handbook of Physics” (McGraw-Hill Book Company, Inc., New York, 1958). Reference also may be had to page 1399 of Sybil P. Parker's “McGraw-Hill Dictionrary of Scientific and Technical Terms,” Fourth Edition (McGraw Hill Book Company, New York, 1989). As is disclosed on this page 1399, permeability is “ . . . a factor, characteristic of a material, that is proportional to the magnetic induction produced in a material divided by the magnetic field strength; it is a tensor when these quantities are not parallel. Reference also maybe had, e.g., to U.S. Pat. Nos. 6,181,232, 5,581,224, 5,506,559, 4,246,586, 6,390,443, and the like. The entire disclosure of each of these United States patents is hereby incorporated by reference into this specification.

Referring again to FIG. 43, and in the preferred embodiment depicted therein, the magnetic hardness of the n anomagnetic material 4104 is preferably at least about 10 times as great as the magnetic hardness of the drug particles 3130. The term “magnetic hardness” is well known to those skilled in the art. Reference may be had, e.g., to the claims and specifications of U.S. Pat. Nos. 6,201,390, 5,595,454, 5,451,162, 6,534,984, 4,967,078, 3,802,854, and the like. The entire disclosure of each of these United States patents is hereby incorporated by reference into this specification.

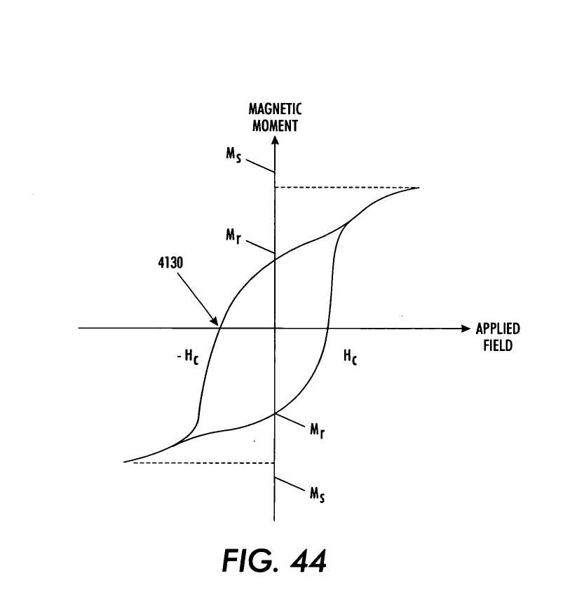

FIG. 44 is graph of a preferred nanomagnetic material and its response to an applied electromagnetic field, in which the applied field is applied against the magnetic moment of the nanomagnetic material.

As will be apparent from this FIG. 44, a certain amount of the applied electromagnetic force is required to overcome the remnant magnetization (Mr) and to change the direction of the remant magnetization from +Mr to −Mr. Thus, e.g., the point −Hc, at point 4130, indicates how much of the field is required to make the magnetic moment be zero.

Referring again to FIGS. 43A and 43B, and in the preferred embodiments depicted therein, the Hc values of the nanomagnetic material chosen will be sufficient to realign to magnetic drug particles 3130 but insufficient to realign the nanomagnetic material. The resulting situation is depcited in FIGS. 43A and 43B.

In FIG. 43A, with the appropriate applied magnetic field, the magnetic drug particle 3130 is attached to the nanomagnetic material 4104 and thus will tend to diffuse nto the polymer 4106. By comparison, in the situation depicted in FIG. 43B, the mangetic drug partigcles will be repelled by the nanomagnetic materail. Thus, and as will be apprent, by the appropriate choice of the applied magneticfield, one can cause the magnetic drug particles either to be attracted to the layer of poolymer mateiral 4106 or to be repelled therefrom.

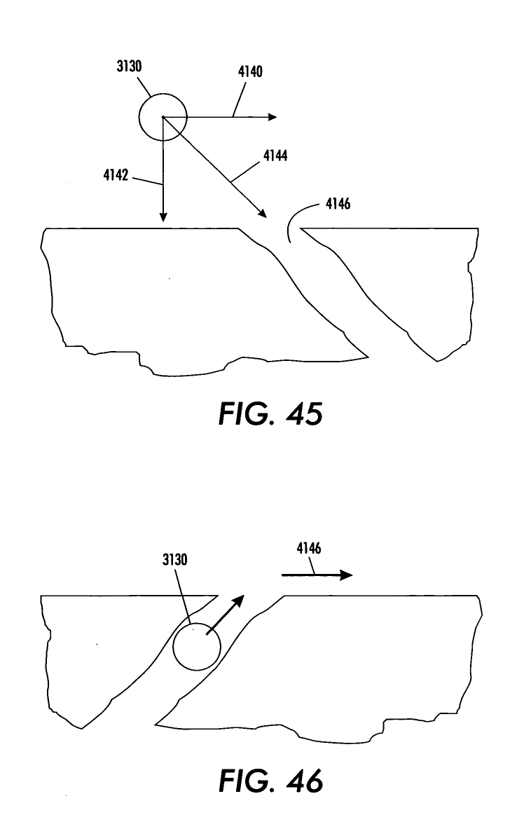

FIG. 45 illustrates the forces acting upon a magnetic drug particle 3130 as it approaches the nanomagnetic material 4104. Referring to FIG. 45, and in the preferred embodiment depicted therein, a certain hydrodynamic force 4140 will be applied to the particle 3130 due to the force of flow of bodily fluid, such as blood. Simultaneously, a certain attractive force 4142 will be created by the attraction of the nanomagnetic material 4104 and the particle 3130. The resulting force vector 4144 will tend to be the direction the particle 3130 will travel in. If the surface of the polymeric material is preferably comprised of a multplicity of pores 4146, the entry of the drug particles 3130 will be facilitated into such pores.

FIG. 46 illustrates the situation that occurs after the drug particles 3130 have migrated into the layer of polymeric material and when one desires to release such drug particles. In this situation (see FIG. 43B), the applied magnetic field will be chosen such that the nanomagnetic material will tend to repel the drug particles 3130 and cause their departure into bodily fluid in the direction of arrow 4148.

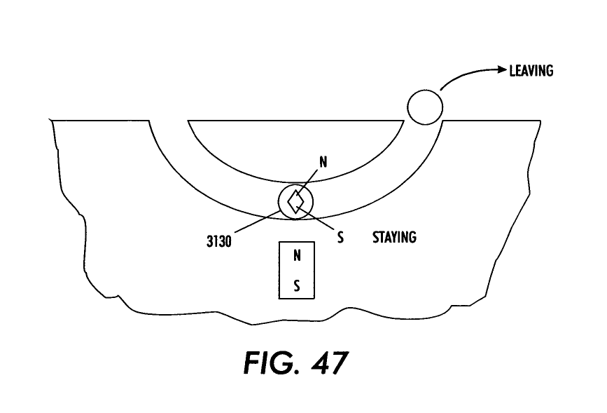

FIG. 47 illustrates the situation that occurs after the drug particles 3130 have migrated into the layer of polymeric material 4106 but when no external electromagnetic field is imposed. In this situation, there will still be an attraction between the nanomagnetric material 4104 and the magnetric drug particles 3130 that will be sufficient to keep such particles bound. However, the attraction will be weak enough such that, when hydrodynamic force 4140 is applied (see FIG. 45), the particles 3130 will elute into the bodily fluid (not shown). As will be apparent, the degree of elution in this case is less than the degree of elution in the case depicted in FIG. 43B. Thus, by the apprpropriate choice of electromagnetic field 4120, one can control the rate of depositoin of the drug particles 3130 onto the polymer 4106, or from the polymer 4106.

Magnetic Drug Compositions

In this section of the specification, applicants will describe certain magnetic drug compositions 3130 that may be used in their preferred process. Each of these drug compositions preferably is comprised of at least one therapeutic agent and has a magnetic moment so that it can be attracted to or repelled from the nanomagnetic coatings upon application of an external electromagnetic field.

One such magnetic composition is disclosed in U.S. Pat. No. 2,971,916, the entire disclosure of which is hereby incorporated by reference into this specification. This patent discloses and claims a microscopic capsule having a wall of hardened organic colloid material enclosing a dispersion of magnetic powder. In one embodiment, the magnetic powder is comprised of the nanomagnetic particles of this invention.

Another such magnetic composition is disclosed in U.S. Pat. No. 3,663,687, the entire disclosure of which is hereby incorporated by reference into this specification. This patet discloses tiny, substantiallyspherular particles comprised of a parenterally metabolizable protein (such as albumin) and which are labeled with a radioisotope. At column 1 of this patent, it is disclosed that: “It has heretofore been known to encapsulate natural products for food or pharmaceutical use in proteinaceous materials such as gelatin and albumin, and small spherical particles of such encapsulated materials have been made, e.g., by processes such as those disclosed in U.S. Pats. 3,137,631; 3016,308; 3,202,731; 2,800,457, and the like.” The entire disclosure of each of these patents is hereby incorporated by reference into this specification.

Another such magnetic drug composition is disclosed in U.S. Pat. No. 4,101,435, the entire disclosure of which is hereby incorporated by reference into this specification. This patent claims “A water dispersable magnetic iron oxide-dextran complex wherein the proportion of the dextran . . . is about 0.1 to about 1 mole per mole of iron oxide . . . . ” This complex is a “magnetic iron oxide sol” is stable and non-toxic. In one embodiment, the magnetic iron oxide material of this patent is replaced by the nanomagnetic material of this invention.

Another such magnetic drug composition is disclosed in U.S. Pat. No. 4,230,685, the entire disclosure of which is hereby incorporated by reference into this specification. This patent discloses “magnetically-responsive microspheres” prepared from a mixture of albumin, magnetic particles (e.g., magnetite), and a protein bound to the outer surfaces of the microspheres. In column 5 of the patent, attachment of specific antibodies (such as staphylococcal Protein A) to the microspheres is discussed. The magnetite of this patent may advantageously be replaced by the nanomagnetic material of this invention.

A similar magnetic drug composition is disclosed in U.S. Pat. No. 4,247,406, the entire disclosure of which is hereby incorporated by reference into this specification. This patent claims (see claim 1) “An intravascularly-administrable, magnetically localizable biodegradable carrier, comprising microspheres formed from an amino acid polmer matrix with magnetic particles embedded therein . . . . ” Example 1 of this patent disclosed the preparation of a microcapsule comprised of 21 percent of magnetite, 73 percent of albumin, and 5 percent of adriamycin. The magnetic particles used in the process of U.S. Pat. No. 4,247,406 may advantageously be replaced by the nanomagnetic particles of this invention.

U.S. Pat. No. 4,247,406 discloses an intravascularly-administrable, magnetically localizable biodegradable carrier that is comprised of microspheres formed from an aminoacid polymer matrix with magnetic particles embedded therein. At column 4 of the patent, it is disclosed that “The carrier of this inventionis believed to be of particular value for administering water-soluble chemotherapeutic agents, such as anti-cancer agents . . . . ” In Example 2 of the patent, the preparation of a microsphere containing 50 percent of magnetite, 46 percent of albumin, and 4 percent of adriamycin is disclosed. The magnetite particles of this patent may advantageously be replaced by the nanomagnetic particles of this invention.

U.S. Pat. No. 4,331,654 discloses and claims: “A magnetically-localizable, biodegradable, substantially water-free drug carrier formulation consisting essentially of lipid microspheres containinga magnetically-responsive substance, one or more biodegradable lipids, and one or more non-toxic surfactants.” The entire disclosure of this United States patent is hereby incorporated by reference into this specification. The magnetically-responsive substance of this patent may be replaced by the nanomagnetic particles of this invention.

At columns 1-3 of this patent a substantial amount of prior art is disclosed regarding magnetically-localizable biodegradable albumin microspheres. Thus, e.g., it is disclosed that: “Magnetically-localizable, biodegradable albumen microspheres have been described by Widder et al., Proc. Soc. Exp. Biol. Med., 58, 141 (1978). The use of such microspheres containing the anticancer drug, adriamycin, in treating rats bearing a Yoshida sarcoma is described in an abstract of a paper by Widder et al., given at the annual meeting of the American Association for Cancer Research in May of 1980 and also at the Federated Societies Meeting in San Francisco, April 1980. Magnetically-localizable, biodegradable albumen microspheres are also described and claimed in the copending application of Senyei and Widder, Ser. No. 32,399 filed Apr.23, 1979, now U.S. Pat. No. 4,247,406.”

“U.S. Pat. No. 4,115,534 discloses a method for determining the concentration of various substances in biological fluids by using magnetically-responsive, permeable, solid, water-insoluble microparticles. The water-insoluble permeable solid matrix can be composed of proteinaceous materials, polysaccharides, polyurethanes or mixtures thereof. The magnetically-responsive material employed is BaFel2 O19. This material is mixed with, for example, bovine serum albumen and the resulting mixture added to a solution comprising a dewatering agent, a cross-linking agent and castor oil. A dispersion of the aqueous material in the oil phase is produced thereby. Particles thus formed are employed in vitro for determining concentrations of various substances in biological fluids.” The water-insoluble microparticles of this patent may be replaced by the nanomagnetic particles of this invention.

“An abstract of a Japanese patent, Chemical Abstracts, 80, 52392a (1974), describes a magnetic material coated with an organic polymer. The combination can be used as a carrier for drugs and x-ray contrast media. For instance, if the material is given orally to an ulcer patient, the magnet localizes the iron-bearing polymer of the lesion and sharp x-ray photos are obtained. Another Japanese advance has been described in the recent press wherein microspheres of a biodegradable nature containing a drug were coated with magnetic particles and the coated microspheres are injected into an animal. The microspheres thus prepared were in excess of 10 microns in diameter.”

“Figge et al, U.S. Pat. No. 3,474,777, disclose and claim finely divided particles of a magnetically-responsive substance having a coating of a therapeutic agent thereon, said particles being injectable. No actual examples are given. Schleicher et al, U.S. Pat. No. 2,971,916, describe the preparation of pressure-rupturable microscopic capsules having contained therein, in suspension in a liquid vehicle, micro-fine particles of a magnetic material useful in printing. U.S. Pat. No. 2,671,451 discloses and claims a remedial pill containing a substance soluble in the human body and including a magnetically-attractable metal element. No specific materials are disclosed. U.S. Pat. No. 3,159,545 discloses a capsule formed of a non-toxic, water-soluble thermoplastic material and a radioactive composition compounded from pharmaceutical oils and waxes in the said capsule. The capsule material is usually gelatin. U.S. Pat. No. 3,190,837 relates to a minicapsule in which the core is surrounded first by a film of a hydrophylic film-forming colloid (first disclosed in U.S. Pat. No. 2,800,457) and a second and different hydrophylic film-forming colloid adherantly surrounding the core plus the first hydrophylic film. Successive deposits of capsule or wall material may also be employed. Among the core materials are mentioned a number of magnetic materials including magnetic iron oxide. A large number of oils may also be employed as core materials but these are, as far as can be seen, not pharmacologically active. Finally U.S. Pat. No. 3,042,616 relates to a process of preparing magnetic ink as an oil-in-water emulsion.”

“There are a number of references which employ lipid materials to encapsulate various natural products. For example, U.S. Pat. No. 3,137,631 discloses a liquid phase process for encapsulating a water-insoluble organic liquid, particularly an oil or fragrance, with albumen. The albumen coating is then denatured, and the whole aerated. Specific examples include the encapsulation of methyl benzoate, pinene or bomyl acetate and the like in egg albumen. U.S. Pat. No. 3,937,668 discloses a similar product useful for carrying radioactive drugs, insecticides, dyes, etc. Only the process of preparing the microspheres is claimed. U.S. Pat. No. 4,147,767 discloses solid serum albumen spherules having from 5 to 30% of an organic medicament homogenously entrapped therein. The spherules are to be administered intravascularly. Zolle, the patentee of U.S. Pat. No. 3,937,668 has also written a definitive article appearing in Int. J. Appl. Radiation Isotopes, 21, 155 (1970). The microspheres disclosed therein are too large to pass into capillaries and are ultimately abstracted from the circulation by the capillary bed of the lungs. U.S. Pat. No. 3,725,113 discloses microencapsulated detoxicants useful on the other side of a semipermeable membrane in a kidney machine. In this application of the microencapsulation art, the solid detoxicant is first coated with a semipermeable polymer membrane and secondly with a permeable outer layer consisting of a blood-compatible protein. U.S. Pat. No. 3,057,344 discloses a capsule to be inserted into the digestive tract having valve means for communicating between the interior of the capsule and exterior, said valve being actuable by a magnet. Finally, German Offenlegungsschrift, No. P. 265631 7.7 filed Dec. 11, 1976 discloses a process wherein cells are suspended in a physiological solution containing also ferrite particles. An electric field is applied thereto thereby causing hemolysis. A drug such as methotrexate is added as well as a suspension of ferrite particles. The temperature of the suspension is then raised in order to heal the hemolysed cells. The final product is a group of cells loaded with ferrite particles and containing also a drug, which cells can be directed to a target in vivo by means of a magnet.”

“Lipid materials, particularly liposomes have also been employed to encapsulate drugs with the object of providing an improved therapeutic response. For example, Rahman et al, Proc. Soc. Exp. Biol. Med., 146, 1173 (1974) encapsulated actinomycin D in liposomes. It was found that actinomycin D was less toxic to mice in the liposome form than in the non-encapsulated form. The mean survival times for mice treated with actinomycin D in this form were increased for Ehrlich ascites tumor. Juliano and Stamp, Biochemical and Biophysical Research Communications, 63, 651 (1975) studied the rate of clearance of coichicine from the blood when encapsulated in a liposome and when non-encapsulated.”

“Among the major contributors to this area of research—use of liposomes—has been Gregoriades and his co-workers. Their first paper concerned the rate of disapparence of protein-containing liposomes injected into a rate [Brit. J. Biochem., 24, 485 (1972)]. This study was continued in Eur. J. Biochem., 47, 179 (1974) where the rate of hepatic uptake and catabolism of the liposome-entrapped proteins was studied. The authors believed that therapeutic enzymes could be transported via liposomes into the lysosomes of patients suffering from various lysosomal diseases. In Biomedical and Biophysical Research Communications 65, 537 (1975), the group studied the possibility of holding liposomes to target cells using liposomes containing an antitumor drug. The actual transport of an enzyme, horseradish peroxidase, to the liver via liposomes was discussed in an abstract for 7th International Congress of the Reticuloendothelial Society, presented at Pamplona, Spain, Sep. 15-20, 1975.”

By way of further illustration, U.S. Pat. No. 4,345,588 dislcoses a method of delivering a water-soluble anti-cancer agent to a target capillary bed of a body associated with a tumor, comprising the step of incorporating the water-soluble anti-cancer agent into microspheres formed from a biodegradable matrix material, and thereafter applying a magnetic field to immobilize the microspheres. Claim 4 of this patent, which is typical, describes: “The method of delivering a water soluble anti-cancer agent to a target capillary bed of the body associated with a tumor, comprising the steps of: (a) incorporating the water-soluble anti-cancer agent in microspheres formed from a biodegradable matrix material with magnetic particles embedded therein, said magnetic particles having an average size of not over 300 Angstroms, said microspheres having an average size of less than 1.5 microns and passing into said capillary bed with the blood flowing therethrough, said microspheres containing from 10 to 150 parts by weight of said magnetic particles per 100 parts of said matrix material; (b) introducing said anit-cancer agent containing microspheres into an artery upstream of said capillary bed; (c) applying a magnetic field to the area of the body of said capillary bed and artery, said magnetic field being of a strength capable of immobilizing said microspheres at the blood flow rate of said capillary bed while permitting said microspheres to pass through said artery at the blood flow rate therein; (d) immobilizing at least part of said microspheres in capillaries of said target bed by said magnetic field application while blood continues to perfuse therethrough; and (e) removing said magnetic field before said anti-cancer agent is released from said microspheres, said microspheres being retained in said capillary bed after said removal of said magnetic field for release of said anti-cancer agent in effective therapeutic relation to said tumor.” The operation of this claimed invention is described in part at column 2 of the patent, wherein it is disclosed that: “The present invention provides a novel method of delivering a therapeutic agent to a target capillary bed of the body. The method takes advantage of the difference in blood flow rates between arteries and capillaries. The magnetic microspheres used for administering the therapeutic agent are selectively localized in the target capillary bed by applying a magnetic field which immobilizes the microspheres at the much slower blood flow rate of the capillaries but not at the flow rate of the arteries into which the microspheres are initially introduced. Moveover, the magnetic field need be applied only for a short time, after which it can be removed. This is based on the discovery that microspheres of sufficiently small size can be permanently localized in the capillaries, once they have been magnetically attracted to the walls of the capillaries and immobilized thereon, even though the blood continues to flow through the capillary bed in a substantially normal manner. In other words, the immobilized microspheres do not plug-up or block the capillaries as described in the method of U.S. Pat. No. 3,663,687 . . . . For effective magnetic control, the microspheres are introduced into an artery upstream of the capillary bed where they are to be localized, the selected capillary bed being associated with the target site. It is therefore of critical importance that the microspheres have a degree of magnetic responsiveness which permit them to pass through the arteries without significant holdup under the applied magnetic field while being immobilized and retained in the capillaries. The present invention achieves this objective by utilizing the difference in flow rates of the blood in the larger arteries and in the capillaries. In addition, the albumin surface prevents clump formation, thus allowing relatively normal blood perfusion at the area of retention.”

One may use the process of this patent with the nanomagnetic particles of this invention in substantial accordance with the procedure of such patent. Once the nanomagnetic particles have been delivered to the desired site, another electromagnetic field may be applied to cause such particles to heat up to a certain specified temperature at which one or more therapeutic objectives may be attained. Once the temperature of the naoparticles exceeds the desired temperature, the heating of such particles ceases (see FIG. 3C).

U.S. Pat. No. 4,357,259 discloses a process for incorporating water-soluble therapeutic agents into albumin microspheres. Among the agents that may be so incorporated are included enzymes (such as, e.g., trypsinogen, chymotrypsinogen, plasminogen, streptokinase, adenyl cyclase, insulin, glucagons, coumarin, heparin, histamine, and the like), chemotherapeutic agents (such as, e.g., tetracycline, aminoglycosides, penicillin group of drugs, +Cephalosporins, sulfonamide drugs, chloramphenicol sodium succinate, erythromycin, vancomycin, lincomycin, clindamycin, nystatin, amphotericin B, amantidine, idoxuridine, p-Amino salicyclic acid, isoniazid, rifampin, water-soluble alkylating agents in Ca therapy, water-soluble antimetabolites, antinomycin D, mithramycin, daunomycin, adriamycin, bleomycin, vinblastine, vincristine, L-asparaginase, procarbazine, imidazole carboxamide, and the like), immunological adjuvans (such as, e.g., concanavalin A, BCG, levamisole, and the like), natural products (such as, e.g., prostaglandins, PGE1, PGE2, cyclic nucleotides, TAF antagonists, water-soluble hormones, lymphocyte inhibitors, lymphocyte stimulatory products, and the like), etc. In addition to such therapeutic agents, one may also incorporate the nanomagnetic particles of this invention into such microspheres.

Claim 1 of U.S. Pat. No. 4,357,259 is typical of the process of the patent. Such claim 1 describes: “The method of incorporating a water-soluble heat-sensitive therapeutic agent in albumin microspheres, in which all steps thereof are carried out at a temperature within the range from 1° to 45° C., said method including the steps of preparing an aqueous albumin solution of the said therapeutic agent, said albumin solution containing from 5 to 50 parts by weight of albumin per 100 parts of water and from 1 to 20 parts by weight of said therapeutic agent per 100 parts of albumin, emulsifying said albumin solution with a vegetable oil to form a water-in-oil emulsion containing dispersed droplets of the albumin solution, removing the oil by washing the dispersed droplets with an oil-soluble water-immiscible organic solvent, and recovering the resulting microspheres, wherein said method also includes the step of contacting said microspheres with an organic solvent solution of an aldehyde hardening agent to increase the stability of said microspheres and to decrease the release rate of said drug therefrom.” claim 3 of the patent, whichis dependent upon claim 1, further recites that “ . . . the albumin solution also contains magnetic particles.” The “magnetic particles” of such claim 3 may be applicants' nanomagnetic particles.

U.S. Pat. No. 4,501,726 discloses a magnetically responsive nanoparticle made up of a crystalline carbohydrate matrix. Claim 1 of this patent, which is typical, describes: “A nanosphere or nanoparticle for intravascular administration, which is magnetically responsive and biologically degradable and which is made up of a matrix in which a magnetic material is enclosed, characterized in that said nanosphere or nanoparticle has an average diameter which does not exceed 1500 nm, and circulates in the vascular system after administration thereto, said matrix comprising a hydrophillic, crystalline carbohydrate.”

The carbohydrate matrix of the particle of U.S. Pat. No. 4,501,726 is biodegradable. Furthermore, one or more drugs may be adsorbed to the carbohydrate after the nanoparticles have been produced. As is disclosed in column 2 of U.S. Pat. No. 4,501,726, “Carbohydrate polymers containing alpha(1-4) bonds are especially useful because they can be degraded by the alpha-amylase in the body. Although starch is preferred, also pullullan, glycogen and dextran may be used. It is also possible to modify the carbohydrate polymer with, for example, hydroxyethyl, hydroxypropyl, acetyl, propionyl, hydroxypropanoyl, various derivatives of acrylic acid or like substituents. Also carbohydrates which are not polymeric, may be used in the context of this invention. Examples of such carbohydrates are glucose, maltose and lactose. Pharmaceuticals may be adsorbed to the carbohydrates after the nanosphere has been produced. This may be important in such cases where the pharmaceutical in question is damaged by the treatment in connection with the production of the magnetic nanospheres. If the matrix is a carbohydrate, it is also possible to modify the matrix by covalently coupling to the carbohydrate e.g. amino groups or carboxylic acid groups, thereby to create an adsorption matrix. High molecular substances of the type proteins may be enclosed within the matrix for later release.”

In one embodiment of the instant invention, an anti-microtubule agent (such as, e.g., paclitaxel), is adsorbed onto the surfaces of the nanoparticles. In one aspect of this embodiment, the release rate of the paclitaxel is varied by cross-linking the carbohydrate matrix after crystallization. As is disclosed in column 4 of U.S. Pat. No. 4,501,726, “It is also possible to vary the release rate of the pharmacologically active substance by cross-linking the matrix after crystallization. The tighter the matrix is cross-linked, the longer are the release times. Different types of cross-linking agents can be used, depending upon whether or not water is present at the cross-linkage. In aqueous environment, it is possible to use, inter alia, divinyl sulphone, epibromohydrin or BrCN. In the anhydrous phase, it is possible to activate with tresyl reagent, followed by cross-linking with a dianine.”

The constructs of U.S. Pat. No. 4,501,726 may advantageously use applicants nanomagnetic particles which provide a superior magnetic moment per unit volume.

By way of further illustration, one may use the delivery system of U.S. Pat. No. 4,652,257 to deliver an anti-microtubule agent (such as paclitaxel) to a site within a human body, such as, e.g., an implanted medical device; the entire disclosure of this United States patent is hereby incorporated by reference into this specification.

Claim 1 of U.S. Pat. No. 4,652,257 describes: “A method of delivering a therapuetic agent to a target site within the body, comprising the steps of: introducing ferromagnetic particle embedded vesicles containing said therapuetic agent into the blood stream upstream of said target site; applying a magnetic field having sufficient strength to immobilize said vesicles at said target site; immobilizing said vesicles at said target site; and oscillating said magnetic field at a rate sufficient to vibrate said ferromagnetic particles such that said vesicles's membrane is destabilized or lysed thereby controlling the rate of release of said therapuetic agent at said target site.” The “ferromagnetic particle” of U.S. Pat. No. 4,652,257 may be replaced with applicants' nanomagnetic particle of this invention.

The lysing of the vesicle by the application of a magnetic field is described at column 5 of the patent, wherein it is disclosed that: “In the present invention, the vesicles are formed using polymerizable lipids which are subsequently polymerized by exposing the vesicles to ultra-violet light. Using a Rayonet Photochemical Reactor Chamber (model RPR-100), it takes between 5-30 minutes at a UV strength of about 25 watts. Alternatively, the vesicles can be formed from lipid/polymerizable lipid mixtures so as to vary the permability of the vesicle membrane. Once formed, the vesicles, containing the therapeutic agent and ferromagnetic particles, can be injected upstream from the target site. The vesicles migrate through the blood stream to the target area where they can be immobilized by an 8000 gauss magnetic field. Once immobilized, the vesicle's contents can be released by oscillating the magnetic field at a rate sufficient to vibrate the embedded ferromagnetic particles. The total contents of the vesicle can be released by oscillating the magnetic field sufficiently to lyse the membrane. Alternatively, particularly with the mixed lipid/polymerizable lipid vesicle, the contents can be released at a controlled rate by varying the oscillation rate so as to destabilize the membrane making it more permeable to the therapeutic agent but not so as rupture the membrane. The magnetic field can be oscillated at a rate between 10 and 1200 cycles per second but a range between 500 and 1000 cycles per second is prefered. The magnetic field can have any strength necessary to immobilize the vesicles. A range between 5000 and 12000 Gauss is prefered with 7000 to 9000 Gauss being most preferred.” As will be apparent, the lysing of the vesicle will be more readily attained with applicant's nanomagnetic particles, which have superior magnetic moments per unit volume.

In one embodiment, the coercive force and the remnant magnetization of applicants' nanomagnetic particles are preferably adjusted to optimize the magnetic responsiveness of the particles so that the coercive force is preferably from about 1 Gauss to about 1 Tesla and, more preferably, from about 1 to about 100 Gauss.

Some of the therapeutic agents that may be used in the process of U.S. Pat. No. 4,652,257 are described at columns 5-6 of this patent, wherein it is disclosed that: “For example, vesicles containing oncolytic agents could be injected intra-arterially upstream from a tumor, localized in the tumor by the magnetic field, and disrupted by oscillating the magnetic field. The toxicity of the oncolytic agents is, therefore, confined to the area where the tumor is located. Therapeutic agents which can be encapsulated in the vesicles include hydrophillic materials such as vindesine sulfate, fluorouracil, antinomycin D, and the like. Basically, any known oncolytic agent, anti-inflamatory agent, anti-arthritic agent or similar agent which is hydrophillic can be incorporated into the vesicles.”

In one embodiment of this invention, an anti-microtubule agent (such as, e.g., paclitaxel) is incorporated into the vesicle of U.S. Pat. No. 4,652,257 and delivered to the situs of an implantable medical device, wherein the paclitaxel is released at a controlled release rate. Such a situs might be, e.g., the interior surface of a stent wherein the paclitaxel, as it is slowly relesased, will inhibit restenosis of the stent.

U.S. Pat. No. 4,674,480 also a magnetic drug composition that is “ . . . operable in the presence of the body fluid to degrade and release the drug contents of said microcapsules after a time delay once said drug units have entered the body and said drug units are targeted to a select cancer site in the body of the living being to whom said medical dose has been administered” (see claim 9 of the patent). The entire disclosure of this United States patent is hereby incorporated by reference into this specification.

claim 1 of U.S. Pat. No. 4,674,480 describes one preferred process of this patent. This claim 1 discloses: “A method of effecting a medical treatment or diagnosis, said method comprising: (a) forming a multitude of drug units, each containing a quantity of a drug encapsulated by a carrier material within the drug unit formed, (b) administering a select quantity of said drug units to the body of a living being, (c) allowing at least a portion of said administered drug units to travel through the body to a select location in the body and to become disposed adjacent select tissue at said select location to allow said select tissue at said select location to be treated with the encapsulated drug thereof, and (d) after a substantial quantity of said drug units are so disposed, causing the drug contained in each unit to be released from the carrier material encapsulation and to flow to tissue adjacent which said units are disposed.”

Various means are disclosed in U.S. Pat. No. 4,674,480 for “ . . . causing the drug contained in each unit to be released . . . . ” Thus, e.g., in claim 2 of the patent, it is disclosed that “ . . . the quantities ofdrug contained by such drug units are released by causingsaid encapsulating carrier material of said units to become ruptured to destroy the encapsulating effect.” Thus, e.g., claim 3 of the patent describes a method in which “ . . . the quantities of drug contained by said drug units are released from encapsulation by causing said encapsulating carrier material of said drug units to become porous and release drug contained thereby . . . . ” Thus, e.g., claim 4 describes a method in which “ . . . the quantities of drug contained by said drug units are released from the drug units by causing said encapsulating carrier material of said drug units to dissolve or biodegrade in body fluid . . . . ” Thus, e.g., claim 5 describes a method in which “ . . . the quantities of drug contained by said drug units are released from the drug units by causing said encapsulating carrier material of said units to biodegrade within said living being at a select time after being administered to the body of said living being . . . . ” Thus, e.g., claim 6 describes a method in which” . . . the quantities of said drug contained by said drug units are released therefrom by causing a quantity of a nuclide contained in at least certain of said units to become radioactive and, in so becoming, to explosively destroy at least a portion of the encapsulating carrier material to release the encapsulated drug from the units . . . . ” Thus, e.g., claim 7 describes a method in which “ . . . a substantial portion of said administered drug units are permitted to travel in the bloodstream of said living being and to flow with the blood of said living being to the tissue of the body to be treated when the drug encapsulated in said drug units is released from encapsulation by said drug units at the site of said tissue . . . ”.

Some of the preferred “releasing means” of U.S. Pat. No. 4,674,480 are described in columns 5-9 of such patent.

Thus, and referring to columns 5-6 of U.S. Pat. No. 4,674,480,“ . . . a drug unit 10 . . . may comprise one of a multitude of such units disposed in a liquid or capsule which is administered to a living being. The drug unit 10 comprises a bulbous capsule 11, shown as having a spherical or ellipsoidal shape, although it may have any other suitable shape. A side wall 12 completely surrounds contents 15 which may comprise any suitable type of medication such as an organic or inorganic liquid chemical, a plurality of such chemicals, a biological material, such as an antibiotic or a liquid containing one or more living or dead virus, bacteria, antibodies, phages, or other material which is desired to be dispensed within or in the immediate vicinity of disease tissue or disease cells existing within a living being.”

United States patent then goes on to describe “nuclide particle 14,” stating that: “A small particle 14 is supported against a portion of the outside surface 13 of the wall 12. Particle 14 is a nuclide material, such as boron-10 . . . . Such paricle 14 may comprise a plurality of particles bonded by a suitable resin or other material coating the outside surface 13 of capsule 11. Particle 14 may be rendered radioactive and caused to generate radiation or explode as illustrated in FIG. 2, to rupture a portion of the wall 12 to permit the contents 15 of capsule 11 to flow through the opening 12R. A plurality of openings may be formed in the wall when particles of such nuclide are simultaneously rendered radioactive. Such particle 14 may be so rendered radioactive when the drug unit 10 is disposed or flows to a select location within a living being, such as a location of diseased tissue, dead or calcified tissue or bone desired to be subjected to a chemical or biological agent, such as the contents 15 of the capsule 11.”

“The contents 15 may be under slight pressure during the formation of the capsule 11 or may be pressurized as the result of the heat or pressure of the radiation generated when the particle or particles 14 become radioactive. Accordingly, one or more of such particles may also be disposed within the body of the contents 15 or against the inside surface of the wall 12 or within such wall for such purpose and/or to render the wall 12 ruptured or porous to permit flow of the contents 15 from the capsule and/or absorption of body fluid into the capsule to mix or react with its contents.”

“The capsule 11 may vary in size from less than a thousandth of an inch in diameter to several thousandths of an inch in diameter or more, if a multitude of such capsules are utilized to deliver a chemical or biological agent to a particular location within a living being via the bloodstream or by direct injection to such location. It may also comprise a larger capsule which is injested by mouth, inserted by catheter or implanted by of surgery at a select location in tissue or a body duct. Wall 12 may be made of a synthetic polymer, such as a suitable plastic resin, a starch, protein, fat, cell tissue, a combination of such materials or other organic matter. It may be employed per se or in combination with other elements as described hereafter. Similar or differently shaped capsules of the types illustrated in the drawings may be combined or mixed and may contain a plurality of different elements or drugs mixed in each or provided in separate such elements or drugs cooperate in alleviating a malady such as by attacking or destroying bacteria or diseased tissue, improving the condition of living cells, changing the structure of living tissue or cells, dissolving or destroying tissue cells, repairing cells or cell damage, etc.”

“In FIG. 3, a drug unit 20 of the type shown in FIGS. 1 and 2, comprises a spherically shaped container or shell 21 of one or more of the materials described with a spherical sidewall 22. The outer surface 23 may contain one or more particles of a nuclide of the type described and/or one or more antibodies, such as monoclonal antibodies, attached thereto by a suitable resin or assembled with the container 21 by a suitable derivatizing agent. Disposed within the hollow interior of spherically shaped container 21 is a liquid material or drug 25 having one or more particles 24 of a nuclide or a plurality of nuclides floating or supported therein. Such nuclide or nuclide particles 24 may be rendered radioactive, as in FIG. 2, by directing a beam or beams of neutrons at the drug unit 20, such a neutron beam source may be located outside the body in which the drug units are disposed. The neutrons render the one or more particles 24 radioactive in a manner to either explode or generate sufficient radiant energy to cause the liquid contents 24 to at least partially evaporate or otherwise expand in a manner to force such contents through the wall 22, which may be porous or rendered porous or may be ruptured by the internal pressure effected when the particle or particles 24 become radioactive. In such a manner, the contents 25 may be completely or partially expelled from the container and applied to adjacent or ambient tissue or disease matter located within a human living being adjacent the drug unit 20. In a particular form of FIG. 3, one or more particles of a nuclide disposed on the outer surface 23 of the wall 22 may be rendered radioactive and explode to rupture a portion or portions of the wall, rendering same porous or providing an opening therein or destroying such wall so that the contents 25 may flow therefrom to surrounding material.”

“In FIG. 4 is shown a modified form of drug unit 30 formed of a capsule 31 of the type illustrated in FIGS. 1 and 2 or 3. A sperhical or ellipsoidally shaped sidewall 31 completely surrounds a liquid, cream or solid drug or chemical 33 having one or more particles 34 of a nuclide . . . . Bonded or otherwise attached to a portion of the exterior surface 32 of wall 31 is an antibody 36, such as a monoclonal antibody, which is targeted to a specific antigen located within a living being. Such antigen may comprise, for example, the surface of a cancer cell, bacteria, disease tissue or other material desired to be affected by the chemical or agent 33 released from the drug unit 30 when the nuclide particle or particles 34 located within the contents 33 or disposed within or against the surface 32 of the wall 31 of the capsule, are rendered radioactive and explode or generate sufficient heat or radiation to effect one or more of the described actions with respect to the wall 31 of the capsule, such as render same porous or ruptured. A polymer or other derivatizing agent 35 is employed to bond the antibody or monoclonal antibody 36 to a portion of the surface 32 of the capsule.”

“In FIG. 5 is shown a modified form of FIG. 4 wherein a drug unit 40 is composed of a base unit or container 41 which is illustrated as a porous spherical body, the cells 43 of which contain a drug or chemical dispensed therefrom to surrounding fluid or tissue. One or more particles 44 of a nuclide of the type described above, are disposed within the body of the spherical container 41 and/or against the outside surface thereof to be rendered radioactive when a beam or beams of radiation, such as neutrons, are directed thereat. The radiation is absorbed by the particle or particles to effect such radioactivity which may comprise explosive and/or nonexplosive radiation. Thus, liquid or particulate drug material (1) may be forced from the cells of the container 41, (2) effect a chemical reaction resulting in such action or (3) partially or completely destroy the container 41 to release its contents.”

“A plurality of antibodies 45 as disposed against and bonded to the outside surface 42 of the container 41. In this embodiment, monoclonal antibodies 45 are targeted to a particular antigen, such as a disease or cancer cell or other cell located within the body of a living being to be treated, destroyed or otherwise affected by the action of chemical or biological agent carried by the container 41 and, if so constructed, by the radioactivity generated when the nuclide particle or particles 44 are rendered radioactive as described.”

“In FIG. 6 is shown a container assembly 50, which may be a preformed capsule or otherwise shaped implant having a container body 51 with a suitable sidewall 52 and having contents 56, such as one of the chemicals or biological agents described above, which contents are desired to be dispensed from a neck portion 53 of the container. Supported within the neck portion 53 is a solid material 54 containing one or more particles 55 of a nuclide of the type described. When such particle or particles 55 are rendered radioactive by externally applied radiation, they may heat and melt the material 54 or explode and rupture such material and a portion of the neck 53 of the container. Thus, contents 56 flow from container 50, either by capillary action if the neck 53 is of a capillary construction, by internal pressure created by the heat of radiation or existing within the container, by gravity or osmosis effected when the wall 52 of the container and/or the filling material 54 is rendered porous or when porous filling material 54 is exposed to the exterior of the container when a portion of the neck wall 52 neck is ruptured or destroyed when a particle or particles 55 become radioactive.”

“In FIG. 7 is shown a portion of a container 60 having a sidewall 61 and a plurality of interior wall portions 65 extending completely through the container to provide a plurality of separate chambers 66. Each chambers 66 may contain different portions of the same chemical or biological agent or different chemicals or biological agents. Disposed against select portions of the sidewalls 61 and either bonded to the exterior surface 62 of the container 60 or supported within a material 63 coating of such sidewall, are a plurality of particles 64 of a nuclide. In FIG. 7, one particle 64 is shown aligned with each chamber 66 of although a multiple of such particles may be so aligned and disposed. When a beam or beams or radiation, such as neutrons, are selectively directed at selected portions of the sidewall 61 and the particle or particles 64 aligned therewith, the selected portions of the sidewall may be ruptured, rendered porous or have small openings formed therein when the particle or particles of nuclide are rendered active as described. Thus, contents 67 are selectively disposed when the sidewall portions of the chamber or chambers 66 are ruptured or rendered porous when the selected nuclide particle or particles become radioactive.”

“Nuclides will provide miniature explosive atomic reactions capable of rendering microcapsules such as liposomes, starch, protein or fat microballoons in the order of one to ten microns or greater in diameter porous or ruptured to release their liquid medication contents to surrounding tissue or cells, may include boron-10, cadmium-113, lithium-6, samarium-149, mercury-199, gadolinium-155 and gadolinium-157. Nuclides which may be attached or coated on or disposed within the described microcapsules for diagnostic and indicating purposes include such radioactive elements as cobalt 57; galium 67, cesium 131, iodine 131, iodine 125, thalium 201, technicium 99 m, indium 111, selenium 75, carbon 11, nitrogen 13 or a combination of such radioactive elements. In a particular form of the invention, both a neutron activated and atomically explosive particle or particles, such as atoms, of a nuclide and a normally radioactive nuclide of the groups above may be provided in a single drug unit per se or in combination with a chemical as described.”

U.S. Pat. No. 4,690,130 discloses a process in which electromagnetic radiation is selectively applied to a patient in every area except for a “treatment zone.” Thus, and as is described in claim 1 of such patent, there is provided a method for “ . . . A method for applying a therapeutic agent to a treatment zone in a patient, which treatment zone is not adjacent the skin of the patient, comprising: applying a steady or low frequency magnetic field to the patient to include the treatment zone; supplying microspheres for circulation through the patient to include said zone, said microspheres including a therapeutic agent, and also includes medically bodily compatible magnetic material having a Curie point at which the magnetic material becomes substantially non-magnetic slightly above the normal body temperature of the patient; and applying high frequency electromagnetic field energy to said patient where said magnetic field is applied to said patient, except to said treatment zone, to heat up said magnetic material to demagnetize it so the microspheres are not restrained by said magnetic field except in said treatment zone.”

The rationale for the invention of U.S. Pat. No. 4,690,130 is described in column 3 of such patent, wherein it is disclosed that “ . . . the present invention involves the selective restraint of magnetic material having an accessible Curie point temperature, and the use of (1) a magnetic field to hold the magnetic material and (2) the use of a high frequency electromagnetic field to selectively heat the magnetic particles to a temperature above the Curie point. In order to effect restraint of particles within a selected field zone, two conditions must be simultaneously met therein—(1) the particles must be magnetically responsive i.e., at a temperature sufficiently below the Curie point to exhibit substantial ferromagnetic exchange coupling, and (2) the static magnetic field gradient must be of adequate strength to restrain magnetically responsive particles within capillary vessels in the selected field zone. It is necessary and sufficient that either one of these conditions be absent at sites external to the selected field zone (where it is desired to concentrate the microspheres) in order to effect free unrestrained flow of the particles. The appropriate presence and absence of these conditions is regulated by the geometrical intersection of an oscillatory electromagnetic field and the static magnetic field, as set forth below. The effect of the oscillatory electromagnetic field is to heat up the magnetic particles and render them substantially nonmagnetic.”

“It is a general feature of this invention that the oscillatory electromagnetic wave intensity be absent or of negligible value in the selected target zone. Oscillatory electromagnetic waves may be locally diminished (1) by natural exponential attenuation upon passage through lossy material, and (2) cancellation of waves oppositely phased emanating from two or more sources.”

In the section of U.S. Pat. No. 4,690,130 appearing at column 6 thereof and relating to “ENERGY ABSORPTION IN PARTICLES,” it is disclosed that: “A central feature of this invention is the spatially controlled disposition of oscillatory electromagnetic energy in said particles. In an idealized circumstance, such energy disposition would be zero at the targeted field zone and abruptly very high elsewhere. Specific physical interactions mediate to diminish the abruptness of the absorption transition in and out of the target field zone. However, using the techniques as described herein, together with materials having appropriate absorption characteristics and moderately abrupt Curie temperature, effective restraint in the target zone is achieved.”

U.S. Pat. No. 4,690,130 then goes on to discuss absorption phenomena, stating that (at column 6 et seq.) “The absorption of oscillatory electromagnetic radiations in magnetic and in conductive matter will now be considered. For example, from the American Institute of Physics Handbook (McGraw-Hill, New York, 1957), Sec. 5 p. 90, tin and magnetic iron have very similar conductivities, being in a ratio of 1:1.2. Nevertheless, the absorption of energy flux is in a ratio of 1:16 based upon the relative penetration depths at which the flux has diminished to 1/e squared for radiation in the range of 1 to 3000 MHz. This rather marked absorption difference is attributed to the relative magnetic permeabilities which are in a ratio of 1:200. Electromagnetic radiation, which consists of oscillatory electric E and magnetic B vector components, is absorbed in relation to electric conductivity and magnetic permeability, respectively. Accordingly, it may be understood that tin and magnetic iron both absorb a certain similar proportion of the electric component but the magnetic iron additionally absorbs a very large proportion of the magnetic component. If both components are radiated at equal amplitudes, it may be expected that magnetically responsive substances will absorb energy predominantly from the magnetic component.”

“The relevance of this interaction to the present invention may now be understood. The particles of this invention have a magnetic permeability which is very sensitively temperature dependent. In the targeted field zone, the particles are to be maximally magnetically responsive in order to effect restraint with respect to the static magnetic field. In regions immediately exterior to this zone, the particles are to be minimally magnetically responsive in order to allow unrestrained flow into the zone.”

“If, for example, the electromagnetic radiation immediately exterior to the zone were ten times as high as in the zone, then the particles would be expected to sustain a ten-fold higher energy absorption and a concurrent temperature rise outside the zone. However, since the particles are deliberately designed to exhibit a substantial reduction in magnetic permeability in response to a substantial temperature rise, the absorption of the magnetic component of oscillatory electromagnetic energy is severely diminished. If the magnetic component is the predominant source of energy, then the desired effect partially cancels the means to achieve that effect. That is, an initially high temperature rise brought about by a strong absorption of the magnetic component is quickly followed in equilibrium by a partial loss in temperature as the magnetic component is less strongly absorbed. Since the final equilibrium temperature is not as high as the brief initial temperature, the particles immediately exterior to the zone sustain only a partially reduced magnetic responsiveness and may exhibit a degree of undesired restraint in response to the static magnetic field. Effectively, the minimum size of the targeted field zone is increased somewhat and the concentration of restrained particles is not as abruptly delineated by the zone.”

“As developed below, however, the multiplicity of antenna elements may be so configured and phased so as to substantially cancel the oscillatory magnetic components and augment the oscillatory electric components in the aforementioned regions exterior to the targeted field zone. Since the interaction of the particles with regard to the oscillatory electric component is effectively independent of temperature, the energy absorption of the electric-enhanced oscillatory field is essentially proportional to the intensity of the field.”

“This type of arrangement increases the sharp delineation of the particle restraint zone. Specifically, consider FIG. 6 where the instantaneous oscillatory field components are generated from a pair of equally driven antenna dipole elements 52(a) and 54(b). The respective resultant magnetic components Ba and Bb at the point 56 are oppositely oriented, perpendicular to the plane of the page, thereby cancelling. The electric components add vectorially giving a value Etot significantly larger than the components themselves. Extending this configuration to a second pair of antenna elements 58 and 60, where all four elements are on the vertical edges of a box-like geometrical shape of square cross section, as shown in FIG. 7, allows the generation of a strong electric oscillatory field located centrally above as indicated at reference numeral 62. The corresponding net magnetic component remains at a constant zero magnitude.”

In one embodiment of the instant invention, and as described elsewhere in this specification, a multiplicity of nanomagnetic particles and/or nanomagentic coatings are used instead of, or in addition to, the “antenna elements” of U.S. Pat. No. 4,690,130 so that the electromagnetic fields disposed about an implanted medical device (such as, e.g., an implanted stent) cooperate to cause a therapeutic agent to travel into the surface of the stent.

Referring again to U.S. Pat. No. 4,690,130, at columns 7-9 such patent discusses the properties of the particles used in the process of their invention. It is disclosed that: “A number of substances called ferromagnetics, such as iron, may be very strongly magnetized while in the presence of a magnetic field. Most of these substances exhibit magnetization versus temperature curves similar in shape to FIG. 8 but differing in scale. For example, the magnitude of the maximum magnetization Mm and the temperature Tc on the absolute scale varies considerably among the known ferromagnetics. The value Tc is the temperature at which the extrapolated curve intersects the axis, and is known as the Curie point. A substance responding as in FIG. 8 is said to be ferromagnetic when below the Curie point, Tc. At temperatures above the Curie point Tc, the curve descent levels off somewhat wherein a substance is said to be paramagnetic.”

“The very large magnetization exhibited by ferromagnetic substances is a collective quantum mechanical phenomenon known as exchange coupling. When aggregates of certain atomic species are formed, a very large percentage of the individual atomic magnetic moments align together. The broad gradually sloping region of FIG. 8 below Tc shown in FIG. 8, indicates nearly 100% alignment. As temperature increases up to Tc, this exchange coupling is disrupted by thermal agitation with a concurrent decrease in magnetization. The paramagnetic state, above Tc, is said to exist when sufficient disruption occurs such that the coupling is totally broken and the atoms act independently in their alignment response. The maximum magnetization Mm for the purposes of this invention, should be substantial, ideally comparable to iron and other strong ferromagnetics. The particles of this invention should also exhibit response wherein human body temperature, which is 310 degrees K., or 98.6 degrees Fahrenheit, should fall at a point TO on the shoulder of the curve at the onset of rapid descent as in FIG. 8. For a value of TO so situated, Tc is typically a modest increment higher on the order of magnitude of 10 degrees Kelvin. While it is not necessary that the induced temperature increase actually reach or exceed Tc, it is essential that a very large relative decrease in magnetization be effected. Nevertheless, substances having Curie points slightly above 310 degrees K. are indicative of good candidates for the particles.”

U.S. Pat. No. 4,690,130 then goes on to disclose that: “Pure iron for example is inappropriate, having a Curie temperature of 1040 degrees K. Several possible choices and their Curie temperature in degrees Kelvin include, CrTe, 320; Cr3 Te4, 325; Nd2 Fe7, 327; Ni—Cr (5.6% atomic % Cr), 324; and Fe—Ni (about 30% Ni) 340 as well as many other combinations. Furthermore, it is known in the art that small percentage variations in composition can increase or decrease the Curie temperature by several degrees. For instance, the Fe—Ni alloy can be altered to provide a lower Curie temperature of perhaps 320. The Fe—Ni alloy is also desirable since it is a moderately good conductor, essential to absorption of the oscillatory electric component. Fe—Ni also exhibits magnetization comparable to that of pure iron, Fe. Biologically, the elements Fe and Ni do not exhibit the undesirable toxicity common to an element such as chromium, Cr, included in some of the afore-mentioned combinations, and the material is therefore substantially medically inert.”

In the process of U.S. Pat. No. 4,690,130, an “oscillatory wave generator” is used to raise the temperature of some of the particles used in such process. As is disclosed at lines 63 et seq. of column 8 of such patent, “The purpose of the oscillatory wave generator is to significantly raise the particle temperature in regions exterior to the targeted zone. The temperature rise is caused by the preferential conversion of electromagnetic energy to thermal energy by the particles. Conversely, the temperature of surrounding tissue is not significantly raised when subjected to the same oscillatory waves.”

“The underlying physical principles are readily understood in conjunction with the relative absorptivity of good conductors and patient tissue. For example, at 100 MHz, the intensity decreases by a factor l/e squared in 0.0007 cm of copper and in 7 cm of tissue, indicating that a good conductor such as copper is 10,000 times as absorptive as tissue. The thermal energy of the particles is subsequently dissipated to surrounding tissue. However, the total mass of injected particles is many orders of magnitude less than that of the patient. Consequently, the patient is effectively an infinite heat sink negligibly increased in temperature by the relatively small total heat content transferred from the particles. Thereby, the particles are readily increased in temperature whereas direct and indirect energy transfer to tissue is negligible resulting in an insignificant rise in overall patient temperature.”

U.S. Pat. No. 4,690,130 then discloses (at column 9 et seq.) various devices that may be used to provide the desired oscillatory electromagnetic field. It states that: “The oscillatory electromagnetic field may be provided by devices such as a MA-150 waveguide antenna horn coupled to a BSD-1000 RF power generator, both manufactured by BSD Medical Corporation, Salt Lake City, Ut. These devices are conventionally used to achieve regional hyperthermia by selectively directing radio frequency (RF) electromagnetic waves of high intensity at a tumor site within a patient. Certain tumor types are temperature sensitive compared to normal tissue. In this regard, a temperature increase of about 5 degrees K. sustained for approximately 20 minutes is often effective in killing tumor cells, while normal cells are left undamaged.”

“A coaxial conductor cable interconnects the BSD-1000 to a termination within the MA-150 waveguide antenna horn consisting of plate electrodes across a dielectric layer. The antenna horn facilitiates directivity of the projected electromagnet waves. A flexible water bag affixed to the mouth of the antenna horn is pressed against the patient over the site targeted for the application of electromagnetic energy. The water efficiently couples the RF waves into tissue and minimizes reflections. Thermal energy generated in the water is continuously removed by pumping through an ice-filled heat exchanger. By this means, the surface of the patient is cooled through a thermal conductive process which allows for additional control of temperature within the patient.”

“The BSD-1000 RF power generator provides fully adjustable power from 5 watts to 250 watts over the frequency range of 95 MHz to 1000 MHz. Although heating may be obtained over a wider range, for the purposes of the present invention, a frequency range of about 50 megahertz or 50,000,000 cycles per second, up to about 200 megahertz is preferred. The reason that this range is preferred is that above 50 megahertz, there is more absorption by the particles and less by the human body; and above 200 megahertz, hot spots may develop near the horns. However, effective heating may be accomplished over a much broader range of frequencies.”

“More than one MA-150 antenna horn may be driven by the BSD-1000 using power splitters. The MA-150 units may be arranged in an array such that each unit represents an antenna element of this invention. The power output from the BSD-1000 to each MA-150 unit may be phase shifted and attenuated to control of the oscillatory wave intensity as described with respect to this invention. E-field sensors available from BSD are placed in skin contact on the patient to monitor the incident electric field and estimate the resultant internal temperature distribution.”

“The MA-150 horns project electromagnetic waves with the electric and magnetic vectors mutually perpendicular to each other and also to the direction of the wavefront propagation as is common to all such electromagnetic propagation. Thereby, as described hereinabove, two adjacent MA-150 horn units may be placed to produce total cancellation of the magnetic vector and augment the electric vector in the neighborhood of a mid-plane between the units. Correspondingly, opposing MA-150 units produce an intermediate null plane by destructive interference, as described herein, using opposite relative phase.”

“The component devices used in hyperthermia are necessarily operated at high power levels to produce gross regional temperature increases of about 5 degrees K. in and around targeted tissue. For the purposes of this invention, sub-therapeutic power levels with respect to hyperthermia, are used such that actual regional tissue temperature at all sites is never increased by more than 2 degrees K., and generally by less than 1 degree K. Nevertheless, when such tissue contains particles as described herein, then said particles locally sustain a substantially higher temperature increase of approximately 10 degrees K. as demonstrated by loss of magnetic responsiveness.”

“Furthermore, the objective of hyperthermia is, ideally, a focal heating of targeted tissue e.g., a tumor. This focal heating may be augmented by constructive interference of horn antennae at the depth of the tumor whereas in the context of the present invention, a significantly reduced RF intensity exists at the targeted tissue. It may be appreciated that attenuation by tissue absorption, and by phase inversion of the electric vectors from opposing horn antennae and destructive interference, or cancellation, may be used to produce this reduced RF intensity. The static magnetic field may be produced by Model HS-1785-4A DC power supplies combined with circular coil elements such as those in the Model M-4074 assembly, both available from Walker Scientific Inc., Rockdale Street, Worcester, Mass. 01606. The power supply generates 0-85 amps at 0-170VDC. The coil elements are wound with aluminum foil 6 inches wide with plastic film insulation between the turns. Each wound coil is affixed to a flat aluminum plate by epoxy resin and water channels milled into the plate facilitate cooling of the coil during operation.”

“A concentric pair of such coils with diameters of twenty inches and eight inches provides an effective depth controllable gradient with magnetic strength in excess of 1000 gauss. Each coil is driven by a separate power supply so that current and polarity is individually controllable.”

“The magnetic field may be mapped with a gaussmeter such as the Model MG-3D Hall effect unit available from Walker Scientific, Inc. This instrument can measure fields in the range of 10 to 100,000 gauss with an accuracy of ±0.1%.”

In columns 11-12 of U.S. Pat. No. 4,690,130, preparation of the particles used in theprocess of such invention is discussed. It is stated that: “A large variety of appropriate metallic alloys in powder form are available from manufacturers such as Ashland Chemical Co., P.O. Box 2219, Columbus, Ohio 43216. A comprehensive reference text prepared by R. M. Bozorth lists several hundred alloys and their respective Curie temperatures. Bozorth's references indicate that an alloy such as 70% Fe, 30% Ni has an appropriate Curie temperature. However, the Curie temperature exhibits a very strong compositional sensitivity, increasing several tens of degrees for each additional percent of Ni. Accordingly, commercially supplied powder consisting of approximately 100 Angstrom size particles exhibits a wide dispersion of Curie temperatures. Particles in an appropriate Curie temperature range such as 320±5 degrees K. may be separated from the particles of inappropriate Curie temperature, by the following steps. The particles are first coated with a fluorocarbon suspension agent available from Ferrofluidics Corporation of Burlington, Mass. The resultant ferrofluid is then heated in a water bath to 340 degrees K. A permanent magnet is used to extract those particles from the ferrofluid which are still magnetically responsive. This process is repeated at 5 degree K. cooling increments down to 315 degrees K. Thereby, the singular extraction at 315 degrees K. exhibits the appropriate Curie transition temperature and is retained, the other extractions being discarded.”

“Senyei and Widder in U.S. Pat. No. 4,247,406 have suggested the use of human serum albumin (HSA) microspheres as carriers of magnetically responsive particles and therapeutic substances such as chemotherapy agents, since HSA is not readily extracted from the blood by the body's defense systems. Thereby, sufficient time is allowed for an externally applied static magnetic field to trap a substantial quantity of such HSA microspheres flowing in the bloodstream. Microspheres for this invention are prepared as described by Widder and Senyei in U.S. Pat. No. 4,247,406 Example I, page 7 except that in place of Fe3 O4, particles, Fe—Ni alloy particles of 320 degrees K. Curie temperature are used.”

By way of yet further illustration, U.S. Pat. No. 4,849,210 discloses a superparagrnagnetic contrast agent and its use in imaging a tumor. Claim 1 of this patent describes “The method of imaging a tumor in the liver or spleen of a human subject, comprising parenterally administering to the human subject prior to magnetic resonance imaging (MRI) examination an aqueous suspension composed essentially of microspheres having diameters of less than 1.5 microns, said microspheres being composed of a biodegradable matrix material with a particulate superparamagnetic contrast agent therein, said superparamagnetic contrast agent consisting essentially of ferromagnetic particles of not over 300 angstroms diameter, the quantity of said microspheres administered being effective to appreciably reduce the T2 relaxation time of the subject's liver or spleen; (b) delaying the examination until the microspheres have been segregated by the reticuloendothelial system and are concentrated in the liver and spleen; and then (c) carrying out an MRI examination of the liver or spleen by T2 imaging or mixed T1 and T2 imaging to obtain an image in which the normal liver or spleen tissues appear dark and the tumor appears light with distinct margins therebetween.”

The paramagnetic contrast agents of U.S. Pat. No. 4,849,20 are described in columns 3-4 of this patent, wherein it is stated that: “The superparamagnetic contrast agent is used in particulate form, for example, as particles of 50 to 300 Angstroms diameter. Particle size of not over 300 Angstroms provides ferromagnetic iron compounds with the desired superparamagnetic characteristics; namely, enhanced magnetic susceptibility and low residual magnetization. Preferably, the particulate forms are substantially water-insoluble, such as insoluble oxides or salts. The superparamagnetic contrast agent may also be in the form of particles of an elemental metal such as particularly iron particles sized below 300 Angstroms. A preferred particulate contrast agent is magnetite, which is a magnetic iron oxide sometimes represented as Fe3 O4 (or as FeO.Fe2 O3 .) Commercially, fine powders or suspensions of magnetite are available from Ferrofluidics Corporation, Burlington, Mass. The size range of the particles is submicron, viz. 50 to 200 Angstroms. Other water-insoluble superparamagnetic iron compounds can be used such as ferrous oxide (Fe2 O3), iron sulfide, iron carbonate, etc. For purposes of this invention, the microspheres comprise relatively spherical particles consisting of protein, carbohydrate or lipid as the biodegradable matrix for the paramagnetic contrast agent. For effective targeting to the liver and spleen, the microspheres comprising the encapsulated contrast agents should have diameters up to about a maximum size of 8 microns. An advantageous size range appears to be from about 2 to 5 micro diameter. Less than 1.5 micron microspheres can be used as a livery spleen contrast agent (viz. 1.0 micron size), but circulation time is prolonged, that is, fewer spheres will be rapidly taken up by the RES. Microspheres of larger size than 8 microns may be sequestered in the first capillar bed encountered, and thereby prevented from reaching the liver and spleen at all. Large microspheres (viz. 10 microns or more) can be easily trapped in the lungs by arteriolar and capillary blockade. See Wagner et al., J. Clin. Investigation (1963), 42:427; and Taplin, et al., J. Nucl. Medicine (1964) 5:259.”

“The matrix material may be a biodegradable protein, polysaccharide, or lipid. Non-antigenic proteins are preferred such as, for example, human serum albumin. Other amino acid polymers can be used such as hemoglobin, or synthetic amino acid polymers including poly-L-lysine, and poly-L-glutamic acid. Carbohydrates such as starch and substituted (DEAE and sulfate) dextrans can be used. (See Methods in Enzymology, 1985, Vol. 112, pages 119-128). Lipids useful in this invention include lecithin, cholesterol, and various charged phospholipids (stearyl amines or phosphatidic acid). Microspheres having a lipid matrix are described in U.S. Pat. No. 4,331,564.”

“Microspheres for use in practicing the method of this invention can be prepared from albumin, hemoglobin, or other similar amino acid polymers by procedures heretofore described in literature and patent references. See, for example, Kramer, J. Pharm. Sci. (1974) 63: 646; Widder, et al., J. Pharm. Sci. (1979) 68: 79; Widder and Senyei, U.S. Pat. No. 4,247,406; and Senyei and Widder, U.S. Pat. No. 4,230,685. Briefly, an aqueous solution is prepared of the protein matrix material and the paramagnetic/ferromagnetic contrast agent, and the aqueous mixture is emulsified with a vegetable oil, being dispersed droplets in the desired microsphere size range. Emulsification can be carried out at a low temperature, such as a temperature in the range of 20-30° C., and the emulsion is then added dropwise to a heated body of the same oil. The temperature of the oil may range from 70 to 160° C. The dispersed droplets in the heated oil are hardened and stabilized to provide the microspheres which are then recovered. When most of the microspheres as prepared, such as 80% or more, have sizes within the ranges described above, they can be used as prepared. However, where substantial amounts of oversized or undersized microspheres are present, such as over 10 to 20% mof microspheres larger than 8 microns, or over 10 to 20% of microspheres smaller than 1.5 microns, a size separation may be desirable. By the use of a series of micropore filters of selective sizes, the oversized and undersized microspheres can be separated and the microspheres of the desired size range obtained.”

“The microspheres may contain from 5 to 100 parts by weight of the contrast agent per 100 parts of the matrix material. For example, in preferred embodiments, microspheres can contain from 10 to 30 parts by weight of magnetite particles or another superparamagnetic contrast agent per 100 parts of matrix material such as serum albumin.”

In one preferred embodiment of this invention, one may modify the miscrospheres of U.S. Pat. No. 4,849,210 by replacing the magnetite particles in such microspheres with one or more of the nanomagnetic particles of this invention.

U.S. Pat. No. 4,863,717 describes the use of “stable nitroxide free radicals” as contrast agents for magnetic resonance imaging. The entire disclosure of this United States patent is hereby incorporated by reference into this specification.

Claim 1 of U.S. Pat. No. 4,863,717, which is typical, describes “In an MRI contrast agent which is a liposome having a bound spin label that is subject to reduction, and thus loss of contrast enhancement capability when in a reducing environment, the improvement wherein the liposome incorporates oxidizing means for oxidizing and thereby restoring spin labels that have been reduced” This contrast agent is useful in magnetic resonance imaging (MRI), which is discussed in column 1 of the patent.

As is disclosed in column 1 of U.S. Pat. No. 4,863,717, “Magnetic resonance imaging (MRI) is a powerful noninvasive medical diagnostic technique that is currently in a period of rapid development. Agents which selectively enhance the contrast among various tissues, organs and fluids or of lesions within the body can add significantly to the versatility of MRI.”

Liposomes, with compartments containing entrapped Mn-DTPA or some other paramagnetic substance, have been investigated as potential contrast agents for MRI, as described by Caride et al. in Magn. Reson. Imaging 2: 107-112 (1984). Liposomes tend to be taken up selectively by certain tissues such as the liver and are in general nonantigenic and stable in blood. They are used extensively as experimental drug delivery systems, as described by Poste et al. in “The Challenge of Liposome Targeting in Vivo”, Chapter 1, Lipsome Technology: Volume III, Targeted Drug Delivery and Biological Interaction, G. Gregoriadis, Ed., CRC Press, Boca Raton, Fla. (1984). However, where tested for MRI in the past, liposomes have served merely as vessels to contain encapsulated paramagnetic material.”

“Owing to their paramagnetic nature and thus their ability to affect the relaxation times T1 and T2 of nearby nuclei, nitroxide free radicals constitute a class of potential MRI contrast-enhancing agents which are not toxic at low dosages. There are many examples of nitroxide-containing phospholipids, but these are invariably used in low concentrations merely to dope non-paramagnetic phospholipids for biophysical spin labeling studies, as described, for example, by Berliner, L. J., ed., in Spin Labeling: Theory and Applications, Academic Press, New York, volumes 1 and 2, 1976 and 1979 and by Holtzmann, J. L. in Spin Labeling Pharmacology, Academic Press, New York, 1984. European patent publication EP A 0160552, suggests that free radicals such as organic nitroxides may be enclosed within liposomes. The liposomes are said to be sufficiently leaky to water that, although the paramagnetic material is trapped inside, relaxation of bulk water can nevertheless occur by exchange of bulk water with inside water.”

“A more direct and reliable approach would be to incorporate nitroxide into the bilayer of the liposome. But, one would expect such a use of nitroxide to be hampered by a tendency of the paramagnetic nitroxyl group to accept an electron from the local environment and thus be reduced to a useless diamagnetic N-hydroxy compound, as described in Griffeth et al., Invest. Radiol. 19: 553-562 (1984); Couet, Pharm. Res. 5: 203-209 (1984); and Keana et al., Physiol. Chem. Phys. and Med. NMR 16: 477-480 (1984).”

“In the past, “reduction” problems have been handled by injecting large amounts of conventional nitroxide compounds into a subject with the intent of “swamping” the reduction reaction. Particularly large dosages have been required because there has been no practical way to direct nitroxide to specific tissues other than the liver and spleen. Because such nitroxides are rapidly diluted in body circulatory liquid, massive amounts of the contrast agent must be administered or the dilution effect renders the nitroxides ineffective as general contrast enhancers. The use of large dosages is not only wasteful and expensive, but also the large quantities of nitroxides and their metabolites can cause toxicity problems in sensitive subjects.”

“It would be helpful to target certain tissues, say cardiac tissue or tumor tissue, for contrast enhancement. If nitroxides could be concentrated in certain areas of the body, they would encounter fewer “reducing equivalents” than they would if carried throughout the entire body. To accomplish targeting, one thinks in terms of labeling an antibody or monoclonal antibody which seeks out the target tissue. But, it is clear that one or even a few nitroxides attached to an antibody will not provide enough enhancement. On the other hand, one cannot simply add hundreds directly to the antibody because that would almost surely destroy the antibody's ability to bind selectively to its target. Thus, a specific need has been to find a nontoxic contrast enhancing agent that can be targeted for specific tissues.”

“Prior patent publications such as EP A 0160552 and GB 2137612 describe the combined use of a contrast agent and a targeting agent such as an antibody. Such references do not, however, suggest how such targeting agents may be employed effectively with a nontoxic contrast agent such as a compound which effectively employs nitroxide free radicals.”

Two solutions are presented to the “nitroxide reduction” problem described in U.S. Pat. No. 4,863,717. One of these solutions is described at lines 56 et seq. of column 2 of the patent, wherein it is suggested “ . . . to administer a relatively snall number of large molecules, such as arborols, or assemblies of molecules such as liposomes, that have surfaces covered with numerous persistant nitroxide free radicals. The reduction problem is thus addressed through the sheer number of nitroxides on a given molecule.”

This solution is also described at lines 40 et seq. of column 8 of the patent, wherein it is disclosed that: “A second embodiment of the invention employs large molecules, particularly polymeric molecules, or assemblies of molecules, particularly liposomes, constructed to have numerous, i.e. at least about ten, persistent nitroxide free radicals. Because there are so many persistent nitroxide free radicals, the reduction of a few such free radicals is of little significance. Such large molecules or polymers are not merely carriers of encapsulated contrast agents. They are, themselves, the contrast agents since their surfaces are covered with persistent nitroxide free radicals.”

“One such construction is a nitroxide-doped liposome formed by sonication of amphipathic molecules having persistent nitroxide groups. A suitable amphipathic molecule has a polar head group, at least two chains and a nitroxide group sufficiently near the head group that the nitroxide can contact bulk water when in a liposome. As a general rule, the nitroxide must be ten carbons or less from the head group for there to be effective bulk water contact. Particularly well suited are double chain amphipathic molecules having a nitroxide group near the polar end of each chain. To be effective as a sustained use contrast agent, substantially all the amphipathic molecules that make up the liposome should cntain at least one nitroxide group. Most advantageously, the polar head group will also have at least one nitroxide.”

In one embodiment of the instant invention, a therapeutic agent is modified such that it contains a multiplicity of either “persistent nitroxide free radicals” and/or “reversibly reducible nitroxide groups.” In one preferred aspect of this embodiment, the therapeutic agent so modified is an anti-microtubule agent, such as paclitaxel.

By way of further illustration, one may use the hydrophilic microspheres disclosed in U.S. Pat. No. 4,871,716, the entire disclosure of which is hereby incorporated by reference into this specification. As is disclosed in such patent, many of the “prior art” microspheres a hydrophobic. Thus, and referring to column 1 of this patent, “Insoluble magnetically responsive polypeptide or protein microspheres containing therapeutic agents that enable the controlled releases thereof in biological systems following localization by an externally applied magnetic field have generated growing interest in recent years [Widder et al: Cancer Research, 40, p. 3512 (1980) and Widder et al: J. Pharm. Sci., 68, p. 79 (1979)]. Systems utilizing the microspheres have the potential advantage of prolonging effective drug concentrations in the blood stream or tissue when injected thereby reducing the frequency of administration; localizing high drug concentrations; reducing drug toxicity, and enhancing drug stability. Albumin is a preferred protein or polypeptide for the preparation of such microspheres since it is a naturally occurring product in human serum. Although it is usually necessary to cross-link the albumin when preparing microspheres according to conventional methods, cross-linked albumin may still be degraded depending upon cross-link density thereby enabling the use thereof for drug delivery systems, etc.”

‘Conventional methods for the preparation of magnetically responsive albumin microspheres are generally of two types. In one method, aqueous dispersions of albumin and magnetically responsive material are insolubilized in vegetable oil or isooctane or other hydrocarbon solvent by denaturing at elevated temperatures (110°-165° C.). Another method involves chemical cross-linking of the aqueous dispersion of albumin at room temperature. Typical of these two types of methods are those described in U.S. Pat. Nos. 4,147,767; 4,356,259; 4,349,530; 4,169,804; 4,230,687; 3,937,668; 3,137,631; 3,202,731; 3,429,827; 3,663,685; 3,663,686; 3,663,687; 3,758,678 and Ishizaka et al, J. Pharm. Sci., Vol. 20, p. 358 (1981). See also U.S. Pat. Nos. 4,055,377; 4,115,534; 4,157,323; 4,169,804; 4,206,094; 4,218,430; 4,219,411; 4,247,406; 4,331,654; 4,345,588; 4,369,226; and 4,454,234. These methods, however, result in the formation of relatively hydrophobic microspheres which usually require a surfactant in order to disperse a sufficient quantity thereof in water or other systems for administration to a biological system to ensure the delivery thereto of an effective amount of any biologically active agent entrapped therein. In addition, the hydrophobic nature of conventional polypeptide microspheres make it difficult to “load” large quantites of some water soluble biologically active agents or other material within the microspheres after synthesis. It is an object of the present invention to provide more hydrophilic magnetically responsive polypeptide microspheres which will accept high “loadings” of biologically active substances of other materials especially by addition of such substances after microsphere synthesis, and to prepare such drug loaded microspheres which do not require the utilization of surfactants to enable the preparation of highly concentrated dispersions thereof.”

A method for preparing such “ . . . hydrophilic magnetically responsive polypeptide microspheres . . . ” is described in claim 1 of U.S. Pat. No. 4,871,716. This claim describes: “A method of preparing novel hydrophilic, magnetically responsive microspheres consisting essentially of cross-linked protein or polypeptide particulate and a magnetically responsive material comprising (a) providing a dispersion of an aqueous solution or dispersion of polypeptide or protein microspheres and a particulate magnetically responsive material in an organic, substantially water immiscible solvent solution of a high molecular weight polymer, said organic solvent being substantially a non-solvent for said microspheres and said polymer solution stabilizing the dispersion of microspheres and magnetically responsive material, (b) incorporating a polyfunctional cross-linking agent for said protein or polypeptide in said dispersion, and (c) allowing said cross-linking agent to react with said protein or polypeptide microspheres for a time sufficient to cross-link at least a portion of the microspheres, thereby providing magnetically responsive microspheres containing free reactive functional groups.”

With these hydrophilic moieties, various drugs can be incorporated into the microspheres. Thus, as it disclosed at lines 17 et seq. of column 32 of the patent, “The magnetically responsive microspheres of the present invention, unlike those of the prior art are hydrophilic and may be readily dispersed in aqueous media for injection without the need for surfactants. In addition, they may be readily prepared with the incorporation of very high concentrations of therapeutic agents such as the cancer chemotherapeutic drug adriamycin (up to 50 wt % drug). Previous magnetically responsive hydrophobic albumin microsphere-drug preparations have usually succeeded in incorporating not more than 10-15 wt % of such anti-tumor drugs. Also, the hydrophobic magnetically responsive albumin microsphere preparations known in the art have been compromised by a larger dispersion of sizes, limiting the smallest practical size to μm. In contrast, the method of the present invention enables the preparation of particles as small as 80 nm with a narrow distribution of size.”

“Using a polypeptide cross-linking agent such as glutaraldehyde, reactive aldehyde groups are available on the microspheres for additional chemical reaction. The microspheres may be reacted with amino group containing drugs for covalent coupling, or with the amino acid glycine to enhance hydrophilicity, or coupled covalently to such large protein molecules as lectins, enzymes or antibodies to modify the microsphere surface properties or to provide a carrier system for the coupled proteins. Coupling antibodies to the magnetically responsive microspheres provides methods for the selective removal of cells from cell cultures in suspension by targeting the microspheres to the surface of specific cells, rendering them magnetic, and pulling the cell-microsphere conjugate from solution by means of an externally applied magnetic field, or for use in vivo as a diagnostic aid. Antibodies coupled to magnetically responsive submicron microspheres applied in vivo, i.e., injected intra-arterially, intra-veinously, intra-lymphatically, etc., may localize the microspheres on the surface of specific cells providing a radiopaque element for either radiographic imaging or, magnetic resonance imaging. One type of magnetically responsive microspheres currently used for separation of cell culture suspensions are made of polystyrene which gives a relatively unreactive surface to which antibodies can only be coupled by passive adsorption. As a result, the antibodies tend to dissociate from the microsphere surface with time, necessitating the use of excessive amounts of antibodies and limiting the useful storage life of the microsphere.’

‘The present invention enables the incorporation into the magnetically responsive hydrophilic microspheres of various drugs for localization by means of an extracorporeally applied magnetic field and controlled release, radiographic and magnetic resonance imaging, and selective separation of cell culture suspensions. Various synthetic drugs or enzymes or antibodies or proteins may be incorporated into the microsphere by physical association, by electrostatic interactions, or covalently for altering release kinetics and other property modifications. Such microspheres may also be used for adjuvant compositions incorporating such immunostimulants as interferon or MDP. Albumin may also be combined with various other macromolecules or polypeptides in the course of preparation of the microsphere. For example, polyglutamic acid has been incorporated into magnetically responsive HSA microspheres to enhance the anionic nature of the microsphere and so facilitate the binding of high concentrations of cationic drugs such as adriamycin, bleomycin, or streptomycin. The drugs which may be used in such microspheres include the clinically important antitumor drugs (e.g., adriamycin, mitomycin, bleomycin, etc.) as well as hormones such as cortisone derivatives and antibiotics such as gentamycin, streptomycin, penicillin, etc.”

At columns 16-17 of U.S. Pat. No. 4,871,76, the rate at whichthe microspheres of this patent release the therapeutic agents to which they were bound was measured. In the experiments described in Tables 8, 9, 10, and 11, e.g. (see columns 17 and 18), release rates of the drug varied from about 19 percent to about 50 percent over a period of from about 2 to about 14 hours.

In one embodiment of this invention, the anti-tumor agent used with the microspheres is paclitaxel, and the drug composition so produced is situated near a drug eluting stent and caused to release such paclitaxel to such stent.

By way of yet further illustration, one may use the magnetic drug assembly described in claim 12 of U.S. Pat. No. 5,411,730, the entire disclsoure of which is hereby incorporated by reference into this specification. Such claim 12 is indirectly dependent upon claim 1 of such U.S. patent, which claim describes: A composition comprising particles of an iron oxide and a polymer, said iron oxide being superparamagnetic, the ratio of polymer to iron being 0.1 to 0.5 (w/w), said particles having sedimentation constants in the range of 150-5000S, said particles having at least one of the following magnetic properties:a) specific power absorption rate (SAR) greater than 300 w/g Fe, measured in an electromagnetic field of 1 MHz frequency and 100 Oe field strength; b) initial magnetic susceptibility greater than 0.7 EMU/gFe/Gauss; and c) magnetic moment greater than 10-15 erg/Gauss.” claim 9, which is directly dependent upon claim 1, further specifies that the particles comprise a particle-encapsulating lipid. Claim 12, which is dependent upon claim 9, further specifies hat the particle-encapsulating lipid comprises a therapeutic agent.

At column 3 of U.S. Pat. No. 5,411,730, a discussion of the use of heat to induce the rapid release of pharmaceuticals to a desired site. As is disclosed in this patent, “A different approach to drug targeting has been developed in the works by Yatvin et al. [42,43] and Huang et al. [44]. They used heat to induce rapid release of pharmaceuticals from thermosensitive liposomes composed of phospholipids having transition temperatures slightly above normal physiological temperature. Local hyperthermia, heating of the target area to a temperature of 42°-44° C., would cause the liposome lipids to “melt”, and the liposomes flowing through the vascular bed of a hyperthermized area would rapidly release the entrapped drug into the surrounding medium. Since the drug is released in its intact form, the problems concerning drug extravasation and activity are avoided. So, in the approaches proposed by Yatvin and Huang, the targeted mode of drug delivery substantially depends on the ability to apply hyperthermia to the area of pathology in a targeted manner; unfortunately, none of the existing techniques of hyperthermia offers a general and satisfactory way to do so [10].”

In one embodiment of the invention of U.S. Pat. No. 5,411,730, the patentees incorporated adriamycin into thermosensitive ferroliposomes and caused the release of such an anti-tumor agent by electromagnetic radiation. Thus, as is disclosed in column 20 of the patent, “Adriamycin (doxorubicin hydrochloride) is of great interest as a targeted anticancer drug because the great therapeutic potential of this anticancer drug is limited by its systemic toxicity, especially cardiotoxicity [54]. Thermosensitive ferroliposomes are loaded with adriamycin using the “remote loading” technique [55]. This technique employs the property of weak lipophilic bases or acids to cross the liposomal membrane in response to transmembrane gradient of pH [56]. Adriamycin, a weak base, spontaneously accumulates in the liposomes with an acidic (pH 4) interior when the exterior buffer is kept at pH 7 or higher. The accumulated drug remains inside liposomes until the transmembrane pH gradient is fully relaxed. Specifically, we prepare ferroliposomes using glutamate buffer at pH 4.6 (interior) and pH 7.5 (exterior) as described for regular DPPC liposomes [55]. The liposomes are incubated with adriamycin at approx. 0.1:1 drug to lipid ratio, aliquots are taken at various incubation times, and liposome-bound adriamycin is determined by its intrinsic fluorescence in the void volume fraction after passage of an aliquot through a small gel-filtration column (NP-10, Pharmacia). If the incubation time required for the loading is too high, which is not unlikely for a phospholipid bilayer below its transition temperature, we perform incubation at temperature above Tc and quench the drug-loaded liposomes by injecting them into the ice-cold buffer. These experiments establish the incubation time and temperature for efficient loading of the thermosensitive ferroliposomes with adriamycin. The unbound drug is removed from the loaded ferroliposomes by gel filtration through Sephadex G-25. 5. Spontaneous and RF-field triggered release of Adriamycin from thermosensitive ferroliposomes.”

“We compare the release of adriamycin from thermosensitive ferroliposomes in the physiological saline buffer (PBS), PBS+10% fetal calf serum (FCS), and RPMI 1640 cell culture medium +10% FCS under the following conditions: (a) storage at room temperature and +4° C.; (b) water bath heating to temperatures above Tc; (c) exposure to RF electromagnetic field.”

“This part of the work explores triggering cell death by exposure of cancer cells to RF electromagnetic field in the presence of Adriamycin-loaded thermosensitive ferroliposomes. We use Adriamycin-sensitive human small cell lung cancer cell lines SHP-77 and H345, routinely maintained in our laboratory. The cells are grown in RPMI 1640 medium plus 10% FCS at 37° C. Ferroliposomes and Adriamycin stock solution are diluted with cell medium and sterilized by filtration. Various doses of sterile ferroliposomes and/or Adriamycin, free or ferroliposome-incorporated, are added to the cells in standard cell-culture 96well plates. To observe the effect of RF field, cell suspension is temporarily transferred to a tissue culture plastic tube inserted into the inductor coil. Growth of the cells is evaluated using our routine (3H)Thymidine incorporation assay [57]. Table 8 describes the experimental design for this study.”

“The need for site-specific cancer chemotherapy is evident, and the success in this area is still far below this need. This invention includes a totally novel approach to site-specific chemotherapy. The chemotherapeutic substance is incorporated into thermosensitive liposomes together with ferromagnetic microparticles. Such liposomes normally retain their contents for a long time. However, when such liposomes approach the target site exposed to the source of radiofrequency electromagnetic field, the field heats the ferromagnetic particles; they in turn heat the liposome membrane to reach the transition temperature of the lipid and rapidly release the drug into the vascular bed of the target area. The applications of this approach are multifold. Apart from adriamycin, it is possible to use other anticancer pharmaceuticals in the RF field-dependent ferroliposomal targeted delivery as described here. Such important anatomical areas as head, neck, extremities, and skin are very suitable for RF-field application and therefore for the targeted chemotherapy using the described approach; and the recent development of endoscopic RF-field applicators [58] substantially expand this list to include sites close to the walls of body cavities. It indicates that the approach is practical for its final destination., treatment of human patients.”

In one embodiment of the instant invention, “ . . . other anticancer pharmaceuticals . . . ,” such as, e.g., paclitaxel, are incorporated into the magnetic, thermosensitive liposomes of U.S. Pat. No. 5,41,730 and used to deliver, e.g., paclitaxel to a desired site within a biological organism. In this embodiment, the nanomagnetic film described elsewhere in this specification is utilized.

U.S. Pat. No. 5,441,746 discloses a “wave absorbing magnetic core particle” which is especially adapted to increase its temperature in vivo in response to an external magnetic field and thereby preferentially kill cancer cells; the entire disclosure of this patent is hereby incorporated by reference into this specification. Claim 1 of this patent describes: “A composition comprising a wave absorbing magnetic core particle wherein said magnetic core particle comprises an oxide of the formula M₂ (+3)M(+2)0₄ wherein M(+3) is Al, Cr or Fe, M(+2) is Fe, Ni, Co, Zn, Ca, Ba, Mg, Ga, Gd, Mn or Cd, in combination with an oxide selected from the group consisting of LiO, CdO, NiO, FeO, ZnO, NaO, KO and mixtures thereof, characterized in that said core is capable of adsorbing or coordinating with a hydrophilic moiety, coating with a first amphipathic organic compound, characterized in that said first amphipathic organic compound contains a hydrophilic moiety and a hydrophobic moiety and the hydrophilic moiety is adsorbed or coordinated with the core and the hydrophobic moiety thereby extends outwardly from the inorganic core and further coated with a second amphipathic organic compound wherein said second amphipathic compound contains hydrophobic and hydrophilic moiety and the hydropholic moiety associates with the outwardly extending hydrophobic moiety of said first amphipathic compound to form said wave absorbing composition”

U.S. Pat. No. 5,753,477 discloses a process for transfecting cells which utilizes an external magnetic field. Thus, e.g., claim 1 of this patent describes: “A method for delivery of a composition to cells in vitro, said composition comprising a plurality of substance-carrying superparamagnetic microparticles, comprising: applying a magnetic field in a least two pulses to said composition and cells, wherein said magnetic field is 0.5-50 Teslas in strength, 0.001-200 milliseconds in duration, and insufficient to heat-kill said cells, wherein said magnetic field is applied so as to achieve penetration of the cell membrane by said substance-carrying superparamagnetic microparticles, and said cells are maintainable in viable culture post-delivery.”

The process claimed in U.S. Pat. No. 5,753,477 is related to other “prior art” means for delivering substances into cells, which are discussed incolumns 1 and 2 of U.S. Pat. No. 5,753,477. As is disclosed at lines 30 et seq. of such column 2, “Other previous substance delivery methods have included the use of magnetic nicrospheres to deliver substances into cells. For example, Widder et al. have described the development of a magnetically responsive biodegradable magnetic drug carrier with the capacity to localize both carrier and chemotherapeutic agent by magnetic means to a specific in vivo target site after systemic administration. Widder et al., 58 Proc. Soc. Exp. Bio. & Med. 141 (1978). The carrier consists of albumin microspheres 0.2-2 microns in diameter containing both magnetic Fe3 O4 microparticles (10-20 nm in diameter) and a chemotherapeutic agent entrapped in the albumin matrix. This complex can be held in the desired location via an external static permanent magnet. It has been reported that these complexes are internalized by tumor cells in vitro and in vivo following intra-peritoneal (ip) injection, possibly through passive phagocytosis process.”

The rationale for the process of U.S. Pat. No. 5,753,477 is discussed in column 3 of the patent, at lines 49 et seq. It is disclosed in this column 3 that: “In the absence of an applied magnetic field, superparamagnetic microparticles of size 10 to 100 nm in diameters undergo Brownian motion. When an external magnetic field of moderate strength of 100 to 200 gauss is applied, these particles become magnetized and form into small magneto-needles because of its high initial magnetic susceptibility (0.1 to 0.7 emu/grn Fe/Gauss) and relatively low saturation magnetization (80 emu/gm Fe). In the continual presence of applied field, the small needles can undergo needle-needle interactions and coalesce into bigger needles. These needles generally move past one another until their ends join to each other. Moreover, these needles continue to move slowly toward the applied pole surface of the external magnet. When a stronger magnetic field is applied, the needles move much faster toward the applied magnet. In general, because of the short duration (micro- to milli-seconds) of a pulse in a high magnetic field (2 to 50 Teslas), two stages of magnetic induction are required to act on the particles in order for the particles to accelerate to a high enough velocity to penetrate a single cell membrane or multi-cell layers.”

“First, the superparamagnetic or ferromagnetic microparticles are pre-magnetized with a primary solenoid of 100 to 1000 Gauss briefly for 1 to 10 seconds (although pre-magnetization is not essential for ferromagnetic particles, so long as they are already magnetic) and immediately followed by the secondary high magnetic pulse (2 to 50 Teslas) of 10 to 200 milliseconds produced by a second solenoid, which serves to accelerate the pre-magnetized particles into the target. Also disclosed is a method as above wherein the pulse(s) is 1 microsecond to 200 milliseconds in length. The target and the magnetic microparticles are placed along the Z-axis and at a position of maximum field gradient directly outside of the secondary pulse coil. Since a homogeneous field is not required for the magnetic biolistic process, any coil which produces high field gradients described will function in the present method. Depending on the cell types, ie. single cell or multi-cell layers, single and/or multi-pulses can be applied to the microparticles and the target. In the absence of a high pulsed field device (field strength greater than 2 Teslas), a coil capable of delivering multi-pulses of continuously moderate field strength (0.5 to 2 Teslas) with pulse durations of 10 to 200 milliseconds, can also be used to deliver superparamagnetic and/or ferromagnetic microparticles into a single cell layer. Intervals between pulses should be kept as close as possible. This set up is more suitable for in vitro single cell layer transfection.”

U.S. Pat. No. 6,200,547 claims a magnetically responsive composition comprised of paclitaxel absorbed on its particles; the entire disclosure of this United States patents is hereby incorporated by reference into this specification. Such claim 7 describes: “A magnetically responsive composition comprising: a) a carrier including particles between about 0.5 μm and 5 μm in crossectional size, each particle including a ratio of iron to carbon in the range from about 95:5 to about 50:50 with the carbon distributed throughout the volume of the particle; and b) a therapeutic amount of paclitaxel adsorbed on the particles.”

At columns 1-2 of this patent, “prior art” magnetically responsive compositions were discussed. It was statedin this section of the patent that: “Metallic carrier compositions used in the treatment of various disorders have been heretofore suggested and/or utilized (see, for example, U.S. Pat. Nos. 4,849,209 and 4,106,488), and have included such compositions that are guided or controlled in a body in response to external application of a magnetic field (see, for example, U.S. Pat. Nos. 4,501,726, 4,652,257 and 4,690,130). Such compositions have not always proven practical and/or entirely effective. For example, such compositions may lack adequate capacity for carriage of the desired biologically active agent to the treatment site, have less than desirable magnetic susceptibility and/or be difficult to manufacture, store and/or use.

“One such known composition, deliverable by way of intravascular injection, includes microspheres made up of a ferromagnetic component covered with a biocompatible polymer (albumin, gelatin, polysaccharides) which also contains a drug (Driscol C. F. et al. Prog. Am. Assoc. Cancer Res., 1980, p. 261).”

“It is possible to produce albumen microspheres up to 3.0 μm in size containing a magnetic material (magnetite Fe3 O4) and the anti-tumoral antibiotic doxorubicin (Widder K. et al. J. Pharm. Sci., 68:79-82 1979). Such microspheres are produced through thermal and/or chemical denaturation of albumin in an emulsion (water in oil), with the input phase containing a magnetite suspension in a medicinal solution. Similar technique has been used to produce magnetically controlled, or guided, microcapsules covered with ethylcellulose containing the antibiotic mitomycin-C (Fujimoto S. et al., Cancer, 56: 2404-2410,1985).”

“Another method is to produce magnetically controlled liposomes 200 nm to 800 nm in size carrying preparations that can dissolve atherosclerotic formations. This method is based on the ability of phospholipids to create closed membrane structures in the presence of water (Gregoriadis G., Ryman B. E., Biochem. J., 124:58, 1971).”

“The above compositions require extremely high flux density magnetic fields for their control, and are somewhat difficult to produce consistently, sterilize, and store on an industrial scale without changing their designated properties.”

“To overcome these shortcomings, a method for producing magnetically controlled dispersion has been suggested (See European Patent Office Publication No. 0 451 299 Al, by Kholodov L. E., Volkonsky V. A., Kolesnik N. F. et al.), using ferrocarbon particles as a ferromagnetic material. The ferrocarbon particles are produced by heating iron powder made up of particles 100 μm to 500 μm in size at temperatures of 800° C. to 1200° C. in an oxygen containing atmosphere. The mixture is subsequently treated by carbon monoxide at 400° C. to 700° C. until carbon particles in an amount of about 10% to 90% by mass begin emerging on the surface. A biologically active substance is then adsorbed on the particles. This method of manufacturing ferrocarbon particles is rather complicated and requires a considerable amount of energy. Because the ferromagnetic component is oxidized due to the synthesis of ferrocarbon particles at a high temperature in an oxygen containing atmosphere, magnetic susceptibility of the dispersion obtained is decreased by about one-half on the average, as compared with metallic iron. The typical upper limit of adsorption of a biologically active substance on such particles is about 2.0% to 2.5% of the mass of a ferromagnetic particle. The magnetically controlled particle produced by the above method has a spheroidal ferromagnetic component with a thread-like carbon chain extending from it and is generally about 2.0 μm in size. The structure is believed to predetermine the relatively low adsorption capacity of the composites and also leads to breaking of the fragile thread-like chains of carbon from the ferromagnetic component during storage and transportation.”

The magnetically responsive composition described in claim 7 of United States patent has paclitaxel adsorbed on its particles. A process for producing this composition is disclosed in Example 4 of the patent.

As is disclosed in such Example 4 of U.S. Pat. No. 6,200,547, “The results in Table 3 show that binding of the drug to the carrier particles is highly influenced by the composition of the adsorption solution or medium. Camptothecin is a highly non-polar molecule. In a highly non-polar adsorption medium (chloroform-ethanol), the drug does not preferentially leave the adsorption medium to adsorb to the carbon. However, in a more polar adsorption medium, it is believed that adsorption to the carrier particles would be entirely acceptable. One of the factors that influence the adsorption of the drug in the adsorption medium to the carbon in the carrier particle is the hydrophobic Van der Waals interactions between the drug and the particles. Alternatively, the drug can be dried onto the particles by evaporation techniques similar to those used for adsorption of PAC.”

“The carrier particles used for adsorption of paclitaxel (PAC) have an iron:carbon content of 70:30. The carbon is activated carbon type E. To analytically determine the iron content the following procedure was used. A portion of the sample was weighed (previously dried in a vacuum desiccator) and washed at 1000° C., oxidizing all carbon and iron present. During this procedure carbon was converted quantitatively to CO2 and volatilized, leaving a residue of Fe2 O3. The iron content was calculated by the formula. Fe=Fe2 O3/1.42977, assuming no Fe2 O3 was present initially. Carbon was assumed to be the remaining fraction. A second analysis of another portion of the sample was performed on a LECO carbon combustion analyzer. The sample was combusted and the CO₂ then measured, and total carbon was calculated. Iron and carbon content calculated by both methods gave comparable results of about 69% by weight of elemental iron. A. Binding properties of Paclitaxel to composite particles”

“Drug adsorption was measured in two ways: 1) Initially a UV spectrophotometric assay was developed for screening drug bound to a variety of activated carbons. HPLC or spectrophotometric grade solvents were used throughout. The .lambda.max in ethanol was determined to be 220 nm. A Milton Roy Spectronic 21 spectrophotometer was used with 3 mL quartz cells. The wavelength of 254 nm was selected for UV analysis because it provided good sensitivity for the drug. Little or no contamination from various assay techniques or materials was found at that wavelength. The same wavelength was used for the HPLC analysis. The UV assay was linear for paclitaxel over the range 0.05-3.0 mg/mL.”

“In one test the carrier particles contained the KB-type carbon. It has a small pore size (˜40 nm effective radius), >1000 m2/grn surface areas, and good hardness. PAC adsorption capacity however was limited. A survey of some 20 other candidate activated carbons was reduced to three types with promising drug delivery properties, A, B, and E types of carbon. Iron powder alone was also tested. Each of these materials was used at a concentration of 30 mg in citrated ethanol. The analysis by UV methods gave the following binding results for 3 mg of PAC. Type A carbon—74%, Type B carbon=65%, Type E carbon=33%, and iron powder=0% (no binding) Types A and B carbon are both large pore, large surface area (>=1,800 m2/gm) carbons with drug release characteristics equivalent to the E-type. E-type is a much harder carbon with a smaller surface area and consequently better milling properties. B. Paclitaxel Binding to Different Activated Carbons.”

At column 14 of U.S. Pat. No. 6,200,547, a discussion was presented of the binding affinity of paclitaxel to different types of activated carbons. It was disclosed (at lines 47 et seq.) that “fractional binding (fb) (amount bound of initial amount of PAC) to activated carbon types A, B, and E increased with increasing amount of carbon (at fixed PAC concentration). Types A and B carbon could be shown to bind PAC 100% and to plateau in the binding curve at high activated carbon content. Fractional bind of Type E was only 68%. The binding capacity, Q (expressed as % weight/weight drug carrier) was shown to decrease with an increase in the amount of activated carbon. For type A carbon, the binding capacity, Q, increased from 8% to 44% for a decrease in carbon from 40 mg to 5 mg. The corresponding Q value for AC type E was about 5% to 7%.”

“Other studies of drug binding to type A carbon have suggested that a plateau in the fraction of drug bound as a function of the amount of absorber is a result of multilaminar drug coating on the surface of the carrier. In contrast, a linear increase in fraction bound is indicative of unilaminar coating, thus in keeping with the rules of the Langmuir isotherm analysis.”

“Our studies showed that Types A and E carbon have the ability to adsorb a considerable fraction (fb) of PAC in the adsorption medium and that their binding capacity, Q, is also significant. On the other hand, carrier particles having a iron:carbon ratio of 70:30 (type E carbon) had both reduced capacity and fractional binding. These reduced values are in keeping with the proportionally lower carbon content of the carrier particles as compared with carbon alone. In contrast, both the fb and Q values for the carrier particles with a higher binding capacity type A carbon were less than 2%. This may be due to the inability of the pores in the carbon to withstand the compressive forces of the attrition milling process during manufacture. Despite the extensive binding of activated carbon Types A and B to PAC, use of Type E carbon in carrier particles was preferred due to commercial availability, and the proper balance between binding and release properties. In addition, Type E carbon is the preferred activated carbon for use in a drug carrier because it has been established to have U.S. Pharmacopoeia (22nd edition) quality. FIG. 6 shows Langmuir adsorption plots for PAC binding to (--.largecircle.--) carrier particles with an iron:carbon ratio of 70%:30% Type E carbon and (--.quadrature.--) Type E carbon alone. Data were fit by simple unweighted linear regression. Affinity (Km) and maximal binding (Qm) constants for PAC to the carrier particles having an iron:carbon ratio of 70:30 (Type E carbon) were determined over a range of carrier amounts. Table 4 below shows the results of adsorption isotherms of these compositions. The values were determined graphically from FIG. 6 and Langmuir's equation.’

At column 16 of U.S. Pat. No. 6,200,547, and in summarizing the results obtained in the experiments of Example 4, the patentees concluded that: “These results demonstrated that pharmacologically active paclitaxel can be released from the carrier particles of the invention, and that the chemical analysis of adsorbed and released drug can be confirmed biologically. Similar dose-response curves were obtained for free paclitaxel and paclitaxel desorbed from the carrier particles.”

One may use “ . . . pharmacogically active palitaxel . . . ” adsorbed on “ . . . the carrier particles of the invention . . . . ”

By way of further illustrtion, one may use the magnetically controllable ferrocarbon particle compositions of U.S. Pat. No. 6,482,436 to deliver paclitaxel to an implanted medical device; the entire disclosure of this United States patent is hereby incorporated by reference into this specification.

Claim 1 of U.S. Pat. No. 6,482,436 describes: “A magnetically responsive composition comprising particles including carbon and iron, wherein the carbon is substantially uniformly distributed throughout the particle volume, wherein the cross-sectional size of each particle is less than about 5 μm, and wherein the carbon is selected from the group consisting of types A, B, E, K, KB, and chemically modified versions thereof.”

In column 1 of U.S. Pat. No. 6,482,436, reference is made to “prior art” carrier compositions onto which a therapeutic agent is adsorbed. Thus, as is disclosed at lines 26 et seq. of column 1 of such patent, “Metallic carrier compositions used in the treatment of various disorders have been heretofore suggested and/or utilized (see, for example, U.S. Pat. Nos. 4,849,209 and 4,106,488), and have included such compositions that are guided or controlled in a body in response to external application of a magnetic field (see, for example, U.S. Pat. Nos. 4,501,726, 4,652,257 and 4,690,130). Such compositions have not always proven practical and/or entirely effective. For example, such compositions may lack adequate capacity for carriage of the desired biologically active agent to the treatment site, have less than desirable magnetic susceptibility and/or be difficult to manufacture, store and/or use.”

“One such known composition, deliverable by way of intravascular injection, includes microspheres made up of a ferromagnetic component covered with a biocompatible polymer (albumin, gelatin, and polysaccharides) which also contains a drug (Driscol C. F. et al. Prog. Am. Assoc. Cancer Res., 1980, p. 261).”

“It is possible to produce albumen microspheres up to 3.0 μm in size containing a magnetic material (magnetite Fe3 O4) and the anti-tumoral antibiotic doxorubicin (Widder K. et al. J. Pharm. Sci., 68:79-82 1979). Such microspheres are produced through thermal and/or chemical denaturation of albumin in an emulsion (water in oil), with the input phase containing a magnetite suspension in a medicinal solution. Similar technique has been used to produce magnetically controlled, or guided, microcapsules covered with ethylcellulose containing the antibiotic mitomycin-C (Fujimoto S. et al., Cancer, 56: 2404-2410,1985).”

U.S. Pat. No. 6,482,436 discloses that even biologically active substances that are substantially insoluble inwater can be adsorbed ontothe carrier particles of this patent. As is disclosed in such column 6, commencing at line 29 thereof, “However, adsorption of biologically active substances that are substantially insoluble in water (i.e., with solubility in water less than about 0.1% by weight) requires use of special procedures to adsorb a useful amount of a drug on the particles. Applicants have discovered that adsorption on the carrier particles of this invention of biologically active substances having substantial insolubility in water can be obtained without the use of surfactants, many of which are toxic, by dissolving the water insoluble biologically active substance in a liquid adsorption medium (e.g., aqueous) that includes excipients selected to minimize the hydrophobic Van der Waals forces between the particles and the solution and to prevent agglomeration of the particles in the medium. For example, if the biologically active substance is a highly non-polar molecule, such as camptothecin, and the adsorption medium is a highly non-polar liquid, such as chloroform-ethanol, the drug does not preferentially leave the adsorption medium to adsorb to the carbon. However, in a more polar adsorption medium, adsorption to the carrier particles is entirely acceptable. For example, binding of therapeutic levels of paclitaxel, a highly water-insoluble drug, to carrier particles having an iron:carbon ratio of 70:30 was obtained using citrated ethanol as the adsorption medium, even though paclitaxel is substantially water insoluble. In many cases, it is advantageous if the liquid adsorption medium includes a biologically compatible and biodegradable viscosity-increasing agent (e.g., a biologically compatible polymer), such as sodium carboxymethyl cellulose, to aid in separation of the particles in the medium.”

In Example 5 of this patent (see column 15), an experiment was described in which paclitaxel was absorbed onto carrier particles having an iron/carbon ratio of 70/30. As was disclosed insuch column 15, “The carrier particles used for adsorption of paclitaxel (PAC) have an iron:carbon content of 70:30. The carbon is activated carbon type E. To analytically determine the iron content the following procedure was used. A portion of the sample was weighed (previously dried in a vacuum desiccator) and washed at 2000° C., oxidizing all carbon and iron present. During this procedure carbon was converted quantitatively to CO₂ and volatilized, leaving a residue of Fe2 O3. The iron content was calculated by the formula. Fe=Fe2 O3/1.42977, assuming no Fe2 O3 was present initially. Carbon was assumed to be the remaining fraction. A second analysis of another portion of the sample was performed on a LECO carbon combustion analyzer. The sample was combusted and the CO2 then measured, and total carbon was calculated. Iron and carbon content calculated by both methods gave comparable results of about 69% by weight of elemental iron.”

The use of Externally Applied Energy to Affect an Implanted Medical Device

The prior art discloses many devices in which an externally applied electromagnetic field (i.e., a field originating outside of a biological organism, such as a human body) is generated in order to influence one or more implantable devices disposed within the biological organism. Some of these devices are described below; they may be used in the processes and apparatuses of the instant invention.

U.S. Pat. No. 3,337,776 describes a device for producing controllable low frequency magnetic fields; the entire disclosure of this patent is hereby incorporated by reference into this specification. Thus, e.g., claim 1 of this patent describes a biomedical apparatus for the treatement of a subject with controllable low frequency magnetic fields, comprising solenoid mens for creating the magnetic field.

U.S. Pat. No. 3,890,953 also discloses an apparatus for promoting the growth of bone and other body tissues by the application of a low frequency alternating magnetic field; the entire disclosure of this United States patent is hereby incorporated by reference into this specification. This patent claims “In an electrical apparatus for promoting the growth of bone and other body tissues by the application thereto of a low frequency alternating magnetic field, such apparatus having current generating means and field applicator means, the improvement wherein the applicator means comprises a flat solenoid coil having an axis about which the coil is wound and composed of a plurality of parallel and flexible windings, each said winding having two adjacent elongate portions and two 180° coil bends joining said elongate portions together, said coil being flexible in the coil plane in the region of said elongate portion for being bent into a U-shape, said coil being bent into such U-shape about an axis parallel to the coil axis and adapted for connection to a source of low frequency alternating current.”

The device of U.S. Pat. No. 3,890,953 is described, in part, at lines 52 et seq. of column 2, wherein it is disclosed that: “ . . . The apparatus shown diagrammatically in FIG. 1 comprises a AC generator 10, which supplies low frequency AC at the output terminals 12. The frequency of the AC lies below 150 Hz, for instance between 1 and 50 or 65 Hz. It has been found particularly favorable to use a frequency range between 5 or 10 and 30 Hz, for example 25 Hz. The half cycles of the alternating current should have comparatively gently sloping leading and trailing flanks (rise and fall times of the half cycles being for example in the order of magnitude of a quarter to an eighth of the length of a cycle); the AC can thus be a sinusoidal current with a low non-linear distortion, for example less than 20 percent, or preferably less than 10 percent, or a triangular wave current.”

U.S. Pat. No. 4,095,588 discloses a “vascular cleansing device” adapted to “ . . . effect motion of thered corpuscles in the blood stream of a vascular system . . . wherey these red cells may cleanse the vascular system by scrubbing the walls thereof . . . ;” the entire disclosure of this United States patent is hereby incorporated by reference into this specification. This patent claims (in claim 3) “A means to propel a red corpuscle in a vibratory and rotary fashion, said means comprising an electronic circuit and magnetic means including: a source of electrical energy; a variable oscillator connected to said source; a binary counter means connected to said oscillator to produce sequential outputs; a plurality of deflection amplifier means connected to be operable by the outputs of said binary counter means in a sequential manner, said amplifier means thereby controlling electrical energy from said source; a plurality of separate coils connected in separate pairs about an axis in series between said deflection amplifier means and said source so as to besequentially operated in creating an electromagnetic field from one coil to the other and back again and thence to adjacent separate coils for rotation of the electromagnetic field from one pair of coils to another; and a table within the space encircled by said plurality of coils, said table being located so as to place a person along the axis such that the red corpuscles of the person's vascular system are within the electromagnetic field between the coils creating same.”

U.S. Pat. No. 4,323,075 discloses an implantable defibrillator with a rechargeable power supply; the entire disclosure of this patent is herebyh incorporated by reference into this specification. Claim 1 of this patent describes “A fully implantable power supply for use in a fully implantable defibrillator having an implantable housing, a fibrillation detector for detecting fibrillation of the heart of a recipient, an energy storage and discharge device for storing and releasing defibrillation energy into the heart of the recipient and an inverter for charging the energy storage and discharge device in response to detection of fibrillation by the fibrillation detector, the inverter requiring a first level of power to be operational and the fibrillation detector requiring a second level of power different from said first level of power to be operational, said power supply comprising: implantable battery means positioned within said implantable housing, said battery means including a plurality of batteries arranged in series, each of said batteries having a pair of output terminals, each of said batteries producing a distinctly multilevel voltage across its pair of output terminals, said voltage being at a first level when the battery is fully charged and dropping to a second level at some point during the discharge of the battery; and implantable circuit means positioned within said implantable housing, said circuit means for creating a first conductive path betwen said serially-connected batteries and said fibrillation detector to provide said fibrillation detector with said second level of power, and for creating a second conductive path between said inverter and said battery means by placing only the batteries operating at said first level voltage in said second conductive path, and excluding the remaining batteries from said second conductive path to provide said inverter with said first level of power.”

U.S. Pat. No. 4,340,038 discloses an implanted medical system comprised of magnetic field pick-up means for converting magnetic energy to electrical energy; the entire disclosure of this patentis hereby incorporated by reference into this specification.

In column 1 of U.S. Pat. No. 4,340,038, at lines 12 et seq., it is disclosed that “Many types of implantable devices incorporate a self-contained transducer for converting magnetic energy from an externally-located magnetic field generator to energy usable by the implanted device. In such a system having an implanted device and an externally-located magnetic field generator for powering the device, sizing and design of the power transfer system is important. In order to properly design the power transfer system while at the same time avoiding overdesign, the distance from the implanted device to the magnetic field generator must be known. However for some types of implanted devices the depth of the implanted device in a recipient's body is variable, and is not known until the time of implantation by a surgeon. One example of such a device is an intracranial pressure monitoring device (ICPM) wherein skull thickness varies considerably between recipients and the device must be located so that it protrudes slightly below the inner surface of the skull and contacts the dura, thereby resulting in a variable distance between the top of the implanted device containing a pick-up coil or transducer and the outer surface of the skull. One conventional technique for accommodating an unknown distance between the magnetic field generator and the implanted device includes increasing the transmission power of the external magnetic field generator. However this increased power can result in heating of the implanted device, the excess heat being potentially hazardous to the recipient. A further technique has been to increase the diameter of the pick-up coil in the implanted device. However, physical size constraints imposed on many implanted devices such as the ICPM are critical; and increasing the diameter of the pick-up coil is undesirable in that it increases the size of the orifice which must be formed in the recipient's skull. The concentrator of the present invention solves the above problems by concentrating magnetic lines of flux from the magnetic generator at the implanted pick-up coil, the concentrator being adapted to accommodate distance variations between the implanted device and the magnetic field generator.’

Claim 1 of U.S. Pat. No. 4,340,038 describes “In a system including an implanted device having a magnetic field pick-up means for converting magnetic energy to electrical energy for energizing said implanted device, and an external magnetic field generator located so that magnetic lines of flux generated thereby intersect said pick-up means, a means for concentrating a portion of said magnetic lines of flux at said pick-up means comprising a metallic slug located between said generator and said pick-up means, thereby concentrating said magnetic lines of flux at said pick-up means. ” Claim 5 of this patent further describes the pick-up means as comprising “ . . . a magnetic pick-up coil and said slug is formed in the shape of a truncated cone and oriented so that a plane defined by the smaller of said cone end surfaces is adjacent to said substantially parallel to a plane defined by said magnetic pick-up coil.”

U.S. Pat. No. 4,361,153 discloses an implantable telemetry system; the entire disclosure of such United States patent is hereby incorporated by reference into this specification.

As is disclosed at column 1 of U.S. Pat. No. 4,361,153 (see lines 9 et seq.), “Externally applied oscillating magnetic fields have been used before with implanted devices. Early inductive cardiac pacers employed externally generated electromagnetic energy directly as a power source. A coil inside the implant operated as a secondary transformer winding and was interconnected with the stimulating electrodes. More recently, implanted stimulators with rechargeable (e.g., nickel cadmium) batteries have used magnetic transmission to couple energy into a secondary winding in the implant to energize a recharging circuit having suitable rectifier circuitry. Miniature reed switches have been utilized before for implant communications. They appear to have been first used to allow the patient to convert from standby or demand mode to fixed rate pacing with an external magnet. Later, with the advent of programmable stimulators, reed switches were rapidly cycled by magnetic pulse transmission to operate pulse parameter selection circuitry inside the implant. Systems analogous to conventional two-way radio frequency (RF) and optical communication system have also been proposed. The increasing versatility of implanted stimulators demands more complex programming capabilities. While various systems for transmitting data into the implant have been proposed, there is a parallel need to develop compatible telemetry systems for signalling out of the implant. However, the austere energy budget constraints imposed by long life, battery operated implants rule out conventional transmitters and analogous systems”

The solution provided by U.S. Pat. No. 4,361,153 is “ . . . achieved by the use of a resonant impedance modulated transponder in the implant to modulate thephase of a relatively high energy reflected magnetic carrier imposed from outside of the body.” In particular, and as is described by claim 1 of this patent, there is claimed “An apparatus for communicating variable information to an external device from an electronic stimulator implanted in a living human patient, comprising an external unit including means for transmitting a carrier signal, a hermetically sealed fully implantable enclosure adapted to be implanted at a fixed location in the patient's body, means within said enclosure for generating stimulator outputs, a transponder within said enclosure including tuned resonant circuit means for resonating at the frequency of said carrier signal so as to re-radiate a signal at the frequency of said carrier signal, and means for superimposing an information signal on the reflected signal by altering the resonance of said tuned circuit means in accordance with an information signal, said superimposing means including a variable impedance load connected across said tuned circuit and means for varying the impedance of said load in accordance with an information signal, said external unit further including pickup means for receiving the reflected signal from said transponder and means for recovering the information signal superimposed thereon, said receiving means including means reponsive to said reflected signal from said transponder for producing on associated analog output signal, and said recovering means including phase shift detector means responsive to said analog output signal for producing an output signal related to the relative phase angle thereof.”

U.S. Pat. No. 4,408,607 discloses a rechargeable, implantable capacitive energy source; the entire disclosure of this patent is hereby incorporated into this specification by reference. As is disclosed in column 1 of such patent (at lines 12 et seq.), “Medical science has advanced to the point where it is possible to implant directly within living bodies electrical devices necessary or advantageous to the welfare of individual patients. A problem with such devices is how to supply the electrical energy necessary for their continued operation. The devices are, of course, designed to require a minimum of electrical energy, so that extended operation from batteries may be possible. Lithium batteries and other primary, non-rechargeable cells may be used, but they are expensive and require replacement of surgical procedures. Nickel-cadmium and other rechargeable batteries are also available, but have limited charge-recharge characteristics, require long intervals for recharging, and release gas during the charging process.”

The solution to this problem is described, e.g., in claim 1 of the patent, which describes “An electric power supply for providing electrical energy to an electrically operated medical device comprising: capacitor means for accommodating an electric charge; first means providing a regulated source of unidirectional electrical energy; second means connecting said first means to said capacitor means for supplying charging current to said capacitor means at a first voltage which increases with charge in the capacitor means; third means deriving from said first means a comparison second voltage of constant magnitude; comparator means operative when said first voltage reaches a first value to reduce said first voltage to a second, lower value; and voltage regulator means connected to said capacitor means and medical device to limit the voltage supplied to the medical device.”

U.S. Pat. No. 4,416,283 discloses a implantable shunted coil telemetry transponder employed as a magnetic pulse transducer for receiving externally transmitted data; the entire disclosure of this United States patent is hereby incorporated by reference into this specification.

In particular, a programming system for a biomedical implant is described in claim 1 of U.S. Pat. No. 4,416,283. Such claim 1 discloses “In a programming system for a biomedical implant of the type wherein an external programmer produces a series of magnetic impulses which are received and transduced to form a corresponding electrical pulse input to programmable parameter data registers inside the implant, wherein the improvement comprises external programming pulse receiving and transducing circuitry in the implant including a tuned coil, means responsive to pairs of successive voltage spikes of opposite polarity magnetically induced across said tuned coil by said magnetic impulses for forming corresponding binary pulses duplicating said externally generated magnetic impulses giving rise to said spikes, and means for outputting said binary pulses to said data registers to accomplish programming of the implant.”

U.S. Pat. No. 4,871,351 discloses an implantale pump infusion system; the entire disclosure of this United States patent is hereby incorporated by reference into this specification. These implantable pumps are disussed in column 1 of the patent, wherein it is disclosed that: “Certain human disorders, such as diabetes, require the injection into the body of prescribed amounts of medication at prescribed times or in response to particular conditions or events. Various kinds of infusion pumps have been propounded for infusing drugs or other chemicals or solutions into the body at continuous rates or measured dosages. Examples of such known infusion pumps and dispensing devices are found in U.S. Pat. Nos. 3,731,861; 3,692,027; 3,923,060; 4,003,379; 3,951,147; 4,193,397; 4,221,219 and 4,258,711. Some of the known pumps are external and inject the drugs or other medication into the body via a catheter, but the preferred pumps are those which are fully implantable in the human body.”

“Implantable pumps have been used in infusion systems such as those disclosed in U.S. Pat. Nos. 4,077,405; 4,282,872; 4,270,532; 4,360,019 and 4,373,527. Such infusion systems are of the open loop type. That is, the systems are pre-programmed to deliver a desired rate of infusion. The rate of infusion may be programmed to vary with time and the particular patient. A major disadvantage of such open loop systems is that they are not responsive to the current condition of the patient, i.e. they do not have feedback information. Thus, an infusion system of the open loop type may continue dispensing medication according to its pre-programmed rate or profile when, in fact, it may not be needed.”

“There are known closed loop infusion systems which are designed to control a particular condition of the body, e.g. the blood glucose concentration. Such systems use feedback control continuously, i.e. the patient's blood is withdrawn via an intravenous catheter and analysed continuously and a computer output signal is derived from the actual blood glucose concentration to drive a pump which infuses insulin at a rate corresponding to the signal. The known closed loop systems suffer from several disadvantages. First, since they monitor the blood glucose concentration continuously they are complex and relatively bulky systems external to the patient, and restrict the movement of the patient. Such systems are suitable only for hospital bedside applications for short periods of time and require highly trained operating staff. Further, some of the known closed loop systems do not allow for manually input overriding commands. Examples of closed loop systems are found in U.S. Pat. Nos. 4,055,175; 4,151,845 and 4,245,634.”

“An implanted closed loop system with some degree of external control is disclosed in U.S. Pat. No. 4,146,029. In that system, a sensor (either implanted or external) is arranged on the body to sense some kind of physiological, chemical, electrical or other condition at a particular site and produced data which corresponds to the sensed condition at the sensed site. This data is fed directly to an implanted microprocessor controlled medication dispensing device. A predetermined amount of medication is dispensed in response to the sensed condition according to a pre-programmed algorithm in the microprocessor control unit. An extra-corporeal coding pulse transmitter is provided for selecting between different algorithms in the microprocessor control unit. The system of U.S. Pat. No. 4,146,029 is suitable for use in treating only certain ailments such as cardiac conditions. It is unsuitable as a blood glucose control system for example, since (i) it is not practicable to measure the blood glucose concentration continuously with an implanted sensor and (ii) the known system is incapable of dispensing discrete doses of insulin in response to certain events, such as meals and exercise. Furthermore, there are several disadvantages to internal sensors; namely, due to drift, lack of regular calibration and limited life, internal sensors do not have high long-term reliability. If an external sensor is used with the system of U.S. Pat. No. 4,146,029, the output of the sensor must be fed through the patient's skin to the implanted mechanism. There are inherent disadvantages to such a system, namely the high risk of infection. Since the algorithms which control the rate of infusion are programmed into the implanted unit, it is not possible to upgrade these algorithms without surgery. The extra-corporeal controller merely selects a particular one of several medication programs but cannot actually alter a program.”

“It is an object of the present invention to overcome, or substantially ameliorate the above described disadvantages of the prior art by providing an implantable open loop medication infusion system with a feedback control option”

The solution to this problem is set forth in claim 1 of U.S. Pat. No. 4,871,351, which describes: “A medical infusion system intermittently switchable at selected times between an open loop system without feedback and a closed loop system with feedback, said system comprising an implantable unit including means for controllably dispensing medication into a body, an external controller, and an extra-corporeal sensor; wherein said implantable unit comprises an implantable transceiver means for communicating with a similar external transceiver means in said external controller to provide a telemetry link between said controller and said implantable unit, a first reservoir means for holding medication liquid, a liquid dispensing device, a pump connected between said reservoir means and said liquid dispensing device, and a first electronic control circuit means connected to said implantable transceiver means and to said pump to operate said pump; wherein said external controller comprises a second electronic control circuit means connected with said external transceiver means, a transducer means for reading said sensor, said transducer means having an output connected to said second electronic control circuit means, and a manually operable electric input device connected to said second electronic control circuit means; wherein said pump is operable by said first electonic control circuit means to pump said medication liquid from said first reservoir means to said liquid-dispensing deive at a first predetermined rate independent of the output of said extra-corporeal sensor, and wherein said input device or said transducer means include means which selectively operable at intermittent times to respectively convey commands or output of said transducer representing the reading of said sensor to said second control circuit to instruct said first control circuit via said telemetry link to modify the operation of said pump.”

U.S. Pat. No. 4,941,461 describes an electrically actuated inflatable penile erecton device comprised of an implantable induction coil and an implantable pump; the entire disclosure of this United States patent is hereby incorporated by reference into this specification. The device of this patent is described, e.g., in claim 1 of the patent, which discloses “An apparatus for achieving a penile erection in a human male, comprising: at least one elastomer cylinder having a root chamber and a pendulous chamber, said elastomer cylinder adapted to be placed in the corpus carvenosum of the penis; an external magnetic field generator which can be placed over some section of the penis which generates an alternating magnetic field; an induction coil contained within said elastomer cylinder which produces an alternating electric current when in the proximity of said alternating magnetic filed which is produced by said external magnetic field generator; and a fluid pumping means located within said elastomer cylinder, said pumping means being operated by the electrical power generated in said induction coil to pump fluid from said root chamber to said pendulous chamber in order to stiffen said elastomer cylinder for causing the erect state of the penis.”

U.S. Pat. No. 5,487,760 discloses an implantable signal transceiver disposed in an artificial heart valve; the entire disclosure of this United States patent is hereby incorporated by reference into this specification. Claim 1 of this patent describes: “In combination, an artificial heart valve of the type having a tubular body member, defining a lumen and pivotally supporting at least one occluder, said body member having a sewing cuff covering an exterior surface of said body member; and an electronic sensor module disposed between said sewing cuff and said exterior surface, wherein said sensor module incorporates a sensor element for detecting movement of said at least one occluder between an open and a closed disposition relative to said lumen and wherein said sensor module further includes a signal transceiver coupled to said sensor element, and means for energizing said signal transceiver, and wherein said sensor module includes means for encapsulating said sensor element, signal transceiver and energizing means in a moisture-impervious container.”

U.S. Pat. No. 5,702,430 discloses an implantable power supply; the entire disclosure of such patent is hereby incorporated by reference into this specification. Claim 1 of such patent describes: “A surgically implantable power supply comprising battery means for providing a source of power, charging means for charging the battery means, enclosure means isolating the battery means from the human body, gas holding means within the enclosure means for holding gas generated by the battery means during charging, seal means in the enclosure means arranged to rapture when the internal gas pressure exceeds a certain value and inflatable gas container means outside the enclosure means to receive gas from within the enclosure means when the seal means has been ruptured.”

Columns 1 through 5 of U.S. Pat. No. 5,702,430 presents an excellent discussion of “prior art” implantable pump assemblies. As is disclosed in such portion of U.S. Pat. No. 5,702,430, “The most widely tested and commonly used implantable blood pumps employ variable forms of flexible sacks (also spelled sacs) or diaphragms which are squeezed and released in a cyclical manner to cause pulsatile ejection of blood. Such pumps are discussed in books or articles such as Hogness and Antwerp 1991, DeVries et al 1984, and Farrar et al 1988, and in U.S. Pat. No. 4,994,078 (Jarvik 1991), U.S. Pat. No. 4,704,120 (Slonina 1987), U.S. Pat. No. 4,936,758 (Coble 1990), and U.S. Pat. No. 4,969,864 (Schwarzmann et al 1990). Sack or diaphragm pumps are subject to fatigue failure of compliant elements and as such are mechanically and functionally quite different from the pump which is the subject of the present invention.”

“An entirely different class of implantable blood pumps uses rotary pumping mechanisms. Most rotary pumps can be classified into two categories: centrifugal pumps and axial pumps. Centrifugal pumps, which include pumps marketed by Sarns (a subsidiary of the 3M Company) and Biomedicus (a subsidiary of Medtronic, Eden Prairie, Minn.), direct blood into a chamber, against a spinning interior wall (which is a smooth disk in the Medtronic pump). A flow channel is provided so that the centrifugal force exerted on the blood generates flow.”

“By contrast, axial pumps provide blood flow along a cylindrical axis, which is in a straight (or nearly straight) line with the direction of the inflow and outflow. Depending on the pumping mechanism used inside an axial pump, this can in some cases reduce the shearing effects of the rapid acceleration and deceleration forces generated in centrifugal pumps. However, the mechanisms used by axial pumps can inflict other types of stress and damage on blood cells.”

“Some types of axial rotary pumps use impeller blades mounted on a center axle, which is mounted inside a tubular conduit. As the blade assembly spins, it functions like a fan, or an outboard motor propeller. As used herein, “impeller” refers to angled vanes (also called blades) which are constrained inside a flow conduit; an impeller imparts force to a fluid that flows through the conduit which encloses the impeller. By contrast, “propeller” usually refers to non-enclosed devices, which typically are used to propel vehicles such as boats or airplanes.”

“Another type of axial blood pump, called the “Haemopump” (sold by Nimbus) uses a screw-type impeller with a classic screw (also called an Archimedes screw; also called a helifoil, due to its helical shape and thin cross-section). Instead of using several relatively small vanes, the Haemopump screw-type impeller contains a single elongated helix, comparable to an auger used for drilling or digging holes. In screw-type axial pumps, the screw spins at very high speed (up to about 10,000 rpm). The entire Haemopump unit is usually less than a centimeter in diameter. The pump can be passed through a peripheral artery into the aorta, through the aortic valve, and into the left ventricle. It is powered by an external motor and drive unit.”

“Centrifugal or axial pumps are commonly used in three situations: (1) for brief support during cardio-pulmonary operations, (2) for short-term support while awaiting recovery of the heart from surgery, or (3) as a bridge to keep a patient alive while awaiting heart transplantation. However, rotary pumps generally are not well tolerated for any prolonged period. Patients who must rely on these units for a substantial length of time often suffer from strokes, renal (kidney) failure, and other organ dysfunction. This is due to the fact that rotary devices, which must operate at relatively high speeds, may impose unacceptably high levels of turbulent and laminar shear forces on blood cells. These forces can damage or lyse (break apart) red blood cells. A low blood count (anemia) may result, and the disgorged contents of lysed blood cells (which include large quantities of hemoglobin) can cause renal failure and lead to platelet activation that can cause embolisms and stroke.”

“One of the most important problems in axial rotary pumps in the prior art involves the gaps that exist between the outer edges of the blades, and the walls of the flow conduit. These gaps are the site of severe turbulence and shear stresses, due to two factors. Since implantable axial pumps operate at very high speed, the outer edges of the blades move extremely fast and generate high levels of shear and turbulence. In addition, the gap between the blades and the wall is usually kept as small as possible to increase pumping efficiency and to reduce the number of cells that become entrained in the gap area. This can lead to high-speed compression of blood cells as they are caught in a narrow gap between the stationary interior wall of the conduit and the rapidly moving tips or edges of the blades.”

“An important factor that needs to be considered in the design and use of implantable blood pumps is “residual cardiac function,” which is present in the overwhelming majority of patients who would be candidates for mechanical circulatory assistance. The patient's heart is still present and still beating, even though, in patients who need mechanical pumping assistance, its output is not adequate for the patient's needs. In many patients, residual cardiac functioning often approaches the level of adequacy required to support the body, as evidenced by the fact that the patient is still alive when implantation of an artificial pump must be considered and decided. If cardiac function drops to a level of severe inadequacy, death quickly becomes imminent, and the need for immediate intervention to avert death becomes acute.’

‘Most conventional ventricular assist devices are designed to assume complete circulatory responsibilities for the ventricle they are “assisting.” As such, there is no need, nor presumably any advantage, for the device to interact in harmony with the assisted ventricle. Typically, these devices utilize a “fill-to-empty” mode that, for the most part, results in emptying of the device in random association with native heart contraction. This type of interaction between the device and assisted ventricle ignores the fact that the overwhelming majority of patients who would be candidates for mechanical assistance have at least some significant residual cardiac function.’

‘It is preferable to allow the natural heart, no matter how badly damaged or diseased it may be, to continue contributing to the required cardiac output whenever possible so that ventricular hemodynamics are disturbed as little as possible. This points away from the use of total cardiac replacements and suggests the use of “assist” devices whenever possible. However, the use of assist devices also poses a very difficult problem: in patients suffering from severe heart disease, temporary or intermittent crises often require artificial pumps to provide “bridging” support which is sufficient to entirely replace ventricular pumping capacity for limited periods of time, such as in the hours or days following a heart attack or cardiac arrest, or during periods of severe tachycardia or fibrillation.’

‘Accordingly, an important goal during development of the described method of pump implantation and use and of the surgically implantable reciprocating pump was to design a method and a device which could cover a wide spectrum of requirements by providing two different and distinct functions. First, an ideal cardiac pumping device should be able to provide “total” or “complete” pumping support which can keep the patient alive for brief or even prolonged periods, if the patient's heart suffers from a period of total failure or severe inadequacy. Second, in addition to being able to provide total pumping support for the body during brief periods, the pump should also be able to provide a limited “assist” function. It should be able to interact with a beating heart in a cooperative manner, with minimal disruption of the blood flow generated by the natural heartbeat. If a ventricle is still functional and able to contribute to cardiac output, as is the case in the overwhelming majority of clinical applications, then the pump will assist or augment the residual cardiac output. This allows it to take advantage of the natural, non-hemolytic pumping action of the heart to the fullest extent possible; it minimizes red blood cell lysis, it reduces mechanical stress on the pump, and it allows longer pump life and longer battery life.”

“Several types of surgically implantable blood pumps containing a piston-like member have been developed to provide a mechanical device for augmenting or even totally replacing the blood pumping action of a damaged or diseased mammalian heart.”

“U.S. Pat. No. 3,842,440 to Karlson discloses an implantable linear motor prosthetic heart and control system containing a pump having a piston-like member which is reciprocal within a magnetic field. The piston-like member includes a compressible chamber in the prosthetic heart which communicates with the vein or aorta.”

“U.S. Pat. Nos. 3,911,897 and 3,911,898 to Leachman, Jr. disclose heart assist devices controlled in the normal mode of operation to copulsate and counterpulsate with the heart, respectively, and produce a blood flow waveform corresponding to the blood flow waveform of the heart being assisted. The heart assist device is a pump connected serially between the discharge of a heart ventricle and the vascular system. The pump may be connected to the aorta between the left ventricle discharge immediately adjacent the aortic valve and a ligation in the aorta a short distance from the discharge. This pump has coaxially aligned cylindrical inlet and discharge pumping chambers of the same diameter and a reciprocating piston in one chamber fixedly connected with a reciprocating piston of the other chamber. The piston pump further includes a passageway leading between the inlet and discharge chambers and a check valve in the passageway preventing flow from the discharge chamber into the inlet chamber. There is no flow through the movable element of the piston.”

“U.S. Pat. No. 4,102,610 to Taboada et al. discloses a magnetically operated constant volume reciprocating pump which can be used as a surgically implantable heart pump or assist. The reciprocating member is a piston carrying a tilting-disk type check valve positioned in a cylinder. While a tilting disk valve results in less turbulence and applied shear to surrounding fluid than a squeezed flexible sack or rotating impeller, the shear applied may still be sufficiently excessive so as to cause damage to red blood cells.”

“U.S. Pat. Nos. 4,210,409 and 4,375,941 to Child disclose a pump used to assist pumping action of the heart having a piston movable in a cylindrical casing in response to magnetic forces. A tilting-disk type check valve carried by the piston provides for flow of fluid into the cylindrical casing and restricts reverse flow. A plurality of longitudinal vanes integral with the inner wall of the cylindrical casing allow for limited reverse movement of blood around the piston which may result in compression and additional shearing of red blood cells. A second fixed valve is present in the inlet of the valve to prevent reversal of flow during piston reversal.”

“U.S. Pat. No. 4,965,864 to Roth discloses a linear motor using multiple coils and a reciprocating element containing permanent magnets which is driven by microprocessor-controlled power semiconductors. A plurality of permanent magnets is mounted on the reciprocating member. This design does not provide for self-synchronization of the linear motor in the event the stroke of the linear motor is greater than twice the pole pitch on the reciprocating element. During start-up of the motor, or if magnetic coupling is lost, the reciprocating element may slip from its synchronous position by any multiple of two times the pole pitch. As a result, a sensing arrangement must be included in the design to detect the position of the piston so that the controller will not drive it into one end of the closed cylinder. In addition, this design having equal pole pitch and slot pitch results in a “jumpy” motion of the reciprocating element along its stroke.”

“In addition to the piston position sensing arrangement discussed above, the Roth design may also include a temperature sensor and a pressure sensor as well as control circuitry responsive to the sensors to produce the intended piston motion. For applications such as implantable blood pumps where replacement of failed or malfunctioning sensors requires open heart surgery, it is unacceptable to have a linear motor drive and controller that relies on any such sensors. In addition, the Roth controller circuit uses only NPN transistors thereby restricting current flow to the motor windings to one direction only.’

‘U.S. Pat. No. 4,541,787 to Delong describes a pump configuration wherein a piston containing a permanent magnet is driven in a reciprocating fashion along the length of a cylinder by energizing a sequence of coils positioned around the outside of the cylinder. However, the coil and control system configurations disclosed only allow current to flow through one individual winding at a time. This does not make effective use of the magnetic flux produced by each pole of the magnet in the piston. To maximize force applied to the piston in a given direction, current must flow in one direction in the coils surrounding the vicinity of the north pole of the permanent magnet while current flows in the opposite direction in the coils surrounding the vicinity of the south pole of the permanent magnet. Further, during starting of the pump disclosed by Delong, if the magnetic piston is not in the vicinity of the first coil energized, the sequence of coils that are subsequently energized will ultimately approach and repel the magnetic piston toward one end of the closed cylinder. Consequently, the piston must be driven into the end of the closed cylinder before the magnetic poles created by the external coils can become coupled with the poles of the magnetic piston in attraction.”

“U.S. Pat. No. 4,610,658 to Buchwald et al. discloses an implantable fluid displacement peritoneovenous shunt system. The system comprises a magnetically driven pump having a spool piston fitted with a disc flap valve.”

“U.S. Pat. No. 5,089,017 to Young et al. discloses a drive system for artificial hearts and left ventricular assist devices comprising one or more implantable pumps driven by external electromagnets. The pump utilizes working fluid, such as sulfur hexafluoride to apply pneumatic pressure to increase blood pressure and flow rate.”

U.S. Pat. No. 5,743,854 discloses a device for inducing and localizing epileptiform activity that is comprised of a direct current (DC) magnetic field generator, a DC power source, and sensors adapted to be coupled to a patient's head. In one embodiment of the invention, described in claim 7, the sensors “ . . . comprise Foramen Ovale electrodes adapted to be implanted to sense evoked and natural epileptic firings.”

U.S. Pat. No. 5,803,897 discloses a penile prosthesis system comprised of an implantable pressurized chamber, a reservoir, a rotary pump, a magnetically responsive rotor, and a rotary magnetic field generator. Claim 1 of this patent describes: “A penile prosthesis system comprising: at least one pressurizable chamber including a fluid port, said chamber adapted to be located within the penis of a patient for tending to make the penis rigid in response to fluid pressure within said chamber; a fluid reservoir; a rotary pump adapted to be implanted within the body of a user, said rotary pump being coupled to said reservoir and to said chamber, said rotary pump including a magnetically responsive rotor adapted for rotation in the presence of a rotating magnetic field, and an impeller for tending to pump fluid at least from said reservoir to said chamber under the impetus of fluid pressure, to thereby pressurize said chamber in response to operation of said pump; and a rotary magnetic field generator for generating a rotating magnetic field, for, when placed adjacent to the skin of said user at a location near said rotary pump, rotating said magnetically responsive rotor in response to said rotating magnetic field, to thereby tend to pressurize said chamber and to render the penis rigid; controllable valve means operable in response to motion of said rotor of said rotary pump, for tending to prevent depressurization of said chamber when said rotating magnetic field no longer acts on said rotor, said controllable valve means comprising a unidirectional check valve located in the fluid path extending between said rotary pump and said port of said chamber.”

U.S. Pat. No. 5,810,015 describes an implantable power supply that can convert non-electrical energy (such as mechanical, chemical, thermal, or nuclear energy) into electrical energy; the entire disclosure of this United States patent is hereby incorporated by reference into this specification.

In column 1 of U.S. Pat. No. 5,810,015, a discussion of “prior art” rechargeable power supplies is presented. It is disclosed in this column 1 that: “Modem medical science employs numerous electrically powered devices which are implanted in a living body. For example, such devices may be employed to deliver medications, to support blood circulation as in a cardiac pacemaker or artificial heart, and the like. Many implantable devices contain batteries which may be rechargeable by transcutaneous induction of electromagnetic fields in implanted coils connected to the batteries. Transcutaneous inductive recharging of batteries in implanted devices is disclosed for example in U.S. Pat. Nos. 3,923,060; 4,082,097; 4,143,661; 4,665,896; 5,279,292; 5,314,453; 5,372,605, and many others.”

“Other methods for recharging implanted batteries have also been attempted. For example, U.S. Pat. No. 4,432,363 discloses use of light or heat to power a solar battery within an implanted device. U.S. Pat. No. 4,661,107 discloses recharging of a pacemaker battery using mechanical energy created by motion of an implanted heart valve.”

“A number of implanted devices have been powered without batteries. U.S. Pat. Nos. 3,486,506 and 3,554,199 disclose generation of electric pulses in an implanted device by movement of a rotor in response to the patient's heartbeat. U.S. Pat. No. 3,563,245 discloses a miniaturized power supply unit which employs mechanical energy of heart muscle contractions to generate electrical energy for a pacemaker. U.S. Pat. No. 3,456,134 discloses a piezoelectric converter for electronic implants in which a piezoelectric crystal is in the form of a weighted cantilever beam capable of responding to body movement to generate electric pulses. U.S. Pat. No. 3,659,615 also discloses a piezoelectric converter which reacts to muscular movement in the area of implantation. U.S. Pat. No. 4,453,537 discloses a pressure actuated artificial heart powered by a second implanted device attached to a body muscle which in turn is stimulated by an electric signal generated by a pacemaker.”

“In spite of all these efforts, a need remains for efficient generation of energy to supply electrically powered implanted devices.”

The solution provided by U.S. Pat. No. 5,80,015 is described in claim 1 thereof, which describes: “An implantable power supply apparatus for supplying electrical energy to an electrically powered device, comprising: a power supply unit including: a transcutaneously, invasively rechargeable non-electrical energy storage device (NESD); an electrical energy storage device (EESD); and an energy converter coupling said NESD and said EESD, said converter including means for converting non-electrical energy stored in said NESD to electrical energy and for transferring said electrical energy to said EESD, thereby storing said electrical energy in said EESD.”

An implantable ultrasound communicaton system is disclosed in U.S. Pat. No. 5,861,018, the entire disclosure of which is hereby incorporated by reference into this specification. As is disclosed in the abstract of this patent, there is disclosed in such patent “A system for communicating through the skin of a patient, the system including an internal communication device implanted inside the body of a patient and an external communication device. The external communication device includes an external transmitter which transmits a carrier signal into the body of the patient during communication from the internal communication device to the external communication device. The internal communication device includes an internal modulator which modulates the carrier signal with information by selectively reflecting the carrier signal or not reflecting the carrier signal. The external communication device demodulates the carrier signal by detecting when the carrier signal is reflected and when the carrier signal is not reflected through the skin of the patient. When the reflected carrier signal is detected, it is interpreted as data of a first state, and when the reelected carrier signal is not detected, it is interpreted as data of a second state. Accordingly, the internal communication device consumes relatively little power because the carrier signal used to carry the information is derived from the external communication device. Further, transfer of data is also very efficient because the period needed to modulate information of either the first state or the second state onto the carrier signal is the same. In one embodiment, the carrier signal operates in the ultrasound frequency range.”

U.S. Pat. No. 5,861,019, the entire disclosure of which is hereby incorporated by reference into this specification, discloses a telemetry system for communications between an external programmer and an implantable medical device. Claim 1 of this patent describes: “A telemetry system for communications between an external programmer and an implantable medical device, comprising:the external programmer comprising an external telemetry antenna and an external transceiver for receiving uplink telemetry transmissions and transmitting downlink telemetry transmission through the external telemetry antenna; the implantable medical device comprising an implantable medical device housing, an implantable telemetry antenna and an implantable transceiver for receiving downlink transmissions and for transmitting uplink telemetry transmission through the implantable telemetry antenna, the implantable medical device housing being formed of a conductive metal and having an exterior housing surface and an interior housing surface; the implantable medical device housing being formed with a housing recess extending inwardly from the exterior housing surface to a predetermined housing recess depth in the predetermined substrate area of the exterior housing surface for receiving the dielectric substrate therein; wherein the implantable telemetry antenna is a conformal microstrip antenna formed as part of the implantable medical device housing, the microstrip antenna having electrically conductive ground plane and radiator patch layers separated by a dielectric substrate, layer the conductive radiator patch layer having a predetermined thickness and predetermined radiator patch layer dimensions, the patch layer being formed upon one side of the dielectric substrate layer.”

“An extensive description of the historical development of uplink and downlink telemetry transmission formats” is set forth at columns 2 through 5 of U.S. Pat. No. 5,861,019. As is disclosed in these columns: “An extensive description of the historical development of uplink and downlink telemetry transmission formats and is set forth in the above-referenced '851 and '963 applications and in the following series of commonly assigned patents all of which are incorporated herein by reference in their entireties. Commonly assigned U.S. Pat. No. 5,127,404 to Grevious et al. sets forth an improved method of frame based, pulse position modulated (PPM) of data particularly for uplink telemetry. The frame-based PPM telemetry format increases bandwidth well above simple PIM or pulse width modulation (PWM) binary bit stream transmissions and thereby conserves energy of the implanted medical device. Commonly assigned U.S. Pat. No. 5,168,871 to Grevious et al. sets forth an improvement in the telemetry system of the '404 patent for detecting uplink telemetry RF pulse bursts that are corrupted in a noisy environment. Commonly assigned U.S. Pat. No. 5,292,343 to Blanchette et al. sets forth a further improvement in the telemetry system of the '404 patent employing a hand shake protocol for maintaining the communications link between the external programmer and the implanted medical device despite instability in holding the programmer RF head steady during the transmission. Commonly assigned U.S. Pat. No. 5,324,315 to Grevious sets forth an improvement in the uplink telemetry system of the '404 patent for providing feedback to the programmer to aid in optimally positioning the programmer RF head over the implanted medical device. Commonly assigned U.S. Pat. No. 5,117,825 to Grevious sets forth an further improvement in the programmer RF head for regulating the output level of the magnetic H field of the RF head telemetry antenna using a signal induced in a sense coil in a feedback loop to control gain of an amplifier driving the RF head telemetry antenna. Commonly assigned U.S. Pat. No. 5,562,714 to Grevious sets forth a further solution to the regulation of the output level of the magnetic H field generated by the RF head telemetry antenna using the sense coil current to directly load the H field. Commonly assigned U.S. Pat. No. 5,354,319 to Wybomey et al. sets forth a number of further improvements in the frame based telemetry system of the '404 patent. Many of these improvements are incorporated into MEDTRONIC® Model 9760, 9766 and 9790 programmers. These improvements and the improvements described in the above-referenced pending patent applications are directed in general to increasing the data transmission rate, decreasing current consumption of the battery power source of the implantable medical device, and increasing reliability of uplink and downlink telemetry transmissions.”

“The current MEDTRONIC® telemetry system employing the 175 kHz carrier frequency limits the upper data transfer rate, depending on bandwidth and the prevailing signal-to-noise ratio. Using a ferrite core, wire coil, RF telemetry antenna results in: (1) a very low radiation efficiency because of feed impedance mismatch and ohmic losses; 2) a radiation intensity attenuated proportionally to at least the fourth power of distance (in contrast to other radiation systems which have radiation intensity attenuated proportionally to square of distance); and 3) good noise immunity because of the required close distance between and coupling of the receiver and transmitter RF telemetry antenna fields.”

“These characteristics require that the implantable medical device be implanted just under the patient's skin and preferably oriented with the RF telemetry antenna closest to the patient's skin. To ensure that the data transfer is reliable, it is necessary for the patient to remain still and for the medical professional to steadily hold the RF programmer head against the patient's skin over the implanted medical device for the duration of the transmission. If the telemetry transmission takes a relatively long number of seconds, there is a chance that the programmer head will not be held steady. If the uplink telemetry transmission link is interrupted by a gross movement, it is necessary to restart and repeat the uplink telemetry transmission. Many of the above-incorporated, commonly assigned, patents address these problems.”

“The ferrite core, wire coil, RF telemetry antenna is not bio-compatible, and therefore it must be placed inside the medical device hermetically sealed housing. The typically conductive medical device housing adversely attenuates the radiated RF field and limits the data transfer distance between the programmer head and the implanted medical device RF telemetry antennas to a few inches.”

“In U.S. Pat. Nos. 4,785,827 to Fischer, 4,991,582 to Byers et al., and commonly assigned U.S. Pat. No. 5,470,345 to Hassler et al. (all incorporated herein by reference in their entireties), the metal can typically used as the hermetically sealed housing of the implantable medical device is replaced by a hermetically sealed ceramic container. The wire coil antenna is still placed inside the container, but the magnetic H field is less attenuated. It is still necessary to maintain the implanted medical device and the external programming head in relatively close proximity to ensure that the H field coupling is maintained between the respective RF telemetry antennas.”

“Attempts have been made to replace the ferrite core, wire coil, RF telemetry antenna in the implantable medical device with an antenna that can be located outside the hermetically sealed enclosure. For example, a relatively large air core RF telemetry antenna has been embedded into the thermoplastic header material of the MEDTRONIC® Prometheus programmable IPG. It is also suggested that the RF telemetry antenna may be located in the IPG header in U.S. Pat. No. 5,342,408. The header area and volume is relatively limited, and body fluid may infiltrate the header material and the RF telemetry antenna.”

“In U.S. Pat. Nos. 5,058,581 and 5,562,713 to Silvian, incorporated herein by reference in their entireties, it is proposed that the elongated wire conductor of one or more medical lead extending away from the implanted medical device be employed as an RF telemetry antenna. In the particular examples, the medical lead is a cardiac lead particularly used to deliver energy to the heart generated by a pulse generator circuit and to conduct electrical heart signals to a sense amplifier. A modest increase in the data transmission rate to about 8 Kb/s is alleged in the '581 and '713 patents using an RF frequency of 10-300 MHz. In these cases, the conductor wire of the medical lead can operate as a far field radiator to a more remotely located programmer RF telemetry antenna. Consequently, it is not necessary to maintain a close spacing between the programmer RF telemetry antenna and the implanted cardiac lead antenna or for the patient to stay as still as possible during the telemetry transmission.”

“However, using the medical lead conductor as the RF telemetry antenna has several disadvantages. The radiating field is maintained by current flowing in the lead conductor, and the use of the medical lead conductor during the RF telemetry transmission may conflict with sensing and stimulation operations. RF radiation losses are high because the human body medium is lossy at higher RF frequencies. The elongated lead wire RF telemetry antenna has directional radiation nulls that depend on the direction that the medical lead extends, which varies from patient to patient. These considerations both contribute to the requirement that uplink telemetry transmission energy be set artificially high to ensure that the radiated RF energy during the RF uplink telemetry can be detected at the programmer RF telemetry antenna. Moreover, not all implantable medical devices have lead conductor wires extending from the device.”

“A further U.S. Pat. No. 4,681,111 to Silvian, incorporated herein by reference in its entirety, suggests the use of a stub antenna associated with the header as the implantable medical device RF telemetry antenna for high carrier frequencies of up to 200 MHz and employing phase shift keying (PSK) modulation. The elimination of the need for a VCO and a bit rate on the order of 2-5% of the carrier frequency or 3.3-10 times the conventional bit rate are alleged.”

“At present, a wide variety of implanted medical devices are commercially released or proposed for clinical implantation. Such medical devices include implantable cardiac pacemakers as well as implantable cardioverter-defibrillators, pacemaker-cardioverter-defibrillators, drug delivery pumps, cardiomyostimulators, cardiac and other physiologic monitors, nerve and muscle stimulators, deep brain stimulators, cochlear implants, artificial hearts, etc. As the technology advances, implantable medical devices become ever more complex in possible programmable operating modes, menus of available operating parameters, and capabilities of monitoring increasing varieties of physiologic conditions and electrical signals which place ever increasing demands on the programming system.”

“It remains desirable to minimize the time spent in uplink telemetry and downlink transmissions both to reduce the likelihood that the telemetry link may be broken and to reduce current consumption.”

“Moreover, it is desirable to eliminate the need to hold the programmer RF telemetry antenna still and in proximity with the implantable medical device RF telemetry antenna for the duration of the telemetry transmission. As will become apparent from the following, the present invention satisfies these needs.”

The solution to this problem is presented, e.g., in claim 1 of U.S. Pat. No. 5,861,019. This claim describes “A telemetry system for communications between an external programmer and an implantable medical device, comprising:the external programmer comprising an external telemetry antenna and an external transceiver for receiving uplink telemetry transmissions and transmitting downlink telemetry transmission through the external telemetry antenna; the implantable medical device comprising an implantable medical device housing, an implantable telemetry antenna and an implantable transceiver for receiving downlink transmissions and for transmitting uplink telemetry transmission through the implantable telemetry antenna, the implantable medical device housing being formed of a conductive metal and having an exterior housing surface and an interior housing surface; the implantable medical device housing being formed with a housing recess extending inwardly from the exterior housing surface to a predetermined housing recess depth in the predetermined substrate area of the exterior housing surface for receiving the dielectric substrate therein; wherein the implantable telemetry antenna is a conformal microstrip antenna formed as part of the implantable medical device housing, the microstrip antenna having electrically conductive ground plane and radiator patch layers separated by a dielectric substrate, layer the conductive radiator patch layer having a predetermined thickness and predetermined radiator patch layer dimensions, the patch layer being formed upon one side of the dielectric substrate layer.”

U.S. Pat. No. 5,945,762, the entire disclosure of which is hereby incorporated by reference into this specification, discloses an external transmitter adapted to magnetically excite an implanted receiver coil. Claim 1 of this patent describes “An external transmitter adapted for magnetically exciting an implanted receiver coil, causing an electrical current to flow in the implanted receiver coil, comprising: (a) a support; (b) a magnetic field generator that is mounted to the support; and (c) a prime mover that is drivingly coupled to an element of the magnetic field generator to cause said element of the magnetic field generator to reciprocate, in a reciprocal motion, said reciprocal motion of said element of the magnetic field generator producing a varying magnetic field that is adapted to induce an electrical current to flow in the implanted receiver coil.”

U.S. Pat. No. 5,954,758, the entire disclosure of which is hereby incorporated by reference into this specification, claims an implantable electrical stimulator comprised of an implantable radio frequency receiving coil, an implantable power supply, an implantable input singal generator, an implantable decoder, and an implantable electrical stimulator. Claim 1 of this patent describes “A system for transcutaneously telemetering position signals out of a human body and for controlling a functional electrical stimulator implanted in said human body, said system comprising: an implantable radio frequency receiving coil for receiving a transcutaneous radio frequency signal; an implantable power supply connected to said radio frequency receiving coil, said power supply converting received transcutaneous radio frequency signals into electromotive power; an implantable input signal generator electrically powered by said implantable power supply for generating at least one analog input movement signal to indicate voluntary bodily movement along an axis; an implantable encoder having an input operatively connected with said implantable input signal generator for encoding said movement signal into output data in a preselected data format; an impedance altering means connected with said encoder and said implantable radio frequency signal receiving coil to selectively change an impedance of said implantable radio frequency signal receiving coil; an external radio frequency signal transmit coil inductively coupled with said implantable radio frequency signal receiving coil, such that impedance changes in said implantable radio frequency signal receiving coil are sensed by said external radio frequency signal transmit coil to establish a sensed modulated movement signal in said external transmit coil; an external control system electrically connected to said external radio frequency transmit coil for monitoring said sensed modulated movement signal in said external radio frequency transmit coil, said external control system including: a demodulator for recovering the output data of said encoder from the sensed modulated ovement signal of said external transmit coil, a pulse width algorithm means for applying a preselected pulse width algorithm to the recovered output data to derive a first pulse width, an amplitude algorithm means for applying an amplitude algorithm to the recovered output data to derive a first amplitude therefrom, an interpulse interval algorithm means for applying an interpulse algorithm to the recovered output data to derive a first interpulse interval therefrom; and, a stimulation pulse train signal generator for generating a stimulus pulse train signal which has the first pulse width and the first pulse amplitude; an implantable functional electrical stimulator for receiving said stimulation pulse train signal from said stimulation pulse train signal generator and generating stimulation pulses with the first pulse width, the first pulse amplitude, and separated by the first interpulse interval; and, at least one electrode operatively connected with the functional electrical stimulator for applying said stimulation pulses to muscle tissue of said human body.”

U.S. Pat. No. 6,006,133, the entire disclosure of which is hereby incorporated by reference into this specification, describes an implantable medical device comprised of a hermetically sealed housing.

U.S. Pat. No. 6,083,166, the entire disclosure of which is hereby incorporated by reference into this specification, discloses an ultrasound transmitter for use with a surgical device.

U.S. Pat. No. 6,152,882, the entire disclosure of which is hereby incorporated by reference into this specification, discloses an implantable electroporation unit, an implantable proble electrode, an implantable reference electrode, and an an amplifier unit. Claim 35 of this patent describes: “Apparatus for measurement of monophasic action potentials from an excitable tissue including a plurality of cells, the apparatus comprising: at least one probe electrode placeable adjacent to or in contact with a portion of said excitable tissue; at least one reference electrode placeable proximate said at least one probe electrode; an electroporating unit electrically connected to said at least one probe electrode and said at least one reference electrode for controllably applying to at least some of said cells subjacent said at least one probe electrode electrical current pulses suitable for causing electroporation of cell membranes of said at least some of said cells; and an amplifier unit electrically connected to said at least one probe electrode and to said at least one reference electrode for providing an output signal representing the potential difference between said probe electrode and said reference electrode”

U.S. Pat. No. 6,169,925, the entire disclosure of which is hereby incorporated by reference into this specification, describes a transceiver for use in communication with an implantable medical device. Claim 1 of this patent describes: “An external device for use in communication with an implantable medical device, comprising: a device controller; a housing; an antenna array mounted to the housing; an RF transceiver operating at defined frequency, coupled to the antenna array; means for encoding signals to be transmitted to the implantable device, coupled to an input of the transceiver; means for decoding signals received from the implantable device, coupled to an output of the transceiver; and means for displaying the decoded signals received from the implantable device; wherein the antenna array comprises two antennas spaced a fraction of the wavelength of the defined frequency from one another, each antenna comprising two antenna elements mounted to the housing and located orthogonal to one another; and wherein the device controller includes means for selecting which of the two antennas is coupled to the transceiver.”

U.S. Pat. No. 6,185,452, the entire disclosure of which is hereby incorporated by reference into this specification, claims a device for stimulating internal tissue, wherein such device is comprised of: “a sealed elongate housing configured for implantation in said patient's body, said housing having an axial dimension of less than 60 mm and a lateral dimension of less than 6 mm; power consuming circuitry carried by said housing including at least one electrode extending externally of said housing, said power consuming circuitry including a capacitor and pulse control circuitry for controlling (1) the charging of said capacitor and (2) the discharging of said capacitor to produce a current pulse through said electrode; a battery disposed in said housing electrically connected to said power consuming circuitry for powering said pulse control circuitry and charging said capacitor, said battery having a capacity of at least one microwatt-hour; an internal coil and a charging circuit disposed in said housing for supplying a charging current to said battery; an external coil adapted to be mounted outside of said patient's body; and means for energizing said external coil to generate an alternating magnetic field for supplying energy to said charging circuit via said internal coil.”

U.S. Pat. No. 6,235,024, the entire disclosure of which is hereby incorporated by reference into this specification, discloses an implantable highfrequency energy generator. Claim 1 of this patent describes: “A catheter system comprising: an elongate catheter tubing having a distal section, a distal end, a proximal end, and at least one lumen extending between the distal end and the proximal end; a handle attached to the proximal end of said elongate catheter tubing, wherein the handle has a cavity; an ablation element mounted at the distal section of the elongate catheter tubing, the ablation element having a wall with an outer surface and an inner surface, wherein the outer surface is covered with an outer member made of a first electrically conductive material and the inner surface is covered with an inner member made of a second electrically conductive material, and wherein the wall comprises an ultrasound transducer; an electrical conducting means having a first and a second electrical wires, wherein the first electrical wire is coupled to the outer member and the second electrical wire is coupled to the inner member of the ablation element; and a high frequency energy generator means for providing a radiofrequency energy to the ablation element through a first electrical wire of the electrical conducting means.”

An implantable light-generating apparatus is described in claim 16 of U.S. Pat. No. 6,363,279, the entire disclosure of which is hereby incorporated by reference into this specification. As is disclosed in such claim 16, this patent provides a “Heart control apparatus, comprising circuitry for generating a non-excitatory stimulus, and stimulus application devices for applying to a heart or to a portion thereof said non-excitatory stimulus, wherein the circuitry for generating a non-excitatory stimulus generates a stimulus which is unable to generate a propagating action potential and wherein said circuitry comprises a light-generating apparatus for generating light.

An implantable ultrasound probe is described in claim 1 of U.S. Pat. No. 6,421,565, the entire disclosure of which is hereby incorporated by reference into this specifcation. This claim 1 describes “An implantable cardiac monitoring device comprising: an A-mode ultrasound probe adapted for implantation in a right ventricle of a heart, said ultrasound probe emitting an ultrasound signal and receiving at least one echo of said ultrasound signal from at least one cardiac segment of the left ventricle; a unit connected to said ultrasound probe for identifying a time difference between emission of said ultrasound signal and reception of said echo and, from said time difference, determining a position of said cardiac segment, said cardiac segment having a position which, at least when reflecting said ultrasound signal, is correlated to cardiac performance, and said unit deriving an indication of said cardiac performance from said position of said cardiac segment.”

An implantalbe stent that contains a tube and several optical emitters located on the innser surface of the tube is disclosed in U.S. Pat. No. 6,488,704, the entire disclosure of which is hereby incorporated by reference into this specification. Claim 1 of this patent describes “1. An implantable stent which comprises: (a) a tube comprising an inner surface and an outer surface, and (b) a multiplicity of optical radiation emitting means adapted to emit radiation with a wavelength from about 30 nanometers to about 30 millimeters, and a multiplicity of optical radiation detecting means adapted to detect radiation with a wavelength of from about 30 nanometers to about 30 millimeters, wherein said optical radiation emitting means and said optical radiation detecting means are disposed on the inside surface of said tube.”

Many other implantable devices and configurations are described in the claims of U.S. Pat. No. 6,488,704.

Thus, e.g., claim 2 of such patent disloses that the “ . . . implantable stent is comprised of a flexible casing with an inner surface and an outer surface.” claim 3 of such patent discloses that the case may be “ . . . comprised of fluoropolymer.” claim 4 of such patent discloses that the casing may be “ . . . optically impermeable.”

Thus, e.g., claim 10 of U.S. Pat. No. 6,488,704 discloses an embodiment in which an implantable stent contains “ . . . telemetry means for transmitting a signal to a receiver located external to said implantable stent.” The telemetry means may be adated to receive “ . . . a signal from a transmitter located external to said implantable stent (see claim 11); and such signal may be a radio-frequency signal (see claims 12 and 13). The implantable stent may also comprise “ . . . telemetry means for transmitting a signal to a receiver located external to said implantable stent” (see claim 22), and/or “ . . . telemetry means for receiving a signal from a transmitter located external to said implantable stent” (see claim 23), and/or “ . . . a controller operatively connected to said means for transmitting a signal to said receiver, and operatively connected to said means for receiving a signal from said transmitter” (see claim 24).

Thus, e.g., claim 14 of U.S. Pat. No. 6,488,704 describes an implantable stent that contains a waveguide array. The waveguide array may contain “ . . . a flexible optical waveguide device” (see claim 15), and/or “ . . . means for transmitting optical energy in a specified configuration” (see claim 16), and/or “ . . . a waveguide interface for receiving said optical energy transmitted in said specified configuration by said waveguide array” (see claim 17), and/or “ . . . means for filtering specified optical frequencies” (see claim 18). The implantalbe stent may be comprised of “ . . . means for receiving optical energy from said waveguide array” (see claim 19), and/or “ . . . means for processing said optical energy received from waveguide array” (see claim 20). The implantable stent may comprise “ . . . means for processing said radiation emitted by said optical radiation emitting means adapted with a wavelength from about 30 nanometers to about 30 millimeters” (see claim 21).

The implantable stent may be comprised of implantable laser devices. Thus, e.g., and referring again to U.S. Pat. No. 6,488,704, the implantable stent may be comprised of a multiplicity of vertical cavity surface emitting lasers and photodetectors arranged in a monolithic configuration” (see claim 27), wherein “ . . . said monolithic configuration further comprises a multiplicity of optical drivers operatively connected to said vertical cavity surface emitting lasers” (see claim 28) and/or wherein “ . . . said vertical cavity surface emitting lasers each comprise a multiplicity of distributed Bragg reflector layers” (see claim 29), and/or wherein “ . . . each of said photodetectors comprises a multiplicity of distributed Bragg reflector layers” (see claim 30), and/or wherein “ . . . each of said vertical cavity surface emitting lasers is comprised of an emission layer disposed between a first distributed Bragg reflector layer and a second distributed Bragg reflector layer” (see claim 31), and/or wherein “ . . . said emission layer is comprised of a multiplicity of quantum well structures” (see claim 32), and/or wherein . . . each of said photodetectors is comprised of an absorption layer disposed between a first distributed Bragg reflector layer and a second distributed Bragg reflector layer” (see claim 33), and/or wherein “ . . . each of said vertical cavity surface emitting lasers and photodetectors is disposed on a separate semiconductor substrate” (see claim 34), and/or wherein “ . . . said semiconductor substrate comprises gallium arsenide.”

Referring again to U.S. Pat. No. 6,488,704, the entire disclosure of which is hereby incorporated by reference into this specification, the implantable stent may be comprised of an arithmetic unit (see claim 37 of such patent), and such arithmetic unit may be “ . . . comprised of means for receiving signals from said optical radiation detecting means” (see claim 38), and/or “ . . . means for calculating the concentration of components in an analyte disposed within said implantable stent (see claim 39). In one embodiment, “said means for calculating the concentration of components in said analyte calculates concentrations of said components in said analyte based upon optimum optical path lengths for different wavelengths and values of transmitted light (see claim 40).

Referring again to U.S. Pat. No. 6,488,704, the implantalbe stent may contain a power supply (see claim 41 thereof) which may contain a battery (see claim 42) which, in one embodiment, is a lithium-iodine battery (see claim 43).

U.S. Pat. No. 6,585,763, the entire disclosure of which is hereby incorporated by reference into this specification, describes in its claim 1 “ . . . a vascular graft comprising: a biocompatible material formed into a shape having a longitudinal axis to enclose a lumen disposed along said longitudinal axis of said shape, said lumen positioned to convey fluid through said vascular graft; a first transducer coupled to a wall of said vascular graft; and an implantable circuit for receiving electromagnetic signals, said implantable circuit coupled to said first transducer, said first transducer configured to receive a first energy from said circuit to emit a second energy having one or more frequencies and power levels to alter said biological activity of said medication in said localized area of said body subsequent to implantation of said first transducer in said body near said localized area.” The transducer may be selected from the group consisting of “ . . . an ultrasonic transducer, a plurality of light sources, an electric field transducer, an electromagnetic transducer, and a resistive heating transducer” (see claim 2), it may comprise a coil (see claim 3), it may comprise “ . . . a regular solid including piezoelectric material, and wherein a first resonance frequency, being of said one or more frequencies, is determined by a first dimension of said regular solid and a second resonance frequency, being of said one or more frequencies, is determined by a second dimension of said regular solid and further including a first electrode coupled to said regular solid and a second electrode coupled to said regular solid” (see claim 4).

U.S. Pat. No. 6,605,089, the entire disclosure of which is hereby incorporated by reference into this specification, discloses an implantable bone growth promoting device. Claim 1 of this patent describes “A device for placement into and between at least two adjacent bone masses to promote bone growth therebetween, said device comprising: an implant having opposed first and second surfaces for placement between and in contact with the adjacent bone masses, a mid-longitudinal axis, and a hollow chamber between said first and second surfaces, said hollow chamber being adapted to hold bone growth promoting material, said hollow chamber being along at least a portion of the mid-longitudinal axis of said implant, each of said first and second surfaces having at least one opening in communication with said hollow chamber into which bone from the adjacent bone masses grows; and an energizer for energizing said implant, said energizer being sized and configured to promote bone growth from adjacent bone mass to adjacent bone mass through said first and second surfaces and through at least a portion of said hollow chamber at the mid-longitudinal axis.” The implant may have a coil wrapped around it (see claim 6), a portion of the coil may be “ . . . in the form of an external thread on at least a portion of said first and second surfaces of said implant” (see claim 7), the “external thread” may be energized by the “energizer” (claim 8) by conducting “ . . . electromagnetic energy to said interior space . . . ” of the energizer (claim 9).

Referring again to U.S. Pat. No. 6,605,089, and to the implant claimed therein, the implant may contain “ . . . a power supply delivering an electric charge” (see claim 14), and it may comprise “ . . . a first portion that is electrically conductive for delivering said electrical charge to at least a portion of the adjacent bone masses and said energizer delivers negative electrical charge to said first portion of said implant” (see claim 15). Additionally, the implant may also contain “ . . . a controller for controlling the delivery of said electric charge” that is disposed within the implant (see claim 18), that “ . . . includes one of a wave form generator and a voltage generator” (see claim 19), and that “ . . . provides for the delivery of one of an alternating current, a direct current, and a sinusoidal current” (see claim 21).

U.S. Pat. No. 6,641,520, the entire disclosure of which is hereby incorporated by reference into this specification, discloses a magnetic field generator for providing a static or direct durrent magnetic field generator. In column 1 of this patent, some “prior art” magnetic field generators were described. It was stated in such column 1 that: “There has recently been an increased interest in therapeutic application of magnetic fields. There have also been earlier efforts of others in this area. The recent efforts, as well as those earlier made, can be categorized into three general types, based on the mechanism for generating and applying the magnetic field. The first type were what could be generally referred to as systemic applications. These were large, tubular mechanisms which could accommodate a human body within them. A patient or recipient could thus be subjected to magnetic therapy through their entire body. These systems were large, cumbersome and relatively immobile. Examples of this type of therapeutic systems included U.S. Pat. Nos. 1,418,903; 4,095,588; 5,084,003; 5,160,591; and 5,437,600. A second type of system was that of magnetic therapeutic applicator systems in the form of flexible panels, belts or collars, containing either electromagnets or permanent magnets. These applicator systems could be placed on or about portion of the recipient's body to allow application of the magnetic therapy. Because of their close proximity to the recipients body, considerations limited the amount and time duration of application of magnetic therapy. Examples of this type system were U.S. Pat. Nos. 4,757,804; 5,084,003 and 5,344,384. The third type of system was that of a cylindrical or toroidal magnetic field generator, often small and portable, into which a treatment recipient could place a limb to receive electromagnetic therapy. Because of size and other limitations, the magnetic field strength generated in this type system was usually relatively low. Also, the magnetic field was a time varying one. Electrical current applied to cause the magnetic field was time varying, whether in the form of simple alternating current waveforms or a waveform composed of a series of time-spaced pulses.”

The magnetic field generator claimed in U.S. Pat. No. 6,641,520 comprised “ . . . a magnetic field generating coil composed of a wound wire coil generating the static magnetic field in response to electrical power; a mounting member having the coil mounted thereon and having an opening therethrough of a size to permit insertion of a limb of the recipient in order to receive electromagnetic therapy from the magnetic field coil; an electrical power supply furnishing power to the magnetic field coil to cause the coil to generate a static electromagnetic field within the opening of the mounting member for application to the recipient's limb; a level control mechanism providing a reference signal representing a specified electromagnetic field strength set point for regulating the power furnished to the magnetic field coil; a field strength sensor detecting the static electromagnetic field strength generated by the magnetic field coil and forming a field strength signal representing the detected electromagnetic field strength in the opening in the mounting member; a control signal generator receiving the field strength signal from the field strength sensor and the reference signal from the level control mechanism representing a specified electromagnetic field strength set point; and the control signal generator forming a signal to regulate the power flowing from the electrical power supply to the magnetic field coil.”

An implantable sensor is disclosed in U.S. Pat. No. 6,491,639, the entire disclosure of which is hereby incorporated by reference into this specification. Claim 1 of such patent describes: “An implantable medical device including a sensor for use in detecting the hemodynamic status of a patient comprising: a hermetic device housing enclosing device electronics for receiving and processing data; and said device housing including at least one recess and a sensor positioned in said at least one recess.” Claim 10 of such patent describes “10. An implantable medical device including a hemodynamic sensor for monitoring arterial pulse amplitude comprising: a device housing; a transducer comprising a light source and a light detector positioned exterior to said device housing responsive to variations in arterial pulse amplitude; and wherein said light detector receives light originating from said light source and reflected from arterial vasculature of a patient and generates a signal which is indicative of variations in the reflected light caused by the expansion and contraction of said arterial vasculature.” Claim 14 of such patent describes: “14. An implantable medical device including a hemodynamic sensor for monitoring arterial pulse amplitude comprising: a device housing; and an ultrasound transducer associated with said device housing responsive to variations in arterial pulse amplitude.” claim 15 of such patent describes: “15. An implantable medical device including a hemodynamic sensor for monitoring arterial pulse amplitude comprising: a device housing; and a transducer associated with said device housing responsive to variations in arterial pulse amplitude, said device housing having at least one substantially planar face and said transducer is positioned on said planar face.” claim 17 of such patent describes “ . . . an implantable pulse generator . . . ’

U.S. Pat. No. 6,663,555, the entire disclosure of which is incorporated by reference into this specification, also claims a magnetic field generator. Claim 1 of this patent describes: “A magnet keeper-shield assembly for housing a magnet, said magnet keeper-shield assembly comprising: a keeper-shield comprising a material substantially permeable to a magnetic flux; a cavity in the keeper-shield, said cavity comprising an inner side wall and a base, and said cavity being adapted to accept a magnet having a front and a bottom face; an actuator extending through the base; a plurality of springs extending through the base, said springs operative to exert a force in a range from about 175 pounds to about 225 pounds on the bottom face of the magnet in a retracted position, and wherein said magnet produces at least about 118 gauss at a distance of about 10 cm from the front face in the extended position and produces at most about 5 gauss at a distance less than or equal to about 22 cm from the front face in the retracted position.”

Published United States patent application US2002/0182738 discloses an implantable flow cytometer the entire disclosure of this published United States patent application is hereby incorporated by reference into this specification. Claim 1 of this patent describes “A flow cytometer comprising means for sampling cellular material within a body, means for marking cells within said bodily fluid with a marker to produce marked cells, means for analyzing said marked cells, a first means for removing said marker from said marked cells, a second means for removing said marker from said marked cells, means for sorting said cells within said bodily fluid to produce sorted cells, and means for maintaining said sorted cells cells in a viable state.”

Referring again to published United States patent application US 2002/0182738, the implantable flow cytometer may contain “ . . . a a first control valve operatively connected to said first means for removing said marker from said marked cells and to said second means for removing said marker from said marked cells . . . ” (see claim 3), a controller connected to the first control valve (claim 4), a second control valve (claim 5), a third control valve (claim 6), a dye separator (claims 7 and 8), an analyzer for testing blood purity (claim 9), etc.

A similar flow cytometer is disclosed in published United States patent application US 2003/0036718, the entire disclosure of which is also hereby incorporated by reference into this specification.

Published United States patent application US 2003/0036776, the entire disclosure of which is hereby incorporated by reference into this specification, discloses an MRI-compatible implantable device. Claim 1 of this patent describes “A cardiac assist device comprising means for connecting said cardiac assist device to a heart, means for furnishing electrical impulses from said cardiac assist device to said heart, means for ceasing the furnishing of said electrical impulses to said heart, means for receiving pulsed radio frequency fields, means for transmitting and receiving optical signals, and means for protecting said heart and said cardiac assist device from currents induced by said pulsed radio frequency fields, wherein said cardiac assist device contains a control circuit comprised of a parallel resonant frequency circuit and means for activating said parallel resonant frequency circuit.” The “ . . . means for activating said parallel resonant circuit . . . ” may contain “ . . . comprise optical means (see claim 2) such as an optical switch (claim 3) comprised of “ . . . a pin type diode . . . ” (claim 4) and connected to an optical fiber (claim 5). The optical switch may be “ . . . activated by light from a light source . . . ” (claim 6), and it may be located with a biological organism (claim 7). The light source may be located within the biological organism (claim 9), and it may provide “ . . . light with a wavelength of from about 750 to about 850 nanometers . . . . ”

Other Compositions Comprised of Nanomagnetic Particles

In addition to the compositions already mentioned in this specification, other compositions may advantageous incorporate the nanomagnetic particles of this invention. Thus, by way of illustration and not limitation, one may replace the magnetic particles in prior art compositions with the nanomagnetic materials of this invention.

In many of the prior art patents, the term “comprising magnetic particles” appears in the claims; some of these patents are described below. In the compositions and processes described in the patents described below, one may replace the “magnetic particles” used in such patents with the nanomagnetic particles of this invention. Thus, e.g., one may use such nanomagnetic particles in the compositions and processes of U.S. Pat. Nos. 3,777,295 (magnetic particle core), U.S. Pat. No. 3,905,841 (magnetic particles disposed in organic resin binders), U.S. Pat. No. 4,0188,886 (protein-coated magnetic particles), U.S. Pat. No. 4,145,300 (developers containing magnetic particles and a sublimable dyestuff), U.S. Pat. No. 4,171,274 (tessellated magnetic particles), U.S. Pat. No. 4,177,089 (magnetic particles and compacts thereof), U.S. Pat. No. 4,177,253 (magnetic particles for immunoassay), U.S. Pat. No. 4,189,514 (high-temperature magnetic tape), U.S. Pat. No. 4,197,563 (magnetic particles disposed in a polymerizable ink), U.S. Pat. No. 4,271,782 (apparatus for disorienting magnetic particles), U.S. Pat. No. 4,283,476 (photographic element having a magnetic recording stripe), U.S. Pat. No. 4,379,183 (cobalt-modified magnetic particles), U.S. Pat. No. 4,382,982 (process for protecting magnetic particles with chromium oxide), U.S. Pat. No. 4,419,383 (method for individually encapsulating magnetic particles), U.S. Pat. No. 4,433,289 (mixture of magnetic particles and a water soluble carrier solid), U.S. Pat. No. 4,438,179 (resin particles with magnetic particles bonded to surface), U.S. Pat. No. 4,448,870 (magnetic color toner), U.S. Pat. No. 4,486,523 (magnetic toner particles coated with opaque polymer particles), U.S. Pat. No. 4,505,990 (coating compositions), U.S. Pat. No. 4,532,153 (method of bonding magnetic particles to a resin particles), U.S. Pat. No. 4,546,035 (polymeric additives for magnetic coating materials), U.S. Pat. No. 4,628,037 (binding assays employing magnetic particles), U.S. Pat. No. 4,638,032 (magnetic particles as supports for organic synthesis), U.S. Pat. No. 4,651,092 (resin/solvent mixture containing magnetic particles), U.S. Pat. No. 4,698,302 (enzymatic reactions using magnetic particles), U.S. Pat. No. 4,701,024 (liquid crystal material including magnetic particles), U.S. Pat. No. 4,707,523 (magnetic particles), U.S. Pat. No. 4,728,363 (acicular magnetic particles), U.S. Pat. No. 4,731,337 (fluorometric immunological assay with magnetic particles), U.S. Pat. No. 4,777,145 (immunological assay method using magnetic particles), U.S. Pat. No. 4,857,417 (cobalt-containing magnetic particles), U.S. Pat. No. 4,882,224 (magnetic particles, method for making, and an electromagnetic clutch using the same), U.S. Pat. No. 5,001,424 (measurement of magnetic particles suspended in a fluid), U.S. Pat. No. 5,019,272 (filters having magnetic particles thereon), U.S. Pat. No. 5,021,315 (magnetic particles with improved conductivity), U.S. Pat. No. 5,051,200 (flexible high energy magnetic blend compositions based on rare earth magnetic particles in highly saturated nitrile rubber), U.S. Pat. No. 5,061,571 (magnetic recording medium comprising magnetic particles and a polyester resin), U.S. Pat. No. 5,071,724 (method for making colored magnetic particles), U.S. Pat. No. 5,082,733 (magnetic particles surface treated with a glycidyl compound), U.S. Pat. No. 5,104,582 (electrically conductive fluids), U.S. Pat. No. 5,142,001 (polyurethane composition), U.S. Pat. No. 5,158,871 (method of using magnetic particles for isolating, collecting, and assaying diagnostic ligates), U.S. Pat. No. 5,178,953 (magnetic recording media), U.S. Pat. No. 5,180,650 (toner compositions with conductive colored magnetic particles between core segments), U.S. Pat. No. 5,204,653 (electromagnetic induction device with magnetic particles between core segments), U.S. Pat. No. 5,209,946 (gelatin containing magnetic particles), U.S. Pat. No. 5,217,804 (magnetic particles), U.S. Pat. No. 5,230,964 (magnetic particle binder), U.S. Pat. No. 5,242,837 (light attenuating magnetic particles), U.S. Pat. No. 5,264,157 (an electronic conductive polymer incorporating magnetic particles), U.S. Pat. No. 5,316,699 (magnetic particles dispersed in a dielectric matrix), U.S. Pat. No. 5,328,793 (magnetic particles for magnetic toner), U.S. Pat. No. 5,330,669 (magnetic coating formulations), U.S. Pat. No. 5,350,676 (method for performing fibrinogen assays using dry chemical reagents containing magnetic particles), U.S. Pat. No. 5,362,027 (flow regulating valve for magnetic particles), U.S. Pat. No. 5,371,166 (polyurethane composition), U.S. Pat. No. 5,384,535 (electric magnetic detector of magnetic particles in a steam of fluid), U.S. Pat. No. 5,405,743 (reversible agglutination mediators), U.S. Pat. No. 5,428,332 (magnetized material having enhanced magnetic pull strength), U.S. Pat. No. 5,441,746 (electromagnetic wave absorbing, surface modified magnetic particles for use in medical applications), U.S. Pat. No. 5,443,654 (ferrofluid paint removal system), U.S. Pat. No. 5,445,881 (magnetic tape), U.S. Pat. No. 5,508,164 (isolation of biological materials using magnetic particles), U.S. Pat. No. 5,512,332 (process of making resuspendable coated magnetic particles), U.S. Pat. No. 5,512,439 (oligonucleotide-linked magnetic particles), U.S. Pat. No. 5,543,219 (encapsulated magnetic particles pigments), U.S. Pat. No. 5,670,077 (aqueous magnetorheological materials), U.S. Pat. No. 5,843,567 (electrical component containing magnetic particles), U.S. Pat. No. 5,843,579 (magnetic thermal transfer ribbon with aqueous ferroflids), U.S. Pat. No. 5,855,790 (magnetic particles for use in the purification of solutions), U.S. Pat. No. 5,858,595 (magnetic toner and ink jet compositions), U.S. Pat. No. 5,861,285 (fusion protein-bound magnetic particles), U.S. Pat. No. 5,898,071 (DNA purification and isolation using magnetic particles), U.S. Pat. No. 5,932,097 (microfabricated magnetic particles for applications to affinity binding), U.S. Pat. No. 5,919,490 (preparation for improving the blood supply containing hard magnetic particles), U.S. Pat. No. 5,935,886 (preparation of molecular magnetic switches), U.S. Pat. No. 5,938,979 (electromagnetic shielding), U.S. Pat. No. 5,981,095 (magnetic composites and methods for improved electrolysis), U.S. Pat. No. 5,945,525 (method for isolating nucleic acids using silica-coated magnetic particles), U.S. Pat. No. 5,958,706 (fine magnetic particles containing useful proteins bound thereto), U.S. Pat. No. 6,033,878 (protein-bound magnetic particles), U.S. Pat. No. 6,045,901 (magnetic recording medium), U.S. Pat. No. 6,090,517 (two component type developer for electrostatic latent image), U.S. Pat. No. 6,096,466 (developer), U.S. Pat. No. 6,099,999 (binder carrier comprising magnetic particles and resin), U.S. Pat. No. 6,130,019 (binder carrier), U.S. Pat. No. 6,157,801 (magnetic particles for charging), U.S. Pat. No. 6,165,795 (methods for performing fibrinogen assays using chemical reagents containing ecarin and magnetic particles), U.S. Pat. No. 6,174,661 (silver halide photographic elements), U.S. Pat. No. 6,190,573 (extrusion-molded magnetic body), U.S. Pat. No. 6,203,487 (use of magnetic particles in the focal delivery of cells), U.S. Pat. No. 6,204,033 (polyvinyl alcohol-based magnetic particles for binding biomolecules), U.S. Pat. No. 6,207,003 (fabrication of sturcutre having structural layers and layers of controllable electricalor magnetic properties), U.S. Pat. No. 6,207,313 (magnetic composites), U.S. Pat. No. 6,210,572 (filter comprised of magnetic particles), U.S. Pat. No. 6,231,760 (apparatus for mxing and separation employing magnetic particles), U.S. Pat. No. 6,274,386 (reagent preparation containing magnetic particles in tablet form), U.S. Pat. No. 6,280,618 (multiplex flow assays with magnetic particles as solid phase), U.S. Pat. No. 6,297,062 (separation by magnetic particles), U.S. Pat. No. 6,285,848 (toner), U.S. Pat. No. 6,315,709 (magnetic vascular defect treatement system), U.S. Pat. No. 6,344,273 (treatment solution for forming insulating layers on magnetic particles, process of forming the insulating layers, and electric device with a soft magnetic powder composite core), U.S. Pat. No. 6,337,215 (magnetic particles having two antiparallel ferromagnetic layers and attached affinity recognition molecules), U.S. Pat. No. 6,348,318 (methods for concentrating ligands using magnetic particles), U.S. Pat. No. 6,368,800 (kits for isolating biological target materials using silica magnetic particles), U.S. Pat. No. 6,372,338 (spherical magnetic particles for magnetic recording media), U.S. Pat. No. 6,372,517 (magnetic particles with biologically active receptors), U.S. Pat. No. 6,402,978 (magnetic polishing fluids), U.S. Pat. No. 6,405,007 (magnetic particles for charging), U.S. Pat. No. 6,464,968 (magnetic fluids), U.S. Pat. No. 6,479,302 (method for the immunological determination of an analyte), U.S. Pat. No. 6,527,972 (magnetorehologoical polymer gels), U.S. Pat. No. 6,521,341 (magnetic particles for separating molecules), U.S. Pat. No. 6,545,143 (magnetic particles for purifying nucleic acids), U.S. Pat. No. 6,569,530 (magnetic recording medium), U.S. Pat. No. 6,639,291 (spin dependent tunneling barriers doped with magnetic particles), U.S. Pat. No. 6,705,874 (colored magnetic particles), and the like. The entire disclosure of each and every one of these United States patent applications is hereby incorporated by reference into this specification.

By way of further illustration, one may substitute applicants' nanomagnetic particles for the magnetic particles used in prior art drug formulations.

Preparation and use of Magnetic Taxanes

In this portion of the specification, applicants will be describe the preparation of certain magnetic taxanes that may be used in one or more of the processes of their invention.

In one embodiment of the invention, a biologically active substrate is linked to a magnetic carrier particle. An external magnetic field may then be used to increase the concentration of a magnetically linked drug at a predetermined location.

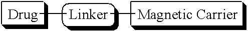

One method for the introduction of a magnetic carrier particle involves the linking of a drug with a magnetic carrier. While some naturally occurring drugs inherently carry magnetic particles (ferrimycin, albomycin, salmycin, etc.), it is more common to generate a synthetic analog of the target drug and attach the magnetic carrier through a linker.

Functionalized Taxanes

Paclitaxel and docetaxel are members of the taxane family of compounds. A variety of taxanes have been isolated from the bark and needles of various yew trees In one embodiment of the invention, such a linker is covalently attached to at least one of the positions in taxane.

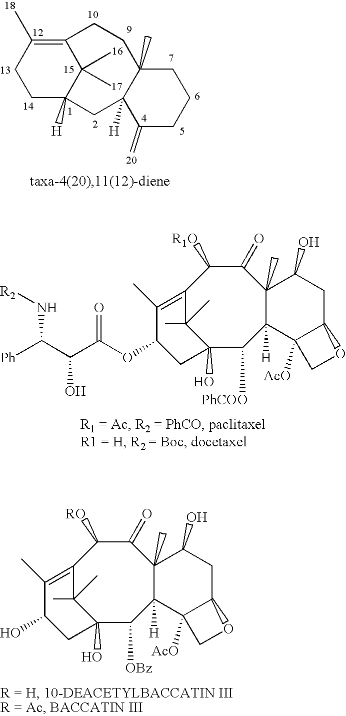

It is well known in the art that the northern hemisphere of taxanes has been altered without significant impact. on the biological activity of the drug. Reference may be had to Chapter 15 of Taxane Anticancer Agents, Basic Science and Current Status, edited by G. George et al., ACS Symposium Series 583, 207^{th} National Meeting of the American Chemical Society, San Diego, Calif. (1994). Specifically the C-7, C-9, and C-10 positions of paclitaxel have been significantly altered without degrading the biological activity of the parent compound. Likewise the C-4 position appears to play only a minor role. The oxetane ring at C-4 to C-5 has been shown to be critical to biological activity. Likewise, certain functional groups on the C-13 sidechain have been shown to be of particular importance.

In one embodiment of the invention, a position within paclitaxel is functionalized to link a magnetic carrier particle. A number of suitable positions are presented below. It should be understood that paclitaxel is illustrated in the figures below, but other taxane analogs may also be employed.

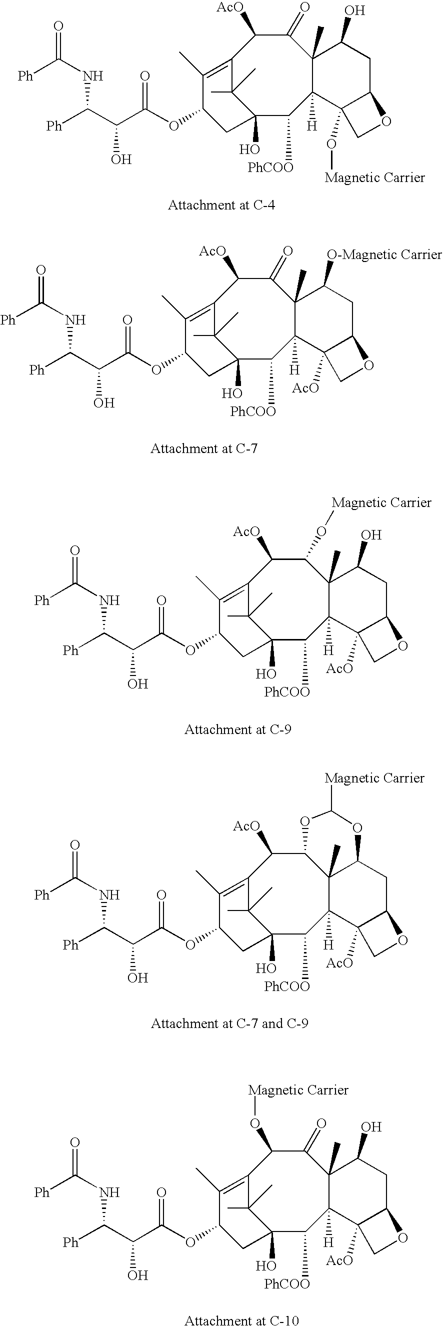

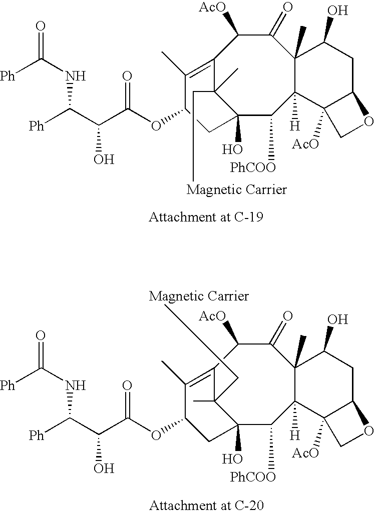

Attachment at C-4

C-4 taxane analogs have been previously generated in the art. A wide range of methodologies exist for the introduction of a variety of substituents at the C-4 position. By way of illustration, reference may be had to “Synthesis and Biological Evaluation of Novel C-4 Aziridine-Bearing Paclitaxel Analogs” by S. Chen et al., J. Med. Chem. 1995, vol 38, pp 2263.

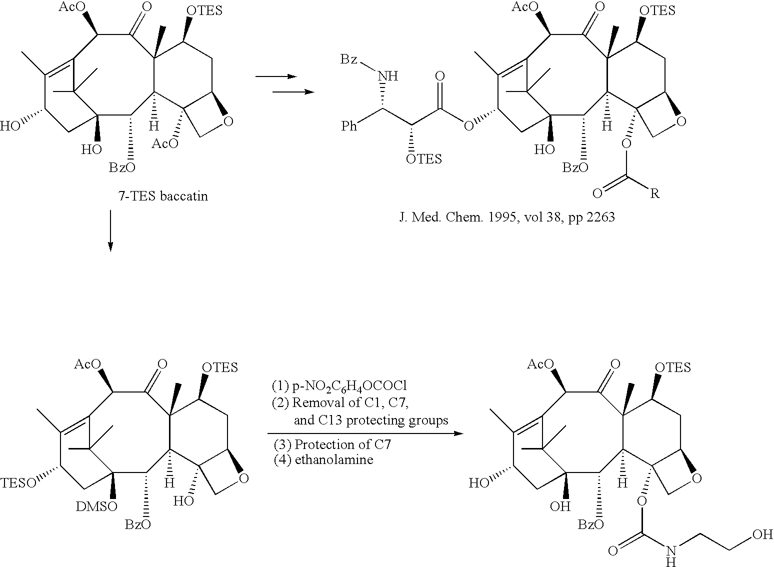

The secondary (C-13) and tertiary (C-1) alcohols of 7-TES baccatin were protected using the procedure of Chen (J. Org. Chem. 1994, vol 59, p 6156) while simultaneously unmasking the alcohol at C-4. The resulting product was treated with a chloroformate to yield the corresponding carboxylate. Removal of the silyl protecting groups at C-1, C-7, and C-13, followed by selective re-protection of the C-7 position gave the desired activated carboxylate. The compound was then treated with a suitable nucleophile (in the author's case, ethanolamine) to produce a C-4 functionalized taxane. The C-13 sidechain was installed using standard lactam methodology.

This synthetic scheme thus provides access to a variety of C-4 taxane analogs by simply altering the nucleophile used. In one embodiment of the instant invention, the nucleophile is selected so as to allow the attachment of a magnetic carrier to the C-4 position.

Attachment at C-7

The C-7 position is readily accessed by the procedures taught in U.S. Pat. No. 6,610,860. The alcohol at the C-10 position of 10-deacetylbaccatin III was selectively protected. The resulting product was then allowed to react with an acid halide to produce the corresponding ester by selectively acylating the C-7 position over the C-13 alcohol. Standard lactam methodology allowed the installation of the C-13 sidechain. In another embodiment, baccatin III, as opposed to its deacylated analog, is used as the starting material.

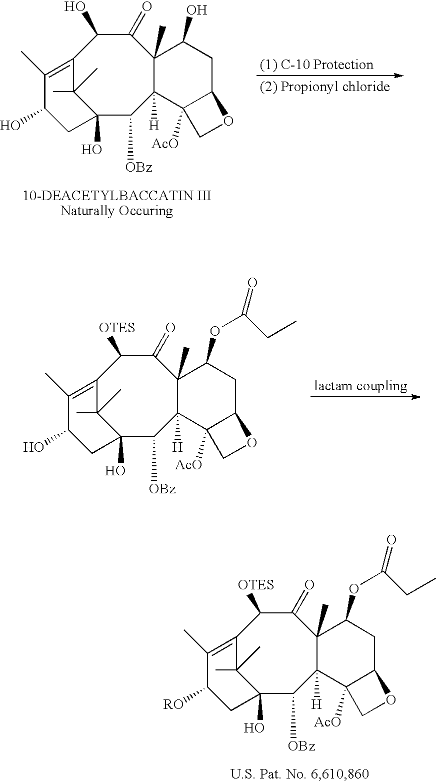

Other C-7 taxane analogs are disclosed in U.S. Pat. Nos. 6,610,860; 6,359,154; and 6,673,833, the contents of which are hereby incorporated by reference.

Attachment at C-9

It has been established that the C-9 carbonyl of paclitaxel is relatively chemically inaccessible, although there are exceptions (see, for example, Tetrahedron Lett. Vol 35, p 4999). However, scientists gained access to C-9 analogs when 13-acetyl-9-dihydrobaccatin III was isolated from Taxus candidensis (see J. Nat. Products, 1992, vol 55, p 55 and Tetrahedron Lett. 1992, vol 33, p 5173). This triol is currently used to provide access to a variety of such C-9 analogues.

In chapter 20 of Taxane Anticancer Agents, Basic Science and Current Status, (edited by G. George et al., ACS Symposium Series 583, 207^{th} National Meeting of the American Chemical Society, San Diego, Calif. (1994)) Klein describes a number of C-7/C-9 taxane analogs. One of routes discussed by Klein begins with the selective deacylation of 13-acetyl-9-dihydrobaccatin III, followed by the selective protection of the C7 alcohol as the silyl ether. A standard lactam coupling introduced the C-13 sidechain. The alcohols at C-7 and C-9 were sufficiently differentiated to allow a wide range of analogs to be generated. “In contrast to the sensitivity of the C-9 carbonyl series under basic conditions, the 9(R)-dihydro system can be treated directly with strong base in order to alkylate the C-7 and/or the C-9 hydroxyl groups.”

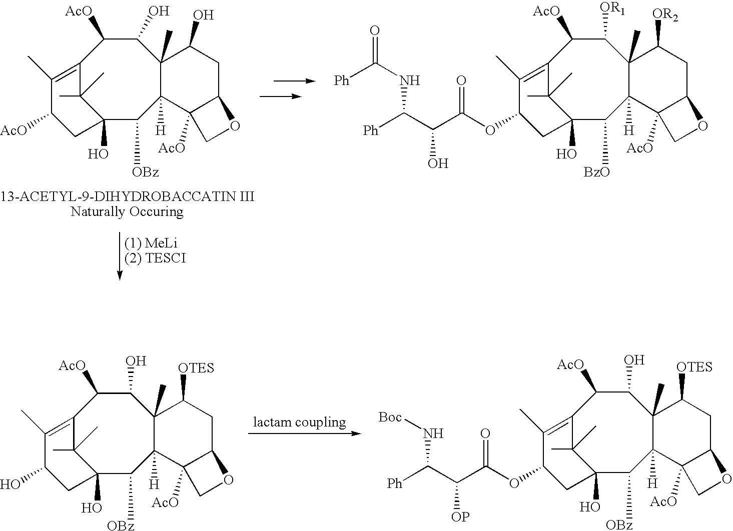

One skilled in the art may adapt Klein's general procedures to install a variety of magnetic carriers at these positions. Such minor adaptations are routine for those skilled in the art.

Attachment at C-7 and C-9

Klein also describes a procedure wherein 13-acetyl-9-dihydrobaccatin III is converted to 9-dihydrotaxol. Reference may be had to “Synthesis of 9-Dihydrotaxol: a Novel Bioactive Taxane” by L. L. Klein in Tetrahedron Lett. Vol 34, pp 2047-2050. An intermediate in this synthetic pathway is the dimethylketal of 9-dihydrotaxol.

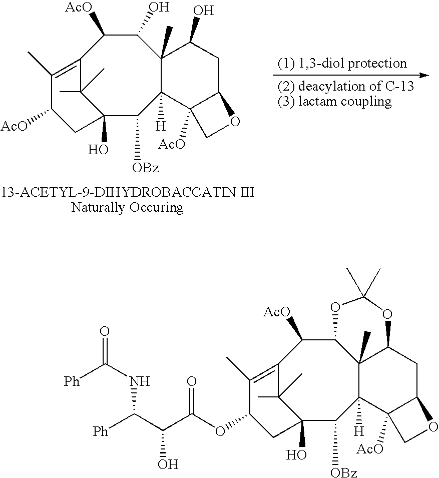

In one embodiment, the procedure of Klein is followed with a carbonyl compound other than acetone to bind a wide variety of groups to the subject ketal. Supplemental discussion of C-9 analogs is found in “Synthesis of 9-Deoxotaxane Analogs” by L. L. Klein in Tetrahedron Lett. Vol 35, p 4707 (1994).

Attachment at C-10

In one embodiment of the invention, the C-10 position is functionalized using the procedure disclosed in U.S. Pat. No. 6,638,973. This patent teaches the synthesis of paclitaxel analogs that vary at the C-10 position. A sample of 10-deacetylbaccatin III was acylated by treatment with propionic anhydride. The C-13 sidechain was attached using standard lactam methodology after first performing a selective protection of the secondary alcohol at the C-7 position. In one embodiment of the invention, this procedure is adapted to allow access to a variety of C-10 analogues of paclitaxel.

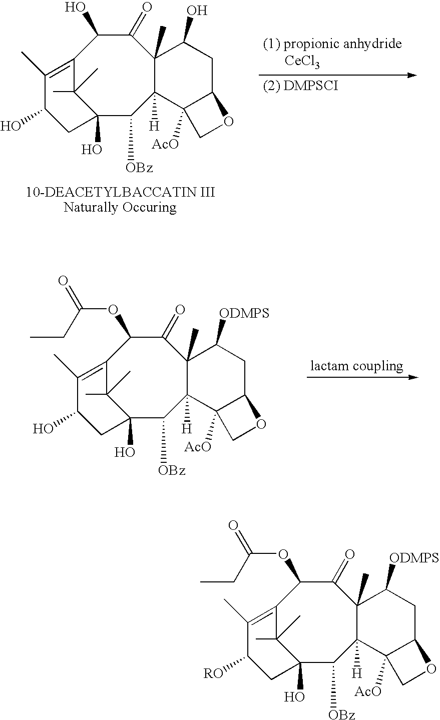

In one embodiment an anhydride is used as an electrophile. In another embodiment, an acid halide is used. As would be apparent to one of ordinary skill in the art, a variety of electrophiles could be employed.

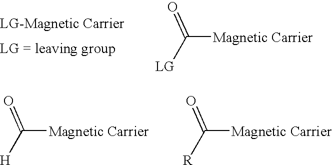

Siderophores

In one embodiment, a member of the taxane family of compounds is attached to a magnetic carrier particle. Suitable carrier particles include siderophores (both iron and non-iron containing), nitroxides, as well as other magnetic carriers.

Sidephores are a class of compounds that act as chelating agents for various metals. Most organisms use sidephores to chelate iron (III) although other metals may be exchanged for iron (see, for example, Exchange of Iron by Gallium in Siderophores by Emergy, Biochemistry 1986, vol 25, pages 4629-4633). Most of the siderophores known to date are either catecholates or hydroxamic acids.

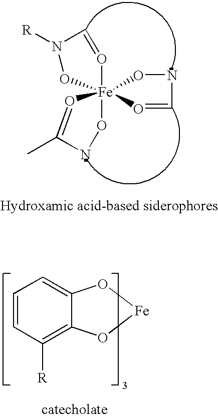

Representative examples of catecholate siderophores include the albomycins, agrobactin, parabactin, enterobactin, and the like.

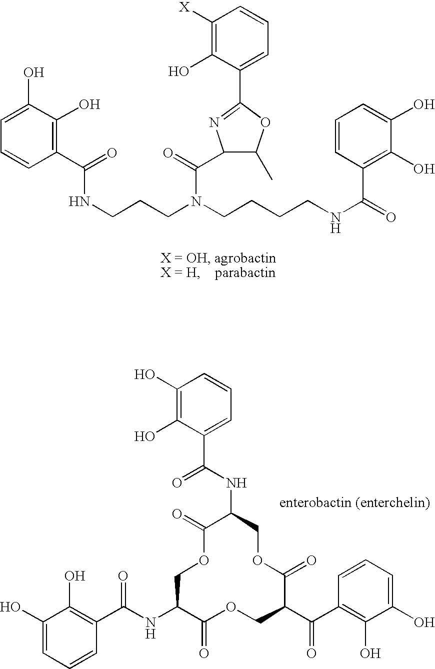

Examples of hydroxamic acid-based siderophores include ferrichrome, ferricrocin, the albomycins, ferrioxamines, rhodotorulic acid, and the like. Reference may be had to Microbial Iron Chelators as Drug Delivery Agents by M. J. Miller et al., Acc. Chem. Res. 1993, vol 26, pp 241-249; Structure of Des(diserylglycyl)ferrirhodin, DDF, a Novel Siderophore from Aspergillus ochraceous by M. A. F. Jalal et al., J. Org. Chem. 1985, vol 50, pp5642-5645; Synthesis and Solution Structure of Microbial Siderophores by R. J. Bergeron, Chem. Rev. 1984, vol 84, pp 587-602; and Coordination Chemistry and Microbial Iron Transport by K. N. Raymond, Acc. Chem. Res., 1979, vol 12, pp 183-190. The synthesis of a retrohydroxamate analog of ferrichrome is described by R. K. Olsen et al. in J. Org. Chem. 1985, vol 50, pp 2264-2271.

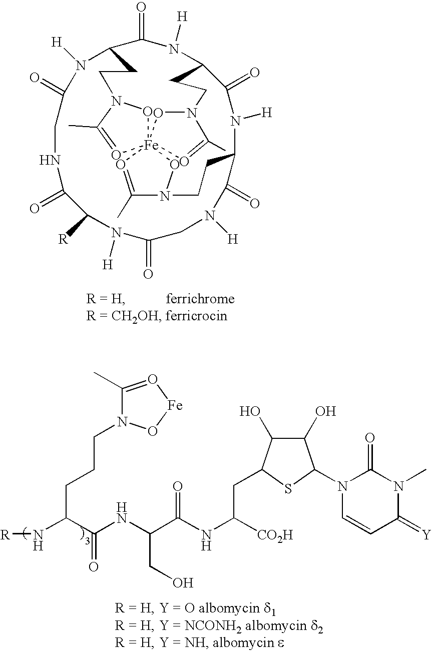

In “Total Synthesis of Desferrisalmycin” (M. J. Miller et al. in J. Am. Chem. Soc. 2002, vol 124 pp 15001-15005), a natural product is synthesized that contains a siderophore. The author states “siderophores are functionally defined as low molecular mass molecules which acquire iron (III) from the environment and transport it into microganisms. Because of the significant roles they play in the active transport of physiologically essentially iron (III) through microbe cell members, it is not surprising that siderophores-drug conjugates are attracting more and more attention from both medicinal chemists and clinical researchers as novel drug delivery systems in the war against microbial infections, especially in an area of widespread emergency of multidrug-resistance (MDR) strains. There have been three families of compounds identified as natural siderophore-drug conjugates, including ferrimycin, albomycin, and salmycin.” In a related paper, Miller describes the use of siderophores as drug delivery agents (Acc. Chem. Res. 1993, vol 26, pp 241-249. Presumably, the siderophore acts as a “sequestering agents [to] facilitate the active transport of chelated iron into cells where, by modification, reduction, or siderophore decomposition, it is released for use by the cell.” Miller describes the process of tethering a drug to a sidrophore to promote the active transport of the drug across the cell membrane.

In “The Preparation of a Fully Differentiated ‘Multiwarhead’ Sidrophore Precursor”, by M. J. Miller et al (J. Org. Chem. 2003, vol 68, pp 191-194) a precursor is disclosed which allows for a drug to be tethered to a sidrophore. In one embodiment, the route disclosed by Miller is employed to provide a variety of siderophores of similar structure. The synthesis of similar hydroxamic acid-based siderophores is discussed in J. Org. Chem. 2000, vol 65 (Total Synthesis of the Siderophore Danoxamine by M. J. Miller et al.), pp 4833-4838 and in the J. of Med. Chem. 1991, vol 32, pp 968-978 (by M. J. Miller et al.).

A variety of fluorescent labels have been attached to ferrichrome analogues in “Modular Fluorescent-Labeled Siderophore Analogues” by A. Shanzer et al. in J. Med. Chem. 1998, vol 41, 1671-1678. The authors have developed a general methodology for such attachments.

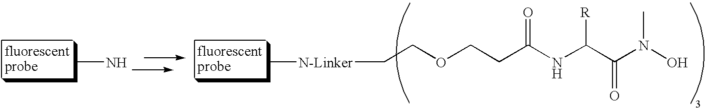

As discussed above, functionalized ferrichrome analogs have been previous generated, usually using basic amine acids (glycine). In one embodiment, functionality is introduced using an alternative amine acid (such as serine) in place of the central glycine residue. This provides a functional group foothold from which to base a wide variety of analogs. Using traditional synthetic techniques, various linkers are utilized so as to increase or decrease the distance between the magnetic carrier and the drug.

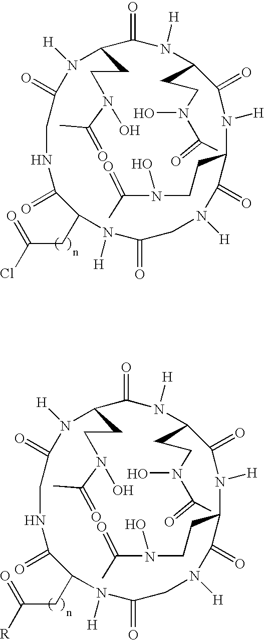

As would be apparent to one of ordinary skill in the art, the above specified techniques are widely applicable to a variety of substrates. By way of illustration, and not limitation, a number of magnetic taxanes are shown below.

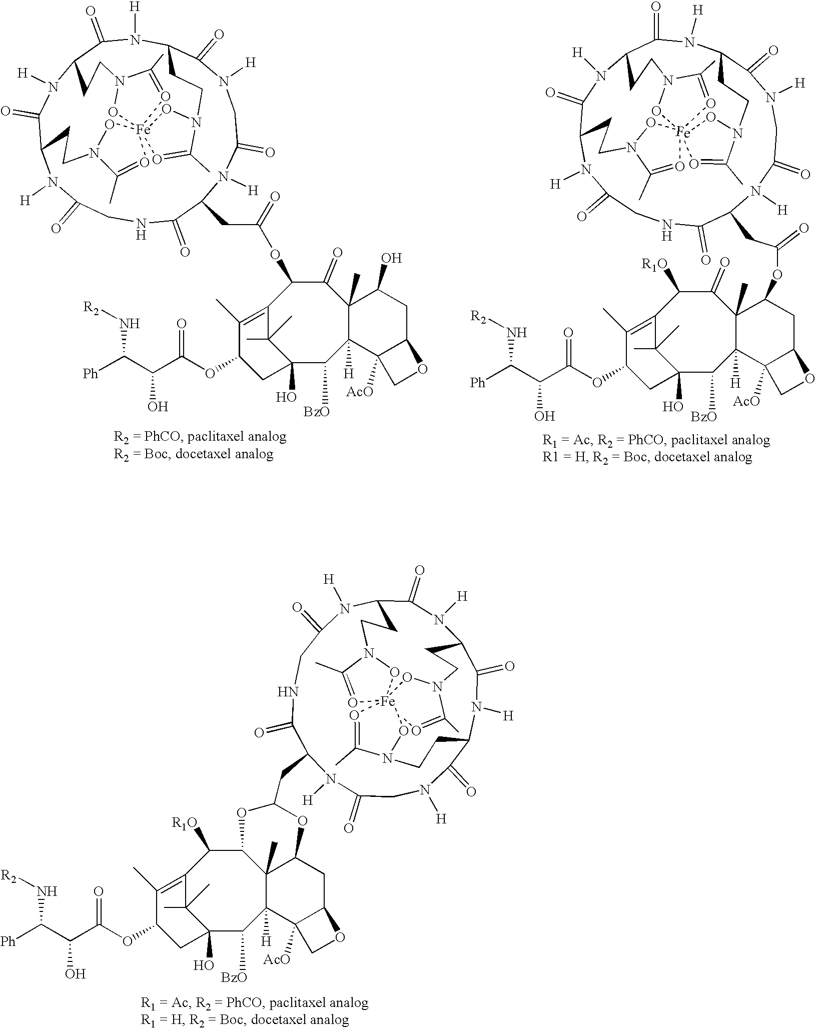

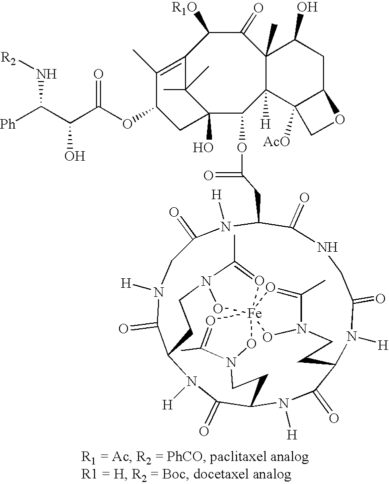

Nitroxides

Another class of magnetic carriers is the nitroxyl radicals (also known as nitroxides). Nitroxyl radicals a “persistent” radials that are unusually stable. A wide variety of nitroxyls are commercially available. Their paramagnetic nature allows them to be used as spin labels and spin probes.

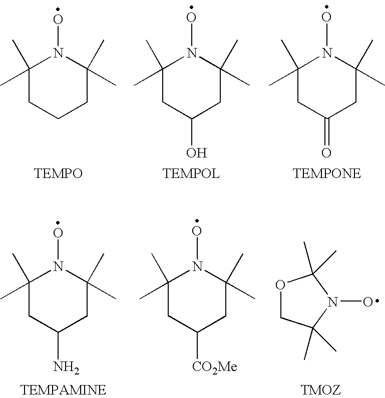

In addition to the commercially available nitroxyls, other paramagnetic radical labels have been generated by acid catalyzed condensation with 2-Amino-2-methyl-1-propanol followed by oxidation of the amine.

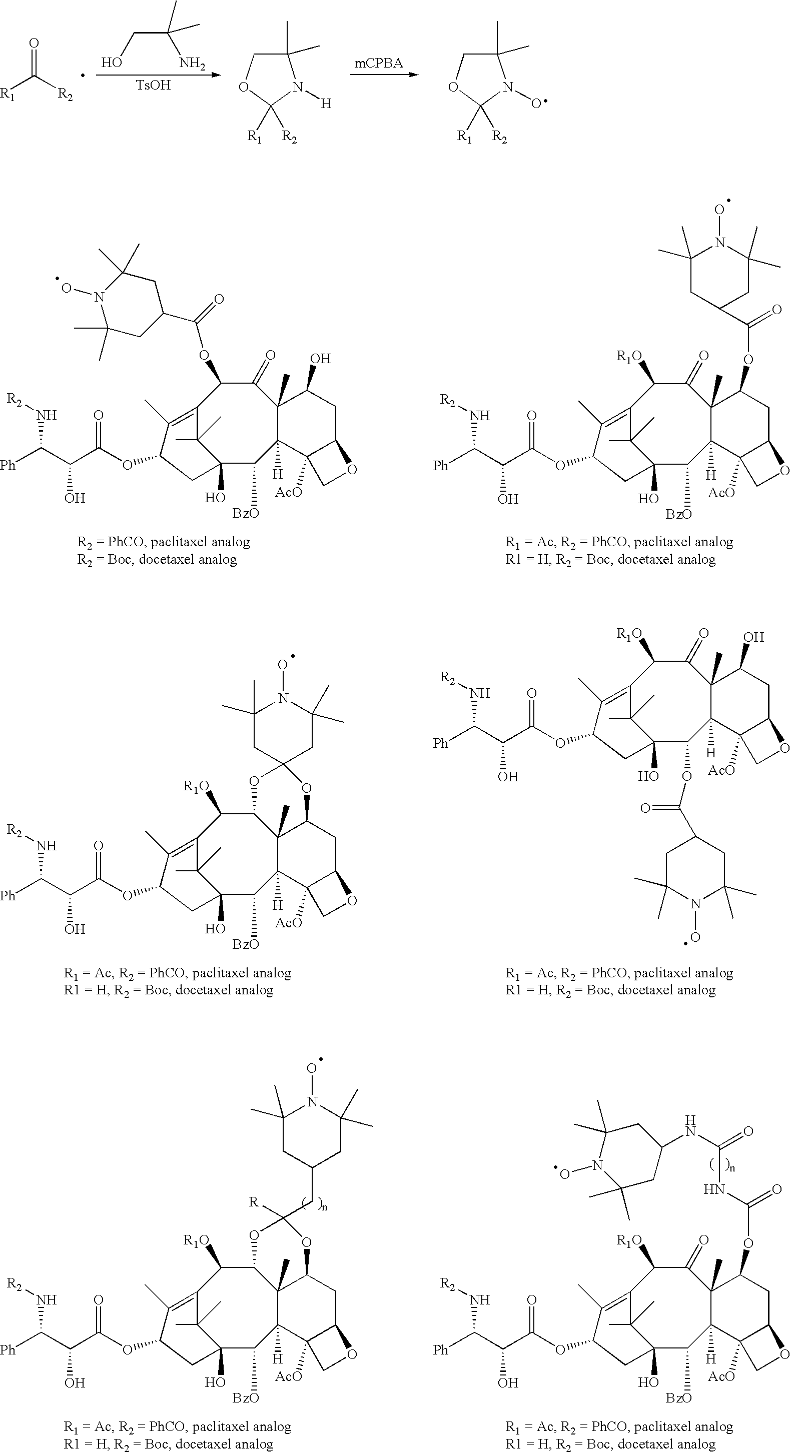

One of ordinary skill in the art could use the teachings of this specification to generate a wide variety of suitable carrier-drug complexes. The following table represents but a small sampling of such compounds.

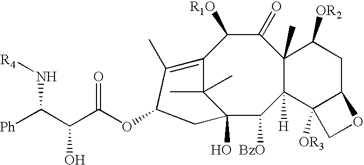

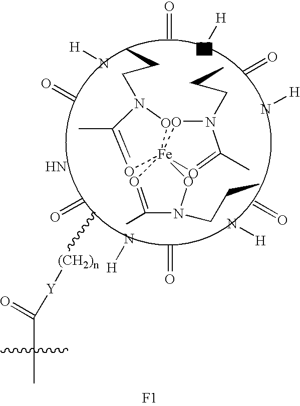

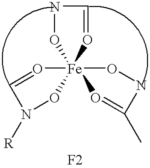

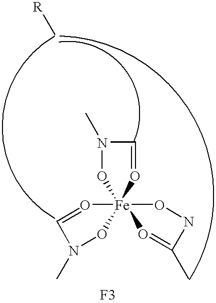

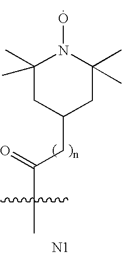

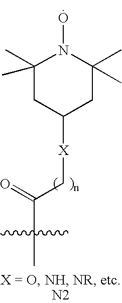

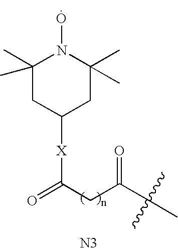

| R1 | R2 | R3 | R4 |
|---|---|---|---|
| F1, Y = CH₂, | H | Ac | COPh |
| n = 0 to 20 | | | |
| Ac | F1, Y = CH₂, | Ac | COPh |
| | n = 0 to 20 | | |
| Ac | H | F1, Y = CH₂, | COPh |
| | | n = 0 to 20 | |
| Ac | H | Ac | F1, Y = CH₂, |
| | | | n = 0 to 20 |
| H | H | Ac | Boc |
| F1, Y = CH₂, | H | Ac | Boc |
| n = 0 to 20 | | | |
| H | F1, Y = CH₂, | Ac | Boc |
| | n = 0 to 20 | | |
| H | H | F1, Y = CH₂, | Boc |
| | | n = 0 to 20 | |
| H | H | Ac | F1, Y = CH₂, |
| | | | n = 0 to 20 |
| F1, Y = NH or | H | Ac | COPh |
| NR, | | | |
| n = 0 to 20 | | | |
| Ac | F1, Y = NH or | Ac | COPh |
| | NR, n = 0 to 20 | | |
| Ac | H | F1, Y = NH or | COPh |
| | | NR, n = 0 to 20 | |
| Ac | H | Ac | F1, Y = NH or |
| | | | NR, n = 0 to 20 |
| H | H | Ac | Boc |
| F1, Y = NH or | H | Ac | Boc |
| NR, | | | |
| n = 0 to 20 | | | |
| H | F1, Y = NH or | Ac | Boc |
| | NR, n = 0 to 20 | | |
| H | H | F1, Y = NH or | Boc |
| | | NR, n = 0 to 20 | |
| H | H | Ac | F1, Y = NH or |
| | | | NR, n = 0 to 20 |
| N1, n = 0 to 20 | H | Ac | COPh |
| Ac | N1, n = 0 to 20 | Ac | COPh |
| Ac | H | N1, n = 0 to 20 | COPh |
| Ac | H | Ac | N1, n = 0 to 20 |
| H | H | Ac | Boc |
| N1, n = 0 to 20 | H | Ac | Boc |
| H | N1, n = 0 to 20 | Ac | Boc |
| H | H | N1, n = 0 to 20 | Boc |
| H | H | Ac | N1, n = 0 to 20 |
| N2, n = 0 to | H | Ac | COPh |
| 20, X = O or | | | |
| NH | | | |
| Ac | N2, n = 0 to | Ac | COPh |
| | 20, X = O or | | |
| | NH | | |
| Ac | H | N2, n = 0 to | COPh |
| | 20, X = O or | | |
| | | NH | |
| Ac | H | Ac | N2, n = 0 to |
| | | | 20, X = O or |
| | | | NH |
| H | H | Ac | Boc |
| N2, n = 0 to | H | Ac | Boc |
| 20, X = O or | | | |
| NH | | | |
| H | N2, n = 0 to | Ac | Boc |
| | 20, X = O or | | |
| | NH | | |
| H | H | N2, n = 0 to | Boc |
| | | 20, X = O or | |
| | | NH | |
| H | H | Ac | N2, n = 0 to |
| 20, X = O or | | | |
| | | | NH |
| N3, n = 0 to | | | |
| 20, X = O or | | | |
| NH | | | |
| Ac | N3, n = 0 to | Ac | COPh |
| | 20, X = O or | | |
| | NH | | |
| Ac | H | N3, n = 0 to | COPh |
| | | 20, X = O or | |
| | | NH | |
| Ac | H | Ac | N3, n = 0 to |
| | | | 20, X = O or |
| | | | NH |
| H | H | Ac | Boc |
| N3, n = 0 to | H | Ac | Boc |
| 20, X = O or | | | |
| NH | | | |
| H | N3, n = 0 to | Ac | Boc |
| | 20, X = O or | | |
| | NH | | |
| H | H | N3, n = O to | Boc |
| | | 20, X = O or | |
| | | NH | |
| H | H | Ac | N3, n = 0 to |
| | | | 20, X = O or |
| | | | NH |
| F2 or F3 | H | Ac | COPh |
| Ac | F2 or F3 | Ac | COPh |
| Ac | H | F2 or F3 | COPh |
| Ac | H | Ac | F2 or F3 |
| F2 or F3 | H | Ac | Boc |
| H | F2 or F3 | Ac | Boc |
| H | H | F2 or F3 | Boc |
| H | H | Ac | F2 or F3 |

While the present invention has been described by reference to the above-mentioned embodiments, certain modifications and variations will be evident to those of ordinary skill in the art. These are intended to be comprehended within the scope of the claimed invention.

1. A composition comprised of nanomagnetic particles, wherein said nanomagnetic particles have an average particle size of less than about 100 nanometers, a saturation magnetization of from about 2 to about 2,000 electromagnetic units per cubic centimeter, a phase transition temperature of from about 40 to about 200 degrees Celsius, and a squareness of from about 0.05 to about 1.0; wherein the average coherence length between adjacent nanomagnetic particles is less than about 100 nanometers; and wherein said nanomagnetic particles are at least triatomic, being comprised of a first distinct atom, a second distinct atom, and a third distinct atom.

2. The composition as recited in claim 1, wherein said first distinct atom is an atom selected from the group consisting of atoms of actinium, americium, berkelium, californium, cerium, chromium, cobalt, curium, dysprosium, einsteinium, erbium, europium, fermium, gadolinium, holmium, iron, lanthanum, lawrencium, lutetium, manganese, mendelevium, nickel, neodymium, neptunium, nobelium, plutonium, praseodymium, promethium, protactinium, samarium, terbium, thorium, thulium, uranium, and ytterbium.

3. The composition as recited in claim 2, wherein said first distinct atom is an atom selected from the group consisting of iron, nickel, and cobalt.

4. The composition as recited in claim 2, wherein said nanomagnetic particles have a squareness of from about 0.1 to about 0.9.

5. The composition as recited in claim 2, wherein said nanomagnetic particles have a squareness of from about 0.2 to about 0.8.

6. The composition as recited in claim 2, wherein said nanomagnetic particles have a squarness of at least about 0.8.

7. The composition as recited in claim 2, wherein said second distinct atom has a relative magnetic permeability of about 1.0.

8. The composition as recited in claim 2, wherein said second distinct atom is an atom selected from the group consisting of aluminum, antimony, barium, beryllium, boron, bismuth, calcium, gallium, germanium, gold, indium, lead, magnesium, palladium, platinum, silicon, silver, strontium, tantalum, tin, titanium, tungsten, yttrium, zirconium, magnesium, and zinc.

9. The composition as recited in claim 8, wherein said third distinct atom is an atom selected from the group consisting of argon, bromine, carbon, chlorine, fluorine, helium, helium, hydrogen, iodine, krypton, oxygen, neon, nitrogen, phosphorus, sulfur, and xenon.

10. The composition as recited in claim 9, wherein said third distinct atom is an atom selected from the group consisting of oxygen and nitrogen.

11. The composition as recited in claim 10, wherein said third distinct atom is nitrogen.

12. The composition as recited in claim 8, wherein said nanomagnetic particles are represented by the formula AₓB_{y}C_{z}, wherein A is said first distinct atom, B is said second distinct atom, C is said third distinct atom, and x+y+z is equal to 1.

13. The composition as recited in claim 10, wherein said third distinct atom is an atom selected from the group consisting of oxygen and nitrogen.

14. The composition as recited in claim 13, wherein said third distinct atom is nitrogen.

15. The composition as recited in claim 14, wherein said first distinct atom is iron.

16. The composition as recited in claim 15, wherein said second distinct atom is aluminum.

17. The composition as recited in claim 10, wherein at least about 10 weight percent of said composition is comprised of said nanomagnetic particles.

18. The composition as recited in claim 10, wherein at least about 40 weight percent of said composition is comprised of said nanomagnetic particles.

19. The composition as recited in claim 10, wherein at least about 50 weight percent of said composition is comprised of said nanomagnetic particles.

20. The composition as recited in claim 10, wherein said composition is comprised of a ceramic binder.

21. The composition as recited in claim 20, wherein said ceramic binder is selected from the group consisting of a clay binder, an organic colloidal particle binder, and a molecular organic binder.

22. The composition as recited in claim 20, wherein said binder is a synthetic polymeric binder.

23. The composition as recited in claim 10, wherein said composition is a fluid composition.

24. The composition as recited in claim 10, wherein said composition is disposed within a fiber.

25. The composition as recited in claim 24, wherein said fiber is disposed within a fabric.

26. The composition as recited in claim 12, wherein the ratio of x/y is at least 0.1.

27. The composition as recited in claim 26, wherein the ratio of xly is at least 0.2.

28. The composition as recited in claim 27, wherein the ratio of z/x is from about 0.001 to about 0.5.

29. The composition as recited in claim 10, wherein said composition is comprised of at least 0.05 weight percent of said nanomagnetic particles.

30. The composition as recited in claim 10, wherein said composition is comprised of at least 5 weight percent of said nanomagnetic particles.

31. The composition as recited in claim 10, wherein said composition consists essentially of said nanomagnetic particles.

32. The composition as recited in claim 10, wherein said nanomagnetic particles have an average particle size of less than about 20 nanometers.

33. The composition as recited in claim 10, wherein said nanomagentic particles have an average particle size of less than about 15 nanometers.

34. The composition as recited in claim 10, wherein said nanomagentic particles have an average particle size of less than about 10 nanometers.

35. The composition as recited in claim 10, wherein said nanomagentic particles have an average particle size of less than about 3 nanometers.

36. The composition as recited in claim 10, wherein said phase transition temperature is less than about 50 degrees Celsius.

37. The composition as recited in claim 10, wherein said phase transition temperature is less than about 46 degrees Celsius.

38. The composition as recited in claim 10, wherein said phase transition temperature is less than about 45 degrees Celsius.

39. The composition as recited in claim 10, wherein said nanomagnetic particles have a saturation magnetization of at least 100 electromagnetic units per cubic centimeter.

40. The composition as recited in claim 10, wherein said nanomagnetic particles have a saturation magnetization of at least 200 electromagnetic units per cubic centimeter.

41. The composition as recited in claim 10, wherein said nanomagentic particles have a saturation magnetization of at least 1,000 electromagnetic units per cubic centimeter.

42. The composition as recited in claim 10, wherein said composition is comprised of nanomagnetic material with a saturation magnetization of from about 1 to about 36,000 Gauss, a coercive force of from about 0.01 to about 5,000 Oersteds, and a relative magnetic permeability of from about 1 to about 500,000.

43. The composition as recited in claim 42, wherein said nanomagnetic material has a saturation magnetization of from about 200 to about 26,000 Gauss.

44. The composition as recited in claim 42, wherein said nanomagnetic material has a coercive force of from about 0.01 to about 3,000 Oerstends.

45. The composition as recited in claim 42, wherein said nanomagnetic material has a coercive force of from about 0.1 to about 10 Oersteds.

46. The composition as recited in claim 42, wherein said nanomagnetic material has a relative magnetic permeability of from about 1.5 to about 260,000.

47. The composition as recited in claim 42, wherein said nanomagnetic material has a relative magnetic permeability of from about 1.5 to about 2,000.

48. The composition as recited in claim 42, wherein said nanomagnetic material has a mass density of at least 0.001 grams per cubic centimeter

49. The composition as recited in claim 42, wherein said nanomagentic material has a mass density of at least about 1 gram per cubic centimeter.

50. The composition as recited in claim 42, wherein said nanomagnetic material has a mass density of at least about 3 grams per cubic centimeter.

51. The composition as recited in claim 42, wherein said nanomagnetic material has a mass density of at least about 4 grams per cubic centimeter.

52. The composition as recited in claim 42, wherein said nanomagnetic material has a saturation magnetization of from about 500 to about 10,000 Gauss.

53. The composition as recited in claim 10, wherein said composition is comprised of an insulating matrix within which said nanomagnetic particles are disposed.

54. The composition as recited in claim 10, wherein said composition is comprised of cerium oxide.

55. The composition as recited in claim 10, wherein said composition is comprised of calcium oxide.

56. The composition as recited in claim 10, wherein said composition is comprised of silica.

57. The composition as recited in claim 10, wherein said composition is comprised of alumina.

58. The composition as recited in claim 10, wherein said composition is bonded to a therapeutic agent.

59. The composition as recited in claim 58, wherein said therapeutic agent is an anti-microtubule agent.

60. The composition as recited in claim 59, wherein said anti-microtubule agent is a taxane.

61. The composition as recited in claim 59, wherein said anti-microtubule agent is paclitaxel.

62. The composition as recited in claim 58, wherein said therapeutic agent is disposed on or in a polymeric carrier.

63. The composition as recited in claim 62, wherein said polymeric carrier is biodegradable.

64. The composition as recited in claim 63, wherein said polymeric carrier is a temperature sensitive polymeric carrier.

65. The composition as recited in claim 63, wherein said polymeric carrier is comprised of a thermogelling polymer.

66. The composition as recited in claim 10, wherein said composition is bound to an affinity recognition molecule.

67. The composition as recited in claim 66, wherein affinity recognition molecule is selected from the group consisting of antibodies, enzymes, specific binding proteins, nucleic acid molecules, receptors, and mixtures thereof.

68. The composition as recited in claim 10, wherein said composition is disposed within a polymeric carrier.

69. The composition as recited in claim 68, wherein said polymeric carrier is comprised of poly(caprolactone).

70. The composition as recited in claim 68, wherein said polymeric carrier is comprised of polylactic acid.

71. The composition as recited in claim 68, wherein said polymeric carrier is comprised of poly (ethylene-vinyl acetate).

72. The composition as recited in claim 68, wherein said polymeric carrier is comprised of an anti-angiogenic factor that inhibits vascular growth.

73. The composition as recited in claim 68, wherein said polymeric carrier is comprised of a polyvinyl aromatic polymer.

74. The composition as recited in claim 73, wherein said polyvinyl aromatic polymer is polyacrylic acid.

75. The composition as recited in claim 68, wherein said polymeric carrier is comprised of a bioerodible polymer.

76. The composition as recited in claim 10, wherein said composition is comprised of dextran.

77. The composition as recited in claim 10, wherein said composition is comprised of albumen.

78. The composition as recited in claim 10, wherein said composition is comprised of lipid material.

79. The composition as recited in claim 10, wherein said composition is comprised of proteinaceous material.

80. The composition as recited in claim 10, wherein said composition is comprised of a polysaccharide.

81. The composition as recited in claim 10, wherein said composition is comprised of a water-insoluble organic liquid.

82. The composition as recited in claim 10, wherein said composition is comprised of a water-soluble anti-cancer agent.

83. The composition as recited in claim 10, wherein said composition is comprised of a hydrophilic, crystalline carbohydrate.

84. The composition as recited in claim 10, wherein said composition is comprised of nuclide material.

85. The composition as recited in claim 10, wherein said composition is comprised of organic resin binder.

86. The composition as recited in claim 10, wherein said composition is comprised of sublimable dyestuff.

87. The composition as recited in claim 10, wherein said composition is disposed within a tape.

88. The composition as recited in claim 10, wherein said composition is comprised of a polymerizable ink.

89. The composition as recited in claim 10, wherein said composition is comprised of chromium oxide.

90. The composition as recited in claim 10, wherein said composition is comprised of a water soluble material.

91. The composition as recited in claim 10, wherein said composition is comprised of a colorant.

92. The composition as recited in claim 10, wherein said composition is comprised of liquid crystal material.

93. The composition as recited in claim 10, wherein said composition is comprised of nitrile rubber.

94. The composition as recited in claim 10, wherein said composition is comprised of a glycidyl compound.

95. The composition as recited in claim 10, wherein said composition is comprised of a polyurethane.

96. The composition as recited in claim 10, wherein said composition is comprised of an electronic conductive polymer.

97. The composition as recited in claim 10, wherein said composition is comprised of an oligonucleotide.

98. The composition as recited in claim 10, wherein said composition is comprised of a ferrofluid.
